(19) United States
(12) Patent Application Publication
(10) Pub. No.: US 2004/0171047 A1
(43) Pub. Date: Sep. 2, 2004

(54) Target-dependent transcription

(76) Inventors: Gary A. Dahl, Madison, WI (US); Jerome J. Jendrisak, Madison, WI (US); Judith E. Meis, Fitchburg, WI (US); Ramesh Vaidyanathan, Madison, WI (US)

Correspondence Address: QUARLES & BRADY LLP, FIRSTAR PLAZA, ONE SOUTH PINCKNEY STREET, P.O BOX 2113 SUITE 600, MADISON, WI, 53701-2113, US

(21) Appl. No.: 10/744,815

(22) Filed: Dec. 23, 2003

Related U.S. Application Data
(63) Continuation-in-part of application No. 10/153,219, filed on May 22, 2002.
(60) Provisional application No. 60/436,062, filed on Dec. 23, 2002.

(51) Int. Cl. C12Q 1/68; C12P 19/34

(52) U.S. Cl. 435/6; 435/91.2

(57) ABSTRACT

The present invention comprises novel methods, compositions and kits comprising monopartite or bipartite target probes and an RNA polymerase to detect and quantify analytes comprising one or multiple target nucleic acid sequences, including target sequences that differ by as little as one nucleotide, or to detect and quantify non-nucleic acid analytes by detecting a target sequence tag that is joined to an analyte-binding substance. The method, called “target-dependent transcription,” consists of an annealing process, a DNA ligation process, a transcription process, and a detection process. The invention also comprises novel methods, compositions and kits for amplifying RNA, including strand-displacement reverse transcription and rolling circle reverse transcription.

### CROSS-REFERENCE TO RELATED APPLICATION

This application claims priority to U.S. Provisional Patent Application Serial No. 60/436,062 filed Dec. 23, 2002. The entire disclosure of all priority applications is specifically incorporated herein by reference.

### STATEMENT REGARDING FEDERALLY SPONSORED RESEARCH OR DEVELOPMENT

Not Applicable.

### BACKGROUND OF THE INVENTION

I. Field of the Invention

The present invention relates to novel methods, compositions and kits for amplifying, detecting and quantifying one or multiple target nucleic acid sequences in a sample, including target sequences that differ by as little as one nucleotide. The invention has broad applicability for research, environmental and genetic screening, and diagnostic applications, such as for detecting and quantifying sequences that indicate the presence of a pathogen, the presence of a gene or an allele, or the presence of a single nucleotide polymorphism (SNP) or other type of gene mutation or variant. The invention also related to novel methods, compositions and kits for detecting and quantifying a broad range of non-nucleic acid analytes by detecting a target sequence that is joined to an analyte-binding substance.

II. Description of Related Art

Transcription of DNA into mRNA is regulated by the promoter region of the DNA. The promoter region contains a sequence of bases that signals RNA polymerase to associate with the DNA, and to initiate the transcription of mRNA using one of the DNA strands as a template to make a corresponding complementary strand of RNA. RNA polymerases from different species typically recognize promoter regions comprised of different sequences. In order to obtain a transcription product by in vitro or in vivo transcription, the promoter driving transcription of the gene or DNA sequence must be a cognate promoter for the RNA polymerase, meaning that it is recognized by the RNA polymerase.

It is known that a transcription product is obtained by in vitro transcription of short DNA sequences that are joined to a functional double-stranded T7 RNAP promoter (Milligan, J F et al., Nucleic Acids Res., 15: 8783-8798, 1987). A method for synthesizing RNA using a single-stranded DNA template that is non-covalently immobilized on a solid support by annealing to a complementary promoter sequence is disclosed in U.S. Pat. No. 5,700,667.

There are a number of methods in the art for detecting nucleic acid sequences, including point mutations. The presence of a nucleic acid sequence can indicate, for example, the presence of a pathogen, or the presence of particular genes or mutations in particular genes that correlate with or that are indicative of the presence or status of a disease state, such as, but not limited to, a cancer.

Examples of methods that involve in vitro transcription for making probes are described in: Murakawa et al., DNA 7:287-295, 1988; Phillips and Eberwine, Methods in Enzymol. Suppl. 10:283-288, 1996; Ginsberg et al., Ann. Neurol. 45:174-181, 1999; Ginsberg et al., Ann. Neurol. 48:77-87, 2000; VanGelder et al., Proc. Natl. Acad. Sci. USA 87:1663-1667, 1990; Eberwine et al., Proc. Natl. Acad. Sci. USA 89:3010-3014, 1992; U.S. Pat. Nos. 5,021,335; 5,168,038; 5,545,522; 5,514,545; 5,716,785; 5,891,636; 5,958,688; 6,291,170; and PCT Patent Applications WO 00/75356 and WO 02/065093.

Still other methods use in vitro transcription as part of a process for amplifying and detecting one or more target nucleic acid sequences in order to detect the presence of a pathogen, such as a viral or microbial pathogen, that is a causative agent for a disease or to detect a gene sequence that is related to a disease or the status of a disease for medical purposes. Examples of methods that use in vitro transcription for this purpose include U.S. Pat. Nos. 5,130,238; 5,194,370; 5,399,491; 5,409,818; 5,437,990; 5,466,586; 5,554,517; 5,665,545; 6,063,603; 6,090,591; 6,100,024; 6,410,276; Kwoh et al., Proc. Natl. Acad. Sci. USA 86:1173, 1989; Fahy et al, In: PCR Methods and Applications, pp. 25-33, 1991; PCT Patent Application Nos. WO 89/06700 and WO 91/18155; and European Patent Application Nos. 0427073 A2 and 0427074 A2.

Still other methods detect sequences or mutations using methods that involve ligation of adjacently hybridizing oligonucleotide probes or ligation of non-adjacently hybridizing probes following a process such as primer extension. Ligation detection methods include those disclosed in European Patent Application Publication Nos. 0246864 A2 and 0246864 B1 of Carr; U.S. Pat. Nos. 4,883,750; 5,242,794; 5,521,065; 5,962,223; and 6,054,266 of Whiteley, N. M. et al.; U.S. Pat. Nos. 4,988,617 of Landegren and Hood; U.S. Pat. No. 5,871,921 of Landegren and Kwiatkowski; U.S. Pat. No. 5,866,337 of Schon; European Patent Application Publication Nos. 0320308 A2 and 0320308 B1 of Backman and Wang; PCT Publication No. WO 89/09835 of Orgel and Watt and European Patent Publication No. 0336731 B1 of Bruce Wallace; U.S. Pat. No. 5,686,272 of Marshall et al.; U.S. Pat. No. 5,869,252 of Bouma et al.; U.S. Pat. Nos. 5,494,810; 5,830,711; 6,054,564; 6,027,889; 6,268,148; and 6,312,892 of Barany et al.; U.S. Pat. Nos. 5,912,148 and 6,130,073 of F. Eggerding; U.S. Pat. No. 6,245,505 B1 of Todd and Fuery; European Patent Application Publication No. 0357336 A2 of Ullman et al.; U.S. Pat. No. 5,427,930 of Birkenmeyer et al. and U.S. Pat. No. 5,792,607 and European Patent Publication Nos. 0439182 A2 and EP 0439182 B1 of Backman et al.; U.S. Pat. No. 5,679,524; and 5,952,174 of Nikifoorov et al.; U.S. Pat. No. 6,025,139 of Yager and Dunn; and U.S. Pat. No. 6,355,431 B1 of Chee and Gunderson.

In addition, U.S. Pat. No. 6,153,384 of Lynch et al. discloses an assay to identify ligase activity modulators by ligation of a labeled nucleic acid to an immobilized capture nucleic acid in the presence of a potential ligase activity modulator and U.S. Pat. No. 5,976,806 of Mahajan et al. discloses a quantitative and functional DNA ligase assay that uses a linearized plasmid containing a reporter gene, wherein ligase activity is followed by the extent of coupled transcription-translation of the reporter gene.

U.S. Pat. No. 5,807,674 of Sanjay Tyagi discloses detection of RNA target sequences by ligation of the RNA binary probes, wherein a substrate for Q-beta replicase is generated.

In PCT Patent Application No. WO 92/01813, Ruth and Driver disclosed a process for synthesizing circular single-stranded nucleic acids by hybridizing a linear polynucleotide to a complementary oligonucleotide and then ligating the linear polynucleotide. They further disclosed a process for generating multiple linear complements of the circular single-stranded nucleic acid template by extending a primer more than once around the circular template using a DNA polymerase.

Japanese Patent Nos. JP4304900 and JP4262799 of Aono Toshiya et al. disclose detection of a target sequence by ligation of a linear single-stranded probe having target-complementary 3′- and 5′-end sequences which are adjacent when the linear probe is annealed to a target sequence in the sample, followed by either rolling circle replication or in vitro transcription of the circular single-stranded template. The inventors disclose that in vitro transcription is performed by first annealing to the circular single-stranded template a complementary nucleotide primer having an anti-promoter sequence in order to form a double-stranded promoter, and then transcribing the circular single-stranded template having the annealed anti-promoter primer with an RNA polymerase that has helicase-like activity, such as T7, T3 or SP6 RNA polymerase.

In U.S. Pat. Nos. 6,344,329; 6,210,884; 6,183,960; 5,854,033; 6,329,150; 6,143,495; 6,316,229; and 6,287,824, Paul M. Lizardi also used rolling circle replication to amplify and detect nucleic acid sequences. Lizardi describes use of RNA polymerase protopromoters in circular probe so that tandem-sequence single-stranded protopromoter-containing DNA products resulting from rolling circle replication can be transcribed by a cognate T7-type RNA polymerase following conversion of said DNA products to a form containing double-stranded promoters.

In a series of articles and patents, Eric Kool and co-workers disclosed synthesis of DNA or RNA multimers, meaning multiple copies of an oligomer or oligonucleotide joined end to end (i.e., in tandem) by rolling circle replication or rolling circle transcription, respectively, of a circular DNA template molecule. Rolling circle replication uses a primer and a strand-displacing DNA polymerase, such as phi29 DNA polymerase. With respect to rolling circle transcription, it was shown that circular single-stranded DNA (ssDNA) molecules can be efficiently transcribed by phage and bacterial RNA polymerases (Prakash, G. and Kool, E., J. Am. Chem. Soc. 114:3523-3527, 1992; Daubendiek, S. L. et al., J. Am. Chem. Soc. 117: 7818-7819, 1995; Liu, D. et al., J. Am. Chem. Soc. 118: 1587-1594, 1996; Daubendiek, S. L. and Kool, E. T., Nature Biotechnol., 15: 273-277, 1997; Diegelman, A. M. and Kool, E. T., Nucleic Acids Res., 26: 3235-3241, 1998; Diegelman, A. M. and Kool, E. T., Chem. Biol., 6: 569-576, 1999; Diegelman, A. M. et al., BioTechniques 25: 754-758, 1998; Frieden, M. et al., Angew. Chem. Int. Ed. Engl. 38: 3654-3657, 1999; Kool, E. T., Acc. Chem. Res., 31: 502-510, 1998; U.S. Pat. Nos. 5,426,180; 5,674,683; 5,714,320; 5,683,874; 5,872,105; 6,077,668; 6,096,880; and 6,368,802). Rolling circle transcription of these circular ssDNAs occurs in the absence of primers, in the absence of a canonical promoter sequence, and in the absence of any duplex DNA structure, and results in synthesis of linear multimeric complementary copies of the circle sequence up to thousands of nucleotides in length. Transcription of the linear precursor of the circular ssDNA template yielded only a small amount of RNA transcript product that was shorter than the template.

Fire and Xu (U.S. Pat. No. 5,648,245; Fire, A. and Xu, S-Q, Proc. Natl. Acad. Sci. USA, 92: 4641-4645, 1995) also disclose methods for using rolling circle replication of small DNA circles to construct oligomer concatamers.

Other researchers, including, but not limited to, Mahtani (U.S. Pat. No. 6,221,603), Rothberg et al. (U.S. Pat. No. 6,274,320), Dean et al. (Genome Res., 11: 1095-1099, 2001), Lasken et al. (U.S. Pat. No. 6,323,009), and Nilsson et al. (Nucleic Acids Res., 30 (14): e66, 2002) disclose other methods and applications of rolling circle amplification. Pickering et al. (Nucleic Acids Res., 30 (12): e60, 2002) discloses a ligation and rolling circle amplification method for homogeneous end-point detection of single nucleotide polymorphisms (SNPs).

Although a number of nucleic acid amplification methods have been described in the art, there is a continuing need for methods and assays for detecting nucleic acids that are specific and accurate, yet are easier and faster than current methods. The present invention provides novel assays, methods, compositions and kits that are simple in format and very rapid to perform, but that can be used to detect and quantify any of a broad range of analytes with a high degree of specificity and sensitivity, including both nucleic acid analytes and non-nucleic acid analytes. With respect to analytes comprising a target nucleic acid, the invention provides assays, methods and kits that can detect and distinguish between target sequences, including sequences that differ even by only a single nucleotide, such as for analysis of single nucleotide polymorphisms.

### BRIEF SUMMARY OF THE INVENTION

One embodiment of the invention is a method for detecting a target nucleic acid sequence, the method comprising: (a) providing one or more target probes comprising linear single-stranded DNA, the target probes comprising at least two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, and wherein the 3′-end of the first target-complementary sequence is joined to the 5′-end of a sense promoter sequence for an RNA polymerase; (b) contacting the target probes with the target nucleic acid sequence and incubating under hybridization conditions, wherein the target-complementary sequences anneal adjacently on the target nucleic acid sequence to form a complex; (c) contacting the complex with a ligase under ligation conditions to obtain a ligation product comprising the target-complementary sequences of the target probes annealed to the target nucleic acid sequence; (d) contacting the ligation product with an anti-sense promoter oligo and incubating under hybridization conditions, wherein the anti-sense promoter oligo anneals to the sense promoter sequence of the ligation product to form a transcription substrate; (e) contacting the transcription substrate with an RNA polymerase that can bind the promoter under transcription conditions to obtain a transcription product; and (f) detecting the transcription product.

Preferably, the ligase has little or no activity in ligating blunt ends and is substantially more active in ligating ends that are adjacent when annealed to two contiguous regions of a target sequence compared to ends that are not annealed to the target sequence. One suitable ligase that can be used is Ampligase® Thermostable DNA Ligase (EPICENTRE Technologies, Madison, Wis.). In preferred embodiments, the RNA polymerase comprises T7 RNAP, T3 RNAP, SP6 RNAP or another T7-like RNA polymerase or a mutant form of one of these T7-like RNA polymerases.

In some embodiments, the anti-sense promoter oligo is attached to a solid support. In other embodiments, the anti-sense promoter oligo comprises a moiety, such as, but not limited to a biotin moiety that permits binding of the anti-sense promoter oligo to a solid support after annealing to the ligation product to obtain a transcription substrate. In some embodiments, the target probes comprise monopartite target probes comprising a promoter target probe and a signal target probe and/or optionally, one or more simple target probes. In other embodiments, a bipartite target probe and, optionally, one or more simple target probes is used. In some embodiments, the target sequence comprises a target nucleic acid in a sample, whereas in other embodiments the target sequence comprises a target sequence tag that is joined to an analyte-binding substance that binds an analyte in the sample. In some embodiments in which a bipartite target probe is used, the transcription substrate that is transcribed remains catenated to a target nucleic acid. In other embodiments of methods in which a bipartite target probe is used, the target sequence is preferably less than about 150 to about 200 nucleotides from the 3′-end of the target nucleic acid or target sequence tag. In still other embodiments of methods in which a bipartite target probe is used and in which the target sequence is greater than about 150 to about 200 nucleotides from the 3′-end of the target nucleic acid or target sequence tag comprising the target sequence, one or more additional steps is used in order to release the catenated circular ligation product from the target sequence prior to transcription, as described elsewhere herein.

Another embodiment of the invention comprises a method for obtaining transcription products comprising multiple copies of a target nucleic acid sequence in a sample, said method comprising: (a) providing one or more target probes comprising linear single-stranded DNA, the target probes having at least two different target-complementary sequences that are not joined to each other, wherein the 5′-end of a first target-complementary sequence is complementary to the 5′-end of the target nucleic acid sequence and the 3′-end of a second target-complementary sequence is complementary to the 3′-end of the target nucleic acid sequence, and wherein the target probe that comprises a target-complementary sequence that is complementary to the 5′-end of the target nucleic acid sequence also comprises a sense promoter sequence that is joined to the 3′-end of the target-complementary sequence of said target probe, and wherein any additional target probes, if provided, comprise simple target probes having target-complementary sequences that anneal to the target nucleic acid sequence between the annealing sites of the first target-complementary sequence and the second target-complementary sequence, and wherein every free 5′-end of a target-complementary sequence that anneals to a target nucleic acid sequence has a 5′-phosphate and is adjacent to a 3′-end of another target-complementary sequence that has a 3′-hydroxyl end; (b) contacting the target probes with the target sequence and incubating under hybridization conditions so as to permit the target-complementary sequences of said target probes to anneal adjacently on all portions of the target sequence; (c) contacting said target probes annealed to said target sequence with a ligase under ligation conditions so as to obtain a single-stranded DNA ligation product; (d) contacting the ligation product with an anti-sense promoter oligo and incubating under hybridization conditions so as to obtain a transcription substrate for an RNA polymerase that binds the double-stranded promoter; (e) contacting said transcription substrate with the RNA polymerase that can bind said promoter and initiate transcription therefrom under transcription conditions so as to obtain a transcription product that is complementary to said transcription substrate; and (f) detecting synthesis of said transcription product comprising multiple copies of the target sequence obtained from transcription of said transcription substrate under transcription conditions, wherein synthesis of said transcription product indicates the presence of the target sequence. Preferably, the ligase has little or no activity in ligating blunt ends and is substantially more active in ligating ends that are adjacent when annealed to two contiguous regions of a target sequence compared to ends that are not annealed to the target sequence. One suitable ligase that can be used is Ampligase® Thermostable DNA Ligase (EPICENTRE Technologies, Madison, Wis.). Some preferred RNA polymerases are T7 RNAP, T3 RNAP, SP6 RNAP or another T7-like RNA polymerase or a mutant form of one of these T7-like RNA polymerases.

In some embodiments, the anti-sense promoter oligo is attached to a solid support. In other embodiments, the anti-sense promoter oligo comprises a moiety, such as, but not limited to a biotin moiety that permits binding of the anti-sense promoter oligo to a solid support after annealing to the ligation product to obtain a transcription substrate. In some embodiments, the target probes comprise monopartite target probes comprising a promoter target probe and a signal target probe and/or optionally, one or more simple target probes. In other embodiments, a bipartite target probe and, optionally, one or more simple target probes is used. In some embodiments, the target sequence comprises a target nucleic acid in a sample, whereas in other embodiments the target sequence comprises a target sequence tag that is joined to an analyte-binding substance that binds an analyte in the sample. In some embodiments in which a bipartite target probe is used, the transcription substrate that is transcribed remains catenated to a target nucleic acid. In other embodiments of methods in which a bipartite target probe is used, the target sequence is preferably less than about 150 to about 200 nucleotides from the 3′-end of the target nucleic acid or target sequence tag. In still other embodiments of methods in which a bipartite target probe is used and in which the target sequence is greater than about 150 to about 200 nucleotides from the 3′-end of the target nucleic acid or target sequence tag comprising the target sequence, one or more additional steps is used in order to release the catenated circular ligation product from the target sequence prior to transcription, as described elsewhere herein.

Another embodiment of the present invention comprises a method for obtaining a transcription product complementary to a target nucleic acid sequence (target sequence or target), said method comprising: (a) providing a target sequence amplification probe (TSA probe), wherein said TSA probe comprises a linear single-stranded DNA comprising two end portions that are not joined, which end portions are connected by an intervening sequence, wherein the 5′-end target-complementary sequence is complementary to the 5′-end of the target sequence, and wherein the 3′-end target-complementary sequence is complementary to the 3′-end of the target sequence, and wherein joining of the ends of said TSA probe forms a TSA circle; (b) contacting the TSA probe to the target sequence and incubating under hybridization conditions, wherein the target-complementary sequences anneal adjacently on the target sequence; (c) contacting said TSA probe annealed to said target sequence with a ligase under ligation conditions so as to obtain a TSA circle; (d) providing a primer that is complementary to the intervening sequence of the TSA probe; (e) contacting the TSA circle with the primer that is complementary to the intervening sequence of the TSA probe under hybridization conditions so as to obtain a TSA circle-primer complex; (f) contacting said TSA circle-primer complex with a strand-displacing DNA polymerase under strand-displacing polymerization conditions so as to obtain a rolling circle replication product comprising multiple copies of the target sequence; (g) providing target probes comprising linear single-stranded DNA, the target probes comprising at least two target-complementary sequences that are not joined to each other, wherein the 5′-end of a first target-complementary sequence is complementary to the 5′-end of the target sequence, and wherein the 3′-end of a second target-complementary sequence is complementary to the 3′-end of the target sequence, and wherein the 3′-end of the first target-complementary sequence is joined to the 5′-end of a sense promoter sequence for an RNA polymerase; (h) contacting the target probes with the target sequence and incubating under hybridization conditions, wherein the target-complementary sequences anneal adjacently on the target sequence to form a target probe-target complex; (i) contacting the target probe-target complex with a ligase under ligation conditions to obtain a ligation product comprising the target-complementary sequences of the target probes annealed to the target nucleic acid sequence; (j) contacting the ligation product with an anti-sense promoter oligo and incubating under hybridization conditions, wherein the anti-sense promoter oligo anneals to the sense promoter sequence of the ligation product to form a transcription substrate; (k) contacting the transcription substrate with an RNA polymerase that can bind the promoter and incubating under transcription conditions to obtain a transcription product; and (l) detecting the transcription product, wherein said transcription product indicates the presence of said target sequence. In some embodiments, the target probes comprise a bipartite target probe and optionally, one or more simple target probes. In other embodiments, the target probes comprise monopartite target probes comprising a promoter target probe and a signal target probe and/or one or more simple target probes.

Preferably, only one ligase is used for ligating both the TSA probe and the target probes. Preferably, the ligase has little or no activity in ligating blunt ends and is substantially more active in ligating ends that are adjacent when annealed to two contiguous regions of a target sequence compared to ends that are not annealed to the target sequence. One suitable ligase that can be used is Ampligase® Thermostable DNA Ligase (EPICENTRE Technologies, Madison, Wis.). A preferred strand-displacing DNA polymerase that can be used is IsoTherm™ DNA Polymerase (EPICENTRE Technologies, Madison, Wis.). Another suitable strand-displacing DNA polymerase that can be used is RepliPHI™ phi29 DNA polymerase (EPICENTRE Technologies, Madison, Wis.). Some preferred RNA polymerases are T7 RNAP, T3 RNAP, SP6 RNAP or another T7-like RNA polymerase or a mutant form of one of these T7-like RNA polymerases. Preferably, AmpliScribe T7-Flash™ Transcription Kit is used for in vitro transcription of the transcription substrate (EPICENTRE Technologies, Madison, Wis.).

In some embodiments, the anti-sense promoter oligo is attached to a solid support. In other embodiments, the anti-sense promoter oligo comprises a moiety, such as, but not limited to a biotin moiety that permits binding of the anti-sense promoter oligo to a solid support after annealing to the ligation product to obtain a transcription substrate. In some embodiments, the target sequence comprises a target nucleic acid in a sample, whereas in other embodiments the target sequence comprises a target sequence tag that is joined to an analyte-binding substance that binds an analyte in the sample. In some embodiments in which a TSA probe or a bipartite target probe is used, the TSA circle or circular transcription substrate, respectively, remains catenated to a target nucleic acid. In other embodiments of methods in which a TSA probe or a bipartite target probe is used, the target sequence is preferably less than about 150 to about 200 nucleotides from the 3′-end of the target nucleic acid or target sequence tag. In still other embodiments of methods in which the target sequence is greater than about 150 to about 200 nucleotides from the 3′-end of the target nucleic acid or target sequence tag, then one or more additional steps is used in order to release the catenated TSA circles from the target sequence prior to rolling circle replication, as described elsewhere herein. Similarly, in some embodiments, one or more additional steps is used in order to release the catenated circular ssDNA ligation products that result from ligation of bipartite target probes that are annealed to target sequences in the rolling circle replication product more than about 150 nucleotides to about 200 nucleotides from the 3′-end of to the rolling circle replication product.

Yet another embodiment is a method for detecting a target sequence, said method comprising: (a) providing a first bipartite target probe comprising linear single-stranded DNA having two target-complementary sequences that are not joined to each other and that are contiguous when annealed to the target sequence, wherein the 5′-end of the first target-complementary sequence is complementary to the 5′-end of the target nucleic acid sequence, and wherein the 3′-end of a second target-complementary sequence is complementary to the 3′-end of the target nucleic acid sequence, and wherein the 3′-end of the first target-complementary sequence is joined to the 5′-end of a sense promoter sequence for an RNA polymerase; (b) providing a second bipartite target probe comprising linear single-stranded DNA having two end sequences that are not joined to each other and that, when joined, are identical to the target sequence, wherein the 5′-end of the first end sequence is complementary to the target-complementary sequence of the 3′-end of the first bipartite target probe and the 3′-end of the second end sequence is complementary to the target-complementary sequence of the 5′-end of the first bipartite target probe; and wherein the 3′-end of the first end sequence is joined to the 5′-end of a sense promoter sequence for an RNA polymerase; (c) annealing said first bipartite target probe to said target sequence under hybridization conditions; and (d) ligating said first bipartite target probe annealed to said target sequence with a ligase under ligation conditions so as to obtain a first circular ssDNA ligation product; (e) contacting the first circular ligation product with an anti-sense promoter oligo and incubating under hybridization conditions, wherein the anti-sense promoter oligo anneals to the sense promoter sequence of the first circular ligation product to form a first circular transcription substrate; (f) contacting said first circular transcription substrate with an RNA polymerase under transcription conditions so as to synthesize RNA that is complementary to said first circular transcription substrate; (g) annealing to said RNA that is complementary to said first circular transcription substrate a primer, wherein said primer is complementary to said RNA; (h) contacting said RNA to which said primer is annealed with a reverse transcriptase under reverse transcription conditions so as to obtain a first first-strand cDNA; (i) annealing to said first first-strand cDNA said second bipartite target probe under hybridization conditions; (j) contacting said first first-strand cDNA to which said second bipartite target probe is annealed with a a ligase under ligation conditions so as to obtain a second circular ssDNA ligation product; (k) contacting said second circular ligation product with an anti-sense promoter oligo and incubating under hybridization conditions, wherein the anti-sense promoter oligo anneals to the sense promoter sequence of the second circular ligation product to form a second circular transcription substrate; (l) obtaining said second circular transcription substrate; (m) contacting said second circular transcription substrate with an RNA polymerase under transcription conditions so as to synthesize RNA that is complementary to said second circular transcription substrate; (n) annealing to said RNA that is complementary to said second circular transcription substrate a primer, wherein said primer is complementary to said RNA; (o) contacting said RNA to which said primer is annealed with a reverse transcriptase under reverse transcription conditions so as to obtain a second first-strand cDNA; (p) obtaining said second first-strand cDNA; (q) annealing to said second first-strand cDNA said first bipartite target probe under annealing conditions; (r) contacting said second first-strand cDNA to which said first bipartite target probe is annealed with a a ligase under ligation conditions so as to obtain a third circular ssDNA ligation product that is identical to said first circular ligation product; (s) contacting the third circular ligation product with an anti-sense promoter oligo and incubating under hybridization conditions, wherein the anti-sense promoter oligo anneals to the sense promoter sequence of the third circular ligation product to form a third circular transcription substrate; (t) obtaining said third circular transcription substrate that is identical to said first circular transcription substrate; (u) contacting said first and second first-strand cDNA products with an anti-sense promoter oligo and incubating under hybridization conditions, wherein the anti-sense promoter oligo anneals to the sense promoter sequence of the first and second cDNA products to form first and second linear transcription substrates; (v) contacting the first and second linear transcription substrates with an RNA polymerase under transcription conditions so as to synthesize RNA that is complementary to said first and second linear transcription substrates; (w) repeating steps a through w; and (x) detecting the synthesis of RNA resulting from transcription of said first, second and third circular transcription substrates and from said first and second linear transcription substrates, wherein said synthesis of said RNA indicates the presence of said target sequence. Preferably, in this embodiment the target sequence is less than about 150 to about 200 nucleotides from the 3′-end of the target nucleic acid or target sequence tag. However, in other embodiments, if the target sequence is greater than about 150 to about 200 nucleotides from the 3′-end of the target nucleic acid or target sequence tag, one or more additional steps is used in order to release the catenated circular ligation products from the target sequence when the target probe anneals to a sequence in a linear DNA molecule that is greater than about 150 to about 200 nucleotides from the 3′-end of the linear DNA molecule. In still other embodiments, circular transcription substrates that are transcribed remain catenated to a target nucleic acid.

Preferably, only one ligase is used for all ligation reactions. Preferably, the ligase has little or no activity in ligating blunt ends and is substantially more active in ligating ends that are adjacent when annealed to a contiguous complementary sequence compared to ends that are not adjacently annealed to acomplementary sequence. One suitable ligase that can be used is Ampligase® Thermostable DNA Ligase (EPICENTRE Technologies, Madison, Wis.). Suitable reverse transcriptases that can be used are MMLV Reverse Transcriptase or IsoTherm™ DNA Polymerase (EPICENTRE Technologies, Madison, Wis.). Some preferred RNA polymerases are T7 RNAP, T3 RNAP, SP6 RNAP or another T7-like RNA polymerase or a mutant form of one of these T7-like RNA polymerases. Preferably, AmpliScribe™ T7-Flash™ Transcription Kit is used for in vitro transcription of the transcription substrate (EPICENTRE Technologies, Madison, Wis.).

In some embodiments, the anti-sense promoter oligo is attached to a solid support. In other embodiments, the anti-sense promoter oligo comprises a moiety, such as, but not limited to a biotin moiety that permits binding of the anti-sense promoter oligo to a solid support after annealing to the ligation product to obtain a transcription substrate. In some embodiments, the target sequence comprises a target nucleic acid in a sample, whereas in other embodiments the target sequence comprises a target sequence tag that is joined to an analyte-binding substance that binds an analyte in the sample.

In contrast to the above embodiments, the present invention also comprises additional embodiments, described below, that use an RNA polymerase that recognizes a cognate single-stranded transcription promoter or a single-stranded pseudopromoter. The ability to use a single-stranded promoter or pseudopromoter simplifies an assay or method of the invention since a transcription substrate can be obtained without the need to complex an anti-sense promoter oligo with a sense promoter sequence in a product of ligation of one or more target probes annealed to a target sequence. On the other hand, these embodiments obviously cannot use methods that comprise annealing to an anti-sense promoter oligo that is attached to a solid support.

Thus, one embodiment comprises a method to detect a target nucleic acid sequence, the method comprising a DNA ligation operation and a transcription operation, wherein the DNA ligation operation comprises ligation of one or more target probes comprising a promoter that that binds an RNA polymerase that can bind a single-stranded promoter and initiate transcription therefrom, wherein the ligation is dependent on hybridization of the target probes to the target nucleic acid sequence, and wherein the transcription operation comprises contacting the transcription substrate with an RNA polymerase that binds the single-stranded promoter under transcription condition to obtain a transcription product. Preferably, the ligase has little or no activity in ligating blunt ends and is substantially more active in ligating ends that are adjacent when annealed to two contiguous regions of a target sequence compared to ends that are not annealed to the target sequence. One suitable ligase that can be used is Ampligase® Thermostable DNA Ligase (EPICENTRE Technologies, Madison, Wis.). In preferred embodiments of this aspect of the invention, the single-stranded promoter comprises an N4 RNAP promoter and the RNA polymerase comprises an N4 mini-vRNAP enzyme, which comprises a transcriptionally active 1,106-amino acid domain corresponding to amino acids 998-2103 of N4 vRNAP, or a mutant form of N4 mini-vRNAP that comprises a mutation at position number Y678. In other embodiments the single-stranded promoter comprises a pseudopromoter for a T7-type RNAP (e.g., T7 RNAP, T3 RNAP or SP6 RNAP), E. coli RNAP or a Thermus RNAP, and the cognate RNA polymerase for the promoter is used. In preferred embodiments, a bipartite target probe and, optionally, one or more simple target probes is used. In other embodiments, the target probes comprise monopartite target probes comprising a promoter target probe and a signal target probe and/or optionally, one or more simple target probes. In some embodiments, the target sequence comprises a target nucleic acid in a sample, whereas in other embodiments the target sequence comprises a target sequence tag that is joined to an analyte-binding substance that binds an analyte in the sample. In some embodiments in which a bipartite target probe is used, the transcription substrate that is transcribed remains catenated to a target nucleic acid. In other embodiments of methods in which a bipartite target probe is used, the target sequence is preferably less than about 150 to about 200 nucleotides from the 3′-end of the target nucleic acid or target sequence tag. In still other embodiments of methods in which a bipartite target probe is used and in which the target sequence is greater than about 150 to about 200 nucleotides from the 3′-end of the target nucleic acid or target sequence tag comprising the target sequence, one or more additional steps is used in order to release the catenated circular ligation product from the target sequence prior to transcription, as described elsewhere herein.

One aspect of this embodiment of the invention comprises a method for detecting a target nucleic acid sequence, the method comprising: (a) providing one or more target probes comprising linear single-stranded DNA, the target probes comprising at least two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, and wherein the target probe that comprises the first target-complementary sequence also comprises a promoter that is joined to the 3′-end of the first target complementary sequence, which promoter can bind a single-stranded promoter and initiate transcription therefrom; (b) contacting the target probes with the target nucleic acid sequence and incubating under hybridization conditions, wherein the target-complementary sequences anneal adjacently to the target nucleic acid sequence to form a complex; (c) contacting the complex with a ligase under ligation conditions to form a transcription substrate; (d) contacting the transcription substrate with an RNA polymerase that can bind the single-stranded promoter under transcription conditions to obtain a transcription product; and (e) detecting the transcription product. Preferably, the ligase has little or no activity in ligating blunt ends and is substantially more active in ligating ends that are adjacent when annealed to two contiguous regions of a target sequence compared to ends that are not annealed to the target sequence. One suitable ligase that can be used is Ampligase® Thermostable DNA Ligase (EPICENTRE Technologies, Madison, Wis.). In preferred embodiments of this aspect of the invention, the single-stranded promoter comprises an N4 RNAP promoter and the RNA polymerase comprises an N4 mini-vRNAP enzyme, which comprises a transcriptionally active 1,106-amino acid domain corresponding to amino acids 998-2103 of N4 vRNAP, or a mutant form of N4 mini-vRNAP that comprises a mutation at position number Y678. In other embodiments the single-stranded promoter comprises a pseudopromoter for a T7-type RNAP (e.g., T7 RNAP, T3 RNAP or SP6 RNAP), E. coli RNAP or a Thermus RNAP, and the cognate RNA polymerase for the promoter is used. In preferred embodiments, a bipartite target probe and, optionally, one or more simple target probes is used. In other embodiments, the target probes comprise monopartite target probes comprising a promoter target probe and a signal target probe and/or optionally, one or more simple target probes. In some embodiments, the target sequence comprises a target nucleic acid in a sample, whereas in other embodiments the target sequence comprises a target sequence tag that is joined to an analyte-binding substance that binds an analyte in the sample. In some embodiments in which a bipartite target probe is used, the transcription substrate that is transcribed remains catenated to a target nucleic acid. In other embodiments of methods in which a bipartite target probe is used, the target sequence is preferably less than about 150 to about 200 nucleotides from the 3′-end of the target nucleic acid or target sequence tag. In still other embodiments of methods in which a bipartite target probe is used and in which the target sequence is greater than about 150 to about 200 nucleotides from the 3′-end of the target nucleic acid or target sequence tag comprising the target sequence, one or more additional steps is used in order to release the catenated circular ligation product from the target sequence prior to transcription, as described elsewhere herein.

Another aspect of this embodiment of the invention comprises a method for detecting a target nucleic acid sequence, the method comprising: (a) providing one or more target probes comprising linear single-stranded DNA, the target probes comprising at least two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, and wherein the target probe that comprises the first target-complementary sequence also comprises a promoter that is joined to the 3′-end of the first target-complementary sequence, which promoter binds an RNA polymerase that can bind a single-stranded promoter and initiate transcription therefrom; (b) contacting the target probes with the target nucleic acid sequence and incubating under hybridization conditions whereby the target probes anneal to the target nucleic acid sequence to form a complex; (c) contacting the complex with a DNA polymerase under DNA polymerization conditions to form a DNA polymerase extension product that is contiguous with the 5′-end of the first target-complementary sequence; (d) contacting the DNA polymerase extension product complex with a ligase under ligation conditions to form a transcription substrate; (e) contacting the transcription substrate with an RNA polymerase that can bind a single-stranded promoter and initiate transcription therefrom under transcription condition to obtain a transcription product; and (f) detecting the transcription product. Preferably, in this embodiment the ligase has little or no activity in ligating blunt ends and is substantially more active in ligating ends that are adjacent when annealed to two contiguous regions of a target sequence compared to ends that are not annealed to the target sequence. One suitable ligase that can be used is Ampligase® Thermostable DNA Ligase (EPICENTRE Technologies, Madison, Wis.). In preferred embodiments of this aspect of the invention, the single-stranded promoter comprises an N4 RNAP promoter and the RNA polymerase comprises an N4 mini-vRNAP enzyme, which comprises a transcriptionally active 1,106-amino acid domain corresponding to amino acids 998-2103 of N4 vRNAP, or a mutant form of N4 mini-vRNAP that comprises a mutation at position number Y678. In other embodiments the single-stranded promoter comprises a pseudopromoter for a T7-type RNAP (e.g., T7 RNAP, T3 RNAP or SP6 RNAP), E. coli RNAP or a Thermus RNAP, and the cognate RNA polymerase for the promoter is used. In preferred embodiments, a bipartite target probe and, optionally, one or more simple target probes is used. In other embodiments, the target probes comprise monopartite target probes comprising a promoter target probe and a signal target probe and/or optionally, one or more simple target probes. In some embodiments, the target sequence comprises a target nucleic acid in a sample, whereas in other embodiments the target sequence comprises a target sequence tag that is joined to an analyte-binding substance that binds an analyte in the sample. In some embodiments in which a bipartite target probe is used, the transcription substrate that is transcribed remains catenated to a target nucleic acid. In other embodiments of methods in which a bipartite target probe is used, the target sequence is preferably less than about 150 to about 200 nucleotides from the 3′-end of the target nucleic acid or target sequence tag. In still other embodiments of methods in which a bipartite target probe is used and in which the target sequence is greater than about 150 to about 200 nucleotides from the 3′-end of the target nucleic acid or target sequence tag comprising the target sequence, one or more additional steps is used in order to release the catenated circular ligation product from the target sequence prior to transcription, as described elsewhere herein.

Another embodiment of the invention comprises a method for obtaining transcription products comprising multiple copies of a target nucleic acid sequence (target sequence) in a sample, said method comprising: (a) providing one or more target probes comprising linear single-stranded DNA, said one or more target probes having at least two different target-complementary sequences that are not joined to each other, wherein the 5′-end of a first target-complementary sequence is complementary to the 5′-end of the target sequence and the 3′-end of a second target-complementary sequence is complementary to the 3′-end of the target sequence, and wherein the target probe that comprises a target-complementary sequence that is complementary to the 5′-end of the target sequence also comprises a promoter that is 3′-of the target-complementary sequence of said target probe, which promoter is for an RNA polymerase that lacks helicase-like activity and that can bind said single-stranded promoter and initiate transcription therefrom under transcription conditions, and wherein any additional target probes, if provided, comprise simple target probes having target-complementary sequences that anneal to the target sequence between the annealing sites of the first target-complementary sequence and the second target-complementary sequence, and wherein every free 5′-end of a target-complementary sequence that anneals to a target sequence has a 5′-phosphate and is adjacent to a 3′-end of another target-complementary sequence that has a 3′-hydroxyl end; (b) contacting the target probes with the target sequence and incubating under hybridization conditions so as to permit the target-complementary sequences of said target probes to anneal adjacently to all portions of the target sequence; (c) contacting said target probes annealed to said target sequence with a ligase under ligation conditions, wherein said ligase has little or no activity in ligating blunt ends and is substantially more active in ligating ends that are adjacent when annealed to two contiguous regions of a target sequence compared to ends that are not annealed to said target sequence, so as to obtain a ligated single-stranded DNA polynucleotide that comprises a transcription substrate for an RNA polymerase that lacks helicase-like activity and that can bind the single-stranded promoter in said transcription substrate and initiate transcription therefrom under transcription conditions; and (d) obtaining said transcription substrate, wherein said transcription substrate comprises a sequence that is complementary to said target sequence; and (e) contacting said transcription substrate with the RNA polymerase that can bind said promoter and initiate transcription therefrom under transcription conditions so as to obtain transcription product that is complementary to said transcription substrate; and (f) detecting synthesis of said transcription product comprising multiple copies of the target sequence obtained from transcription of said transcription substrate under transcription conditions, wherein synthesis of said transcription product indicates the presence of the target sequence.

Preferably, the ligase has little or no activity in ligating blunt ends and is substantially more active in ligating ends that are adjacent when annealed to two contiguous regions of a target sequence compared to ends that are not annealed to the target sequence. One suitable ligase that can be used is Ampligase® Thermostable DNA Ligase (EPICENTRE Technologies, Madison, Wis.). In preferred embodiments of this aspect of the invention, the single-stranded promoter comprises an N4 RNAP promoter and the RNA polymerase comprises an N4 mini-vRNAP enzyme, which comprises a transcriptionally active 1,106-amino acid domain corresponding to amino acids 998-2103 of N4 vRNAP, or a mutant form of N4 mini-vRNAP that comprises a mutation at position number Y678. In other embodiments the single-stranded promoter comprises a pseudopromoter for a T7-type RNAP (e.g., T7 RNAP, T3 RNAP or SP6 RNAP), E. coli RNAP or a Thermus RNAP, and the cognate RNA polymerase for the promoter is used. In preferred embodiments, a bipartite target probe and, optionally, one or more simple target probes is used. In other embodiments, the target probes comprise monopartite target probes comprising a promoter target probe and a signal target probe and/or optionally, one or more simple target probes. In some embodiments, the target sequence comprises a target nucleic acid in a sample, whereas in other embodiments the target sequence comprises a target sequence tag that is joined to an analyte-binding substance that binds an analyte in the sample. In some embodiments in which a bipartite target probe is used, the transcription substrate that is transcribed remains catenated to a target nucleic acid. In other embodiments of methods in which a bipartite target probe is used, the target sequence is preferably less than about 150 to about 200 nucleotides from the 3′-end of the target nucleic acid or target sequence tag. In still other embodiments of methods in which a bipartite target probe is used and in which the target sequence is greater than about 150 to about 200 nucleotides from the 3′-end of the target nucleic acid or target sequence tag comprising the target sequence, one or more additional steps is used in order to release the catenated circular ligation product from the target sequence prior to transcription, as described elsewhere herein.

Another embodiment of the present invention comprises a method for obtaining a transcription product complementary to a target nucleic acid sequence (target sequence or target), said method comprising: (a) providing a target sequence amplification probe (TSA probe), wherein said TSA probe comprises a linear single-stranded DNA comprising two end portions that are not joined, which end portions are connected by an intervening sequence, wherein the 5′-end target-complementary sequence is complementary to the 5′-end of the target sequence, and wherein the 3′-end target-complementary sequence is complementary to the 3′-end of the target sequence, and wherein joining of the ends of said TSA probe forms a TSA circle; (b) contacting the TSA probe to the target sequence and incubating under hybridization conditions, wherein the target-complementary sequences anneal adjacently on the target sequence; (c) contacting said TSA probe annealed to said target sequence with a ligase under ligation conditions so as to obtain a TSA circle; (d) providing a primer that is complementary to the intervening sequence of the TSA probe; (e) contacting the TSA circle with the primer that is complementary to the intervening sequence of the TSA probe under hybridization conditions so as to obtain a TSA circle-primer complex; (f) contacting said TSA circle-primer complex with a strand-displacing DNA polymerase under strand-displacing polymerization conditions so as to obtain a rolling circle replication product comprising multiple copies of the target sequence; (g) providing target probes comprising linear single-stranded DNA, the target probes comprising at least two target-complementary sequences that are not joined to each other, wherein the 5′-end of a first target-complementary sequence is complementary to the 5′-end of the target sequence, and wherein the 3′-end of a second target-complementary sequence is complementary to the 3′-end of the target sequence, and wherein the 3′-end of the first target-complementary sequence is joined to the 5′-end of a single-stranded promoter for an RNA polymerase than can bind said single-stranded promoter and initiate transcription therefrom under transcription conditions; (h) contacting the target probes with the target sequence and incubating under hybridization conditions, wherein the target-complementary sequences anneal adjacently on the target sequence to form a target probe-target complex; (i) contacting the target probe-target complex with a ligase under ligation conditions to obtain a ligation product comprising the target-complementary sequences of the target probes annealed to the target nucleic acid sequence, wherein said ligation product comprises a transcription substrate for the RNA polymerase; (j) contacting the transcription substrate with an RNA polymerase that can bind the single-stranded promoter and incubating under transcription conditions to obtain a transcription product; and (l) detecting the transcription product, wherein said transcription product indicates the presence of said target sequence. In some embodiments, the target probes comprise a bipartite target probe and optionally, one or more simple target probes. In other embodiments, the target probes comprise monopartite target probes comprising a promoter target probe and a signal target probe and/or one or more simple target probes.

Preferably with respect to the above embodiment, only one ligase is used for ligating both the TSA probe and the target probes. Preferably, the ligase has little or no activity in ligating blunt ends and is substantially more active in ligating ends that are adjacent when annealed to two contiguous regions of a target sequence compared to ends that are not annealed to the target sequence. One suitable ligase that can be used is Ampligase® Thermostable DNA Ligase (EPICENTRE Technologies, Madison, Wis.). A preferred strand-displacing DNA polymerase that can be used is IsoTherm™ DNA Polymerase (EPICENTRE Technologies, Madison, Wis.). Another suitable strand-displacing DNA polymerase that can be used is RepliPHI™ phi29 DNA polymerase (EPICENTRE Technologies, Madison, Wis.). Preferably, the single-stranded promoter comprises an N4 RNAP promoter and the RNA polymerase comprises an N4 mini-vRNAP enzyme, which comprises a transcriptionally active 1,106-amino acid domain corresponding to amino acids 998-2103 of N4 vRNAP, or a mutant form of N4 mini-vRNAP that comprises a mutation at position number Y678. In other embodiments the single-stranded promoter comprises a pseudopromoter for a T7-type RNAP (e.g., T7 RNAP, T3 RNAP or SP6 RNAP), E. coli RNAP or a Thermus RNAP, and the cognate RNA polymerase for the promoter is used. Preferably, a bipartite target probe and, optionally, one or more simple target probes is used. In other embodiments, the target probes comprise monopartite target probes comprising a promoter target probe and a signal target probe and/or optionally, one or more simple target probes. In some embodiments, the target sequence comprises a target nucleic acid in a sample, whereas in other embodiments the target sequence comprises a target sequence tag that is joined to an analyte-binding substance that binds an analyte in the sample. In some embodiments in which a TSA probe or a bipartite target probe is used, the TSA circle or circular transcription substrate, respectively, remains catenated to a target nucleic acid. In other embodiments of methods in which a TSA probe or a bipartite target probe is used, the target sequence is preferably less than about 150 to about 200 nucleotides from the 3′-end of the target nucleic acid or target sequence tag. In still other embodiments of methods in which the target sequence is greater than about 150 to about 200 nucleotides from the 3′-end of the target nucleic acid or target sequence tag, then one or more additional steps is used in order to release the catenated TSA circles from the target sequence prior to rolling circle replication, as described elsewhere herein. Similarly, one or more additional steps can be used in order to release the catenated circular ssDNA ligation products that result from ligation of bipartite target probes that are annealed to target sequences in the rolling circle replication product more than about 150 nucleotides to about 200 nucleotides from the 3′-end of to the rolling circle replication product. In one embodiment, rolling circle replication is carried out using a ratio of dUTP to dTTP that results in incorporation of a dUMP residue about every 100-400 nucleotides and a composition comprising uracil-N-glycosylase and endonuclease IV is used to release catenated DNA molecules that are ligated on the linear rolling circle replication product following annealing of bipartite target probes to the replicated target sequences.

Yet another embodiment is a method for detecting a target sequence, said method comprising: (a) providing a first bipartite target probe, wherein said first bipartite target probe comprises a 5′-portion and a 3′-portion, wherein said 5′-portion comprises: (i) a 5′-end portion that comprises a sequence that is complementary to a target sequence, and (ii) a promoter sequence, wherein said promoter sequence is covalently attached to and 3′-of said target-complementary sequence in said 5′-portion; and wherein said 3′-portion comprises: (i) a 3′-end portion that comprises a sequence that is complementary to a target sequence, wherein said target-complementary sequence of said 3′-end portion, when annealed to said target sequence, is adjacent to said target-complementary sequence of said 5′-end portion of said first bipartite target probe, and (ii) optionally, a signal sequence, wherein said signal sequence is 5′-of said target-complementary sequence of said 3′-portion of said first bipartite target probe; (b) providing a second bipartite target probe, wherein said second bipartite target probe comprises a 5′-portion and a 3′-portion, wherein said 5′-portion comprises: (i) a 5′-end portion that comprises sequence that is complementary to said target-complementary sequence of said 3′-end portion of said first bipartite target probe, and (ii) a promoter sequence, wherein said promoter sequence in said 5′-portion of said second bipartite target probe is 3′-of said target-complementary sequence in said 5′-portion; and wherein said 3′-portion comprises:

(i) a 3′-end portion that comprises sequence that is complementary to said target-complementary sequence of said 5′-end portion of said first bipartite target probe, and (ii) optionally, a signal sequence, wherein said signal sequence in said 3′-portion of said second bipartite target probe is 5′-of said target-complementary sequence in said 3′-portion; (c) annealing said first bipartite target probe to the target sequence under

hybridization conditions; (d) ligating said first bipartite target probe annealed to said target sequence with a ligase under ligation conditions, wherein said ligase has little or no activity in ligating blunt ends and is substantially more active in ligating said ends of said first bipartite target probe if said ends are adjacent when annealed to two contiguous regions of a target sequence than if said ends are not annealed to said target sequence, so as to obtain a circular ssDNA molecule that comprises a first circular

transcription substrate; (e) obtaining said first circular transcription substrate; (f) contacting said first circular transcription substrate with an RNA polymerase under transcription conditions so as to synthesize transcription product that is complementary to said first circular transcription substrate; (g) annealing to said transcription product that is complementary to said first circular transcription substrate a primer, wherein said primer is complementary to said transcription product; (h) contacting said transcription product to which said primer is annealed with a reverse transcriptase under reverse transcription conditions so as to obtain a first first-strand cDNA; (i) obtaining said first first-strand cDNA, wherein said first first-strand cDNA comprises a linear transcription substrate; (j) annealing to said first first-strand cDNA said second bipartite target probe under annealing conditions; (k) contacting said first first-strand cDNA to which said second bipartite target probe is annealed with a ligase under ligation conditions, wherein said ligase has little or no activity in ligating blunt ends and is substantially more active in ligating said ends of said second bipartite target probe if said ends are adjacent when annealed to two contiguous regions of said first first-strand cDNA than if said ends are not annealed to said sequence, so as to obtain a circular ssDNA molecule that comprises a second circular transcription substrate; (l) obtaining said second circular transcription substrate; (m) contacting said second circular transcription substrate with an RNA polymerase under transcription conditions so as to synthesize transcription product that is complementary to said second circular transcription substrate; (n) annealing to said transcription product that is complementary to said second circular transcription substrate a primer, wherein said primer is complementary to said transcription product; (o) contacting said transcription product to which said primer is annealed with a reverse transcriptase under reverse transcription conditions so as to obtain a second first-strand cDNA; (p) obtaining said second first-strand cDNA, wherein said second first-strand cDNA comprises a linear transcription substrate; (q) annealing to said second first-strand cDNA said first bipartite target probe under annealing conditions; (r) contacting said second first-strand cDNA to which said first bipartite target probe is annealed with a a ligase under ligation conditions, wherein said ligase has little or no activity in ligating blunt ends and is substantially more active in ligating said ends of said first bipartite target probe if said ends are adjacent when annealed to two contiguous regions of said second first-strand cDNA than if said ends are not annealed to said sequence, so as to obtain a circular ssDNA molecule that comprises a third circular transcription substrate that is identical to said first circular transcription substrate; (s) obtaining said third circular transcription substrate that is identical to said first circular transcription substrate; (t) repeating steps (a) through (t); (u) detecting the synthesis of transcription products resulting from transcription of said first, second and third circular transcription substrates and from said first and second linear transcription substrates, wherein said synthesis of said transcription products indicates the presence of said target sequence comprising said target nucleic acid. Preferably, in this embodiment, the target sequence is less than about 150 to about 200 nucleotides from the 3′-end of the target nucleic acid or target sequence tag. In other embodiments, if the target sequence is greater than about 150 to about 200 nucleotides from the 3′-end of the target nucleic acid or target sequence tag, one or more additional steps is used in order to release the catenated circular ligation products from the target sequence when the target probe anneals to a sequence in a linear DNA molecule that is greater than about 150 to about 200 nucleotides from the 3′-end of the linear DNA molecule. In still other embodiments, circular transcription substrates that are transcribed remain catenated to a target nucleic acid. Preferably, only one ligase is used for all ligation reactions. Preferably, the ligase has little or no activity in ligating blunt ends and is substantially more active in ligating ends that are adjacent when annealed to a contiguous complementary sequence compared to ends that are not adjacently annealed to acomplementary sequence. One suitable ligase that can be used is Ampligase® Thermostable DNA Ligase (EPICENTRE Technologies, Madison, Wis.). Suitable reverse transcriptases that can be used are MMLV Reverse Transcriptase or IsoTherm™ DNA Polymerase (EPICENTRE Technologies, Madison, Wis.). Preferably, the single-stranded promoter comprises an N4 RNAP promoter and the RNA polymerase comprises an N4 mini-vRNAP enzyme, which comprises a transcriptionally active 1,106-amino acid domain corresponding to amino acids 998-2103 of N4 vRNAP, or a mutant form of N4 mini-vRNAP that comprises a mutation at position number Y678. In other embodiments the single-stranded promoter comprises a pseudopromoter for a T7-type RNAP (e.g., T7 RNAP, T3 RNAP or SP6 RNAP), E. coli RNAP or a Thermus RNAP, and the cognate RNA polymerase for the promoter is used.

One embodiment that uses a double-stranded promoter and generates a circular transcription substrate comprises a method for detecting a target nucleic acid sequence, said method comprising: (a) providing at least two simple target probes comprising at least two target-complementary sequences, wherein the target probes comprise a 5′-phosphate and are adjacent when annealed on the target sequence, and wherein a first simple target probe is complementary to the 5′-end of the target nucleic acid and a second simple target probe is complementary to the 3′-end of the target nucleic acid sequence; (b) annealing the target probes to the target nucleic acid sequence under hybridization conditions; (c) contacting the target probes annealed to the target nucleic acid sequence with a ligase under ligation conditions so as to obtain a linear ligation product; (d) denaturing the ligation product from the target nucleic acid sequence;

(e) providing an open circle probe, wherein the 5′-end portion of the open circle probe comprises a 5′-phosphate group and a sense promoter sequence for a double-stranded transcription promoter that is recognized by a cognate RNA polymerase; (f) providing a first ligation splint comprising a 5′-end unphosphorylated portion that is complementary to the 5′-end portion of the open circle probe and a 3′-end portion that is complementary to the 3′-end portion of the ligation product; (g) providing a second ligation splint comprising a 5′-end unphosphorylated portion that is complementary to the 5′-end of the ligation product and a 3′-end that is complementary to the 3′-end of the open circle probe; (h) incubating the ligation product, the open circle probe, the first ligation splint and the second ligation splint under hybridization conditions so as to obtain a complex;

(i) contacting the complex with a ligase under ligation conditions so as to obtain a circular ligation product comprising the linear ligation product and the open circle probe; 0) annealing an anti-sense promoter oligo to the sense promoter sequence of the circular ligation product so as to obtain a circular transcription substrate; (k) contacting the circular transcription substrate with a cognate RNA polymerase for the promoter under transcription conditions so as to obtain a transcription product; and

(l) detecting the transcription product.

Another embodiment that uses a double-stranded promoter and generates a linear transcription substrate comprises a method for detecting a target nucleic acid sequence, said method comprising: (a) providing at least two simple target probes comprising at least two target-complementary sequences, wherein the target probes comprise a 5′-phosphate and are adjacent when annealed on the target sequence, and wherein a first simple target probe is complementary to the 5′-end of the target nucleic acid and a second simple target probe is complementary to the 3′-end of the target nucleic acid sequence; (b) annealing the target probes to the target nucleic acid sequence under hybridization conditions; (c) contacting the target probes annealed to the target nucleic acid sequence with a ligase under ligation conditions so as to obtain a first linear ligation product; (d) denaturing the ligation product from the target nucleic acid sequence;

(e) providing a promoter oligo comprising an oligodeoxyribonucleotide having a 5′-phosphate group and a sense promoter sequence for a double-stranded transcription promoter that is recognized by a cognate RNA polymerase; (f) optionally, providing a signal oligo comprising an oligodeoxyribonucleotide comprising a signal sequence;

(g) providing a first ligation splint comprising a 5′-end unphosphorylated portion that is complementary to the 5′-end portion of the promoter oligo and a 3′-end portion that is complementary to the 3′-end portion of the ligation product; (h) optionally, if a signal sequence is provided, providing a second ligation splint comprising a 5′-end unphosphorylated portion that is complementary to the 5′-end of the ligation product and a 3′-end that is complementary to the 3′-end of the signal oligo; (i) incubating the ligation product, promoter oligo, the first ligation splint and optionally, the signal oligo and the second ligation splint, under hybridization conditions so as to obtain a complex;

(j) contacting the complex with a ligase under ligation conditions so as to obtain a second linear ligation product comprising the first linear ligation product, the sense promoter and optionally, the signal sequence; (k) annealing an anti-sense promoter oligo to the sense promoter sequence of the second linear ligation product so as to obtain a linear transcription substrate; (l) contacting the linear transcription substrate with a cognate RNA polymerase for the promoter under transcription conditions so as to obtain a transcription product; and (m) detecting the transcription product.

One embodiment that uses a single-stranded promoter and generates a circular transcription substrate comprises a method for detecting a target nucleic acid sequence, said method comprising: (a) providing at least two simple target probes comprising at least two target-complementary sequences, wherein the target probes comprise a 5′-phosphate and are adjacent when annealed on the target sequence, and wherein a first simple target probe is complementary to the 5′-end of the target nucleic acid and a second simple target probe is complementary to the 3′-end of the target nucleic acid sequence; (b) annealing the target probes to the target nucleic acid sequence under hybridization conditions; (c) contacting the target probes annealed to the target nucleic acid sequence with a ligase under ligation conditions so as to obtain a linear ligation product; (d) denaturing the ligation product from the target nucleic acid sequence;

(e) providing an open circle probe, wherein the 5′-end portion of the open circle probe comprises a 5′-phosphate group and a sequence for single-stranded transcription promoter or pseudopromoter that is recognized by a cognate RNA polymerase; (f) providing a first ligation splint comprising a 5′-end unphosphorylated portion that is complementary to the 5′-end portion of the open circle probe and a 3′-end portion that is complementary to the 3′-end portion of the ligation product; (g) providing a second ligation splint comprising a 5′-end unphosphorylated portion that is complementary to the 5′-end of the ligation product and a 3′-end that is complementary to the 3′-end of the open circle probe;

(h) incubating the ligation product, the open circle probe, the first ligation splint and the second ligation splint under hybridization conditions so as to obtain a complex;

(i) contacting the complex with a ligase under ligation conditions so as to obtain a circular transcription substrate comprising the linear ligation product and the open circle probe; (j) contacting the circular transcription substrate with a cognate RNA polymerase for the promoter under transcription conditions so as to obtain a transcription product; and (k) detecting the transcription product.

Another embodiment that uses a single-stranded promoter and generates a linear transcription substrate comprises a method for detecting a target nucleic acid sequence, said method comprising: (a) providing at least two simple target probes comprising at least two target-complementary sequences, wherein the target probes comprise a 5′-phosphate and are adjacent when annealed on the target sequence, and wherein a first simple target probe is complementary to the 5′-end of the target nucleic acid and a second simple target probe is complementary to the 3′-end of the target nucleic acid sequence; (b) annealing the target probes to the target nucleic acid sequence under hybridization conditions; (c) contacting the target probes annealed to the target nucleic acid sequence with a ligase under ligation conditions so as to obtain a first linear ligation product; (d) denaturing the ligation product from the target nucleic acid sequence;

(e) providing a promoter oligo comprising an oligodeoxyribonucleotide having a 5′-phosphate group and a single-stranded promoter or pseudopromoter sequence for a single-stranded transcription promoter that is recognized by a cognate RNA polymerase; (f) optionally, providing a signal oligo comprising an oligodeoxyribonucleotide comprising a signal sequence; (g) providing a first ligation splint comprising a 5′-end unphosphorylated portion that is complementary to the 5′-end portion of the promoter oligo and a 3′-end portion that is complementary to the 3′-end portion of the ligation product; (h) optionally, if a signal sequence is provided, providing a second ligation splint comprising a 5′-end unphosphorylated portion that is complementary to the 5′-end of the ligation product and a 3′-end that is complementary to the 3′-end of the signal oligo; (i) incubating the ligation product, promoter oligo, the first ligation splint and optionally, the signal oligo and the second ligation splint, under hybridization conditions so as to obtain a complex; (O) contacting the complex with a ligase under ligation conditions so as to obtain a linear transcription substrate comprising the first linear ligation product, the sense promoter and optionally, the signal sequence; (k) contacting the linear transcription substrate with a cognate RNA polymerase for the promoter under transcription conditions so as to obtain a transcription product; and (1) detecting the transcription product.

Still another embodiment of the invention is a method for detecting a target nucleic acid sequence, the method comprising: (a) providing a simple bipartite target probe comprising linear single-stranded DNA (ssDNA) that lacks a sequence for a known promoter for an RNA polymerase, the simple bipartite target probe comprising two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, wherein said simple bipartite target probe is transcribed little or not at all by an RNA polymerase under conditions in which a circular ssDNA obtained by intramolecular ligation of the simple bipartite target probe is transcribed efficiently by said RNA polymerase; (b) contacting the simple bipartite target probe with the target nucleic acid sequence and incubating under hybridization conditions, wherein the ends of the target-complementary sequences anneal adjacently on the target nucleic acid sequence to form a complex; (c) contacting the complex with a ligase under ligation conditions so as to obtain a circular ssDNA ligation product comprising a circular transcription substrate for the RNA polymerase; (d) contacting the circular transcription substrate with the RNA polymerase under transcription conditions to obtain a transcription product; and (f) detecting the transcription product. In this embodiment, preferably, the ligase has little or no activity in ligating blunt ends and is substantially more active in ligating ends that are adjacent when annealed to two contiguous regions of a target sequence compared to ends

that are not annealed to the target sequence. One suitable ligase that can be used is Ampligase® Thermostable DNA Ligase (EPICENTRE Technologies, Madison, Wis.). In preferred embodiments, the RNA polymerase comprises an RNA polymerase chosen from among a T7 RNAP, a T3 RNAP, an SP6 RNAP or another T7-like RNA

polymerase, including mutant forms thereof, or E. coli RNA polymerase or Thermus thermophilus RNA polymerase. Another suitable RNA polymerase is an N4 mini-vRNAP.

In some embodiments, the target sequence comprises a target nucleic acid in a sample, whereas in other embodiments the target sequence comprises a target sequence tag that is joined to an analyte-binding substance that binds an analyte in the sample. In some embodiments, the method is used to detect a single-nucleotide polymorphism (SNP) or mutation, in which case the 5′-nucleotide of the first target-complementary sequence or the 3′-end of the second target-complementary sequence of said simple bipartite target probe is complementary to the intended target nucleotide of the target sequence, and ligation only occurs when the ends of both target-complementary sequences are adjacently annealed on the target sequence, including the target nucleotide, under the stringent ligation conditions of the assay or method. The target sequence is preferably less than about 150 to about 200 nucleotides from the 3′-end of the target nucleic acid or target sequence tag. In embodiments in which the target sequence is greater than about 150 to about 200 nucleotides from the 3′-end of the target nucleic acid or target sequence tag comprising the target sequence, one or more additional steps is used in order to release the catenated circular ligation product from the target sequence prior to transcription, as described elsewhere herein. In still other embodiments in which a bipartite target probe is used, the circular transcription substrate that is transcribed remains catenated to a target nucleic acid.

Another embodiment of the present invention is a method for detecting an analyte in a sample, wherein said analyte comprises a biomolecule that is not a nucleic acid, said method comprising: (a) providing an analyte-binding substance comprising a nucleic acid, wherein said nucleic acid binds with selectivity and high affinity to said analyte; (b) providing target probes comprising either (i) a promoter target probe and one or more additional target probes chosen from among a signal target probe and simple target probe; or (ii) a bipartite target probe and, if said target-complementary sequences of said bipartite target probe are not contiguous when annealed to said target sequence in said analyte-binding substance, optionally, one or more simple target probes; wherein said target probes of (i) or (ii) comprise sequences that are complementary to adjacent regions of a target sequence in said analyte-binding substance; (c) contacting said analyte-binding substance to an analyte in a sample; (d) separating said analyte-binding substance molecules that are bound to said analyte from said analyte-binding substance molecules that are not bound to said analyte; (e) contacting said analyte-binding substance molecules that are bound to said analyte with said target probes provided in step b(i) or step b (ii) above under hybridization conditions that permit said target probes that are complementary to said target sequences in said analyte-binding substance to anneal thereto; (f) ligating said adjacent target probes that are annealed to said target sequence of said analyte-binding substance with a ligase under ligation conditions so as to obtain a ligation product; (g) contacting the ligation product with an anti-sense promoter oligo and incubating under hybridization conditions, wherein the anti-sense promoter oligo anneals to the sense promoter sequence of the ligation product to form a transcription substrate; (h) contacting said transcription substrate with an RNA polymerase under transcription conditions so as to synthesize RNA that is complementary to said transcription substrate; (i) optionally, repeating steps a through i; and (j) detecting the synthesis of RNA resulting from transcription of said transcription substrate, wherein said synthesis of said RNA indicates the presence of said analyte in said sample.

The invention also comprises methods, compositions and kits for using ssDNA transcription substrates and RNA polymerases that can transcribe said ssDNA transcription substrates as a signaling system for an analyte of any type, including analytes such as, but not limited to, antigens, antibodies or other substances, in addition to an analyte that is a target nucleic acid.

Thus, the invention comprises a method for detecting an analyte in or from a sample, said method comprising: 1. providing a transcription signaling system, said transcription signaling system comprising a ssDNA comprising: (a) a 5′-portion comprising a sense promoter sequence for a double-stranded promoter for a cognate RNA polymerase; and (b) a signal sequence, wherein said signal sequence, when transcribed by said RNA polymerase, is detectable in some manner; 2. joining said transcription signaling system, either covalently or non-covalently, to an analyte-binding substance, wherein said joining to said substance is not affected by the conditions of the assay and wherein said joining to said substance does not affect the ability of said transcription signaling system to be transcribed using said RNA polymerase under transcription conditions; 3. contacting said analyte-binding substance to which said transcription signaling system is joined with a sample under binding conditions, wherein said analyte, if present in said sample, binds to said analyte-binding substance so as to form a specific binding pair; 4. removing said specific binding pair from said sample so as to separate it from other components in said sample; 5. contacting the specific binding pair with an anti-sense promoter oligo under annealing conditions, wherein the anti-sense promoter oligo anneals to the sense promoter sequence to form a transcription substrate;

6. incubating said specific-binding pair under transcription conditions with an RNA polymerase, wherein said RNA polymerase synthesizes RNA that is complementary to said signal sequence in said ssDNA transcription signaling system under said transcription conditions; 7. obtaining the RNA synthesis product that is complementary to said signal sequence in said ssDNA transcription signaling system; and 8. detecting said RNA synthesis product or a substance that results from said RNA synthesis product.

Another embodiment of the present invention comprises a method for amplifying a target nucleic acid by strand displacement reverse transcription of a linear single-stranded RNA (ssRNA) template, said method comprising: 1. providing a reaction mixture comprising: (a) a reverse transcriptase with strand-displacement activity; (b) optionally, a single-strand binding protein; and (c) multiple oligonucleotide primers, wherein at least the 3′-portion of each said primer comprises a sequence that is complementary to a sequence in said ssRNA; 2. contacting said reaction mixture from step 1 above with a sample comprising a target nucleic acid comprising a ssRNA, wherein said reaction mixture containing said sample is maintained at a temperature wherein said reverse transcriptase, and optionally said single-strand binding protein, are optimally active in combination for strand-displacement reverse transcription and wherein said reverse transcription primers anneal to said target sequence, if present, with specificity, and wherein said temperature of said reaction mixture is maintained for a time sufficient to permit synthesis of first-strand cDNA reverse transcription products complementary to said target nucleic acid, if present in said sample; and 3. obtaining multiple copies of said first-strand cDNA that is complementary to said RNA target nucleic acid.

Still another embodiment of the present invention comprises a method for amplifying a target nucleic acid comprising a circular single-stranded RNA (ssRNA) by strand displacement reverse transcription, said method comprising: 1. providing a reaction mixture comprising: (a) a reverse transcriptase with strand-displacement activity; (b) optionally, a single-strand binding protein; and (c) at least one, and optionally multiple oligonucleotide primers, wherein at least the 3′-portion of each said primer comprises a sequence that is complementary to a sequence in said ssRNA; 2. contacting said reaction mixture from step 1 above with a sample comprising a target nucleic acid comprising a circular ssRNA, wherein said reaction mixture containing said sample is maintained at a temperature wherein said reverse transcriptase, and optionally said single-strand binding protein, are optimally active in combination for strand-displacement reverse transcription and wherein said reverse transcription primers anneal to said target sequence, if present, with specificity, and wherein said temperature of said reaction mixture is maintained for a time sufficient to permit synthesis of first-strand cDNA reverse transcription products complementary to said target nucleic acid, if present in said sample; and 3. obtaining first-strand cDNA multimers comprising multiple tandem copies of a first-strand cDNA oligomer, each of which is complementary to one copy of said circular ssRNA target nucleic acid template.

In some embodiments the primers in the above methods for strand displacement reverse transcription comprise DNA oligonucleotides. In other embodiments, the primers comprise ribonucleotides, and in still other embodiments the primers comprise 2′-fluoro-containing modified oligoribonucleotides or DuraScript™ RNA, for example made using the DuraScribe™ Transcription Kit (EPICENTRE Technologies, Madison, Wis., USA). A primer for strand-displacement reverse transcription can comprise a specific sequence that is complementary to only one RNA sequence, or alternatively, the multiple strand-displacement primers of a strand-displacement reverse transcription reaction of the present invention can also comprise random sequence primers, including but not limited to random hexamers, random octamers, random nonamers, random decamers, or random dodecamers. When random sequence primers are used, the primers can also prime synthesis of second-strand cDNA using first-strand cDNA as a template, and subsequently, can prime the synthesis of third, fourth and other cDNA strands, thereby resulting in additional amplification. In some preferred embodiments, the random sequence primers comprise alpha-thio internucleoside linkages, which are resistant to some exonucleases. In some embodiments, a biotin or other binding moiety is covalently attached to a nucleotide in the 5′-portion of a reverse transcription primer used for strand-displacement reverse transcription. The biotin or other binding moiety enables capture of first-strand cDNA obtained by strand-displacement reverse transcription.

Strand-displacing reverse transcriptases that can be used include, but are not limited to RNaseH-Minus MMLV reverse transcriptase or IsoTherm™ DNA Polymerase (EPICENTRE Technologies, Madison, Wis.). One reverse transcription reaction condition that can increase displacement of first-strand cDNA, and which is included in some embodiments as part of the present invention, is addition of a single-strand binding protein, such as, but not limited to EcoSSB Protein or an SSB Protein from a thermostable bacterium, such as Tth or Bst SSB Protein, to a reverse transcription reaction.

Betaine can also be added to a reverse transcription reaction in order to increase strand displacement. As disclosed in U.S. Pat. Nos. 6,048,696 and 6,030,814, and in German Patent No. DE4411588C1, all of which are incorporated herein by reference and made part of the present invention, it is preferred in many embodiments to use a final concentration of about 0.25 M, about 0.5 M, about 1.0 M, about 1.5 M, about 2.0 M, about 2.5 M or between about 0.25 M and 2.5 M betaine (trimethylglycine) in DNA polymerase or reverse transcriptase reactions in order to decrease DNA polymerase stops and increase the specificity of reactions that use a DNA polymerase.

The invention will be better understood by inspection of several figures and illustrations of the various embodiments, which are intended only as examples and not to limit the scope of the invention.

### BRIEF DESCRIPTION OF THE FIGURES

FIG. 1 illustrates an example of different monopartite target probes and a related composition—an anti-sense promoter oligo—for embodiments of the invention that use a promoter target probe that comprises a sense promoter sequence for an RNA polymerase that uses a double-stranded promoter.

FIG. 2 shows an example of a bipartite target probe and a related composition—an anti-sense promoter oligo—for embodiments of the invention that use a bipartite target probe that comprises a sense promoter sequence for an RNA polymerase that uses a double-stranded promoter.

FIG. 3 shows a basic embodiment of the invention for detecting a target sequence using a bipartite target probe having a sense promoter sequence for a double-stranded promoter and target-complementary sequences that are contiguous when annealed to a target nucleic acid having the target sequence.

FIG. 4 shows an embodiment of a method in which a circular transcription substrate is obtained by annealing a bipartite target probe having a sense promoter sequence for a double-stranded promoter to a target sequence, ligating the annealed bipartite target probe, and then complexing the ligation product with an anti-sense promoter oligo. In the emobodiment illustrated, the circular transcription substrate is amplified by rolling circle transcription.

FIG. 5 shows an embodiment of a method that uses coupled target-dependent rolling circle replication and run-off transcription of a linear transcription substrate that has a double-stranded promoter to amplify the amount of transcription product obtained. The copies of the target sequence in the rolling circle replication product are identical to the target sequence in the sample and provide additional sites for annealing and ligation of target probes in order to obtain more linear transcription substrates. The embodiment shown here uses monopartite target probes to make a linear transcription substrate. The invention also comprises embodiments that use a bipartite target probe to obtain a circular transcription substrate for rolling circle transcription.

FIG. 6 shows an embodiment of a method in which target-complementary sequences of the bipartite target probe are not contiguous when annealed to a target sequence and the gap between the target-complementary sequences is filled using a simple target probe.

FIG. 7 illustrates an embodiment of a method in which target-complementary sequences of the bipartite target probe are not contiguous when annealed to a target sequence and the gap between the target-complementary sequences is filled by DNA polymerase extension.

FIG. 8 shows an embodiment of the invention for detecting a target sequence by generating a linear transcription substrate using monopartite target probes, including a promoter target probe that comprises a sense promoter sequence for an RNA polymerase that uses a double-stranded promoter.

FIG. 9 shows a method to obtain additional amplification of transcription products.

FIG. 10 shows a method for detecting a non-nucleic acid analyte using an analyte-binding substance comprising an antibody that has a covalently-(e.g., chemically attached) or non-covalently-(e.g., using biotin and streptavidin) attached target sequence tag comprising a target sequence. This example uses a bipartite target probe having a sense promoter sequence for a double-stranded promoter, such as a T7 RNAP promoter. In this particular embodiment, the circular transcription substrate has a transcription termination sequence so that multiple single RNA copies are obtained, rather than multimeric tandem copies of an oligomeric RNA, as obtained by rolling circle transcription.

FIG. 11 shows a method for detecting a non-nucleic acid analyte using an analyte-binding substance that has a target sequence tag comprising a target sequence, wherein the the signal for the analyte-binding substance and the analyte is generated by rolling circle transcription. This example also uses a bipartite target probe having a sense promoter sequence for a double-stranded promoter, but does not have a transcription termination sequence so that multimeric tandem copies of an oligomeric RNA is obtained by rolling circle transcription.

FIG. 12 illustrates an example of different monopartite target probes for embodiments of the invention in which the promoter sequence comprises a sequence for an RNA polymerase that binds a single-stranded promoter and initiates transcription therefrom. The target probes are similar to embodiments that use an RNA polymerase that binds a double-stranded promoter except that embodiments that use single-stranded promoters are simpler since they do not use an anti-sense promoter oligo to make a transcription substrate of the invention. Preferably, the single-stranded promoter sequence is a pseudopromoter for an RNA polymerase, such as but not limited to a pseudopromoter for a T7-type RNAP (e.g., T7 RNAP, T3 RNAP or SP6 RNAP), E. coli RNAP or a Thermus RNAP, and the cognate RNA polymerase for the promoter is used. In other embodiments, the single-stranded promoter is an N4 promoter and the RNA polymerase is an N4 mini-vRNAP or a Y678F mutant of an N4 mini-vRNAP.

FIG. 13 shows an example of a bipartite target probe of the invention that comprises a single-stranded promoter sequence for an RNA polymerase that uses a single-stranded promoter for transcription. The bipartite target probe is similar to a bipartite target probe that has a sense promoter sequence for a double-stranded promoter except that a circular transcription substrate is obtained using a bipartite target probe that comprises a single-stranded promoter without annealing of an anti-sense promoter oligo. Preferably, the single-stranded promoter comprising a bipartite target probe is an N4 promoter and the RNA polymerase is an N4 mini-vRNAP or a Y678F mutant of an N4 mini-vRNAP. In other embodiments, the single-stranded promoter is a pseudopromoter for an RNA polymerase, such as but not limited to a pseudopromoter for a T7-type RNAP (e.g., T7 RNAP, T3 RNAP or SP6 RNAP), E. coli RNAP or a Thermus RNAP, and the cognate RNA polymerase for the promoter is used.

FIG. 14 shows a basic embodiment of the invention for detecting a target sequence using a bipartite target probe comprising a single-stranded N4 promoter or a pseudopromoter, wherein the bipartite target probe has target-complementary sequences that are contiguous when annealed to a target nucleic acid sequence of a target nucleic acid or a target sequence tag that is joined to an analyte-binding substance. Preferably, the single-stranded promoter comprising a bipartite target probe is an N4 promoter and the RNA polymerase is an N4 mini-vRNAP or a Y678F mutant of an N4 mini-vRNAP. In other embodiments, the single-stranded promoter is a pseudopromoter for an RNA polymerase, such as but not limited to a pseudopromoter for a T7-type RNAP (e.g., T7 RNAP, T3 RNAP or SP6 RNAP), E. coli RNAP or a Thermus RNAP, and the cognate RNA polymerase for the promoter is used.

FIG. 15 shows an embodiment of a method in which the circular transcription substrate obtained using a bipartite target probe having a single-stranded promoter or pseudopromoter is amplified by rolling circle transcription. Preferably, the single-stranded promoter comprising a bipartite target probe is an N4 promoter and the RNA polymerase is an N4 mini-vRNAP or a Y678F mutant of an N4 mini-vRNAP. In other embodiments, the single-stranded promoter is a pseudopromoter for an RNA polymerase, such as but not limited to a pseudopromoter for a T7-type RNAP (e.g., T7 RNAP, T3 RNAP or SP6 RNAP), E. coli RNAP or a Thermus RNAP, and the cognate RNA polymerase for the promoter is used.

FIG. 16 shows an embodiment of a method that uses coupled target-dependent rolling circle replication and rolling circle transcription to amplify the amount of transcription product obtained. Preferably, IsoTherm™ DNA Polymerase (EPICENTRE Technologies, Madison, Wis.) is used for rolling circle replication. Another strand-displacing DNA polymerase that can be used is RepliPHI™ phi29 DNA polymerase (EPICENTRE Technologies, Madison, Wis.). The copies of the target sequence in the rolling circle replication product are identical to the target sequence in the sample and provide additional sites for annealing and ligation of target probes in order to obtain more transcription substrates. The embodiment shown here uses a bipartite target probe that comprises a single-stranded promoter or pseudopromoter to make a circular transcription substrate for rolling circle transcription by an RNA polymerase that binds the single-stranded promoter. Ligation of the bipartite target probe catenates the circular transcription substrate to the rolling circle replication product comprising the replicated target sequence. The catenated circular transcription substrates must be released from the rolling circle replication product to achieve efficient rolling circle transcription. The method for releasing the catenated circular transcription substrates illustrated here is to include a quantity of dUTP in the rolling circle replication reaction mix in addition to dTTP so that a dUMP residue is incorporated randomly about every 100-400 nucleotides. Uracil-N-glycosylase and endonuclease IV, which cleave the DNA strand wherever dUMP is incorporated, is also included in the reaction mixture. Once the rolling circle replication product is cleaved so that, on average, most of the replicated target sequences are within about 150-200 nucleotides from a free 3′-end, the catenated circular transcription substrates will be released during rolling circle transcription. Preferably, the single-stranded promoter comprising a bipartite target probe is an N4 promoter and the RNA polymerase is an N4 mini-vRNAP or a Y678F mutant of an N4 mini-vRNAP. In other embodiments, the single-stranded promoter is a pseudopromoter for an RNA polymerase, such as but not limited to a pseudopromoter for a T7-type RNAP (e.g., T7 RNAP, T3 RNAP or SP6 RNAP), E. coli RNAP or a Thermus RNAP, and the cognate RNA polymerase for the promoter is used.

FIG. 17 shows an embodiment of a method that uses coupled target-dependent rolling circle replication and run-off transcription of linear transcription substrates obtained by ligation of monopartite target probes annealed to the replicated target sequences in the rolling circle replication product to amplify the amount of transcription product. In embodiments that use linear transcription substrates, preferably the single-stranded promoter is a pseudopromoter for an RNA polymerase, such as but not limited to a pseudopromoter for a T7-type RNAP (e.g., T7 RNAP, T3 RNAP or SP6 RNAP), E. coli RNAP or a Thermus RNAP, and the cognate RNA polymerase for the promoter is used, since these enzymes efficiently displace the RNA product from the DNA template strand. However, an N4 mini-vRNAP enzyme can also be used together with a composition of EcoSSB Protein.

FIG. 18 shows an embodiment of a method in which the target-complementary sequences of a bipartite target probe that comprises a single-stranded promoter or pseudopromoter are not contiguous when annealed to a target sequence and the gap between the target-complementary sequences is filled using a simple target probe. In the embodiment illustrated here, the circular transcription substrate has a transcription termination sequence so that only one copy of the transcription product is obtained, rather than a multimer of tandem oligomers as obtained from rolling circle transcription.

FIG. 19 illustrates an embodiment of a method in which the target-complementary sequences of a bipartite target probe that comprises a single-stranded promoter or pseudopromoter are not contiguous when annealed to a target sequence and the gap between the target-complementary sequences is filled by DNA polymerase extension and subsequent ligation to obtain a circular transcription substrate.

FIG. 20 shows an embodiment of the invention for detecting a target sequence by generating a linear transcription substrate using monopartite target probes, wherein the promoter target probe comprises a single-stranded promoter or pseudopromoter. Preferably the single-stranded promoter is a pseudopromoter for a T7-type RNAP (e.g., T7 RNAP, T3 RNAP or SP6 RNAP) and the RNA polymerase is the cognate T7-type RNAP, or a pseudopromoter for E. coli RNAP or a Thermus RNAP, and the cognate RNA polymerase for the promoter is used, since these enzymes efficiently displace the RNA product from the DNA template strand. However, an N4 mini-vRNAP enzyme can also be used together with a composition of EcoSSB Protein

FIG. 21 shows a method to obtain additional amplification of transcription products. The method uses two bipartite target probes comprising single-stranded promoters or pseudopromoters to generate circular transcription substrates for rolling circle transcription by a cognate RNA polymerase, and reverse transcription of the resulting RNA products to make additional copies of sense or anti-sense target sequences for annealing and ligation of additional first or second bipartite target probes, respectively, which in turn are used to transcribe more RNA, which is detected.

FIG. 22 shows a method for detecting a non-nucleic acid analyte using an analyte-binding substance comprising an antibody that has a covalently-(e.g., chemically attached) or non-covalently-(e.g., using biotin and streptavidin) attached target sequence tag comprising a target sequence. In the embodiment illustrated here, the signal for detection of the analyte-binding substance and the analyte is generated by transcription of a circular transcription substrate obtained by annealing and ligation of a bipartite target probe comprising a single-stranded promoter or pseudopromoter. In this particular embodiment, the circular transcription substrate has a transcription termination sequence so that multiple single RNA copies are obtained, rather than multimeric tandem copies of an oligomeric RNA as obtained by rolling circle transcription.

FIG. 23 shows a method for detecting a non-nucleic acid analyte using an analyte-binding substance that has a target sequence tag comprising a target sequence, wherein the the signal for the analyte-binding substance and the analyte is generated by rolling circle transcription of a circular transcription substrate obtained by annealing and ligation of a bipartite target probe comprising a single-stranded promoter or pseudopromoter. Since the target sequence tag is designed to have a size so that a free 3′-end is less than about 150 nucleotides and preferably less than 50-100 nucleotides from the target sequence, the catenated circular transcription substrates are easily released from the target sequence tag. The analyte can be any of a broad range of analytes for which an analyte-binding substance is available or can be identified. Preferably, the single-stranded promoter comprising a bipartite target probe is an N4 promoter and the RNA polymerase is an N4 mini-vRNAP or a Y678F mutant of an N4 mini-vRNAP. In other embodiments, the single-stranded promoter is a pseudopromoter for an RNA polymerase, such as but not limited to a pseudopromoter for a T7-type RNAP (e.g., T7 RNAP, T3 RNAP or SP6 RNAP), E. coli RNAP or a Thermus RNAP, and the cognate RNA polymerase for the promoter is used.

### DETAILED DESCRIPTION OF THE INVENTION

The present invention discloses novel methods, processes, compositions, and kits for amplifying and detecting one or multiple target nucleic acid sequences in or from a sample, including target sequences that differ by as little as one nucleotide. The target sequence or target sequences can comprise at least a portion of one or more target nucleic acids comprising either RNA or DNA from any source, or a target sequence can comprise a target sequence tag that is attached to an analyte-binding substance, such as, but not limited to, an antibody, thus permitting use of the methods and compositions of the present invention to detect any analyte for which there is a suitable analyte-binding substance. The methods of the invention involve obtaining transcription products using a transcription substrate as a template, wherein the transcription substrate is made by ligating at least two different target-complementary sequences comprising one or more target probes when the target-complementary sequences are annealed adjacently on the target sequence. Since the target sequence is required for annealing and ligation of the target-complementary sequences which makes the transcription substrate, obtaining the transcription product is target-dependent. Therefore, detection of the transcription products is indicative of the presence of the target sequence comprising the target nucleic acid or the target sequence tag joined to an analyte-binding substance in the sample.

In one aspect, the present invention comprises novel methods, compositions and kits for amplifying, detecting and quantifying one or multiple target nucleic acid sequences in a sample, including target sequences that differ by as little as one nucleotide. The target sequence or target sequences can comprise one or more target nucleic acids comprising either RNA or DNA from any source. The methods can also be used to detect an analyte of any type for which an analyte-binding substance (such as, but not limited to, an antibody) can be obtained, provided that a tag comprising a target nucleic acid sequence is coupled or linked to said analyte-binding substance. The method is useful for detecting specific nucleic acids or analytes in a sample with high specificity and sensitivity. The method also has an inherently low level of background signal. Preferred embodiments of the method consist of an annealing process, a DNA ligation process, an optional DNA polymerase extension process, a transcription process, and, optionally, a detection process. The DNA ligation joins a probe which has a first target-complementary sequence and a sense promoter sequence for an RNA polymerase to another probe or another section of the same probe which has a second target-complementary sequence and, optionally, a signal sequence. This step is dependent on hybridization of the target-complementary probe sequences to a target sequence and forms a ligation product which, upon complexing with an anti-sense promoter oligo, makes a transcription substrate for in vitro transcription of the second target-complementary sequence and the signal sequence, if a signal sequence is present, in an amount that is proportional to the amount of target sequence in the sample.

In vitro transcription amplifies the target-complementary sequence and the signal sequence, if present, in proportion to the amount of transcription substrate formed, permitting quantification of the amount of target sequence present. The invention uses an RNA polymerase, preferably a T7-type RNA polymerase, and most preferably, T7 RNA polymerase, and synthesizes a transcription product using the transcription promoter and a single-stranded DNA template which is operably or functionally joined or linked to the promoter using a method of the invention. Joining of the sense promoter sequence to the transcription template, which yields a transcription substrate upon complexing with an anti-sense promoter sequence, is target-dependent because joining by ligation only occurs if the different target-complementary sequences comprising the target probes are adjacent to or abut each when they anneal to a target sequence, if the target sequence is present in the sample. The methods of the invention are therefore referred to herein as “target-dependent transcription.”

The amount of transcription product obtained in a given reaction time can also be increased using a coupled rolling circle replication and target-dependent transcription reaction. The rolling circle replication reaction uses a “target sequence amplification probe” (or a “TSA probe”) having target-complementary sequences at each end and an intervening sequence with a primer binding site. The TSA probe anneals to the target sequence, if present in the sample, and is ligated to form a “TSA circle.” After annealing a primer to the primer binding site, rolling circle replication of the TSA circle by a strand-displacing DNA polymerase under strand-displacing polymerization conditions generates multiple tandem copies of the target sequence, which serve as annealing and ligation sites for one or more target probes of the invention. Ligation joins a sense promoter sequence and a first target-complementary sequence to one or more other target-complementary sequences. Then, after ligation of the target probes and annealing of an anti-sense promoter oligo to the sense promoter sequence, a transcription substrate is obtained for an RNA polymerase that binds the double-stranded promoter and synthesizes transcription products comprising multiple copies of the target sequence.

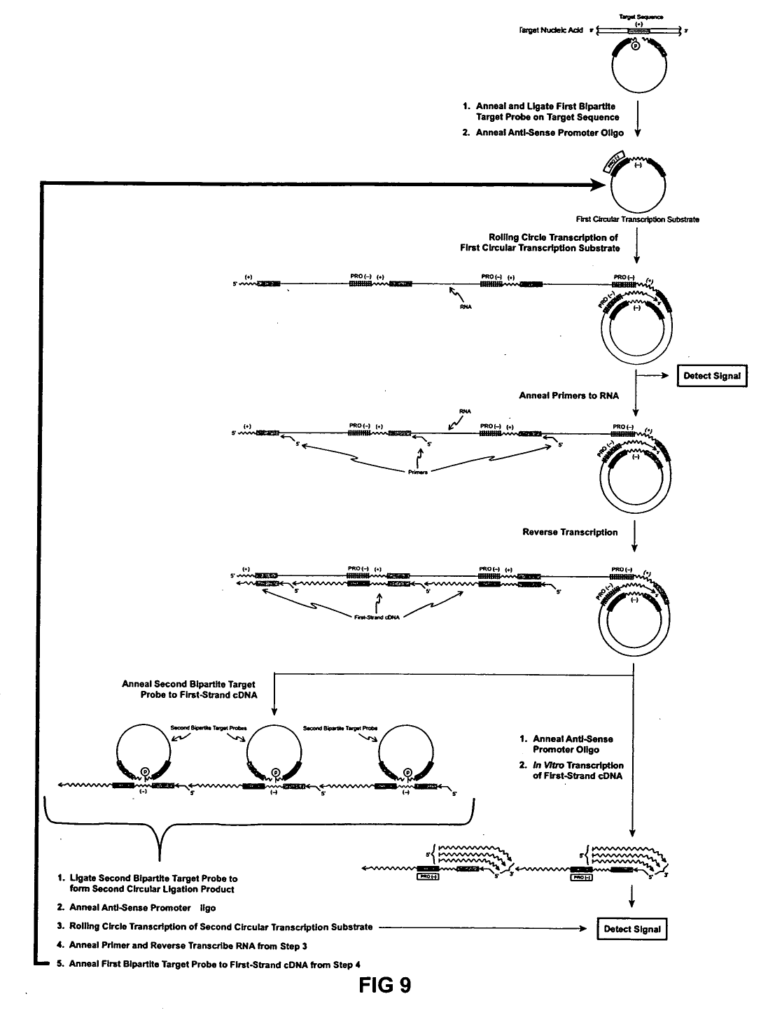

Yet another method for obtaining additional amplification of the target sequence is illustrated schematically in FIG. 9. This method generates annealing and ligation sites for a second bipartite target probe by reverse transcription of the transcription products obtained following annealing of a first bipartite target probe to a target sequence in the sample, ligation of the bipartite target probe, and in vitro transcription of the resulting circular transcription substrate.

Following in vitro transcription, RNA complementary to target-complementary probe sequences can be detected and quantified using any of the conventional detection systems for nucleic acids such as detection of fluorescent labels, enzyme-linked detection systems, antibody-mediated label detection, and detection of radioactive labels. Alternatively, the signal sequence in the transcription substrate can comprise a sequence that is amplifiable and/or detectable by another method. By way of example, but not of limitation, in some embodiments of the invention a signal sequence that encodes a substrate for an enzyme, such as, but not limited to Q-beta replicase is used. In the latter embodiment, in vitro transcription of the ssDNA transcription substrate results in synthesis of a substrate for a replicase, which is used to rapidly and linearly amplify the signal further. Since the amplified product is directly proportional to the amount of target sequence present, quantitative measurements reliably represent the amount of a target sequence in a sample. Major advantages of this method are that the ligation process, or an optional DNA polymerase extension, can be manipulated to obtain single-nucleotide allelic discrimination, the transcription process is isothermal, and signals are strictly quantitative because the transcription reaction is linear and is catalyzed by a highly processive enzyme, and signal amplification can be obtained which is also linear and greatly enhances the sensitivity of an assay or method. In multiplex assays, the transcription promoter sequence used for in vitro transcription can be the same for all target probes.

The invention will be understood from the description of the additional background, compositions, processes, methods and kits described herein below.

A. Nucleic Acids and Polynucleotides of This Aspect of the Invention

A “nucleic acid” or “polynucleotide” or “oligonucleotide” (or “oligo”) of the invention is a polymer molecule comprising a series of “mononucleosides,” also referred to as “nucleosides,” in which the 3′-position of the pentose sugar of one nucleoside is linked by an internucleoside linkage, such as, but not limited to, a phosphodiester bond, to the 5′-position of the pentose sugar of the next nucleoside. A nucleoside linked to a phosphate group is referred to as a “nucleotide.” The nucleotide that is linked to the 5′-position of the next nucleotide in the series is referred to as “5′-of,” or “upstream of,” or the “5′-nucleotide” and the nucleotide that is linked to the 3′-position of said 5′ or upstream nucleotide is referred to as “3′-of,” or “downstream of,” or the “3′-nucleotide.” When two different, non-overlapping polynucleotides or oligonucleotides hybridize or anneal to different regions of the same linear complementary nucleic acid sequence, and the 3′-end of one polynucleotide or oligonucleotide points towards the 5′-end of the other, the former may be called the “upstream” polynucleotide or oligonucleotide and the latter the “downstream” polynucleotide or oligonucleotide.

The terms “3′-of” and “5′-of” are used herein to refer to the position or orientation of a particular nucleic acid sequence or genetic element encoded by a sequence, such as, but not limited to, a transcription promoter, relative to other sequences or genetic elements.

A “portion” or “region,” used interchangeably herein, of a polynucleotide or oligonucleotide is a contiguous sequence of 2 or more bases. In other embodiments, a region or portion is at least about any of 3-5,5-10, 10-15, 15-20, 20-25, 25-50, 50-100, 100-200,200-400, 400-600, 600-800, 800-1000, 1000-1500, or greater than 1500 contiguous nucleotides. As described above, a portion or region can be 5′-of or 3′-of another portion or genetic element or sequence. A portion or region can also comprise a 5′-end portion or a 3′-end portion, meaning it comprises a 5′-end or a 3′-end, respectively, or it can be a portion or region that is between a 5′-portion and a 3′-portion. Although a circular oligonucleotide or polynucleotide does not have an end or an end portion, it can have portions or regions that are 5′-of or 3′-of another portion or region or sequence or genetic element, which permits orientation of one portion or region or sequence or genetic element with respect to another within the circular nucleic acid strand.

Discussions of nucleic acid structure and synthesis are simplified and clarified by adopting terms to name the two complementary strands of a nucleic acid duplex. Traditionally, the strand encoding the sequences used to produce proteins or structural RNAs is designated as the “plus” or “sense” strand, and its complement is designated as the “minus” or “anti-sense” strand. It is now known that in many cases, both strands are functional, and the assignment of the designation “plus” to one and “minus” to the other must then be arbitrary. Nevertheless, the terms are useful for designating the sequence orientation of nucleic acids or for designating the specific mRNA sequences transcribed and/or expressed in a cell.

Those with knowledge in the art will understand these terms in the context of nucleic acid chemistry and structure, particularly related to the 3′- and 5′-positions of sugar moieties of canonical nucleic acid nucleotides, and in the context of enzymatic synthesis of nucleic acids in a 5′-to-3′ direction. Since most descriptions of embodiments of the present invention are referring to single-stranded nucleic acids, in most cases herein the inventors use the terms “3′-” and “5′-of” to refer to the relative position or orientation of a particular nucleic acid sequence or genetic element encoded by a sequence that is located on the same nucleic acid strand. By way of example, a transcription promoter that is “3′-of the target sequence” refers to the position of a promoter relative to a target sequence on the same strand. Those with knowledge in the art will understand that, if a first nucleic acid sequence is 3′-of a second sequence within one strand, the complement of the first sequence will be 5′-of the complement of the second sequence in the complementary strand. The description of the invention will be understood with respect to the relative position and orientation of a sequence or genetic element within a particular strand, unless explicitly stated to the contrary.

The pentose sugar of the nucleic acid can be ribose, in which case, the nucleic acid or polynucleotide is referred to as “RNA,” or it can be 2′-deoxyribose, in which case, the nucleic acid or polynucleotide is referred to as “DNA.” Alternatively, the nucleic acid can be composed of both DNA and RNA mononucleotides. In both RNA and DNA, each pentose sugar is covalently linked to one of four common “nucleic acid bases” (each also referred to as a “base”). Three of the predominant naturally-occurring bases that are linked to the sugars (adenine, cytidine and guanine) are common for both DNA and RNA, while one base is different; DNA has the additional base thymine, while RNA has the additional base uridine. Those in the art commonly think of a small polynucleotide as an “oligonucleotide.” The term “oligonucleotide” as used herein is defined as a molecule comprised of two or more deoxyribonucleotides or ribonucleotides, preferably about 10 to 200 nucleotides, but there is no defined limit to the length of an oligonucleotide. The exact size will depend on many factors, which in turn depends on the ultimate function or use of the oligonucleotide.

Also, for a variety of reasons, a nucleic acid or polynucleotide of the invention may comprise one or more modified nucleic acid bases, sugar moieties, or internucleoside linkages. By way of example, some reasons for using nucleic acids or polynucleotides that contain modified bases, sugar moieties, or internucleoside linkages include, but are not limited to: (1) modification of the Tₘ; (2) changing the susceptibility of the polynucleotide to one or more nucleases; (3) providing a moiety for attachment of a label; (4) providing a label or a quencher for a label; or (5) providing a moiety, such as biotin, for attaching to another molecule which is in solution or bound to a surface.

In order to accomplish these or other goals, the invention does not limit the composition of the nucleic acids or polynucleotides of the invention including any target probes, detection probes, such as, but not limited to molecular beacons (U.S. Pat. Nos. 5,925,517 and 6,103,476 of Tyagi et al. and U.S. Pat. No. 6,461,817 of Alland et al., all of which are incorporated herein by reference); capture probes, oligonucleotides, or other nucleic acids used or detected in the assays or methods, so long as each said nucleic acid functions for its intended use. By way of example, but not of limitation, the nucleic acid bases in the mononucleotides may comprise guanine, adenine, uracil, thymine, or cytidine, or alternatively, one or more of the nucleic acid bases may comprise xanthine, allyamino-uracil, hypoxanthine, 2-aminoadenine, 6-methyl and other alkyl adenines, 2-propyl and other alkyl adenines, 5-halouracil, 5-halo cytosine, 5-propynyl uracil, 5-propynyl cytosine, 7-deazaadenine, 7-deazaguanine, 7-deaza-7-methyl-adenine, 7-deaza-7-methyl-guanine, 7-deaza-7-propynyl-adenine, 7-deaza-7-propynyl-guanine and other 7-deaza-7-alkyl or 7-aryl purines, N2-alkyl-guanine, N2-alkyl-2-amino-adenine, purine 6-aza uracil, 6-aza cytosine and 6-aza thymine, 5-uracil (pseudo uracil), 4-thiouracil, 8-halo adenine, 8-amino-adenine, 8-thiol adenine, 8-thiolalkyl adenines, 8-hydroxyl adenine and other 8-substituted adenines and 8-halo guanines, 8-amino-guanine, 8-thiol guanine, 8-thiolalkyl guanines, 8-hydroxyl guanine and other 8-substituted guanines, other aza and deaza uracils, other aza and deaza thymidines, other aza and deaza cytosine, aza and deaza adenines, aza and deaza guanines or 5-trifluoromethyl uracil and 5-trifluorocytosine. Still further, they may comprise a nucleic acid base that is derivatized with a biotin moiety, a digoxigenin moiety, a fluorescent or chemiluminescent moiety, a quenching moiety or some other moiety. The invention is not limited to the nucleic acid bases listed; this list is given to show the broad range of bases which may be used for a particular purpose in a method.

When a molecule comprising both a nucleic acid and a peptide nucleic acid (PNA) is used in the invention, modified bases can be used in one or both parts. For example, binding affinity can be increased by the use of certain modified bases in both the nucleotide subunits that make up the 2′-deoxyoligonucleotides of the invention and in the peptide nucleic acid subunits. Such modified bases may include 5-propynylpyrimidines, 6-azapyrimidines, and N-2, N-6 and O-6 substituted purines including 2-aminopropyladenine. Other modified pyrimidine and purine base are also expected to increase the binding affinity of macromolecules to a complementary strand of nucleic acid.

With respect to nucleic acids or polynucleotides of the invention, one or more of the sugar moieties can comprise ribose or 2′-deoxyribose, or alternatively, one or more of the sugar moieties can be some other sugar moiety, such as, but not limited to, 2′-fluoro-2′-deoxyribose or 2′-O-methyl-ribose, which provide resistance to some nucleases.

The internucleoside linkages of nucleic acids or polynucleotides of the invention can be phosphodiester linkages, or alternatively, one or more of the internucleoside linkages can comprise modified linkages, such as, but not limited to, phosphorothioate, phosphorodithioate, phosphoroselenate, or phosphorodiselenate linkages, which are resistant to some nucleases.

A variety of methods are known in the art for making nucleic acids having a particular sequence or that contain particular nucleic acid bases, sugars, internucleoside linkages, chemical moieties, and other compositions and characteristics. Any one or any combination of these methods can be used to make a nucleic acid, polynucleotide, or oligonucleotide for the present invention. The methods include, but are not limited to: (1) chemical synthesis (usually, but not always, using a nucleic acid synthesizer instrument); (2) post-synthesis chemical modification or derivatization; (3) cloning of a naturally occurring or synthetic nucleic acid in a nucleic acid cloning vector (e.g., see Sambrook, et al., Molecular Cloning: A Laboratory Approach Second Edition, 1989, Cold Spring Harbor Laboratory Press, Cold Spring Harbor, N.Y. and Sambrook and Russell, Molecular Cloning, A Laboratory Manual, Third Edition, 2001, Cold Spring Harbor Laboratory Press such as, but not limited to a plasmid, bacteriophage (e.g., M13 or lamba), phagemid, cosmid, fosmid, YAC, or BAC cloning vector, including vectors for producing single-stranded DNA; (4) primer extension using an enzyme with DNA template-dependent DNA polymerase activity, such as, but not limited to, Klenow, T4, T7, rBst, Taq, Tfl, or Tth DNA polymerases, including mutated, truncated (e.g., exo-minus), or chemically-modified forms of such enzymes; (5) PCR (e.g., see Dieffenbach, C. W., and Dveksler, eds., PCR Primer: A Laboratory Manual, 1995, Cold Spring Harbor Laboratory Press, Cold Spring Harbor, N.Y.); (6) reverse transcription (including both isothermal synthesis and RT-PCR) using an enzyme with reverse transcriptase activity, such as, but not limited to, reverse transcriptases derived from avian myeloblasosis virus (AMV), Maloney murine leukemia virus (MMLV), Bacillus stearothermophilus (rBst), or Thermus thermophilus (Tth); (7) in vitro transcription using an enzyme with RNA polymerase activity, such as, but not limited to, SP6, T3, or T7 RNA polymerase, Tth RNA polymerase, E. coli RNA polymerase, or SP6 or T7 R&DNA™ Polymerase (EPICENTRE Technologies, Madison, Wis., USA), or another enzyme; (8) use of restriction enzymes and/or modifying enzymes, including, but not limited to exo- or endonucleases, kinases, ligases, phosphatases, methylases, glycosylases, terminal transferases, including kits containing such modifying enzymes and other reagents for making particular modifications in nucleic acids; (9) use of polynucleotide phosphorylases to make new randomized nucleic acids; (10) other compositions, such as, but not limited to, a ribozyme ligase to join RNA molecules; and/or (11) any combination of any of the above or other techniques known in the art. Oligonucleotides and polynucleotides, including chimeric (i.e., composite) molecules and oligonucleotides with modified bases, sugars, or internucleoside linkages are commercially available (e.g., TriLink Biotechnologies, San Diego, Calif., USA or Integrated DNA Technologies, Coralville, Iowa).

The terms “hybridize” or “anneal” and “hybridization” or “annealing” refer to the formation of complexes between nucleotide sequences on opposite or complementary nucleic acid strands that are sufficiently complementary to form complexes via Watson-Crick base pairing. Where a target probe, primer, transcription substrate, or another oligonucleotide or polynucleotide “hybridizes” or “anneals” with target nucleic acid or a template or another oligonucleotide or polynucleotide, such complexes or “hybrids” are sufficiently stable to serve the function required for ligation, DNA polymerase extension, or other function for which it is intended.

With respect to nucleic acid synthesis, a “template” is a nucleic acid molecule that is being copied by a nucleic acid polymerase. The synthesized copy is complementary to the template. Both RNA and DNA are always synthesized in the 5′-to-3′ direction and the two strands of a nucleic acid duplex always are aligned so that the 5′-ends of the two strands are at opposite ends of the duplex (and, by necessity, so then are the 3′-ends). In general, DNA polymerases, including both DNA-dependent (i.e, having a DNA template) and RNA-dependent (i.e., having an RNA template, which enzyme is also called a “reverse transcriptase”) DNA polymerases, require a primer for synthesis of DNA. In general, RNA polymerases do not require a primer for RNA synthesis.

With respect to ligation, a “template” or a “ligation template” or a “template for ligation” is a nucleic acid molecule to which two or more complementary oligonucleotides, target probes, or other nucleic acids that are to be ligated anneal or hybridize prior to ligation, wherein the ends of said nucleic acid molecules that are to be ligated are adjacent to each other when annealed to the ligation template.

B. Samples, Analytes and Target Nucleic Acids of This Aspect of the Invention

A “sample” or a “biological sample” according to the present invention is used in its broadest sense. A sample is any specimen that is collected from or is associated with a biological or environmental source, or which comprises or contains biological material, whether in whole or in part, and whether living or dead. In some embodiments of the invention a sample can also be chemically synthesized or derived in the laboratory, rather than from a natural source.

Biological samples may be plant or animal, including human, fluid (e.g., blood or blood fractions, urine, saliva, sputum, cerebral spinal fluid, pleural fluid, milk, lymph, or semen), swabs (e.g., buccal or cervical swabs), solid (e.g., stool), microbial cultures (e.g., plate or liquid cultures of bacteria, fungi, parasites, protozoans, or viruses), or cells or tissue (e.g., fresh or paraffin-embedded tissue sections, hair follicles, mouse tail snips, leaves, or parts of human, animal, plant, microbial, viral, or other cells, tissues, organs or whole organisms, including subcellular fractions or cell extracts), as well as liquid and solid food and feed products and ingredients such as dairy items, vegetables, meat and meat by-products, and waste. Biological samples may be obtained from all of the various families of domestic plants or animals, as well as wild animals or plants.

Environmental samples include environmental material such as surface matter, soil, water, air, or industrial samples, as well as samples obtained from food and dairy processing instruments, apparatus, equipment, utensils, disposable and non-disposable items. These examples are not to be construed as limiting the sample types applicable to the present invention.

In short, a sample comprises a specimen from any source that contains or may contain a target nucleic acid.

A sample on which the assay method of the invention is carried out can be a raw specimen of biological material, such as serum or other body fluid, tissue culture medium or food material. More typically, the method is carried out on a sample that is a processed specimen, derived from a raw specimen by various treatments to remove materials that would interfere with detection of a target nucleic acid or an amplification product thereof. Methods for processing raw samples to obtain a sample more suitable for the assay methods of the invention are well known in the art.

An “analyte” means a substance or a part of a substance whose presence, concentration or amount in a sample is being determined in an assay. An analyte is sometimes referred to as a “target substance” or a “target molecule” or a “target analyte” of an assay. An analyte may also be referred to more specifically. In some embodiments, the present invention pertains to analytes that are target nucleic acid sequences that comprise or are in a “target nucleic acid” or a “target polynucleotide” or a “target oligonucleotide.” A composition, kit, or method of the invention can be used for an “analyte-specific reagent” to detect an analyte comprising a target nucleic acid sequence in a sample.

With respect to the present invention, an analyte is often associated with a biological entity that is present in a sample if and only if the analyte is present. Such biological entities include viroids (analyte is, e.g., a segment of a viroid nucleic acid sequence); viruses (analyte is, e.g., a sequence in the viral genome); other microorganisms (analyte is, e.g., a sequence in the genome or the RNA of the microorganism); abnormal cells, such as cancer cells (analyte is, e.g., a sequence in an oncogene); or an abnormal gene (analyte is, e.g., a sequence in a gene segment that includes the altered bases which render the gene abnormal or in a messenger RNA segment that includes altered bases as a result of having been transcribed from the abnormal gene).

However, in some embodiments of the invention an analyte can be chemically synthesized sequence or derived in the laboratory for a particular purpose, rather than from a natural source. By way of example, but not of limitation, the analyte can be a chemically synthesized oligonucleotide tag that comprises a target sequence that is covalently or non-covalently attached to an analyte-binding substance such as an antibody in order to indirectly detect another analyte in the sample which is bound by the analyte-binding substance. Alternatively, as discussed in greater detail later in the specification, the oligonucleotide tag that is attached or joined to an analyte-binding substance can be referred to as a “target sequence” or a “target sequence tag,” even though it is used to detect and/or quantify a protein, lipid, carbohydrate or another analyte by detecting the analyte-binding substance to which the target sequence is joined.

From the description of analytes, it is apparent that the present invention has widespread applicability, including in applications in which nucleic acid probe hybridization assays or immunoassays are often employed. Thus, among other applications, the invention is useful in diagnosing diseases in plants and animals, including humans; and in testing products, such as food, blood, and tissue cultures, for contaminants.

A “target” of the present invention is a biological organism or material that is the reason or basis for which a diagnostic assay is performed. By way of example, but not of limitation, an assay of the present invention may be performed to detect a target that is a virus which is indicative of a present disease or a risk of future disease (e.g., HIV which is believed to result in AIDS), or a target that is a gene which is indicative of antibiotic resistance (e.g., an antibiotic resistance gene in an infectious pathogenic bacterium), or a target that is a gene which, if absent, may be indicative of disease (e.g., a deletion in an essential gene). In developing assays according to the present invention, it is important to identify target analytes that yield assay results that are sufficiently specific, accurate, and sensitive to be meaningful related to the presence or condition of the target. A target analyte that is a sequence in a “target polynucleotide” or a “target nucleic acid” comprises at least one nucleic acid molecule or portion of at least one nucleic acid molecule, whether said molecule or molecules is or are DNA, RNA, or both DNA and RNA, and wherein each said molecule has, at least in part, a defined nucleotide sequence. The target polynucleotide may also have at least partial complementarity with other molecules which can be used in an assay, such as, but not limited to, capture probes. By way of example, in one embodiment, a capture probe for this purpose is complementary to a different region of a target nucleic acid than the target sequence and may have a moiety, such as a biotin moiety, that permits immobilization of the target nucleic acid on a surface, such as a surface to which streptavidin is attached.

The target polynucleotide may be single- or double-stranded. A target sequence of the present invention may be of any length. However, it must comprise a sequence of sufficient sequence specificity and length so as to be useful for its intended purpose. By way of example, but not of limitation, a target sequence that is to be detected using target sequence-complementary target probes must have a sequence of sufficient sequence specificity and length so as remain hybridized by said target probes under assay hybridization conditions wherein sequences that are not target sequences are not hybridized. A target sequence in a target polynucleotide having sufficient sequence specificity and length for an assay of the present invention may be identified, using methods known to those skilled in the art, by comparison and analysis of nucleic acid sequences known for a target and for other sequences which may be present in the sample. For example, sequences for nucleic acids of many viruses, bacteria, humans (e.g., for genes and messenger RNA), and many other biological organisms can be searched using public or private databases, and sequence comparisons, folded structures, and hybridization melting temperatures (i.e., Tₘ's) may be obtained using computer software known to those knowledgeable in the art.

A method of the present invention can be carried out on nucleic acid from a variety of sources, including unpurified nucleic acids, or nucleic acids purified using any appropriate method in the art, such as, but not limited to, various “spin” columns, cationic membranes and filters, or salt precipitation techniques, for which a wide variety of products are commercially available (e.g., MasterPure™ DNA & RNA Purification Kits from EPICENTRE Technologies, Madison, Wis., USA). Methods of the present invention can also be carried out on nucleic acids isolated from viroids, viruses or cells of a specimen and deposited onto solid supports as described by Gillespie and Spiegelman (J. Mol. Biol. 12: 829-842, 1965), including solid supports on dipsticks and the inside walls of microtiter plate wells. The method can also be carried out with nucleic acid isolated from specimens and deposited on solid support by “dot” blotting (Kafatos, et al., Nucl. Acids Res., 7: 1541-1552, 1979); White, and Bancroft, J. Biol. Chem., 257: 8569-8572, 1982); Southern blotting (Southern, E., J. Mol. Biol., 98: 503-517, 1975); “northern” blotting (Thomas, Proc. Natl. Acad. Sci. USA, 77: 5201-5205, 1980); and electroblotting (Stellwag, and Dahlberg, Nucl. Acids Res., 8: 299-317, 1980). The method can also be carried out for nucleic acids spotted on membranes, on slides, or on chips as arrays or microarrays. Nucleic acid of specimens can also be assayed by the method of the present invention applied to water phase hybridization (Britten, and Kohne, Science, 161: 527-540, 1968) and water/organic interphase hybridizations (Kohne, et al., Biochemistry, 16: 5329-5341, 1977). Water/organic interphase hybridizations have the advantage of proceeding with very rapid kinetics but are not suitable when an organic phase-soluble linking moiety, such as biotin, is joined to the nucleic acid affinity molecule.

The methods of the present invention can also be carried out on amplification products obtained by amplification of a naturally occurring target nucleic acid, provided that the target sequence in the target nucleic acid is amplified by the method used only if the target nucleic acid is present in the sample. Suitable amplification methods include, but are not limited to, PCR, RT-PCR, NASBA, TMA, 3SR, LCR, LLA, SDA (e.g., Walker et al., Nucleic Acids Res. 20:1691-1696, 1992), RCA, Multiple Displacement Amplification (Molecular Staging), ICAN™ or UCAN™ (TAKARA), Loop-AMP (EIKEN), and SPIA™ or Ribo-SPIA™ (NuGEN Technologies). There are various reasons for using a nucleic acid that is a product of another amplification method as a target nucleic acid for an assay of the present invention, such as, but not limited to, for obtaining more sensitive detection of targets, greater specificity, or to decrease the time required to obtain an assay result.

Nucleic acid used as a template for amplification is isolated from cells contained in the biological sample, according to standard methodologies (Sambrook et al., In: Molecular Cloning: A Laboratory Manual 2 rev.ed., Cold Spring Harbor: Cold Spring Harbor Laboratory Press, 1989). The nucleic acid may be genomic DNA or fractionated or whole cell RNA. Where RNA is used, it may be desired to convert the RNA to a complementary DNA. In one embodiment, the RNA is whole cell RNA and is used directly as the template for amplification.

Pairs of primers that selectively hybridize to nucleic acids are contacted with the isolated nucleic acid under conditions that permit selective hybridization. The term “primer,” as defined herein, is meant to encompass any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label, such as real-time analysis with SYBR® Green dye, or even via a system using electrical or thermal impulse signals (Affymax technology).

A number of template dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159. A reverse transcriptase PCR amplification procedure may be performed in order to quantify the amount of mRNA amplified. Alternative methods for reverse transcription utilize thermostable, RNA-dependent DNA polymerases. These methods are described in WO 90/07641, filed Dec. 21, 1990. Polymerase chain reaction methodologies are well known in the art.

The methods of the invention can also be carried out on nucleic acids isolated from specimens and deposited onto solid supports by dot-blotting, or by adsorption onto walls of microtiter plate wells or solid support materials on dipsticks, on membranes, on slides, or on chips as arrays or microarrays. The amplified target-complementary sequences of target probes of the invention can also be hybridized to oligonucleotides or nucleic acids attached to or deposited on slides, chips or other surfaces, such as, but not limited to arrays or microarrays, for detection and identification.

Still further, the methods of the invention are applicable to detecting target sequences in cellular nucleic acids in whole cells, including single cells, from a specimen, such as a fixed or paraffin-embedded section, or from microorganisms immobilized on a solid support, such as replica-plated bacteria or yeast. In some embodiments, the methods of the invention can be used to amplify and/or detect target nucleic acid sequences in living cells.

The invention is also not limited to detection of analytes comprising a target nucleic acid. The present invention provides assays, methods, compositions and kits for detection and quantification of an analyte of any type in a sample.

C. Target Sequences Comprising Target Nucleic Acids in a Sample or a Target Sequence Tag Joined to an Analyte-Binding Substance

The term “target nucleic acid sequence” or “target sequence” refers to the particular nucleotide sequence of the target nucleic acid(s) that is/are to be detected. A “target sequence” comprises one or more sequences within one or more target nucleic acids, which target nucleic acid can be naturally occurring in a sample or a target sequence tag that is joined or attached to an analyte-binding substance.

The target nucleic acid may be either single-stranded or double-stranded and may include other sequences besides the target sequence. A target nucleic acid is sometimes referred to more specifically by the type of nucleic acid. By way of example, but not of limitation, a target nucleic acid can be a “target RNA” or an “RNA target,” or a “target mRNA,” or a “target DNA” or a “DNA target.” Similarly, the target sequence can be referred to as “a target RNA sequence” or an “RNA target sequence”, or as a “target mRNA sequence” or a “target DNA sequence,” or the like. In some embodiments, the target sequence comprises one or more entire target nucleic acids. In other embodiments, which are more common, the target sequence comprises only a portion of one or more nucleic acid molecules. The term “target sequence” sometimes is also used to refer to the particular target-complementary nucleotide sequences that is/are present in the target-complementary “target probes” used in a method or assay of the invention, but more preferably, these sequences are referred to as “target-complementary sequences.” The term “target sequence” refers only to that portion of the sequence of a target nucleic acid for which a complementary sequence is present in a target probe of the invention. In some embodiments of the invention, multiple target probes are used, including target probe sets that are complementary to other target sequences in a target nucleic acid, which other target sequences can be on the same or the opposite nucleic acid strand of the same target nucleic acid, or on another target nucleic acid in the sample or joined to another analyte-binding substance. In some of those embodiments, a transcription promoter is present in a target probe that is complementary to only one strand of the target sequence, and in other embodiments, a transcription promoter is present in two different target probes—one that is complementary to a target sequence on one strand, and the other that is complementary to a complementary target sequence on the other strand, wherein the transcription promoters can be the same or different in each case. In most embodiments of the invention, the target sequence in a method or assay of the invention will be a known sequence or one of a small number of known sequences, such as, but not limited to one or a small number of sequences comprising known specific mutations or single nucleotide polymorphisms (SNPs), or one of known sequences that are specific for and identify a particular organism or group of closely-related organisms. In some embodiments, wherein a method or assay of the invention is used to distinguish between two or more target sequences that differ by a single nucleotide, we sometimes refer to the specific nucleotide that differs between otherwise identical sequences as a “target nucleotide.” A “target nucleotide” is part of a target sequence and comprises the nucleotide position that differs between “wild-type” or “normal” alleles and single-base “mutant” alleles, or the nucleotide that differs between different “wild-type” alleles that comprise different single-nucleotide polymorphisms (SNPs) for a particular nucleotide position in a target nucleic acid.

A target nucleic acid of the present invention comprising a target sequence to be detected and/or quantified includes nucleic acids from any source in purified or unpurified form. As discussed in greater detail herein above, target nucleic acids can be any DNA, including, but not limited to, dsDNA and ssDNA, such as mitochondrial DNA, chloroplast DNA, chromosomes, plasmids or other episomes, the genomes of bacteria, yeasts, viruses, viroids, mycoplasma, molds, or other microorganisms, or genomes of fungi, plants, animals, or humans, or target nucleic acids can be any RNA, including, but not limited to, tRNA, mRNA, rRNA, mitochondrial RNA, chloroplast RNA, or target nucleic acids can be mixtures of DNA and RNA, including, but not limited to, mixtures of the above nucleic acids or fragments thereof or DNA-RNA hybrids. The target nucleic acid can be only a minor fraction of a complex mixture such as a biological sample and can be obtained from various biological materials by procedures known in the art. As discussed herein above, methods for purification of a target nucleic, if further purification is necessary, are also known in the art.

An initial step prior to amplification of a target nucleic acid sequence is rendering the target nucleic acid single-stranded. If the target nucleic acid is a double-stranded DNA (dsDNA), the initial step is target denaturation. The denaturation step may be thermal denaturation or any other method known in the art, such as alkali treatment. Thus, in some embodiments of the invention in which the target nucleic acid in a sample is DNA, the ssDNA target sequence comprises either ssDNA that is present in a biological sample or ssDNA that is obtained by denaturation of dsDNA in the sample.

In other embodiments, the ssDNA target sequence comprises ssDNA that is obtained as a result of a “primer extension reaction,” meaning an in vitro or in vivo DNA polymerization reaction using either ssDNA or denatured dsDNA that is present in the sample as a template and an oligonucleotide as a primer under DNA polymerization reaction conditions. In some embodiments the target nucleic acid in the sample or the primer extension product, or both, are made into smaller DNA fragments by methods known in the art in order to generate a DNA target sequence for use in the methods of the invention.

If a target nucleic acid is RNA, the initial step may be the synthesis of a single-stranded cDNA. Techniques for the synthesis of cDNA from RNA are known in the art. Thus, in some embodiments of the invention, which are preferred embodiments, the ssDNA target sequence comprises first-strand cDNA obtained by reverse transcription of the RNA target, meaning an in vitro reaction that utilizes an RNA present in a sample as a template and a nucleic acid oligonucleotide that is complementary to at least a portion of a sequence of the RNA template as a primer in order to synthesize ssDNA using an RNA-dependent DNA polymerase (i.e., reverse transcriptase) under reaction conditions. In some embodiments, a first-strand cDNA for use in methods of the invention is synthesized in situ in cells or tissue in a tissue section using methods such as those described in U.S. Pat. Nos. 5,168,038; 5,021,335; and 5,514,545, which are incorporated herein by reference.

D. Target Probes of the Invention: Simple Target Probes; Promoter Target Probes; Signal Target Probes; Monpartite Target Probes; Bipartite Target Probes

A “target probe” of the present invention is a linear single-stranded oligonucleotide that comprises at least one sequence that is “a target-complementary sequence,” meaning a sequence that is complementary to a portion of a target sequence comprising a target nucleic acid or a target sequence tag, and wherein the target probe is used in an assay or method of the invention. In general, target probes comprise deoxyribonucleotides having canonical nucleic acid bases and internucleoside linkages, although modified sugars, bases or internucleoside linkages can be used for a particular purpose as discussed elsewhere herein. The size and nucleotide composition of a target-complementary sequence of a target probe can vary. However, the target-complementary sequences of all target probes of the invention must be of sufficient length and nucleotide composition so as to anneal with specificity to a complementary target sequence with which it is perfectly based-paired under the conditions used in the assay or method for annealing of target probes to the target sequence and for ligation of the target probes that are annealed to the target sequence, under which conditions, target probes that are not complementary to the target sequence do not remain annealed and, if not perfectly basepaired at the ligation junction, do not ligate. In order to meet these conditions, those with knowledge in the art will understand that the length of a target-complementary sequence can vary based in part on its sequence and on the Tₘ of that sequence, and on the temperature and other reaction conditions that are used for annealing of the target probes and ligation of the target probes on the target sequence.

A target-complementary sequence of a target probe will comprise at least four nucleotides if the target-complementary sequences are annealed to the target sequence and ligated at a temperature that is less than or equal to about 30° C., or at least about eight nucleotides if the target-complementary sequences are annealed to the target sequence and ligated at a temperature that is greater than about 30° C. However, in general, a target-complementary sequence of only 4-8 nucleotides is not sufficient to provide the desired nucleotide specificity in an assay or method of the invention. Preferably, a target-complementary sequence of a target probe that is complementary to the 5′-end or to the 3′-end of the target sequence comprises about 10 to about 100 nucleotides, and most preferably, about 15 to about 50 nucleotides. However, based on this description of the invention, those with knowledge in the art will know how to empirically determine the optimal lengths of target-complementary sequences for target probes for particular target sequences, and under the particular conditions, which conditions can vary with respect to factors such as but not limited to temperature, ionic strength, concentration of co-solvents such as but not limited to betaine, or other factors.

Further, the length of one target-complementary sequence of a bipartite target probe can be different than the other. Also, the length of a target-complementary sequence of one monopartite target probe can be different than the lengths of target-complementary sequences of other monopartite target probes used in the assay or method.

In general, the sequence of a target probe that is complementary to the 3′-end of a target sequence is designed to be longer than the sequence of a target probe that is joined to a sense promoter sequence and that is complementary to the 5′-end of the target sequence, although the sequence of a target probe that is complementary to the 3′-end of the target sequence need not be longer, and can be about the same size or even shorter than the sequence that is complementary to the 5′-end of the target sequence. However, by using a target probe with a longer sequence that is complementary to the 3′-end of the target sequence, the hybridization complex between that target probe and the target sequence will be more stable so that the ability to form a ligation junction will be more dependent on the annealing of the target-complementary sequence that is joined to the sense promoter sequence and that anneals to the 5′-end of the target sequence.

It is preferable that the length of the target-complementary sequence that is joined to the sense promoter sequence comprises a sufficient number of nucleotides so as anneal to the 5′-end of the target sequence with specificity under the conditions of the assay or method, but is optimized so that transcription of said target-complementary sequence is minimized unless and until it is ligated to another target-complementary sequence that is adjacently annealed on the target sequence. Without being bound by theory, it appears that the optimal length of the target-complementary sequence that is joined to the sense promoter sequence can vary for different RNA polymerases. By way of example, but not of limitation, it appears that the optimal length of a target-complementary sequence that is joined to a sense T7 RNAP promoter sequence will be the shortest sequence that anneals to the target sequence with specificity. This appears to be due to the fact that T7 RNAP, which has RNA:DNA hybrid unwinding activity, displaces both long and very short transcripts from the template and binding to the promoter and initiation of transcription appear to be the rate limiting steps for transcription. On the other hand, N4 mini-vRNAP does not have RNA:DNA hybrid unwinding activity and EcoSSB Protein appears to be responsible for displacing the RNA transcript from the template strand. The amount of EcoSSB-activated displacement appears to vary with the total length of the template strand and the length of the transcript (Davidova, E. and Rothman-Denes, Proc. Natl. Acad. Sci. USA, 100: 9250-9255, 2003). Therefore, if a target probe comprises an N4 vRNAP promoter, the length of the target-complementary sequence that is joined to the N4 promoter sequence is designed, based on the information of Davidova et al., so that a transcript made by an N4 mini-vRNAP will either not be displaced or at least, EcoSSB-activated displacement is minimized from the target-complementary sequence unless and until this sequence is ligated to an adjacent target-complementary sequence that is annealed to the target sequence to make a transcription substrate of the invention.

If there is a gap between target-complementary sequences of a bipartite target probe or between target-complementary sequences of a promoter target probe and another monopartite target probe when annealed to the 5′-end and the 3′-end, respectively, of the target sequence, and one or more simple target probes is used in the assay or method to fill the gap, then the simple target probe(s) that is/are used to fill the gap can be of any length so long as they provide suitable ligation junctions for joining to the target-complementary sequences that are annealed to the 3′- and 5′-ends of the target sequence.

One type of target probe of the invention, which is called a “simple target probe,” comprises a linear single-stranded oligonucleotide comprising only a sequence that is complementary to one continuous portion of a target sequence.

Another embodiment of a target probe of the invention is a “promoter target probe.” A “promoter target probe” comprises a 5′-end portion that is complementary to the most 5′-portion of a target sequence. The 3′-end of the target-complementary portion is joined to the 5′-end of a sense promoter sequence which, upon complexing with an anti-sense promoter sequence, serves as a functional transcription promoter for an RNA polymerase that synthesizes RNA under transcription conditions using said transcription promoter and ssDNA that is 5′-of said promoter (with respect to the same strand) as a template. Optionally, a promoter target probe can also comprise other “optional sequences” that do not comprise a target-complementary sequence or a promoter sequence, which optional sequences, if present, are 3′-of said promoter sequence in said promoter target probe. Such optional sequences in said promoter target probe can serve other functions in a method or assay of the invention.

In embodiments of the invention that result in linear transcription substrates rather than circular transcription substrates, as discussed below, another embodiment of a target probe of the invention that can be used, but which is optional, is a “signal target probe.” A “signal target probe” comprises a 3′-portion and a 5′-portion, wherein, the 3′-end portion of said signal probe comprises a sequence that is complementary to the most 3′-portion of a target sequence, and said 5′-portion comprises a “signal sequence,” wherein said signal sequence comprises a sequence that is detectable in some way, directly or indirectly, following transcription of said signal sequence that is joined, in the presence of a target sequence, to a target-complementary sequence and a sense promoter sequence during an assay or method of the present invention. Upon annealing of an anti-sense promoter oligo to the sense promoter sequence, in vitro transcription of the signal sequence results in synthesis of RNA that is complementary to said signal sequence, which in turn is detectable in some way (depending on what the signal sequence encodes) in an assay or method of the invention. A signal target probe can also comprise other “optional sequences” that do not comprise the signal sequence, which sequences can serve another function in a method or assay of the invention, or they can have no function, other than to connect the signal sequence to one or more other sequences.

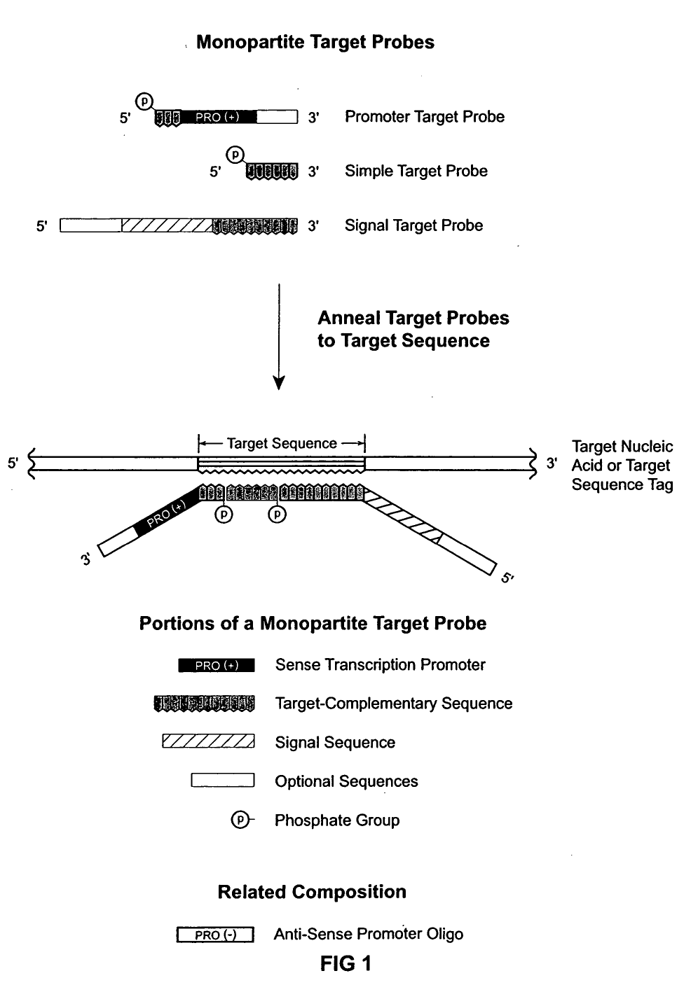

“Monopartite target probes” of the present invention are target probes that comprise only one sequence that is complementary to one portion of a target sequence. The target-complementary sequence in a monopartite target probe is not interrupted by any other sequence that is not complementary to the target sequence. Promoter target probes and signal target probes are monopartite target probes that are used to generate linear transcription substrates in some embodiments of assays and methods of the invention. Simple target probes are monopartite target probes that can be used in embodiments of the invention to generate either linear transcription substrates or circular transcription substrates, as discussed herein below. Simple target probes are monopartite target probes that are used in embodiments of the invention in order to fill at least a portion of a gap region between target-complementary sequences of other target probes that are not contiguous when annealed to a target sequence. For example, one or more simple target probes can be used in methods and assays of the invention that generate a linear transcription substrate by annealing to a target sequence between the sequences of the target sequence to which the target-complementary sequences of a promoter target probe and a signal target probe anneal. FIG. 1 illustrates monopartite target probes and shows one embodiment of how monopartite target probes are oriented when annealed to a target sequence. In still other embodiments of the invention, the target-complementary sequences of the promoter target probe and the signal target probe are contiguous or adjacent when they are annealed to a target sequence and a simple target probe is not used. In still another embodiment, the target-complementary sequences of the promoter target probe and the signal target probe are not contiguous when they are annealed to a target sequence, but rather than using a simple target probe to anneal to the gap region on the target sequence between the the target-complementary sequences of the promoter target probe and the signal target probe, the gap is “filled” by DNA polymerase extension from the 3′-end of the signal target probe.

One or more simple target probes can also be used in embodiments of methods and assays of the invention that generate a circular transcription substrate, in which case, the simple target probe(s) anneal to a target sequence between the regions of the target sequence to which the target-complementary sequences at the ends of a bipartite target probe anneal.

Thus, other embodiments of the invention, which are preferred embodiments, use a bipartite target probe and generate a circular transcription substrate. A “bipartite target probe” is referred to as “bipartite” because it comprises two different target-complementary sequences, each of which is complementary to a different portion of a target sequence, which target-complementary sequences are separated within the bipartite target probe by other sequences that are not complementary to the target sequence. Thus, the target-complementary sequences in a bipartite target probe are in two parts or “bipartite.” A bipartite target probe comprises a ssDNA that has a 5′-end that resembles a promoter target probe and a 3′-end that resembles either a simple target probe or a signal target probe. Thus, the 5′-end of a bipartite target probe has a sequence that is complementary to the most 5′-portion of a target sequence and, then on the same strand, 3′-of the target-complementary sequence, a sense transcription promoter sequence which, upon complexing with an anti-sense promoter sequence, can bind an RNA polymerase that can make a transcription product under transcription conditions using single-stranded DNA that is joined 5′-of said promoter as a template. The 3′-end of a bipartite target probe comprises a sequence that is complementary to the most 3′-end portion of said target sequence. If it is used, a signal sequence can be 5′-of the target-complementary sequence at the 3′-end of a bipartite target probe, although a signal sequence does not need to be contiguous with or immediately adjacent to the target-complementary sequence at the 3′-end of a bipartite target probe. Other sequences that can have other functions or that have no function other than to join the two sequences can be between a target-complementary sequence at the 3′-end and a signal sequence of a bipartite target probe. The target-complementary sequences of a bipartite target probe need not be contiguous or immediately adjacent when they are annealed to a target sequence. If the bipartite target-complementary sequences are not contiguous or immediately adjacent when they are annealed to a target sequence, then one or more simple target probes that are complementary to the portions of the target sequence between the target-complementary sequences of the bipartite target probe can be used in some embodiments of methods or assays of the invention.

Alternatively, in other embodiments of the invention in which the bipartite target-complementary sequences are not contiguous or immediately adjacent when they are annealed to a target sequence, a DNA polymerase can be used to “fill in” where there is no target probe annealed to the target sequence by primer-extending from the 3′-hydroxyl end of a bipartite target probe that is annealed to a target sequence using the target sequence as a template.

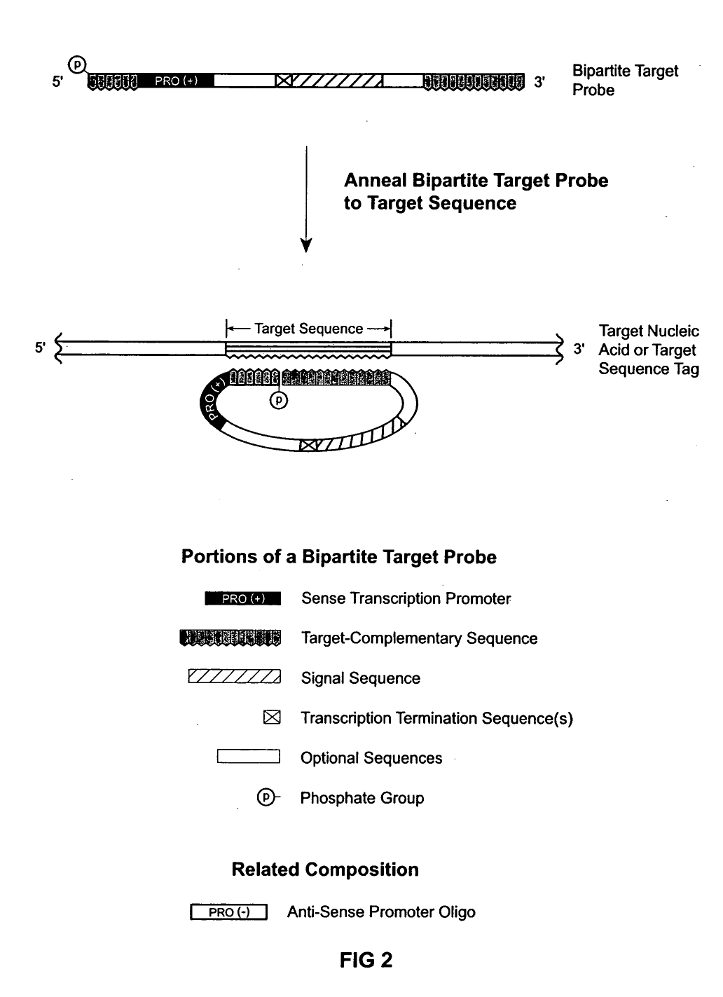

FIG. 2 illustrates a bipartite target probe of the invention and shows how different sequence portions of a bipartite target probe are oriented with respect to each other when said bipartite target probe is free in solution and when it is annealed to a target sequence. The embodiment illustrated in FIG. 2 shows a bipartite target probe comprising target-complementary sequences at each end that are contiguous or adjacent when annealed to a target sequence. As discussed above, the invention also comprises other embodiments of bipartite target probes wherein the target-complementary sequences at each end that are not contiguous or adjacent when annealed to a target sequence. In those embodiments, the “gap” between the target-complementary sequences of the bipartite target probe can be “filled” using one or more simple target probes or by DNA polymerase extension from the 3′-end of the bipartite target probe using the target sequence as a template.

In general, all target probes of the invention, including both monopartite and bipartite target probes that are joined with a ligase in a method or assay of the invention, will have a phosphate group at their 5′-end and a hydroxyl group at their 3′-end. The 5′-ends of target probes that are not joined with a ligase to the 3′-end of another target probe in a method or assay of the invention do not have a phosphate group on their 5′-ends. The only exceptions will be in those embodiments that use another joining method, such as, but not limited to a chemical joining method or a topoisomerase-mediated joining method.

In some embodiments of the invention, such as, but not limited to the embodiment illustrated in FIG. 9, secondary or additional amplification of a target sequence and/or a signal sequence is obtained by using a “second target probe,” which second target probe can comprise either: (i) “second monopartite target probes” comprising a “second promoter target probe” and either a “second signal target probe,” if a signal sequence is present, or a “second simple target probe,” and one or more additional “second simple target probes; or (ii) a “second bipartite target probe.” If a second target probe is used, then the target probes that are complementary to the target sequence are referred to as “first target probes.” A second target probe is generally identical to a first target probe except with respect to the target-complementary sequence of said target probe. Thus, the sequence at the 5′-end of a second promoter target probe or at the 5′-end of a second bipartite target probe, rather than being complementary to a target sequence, is complementary to the target-complementary sequence at the 3′-end of the first signal target probe or to the target-complementary sequence at the 3′-end of the bipartite target probe, respectively. Similarly, the sequence at the 3′-end of a second signal target probe or at the 3′-end of a second bipartite target probe, rather than being complementary to a target sequence, is complementary to the target-complementary sequence at the 5′-end of the first promoter target probe or to the target-complementary sequence at the 5′-end of the bipartite target probe, respectively. A second simple target probe, rather than being complementary to a target sequence, is complementary to a first target probe. A second target probe can also be referred to as an “target amplification probe,” which can comprise either: (i) “monopartite target amplification probes” comprising a “promoter target amplification probe” and either a “signal target amplification probe,” if a signal sequence is present, or a “simple target amplification probe,” and one or more additional “simple target amplification probes; or

(ii) a “bipartite target amplification probe.”

E. Design of Target Probes of the Invention for Detection of Mutations, Including Single Nucleotide Polymorphisms (SNP's)

In embodiments of a method or assay of the invention to distinguish between two or more target sequences that differ by a single nucleotide, wherein the specific nucleotide that differs between otherwise identical sequences is referred to as a “target nucleotide,” the target probes used in said assay or method are designed in order to be able to distinguish said target nucleotide(s). In preferred embodiments of assays and methods to detect a single-nucleotide difference in a target sequence, the nucleotide of a target probe of the invention that is complementary to the target nucleotide comprises either the 5′-end of a promoter target probe if the assay or method generates a linear transcription substrate, or the nucleotide at the 5′-end of a bipartite target probe if the assay or method generates a cirular transcription substrate. Then, if a target nucleotide is present in a target sequence in a sample, the complementary nucleotide at the 5′-end of the respective promoter target probe or the bipartite target probe will anneal thereto and will be ligated, respectively, either to the 3′-end of an adjacently-annealed monopartite target probe or to an adjacently-annealed 3′-end of the bipartite target probe. If the 5′-end of the promoter target probe or the 5′-end of the bipartite target probe is not complementary to the target nucleotide, it will not anneal thereto, and said 5′-end will not be ligated to the 3′-end of an adjacently-annealed monopartite target probe or the 3′-end of the bipartite target probe, respectively, during the ligation process. That is, the non-complementarity of the 5′-end of a target probe with a target nucleotide in a target sequence, when the target probes of an assay or method are annealed to the target sequence prevents ligation of said 5′-end with a 3′-hydroxyl end, so that a transcription substrate is not formed. Although the preferred nucleotide of a target probe of the invention that is complementary to the target nucleotide comprises either the 5′-end of a promoter target probe if the assay or method generates a linear transcription substrate, or the nucleotide at the 5′-end of a bipartite target probe if the assay or method generates a cirular transcription substrate, the invention also comprises other embodiments of target probes in which the nucleotide that is complementary to the target nucleotide comprises a different nucleotide in a monopartite or bipartite target probe. Thus, in embodiments of assays or methods using monopartite target probes in which there is no “gap” between the sites on the target sequence to which a promoter target probe and a signal target probe if a signal sequence is present, or a simple target probe if a signal sequence is not present, then the nucleotide that is complementary to the target nucleotide can comprise either the 3′-end of the signal target probe if a signal sequence is present, or the 3′-end of the simple target probe if a signal sequence is not used. Similarly, in embodiments of assays or methods using a bipartite target probe in which there is no “gap” between the sites on the target sequence to which the 5′-end and the 3′-end of said bipartite target probe anneal, then the nucleotide that is complementary to the target nucleotide can comprise the 3′-end of said bipartite target probe. In embodiments of assays or methods using monopartite target probes in which there is a gap between the sites on the target sequence to which a promoter target probe and a signal target probe if a signal sequence is present, or a simple target probe if a signal sequence is not present, wherein one or more simple target probes are used to “fill the gap,” then the nucleotide that is complementary to the target nucleotide can comprise a nucleotide at either the 3′-end or the 5′-end of one of said simple target probes that is used to fill the gap. Preferably, the nucleotide that is complementary to the target nucleotide comprises a nucleotide at either the 3′-end or the 5′-end of a simple target probe used to fill the gap that anneals to the target sequence adjacent to the promoter target probe, and most preferably, the nucleotide that is complementary to the target nucleotide comprises a nucleotide at the 3′-end of said simple target probe. In embodiments of assays or methods using bipartite target probes in which there is a gap between the sites on the target sequence to which the ends of the bipartite target probe anneal, wherein one or more simple target probes are used to fill the gap, then the nucleotide that is complementary to the target nucleotide can comprise a nucleotide at either the 3′-end or the 5′-end of one of said simple target probes that is used to fill the gap. Preferably, the nucleotide that is complementary to the target nucleotide comprises a nucleotide at either the 3′-end or the 5′-end of a simple target probes used to fill the gap that anneals to the target sequence adjacent to the 5′-end of said bipartite target probe, and most preferably, the nucleotide that is complementary to the target nucleotide comprises a nucleotide at the 3′-end of said simple target probe. It will be understood by those with knowledge in the art that one or more 5′-terminal or 3′-terminal nucleotide positions of a target probe used in an assay or method to detect a particular target nucleotide in a target sequence may not comprise a sequence that will anneal with specificity to said target sequence, such as, when said target nucleotide is part of a target sequence that has a low Tₘ with respect to said target-complementary sequence of said target probe. In such cases, those with knowledge in the art will know how to evaluate and, without undue experimentation, how to choose which of those possible 5′-terminal and 3′-terminal nucleotide positions of all of the target probes used in said method or assay comprises the “best nucleotide” to be complementary to said target nucleotide, wherein said best nucleotide results in the greatest specificity and sensitivity in said assay or method.

F. Signal Sequences in Signal Target Probes or Bipartite Target Probes of the Invention

A method or assay of the invention does not need to use a signal sequence. The use of a signal sequence or a signal target probe in a method or assay of the invention is optional. If a signal sequence is used in an embodiment, the invention is not limited with respect to particular signal sequences that can be used in signal target probes or bipartite target probes of the invention. A signal sequence can comprise any sequence that generates a detectable signal or that enables sensitive and specific detection, whether directly or indirectly, of the generation of an RNA transcription product encoded by the signal sequence. Preferably, a signal sequence encodes an RNA product that results in additional amplification or more sensitive detection.

By way of example, but not of limitation, one signal sequence that can be used in a signal target probe or a bipartite target probe of the present invention is a sequence that encodes a substrate for a replicase, such as, but not limited to, Q-beta replicase or a partial or interrupted sequence for a substrate for a replicase, such as, but not limited to, Q-beta replicase. Q-beta replicase substrates and methods that can be used for making and using signal sequences that encode a partial or interrupted replicase substrate for signal target probes are described in U.S. Pat. No. 6,562,575, incorporated herein by reference. A complete sequence for a replicase substrate is preferred in a signal probe of the present invention, but a sequence of a partial or interrupted replicase substrate is used in embodiments that require reduced background signal (or “noise”) and greater sensitivity. If the time for appearance of a signal in an assay or method is shortened by amplifying the amount of transcription product using other methods described herein, it is less likely that a sequence for a partial or interrupted replicase substrate, rather than for a complete replicase substrate, is needed to obtain a good signal to noise ratio in the assay or method. Once an RNA that is a substrate for Q-beta replicase is synthesized, incubation of said RNA substrate with Q-beta replicase results in replication of the substrate, thereby resulting in additional amplification of the signal and more sensitive, though indirect, detection of the presence of a target sequence.

A “replicase” according to the invention is an enzyme that catalyzes exponential synthesis (i.e., “replication”) of an RNA substrate. The replicase can be from any source for which a suitable exponentially replicatable substrate can be obtained for use in the invention. Preferably, the replicase is an RNA-directed RNA polymerase. In preferred embodiments, the replicase is a bacteriophage replicase, such as Q-beta replicase, MS2 replicase, or SP replicase. In the most preferred embodiment, the replicase is Q-beta replicase. In other preferred embodiments, the replicase is isolated from eucaryotic cells infected with a virus, such as, but not limited to, cells infected with brome mosaic virus, cowpea mosaic virus, cucumber mosaic virus, or polio virus. In another embodiment, the replicase is a DNA-directed RNA polymerase, in which case, a T7-like RNA polymerase (as defined in U.S. Pat. No. 4,952,496) is preferred, and T7 RNA polymerase (Konarska, M. M., and Sharp, P. A., Cell, 63: 609-618, 1990) is most preferred. The replicase can be prepared from cells containing a virus or from cells expressing a gene from a bacteriophage or a eukaryotic virus cloned into a plasmid or other vector.

If Q-beta replicase is used, replication of a Q-beta replicase substrate can be carried out substantially according to the protocol of Kramer et al. (J. Mol. Biol., 89: 719-736, 1974). Briefly, an RNA substrate is incubated at 37° C. in a reaction mixture containing about 20-50 micrograms of Q-beta replicase per ml, 40-100 mM Tris-HCl (pH 7-8), about 10-12 mM MgCl₂, and about 200-400 micromolar each of ATP, CTP, UTP and GTP. If desired, one of the NTPs can be labeled with a fluorescent or other dye, or the replication products can be detected using another method, such as but not limited to by detection of fluorescence that results from intercalation of a dye such as ethidium bromide.

In embodiments that use Q-beta replicase, it is preferred that the sequence of the recombinant substrate or template be derived from the sequence of an RNA in the following group: midivariant RNA (MDV-1 RNA), microvariant RNA, nanovariant RNA, CT RNA, RQ135 RNA, RQ120 RNA, and other variants or Q-beta RNA, which are known in the art. Once a substrate for a replicase is identified, improved substrates can be obtained, if desired, by serial transfer and selection of higher yielding products from successive reactions, including prolonged reactions. Further, improved substrates can be obtained by random or site-directed modification of a known substrate, followed by serial transfer and selection to select new substrates that result in greater incorporation of UTP during replication.

Another signal sequence that can be used is an expressable gene for an enzyme that has a substrate that results in a colored or fluorescent or otherwise detectable product. By way of example, but not of limitation, a gene for a green fluorescent protein (GFP) can be used. In that case, in vitro transcription of a transcription substrate generated by target-dependent annealing and ligation of target probes results in an RNA transcript that encodes a GFP. In the presence of an in vitro translation system, a detectable GFP is synthesized. There are many other genes that encode enzymes that can be used to generate detectable signals following coupled or stepwise in vitro transcription and translation. By way of example, but not of limitation, signal sequences comprising genes for phosphatases or beta-galactosidases can be used, together with a suitable substrate that generates a colored, fluorescent or chemiluminescent product. A large number of enzymes and coenzymes, as well as enzyme combinations that are useful in a signal producing system are indicated In U.S. Pat. No. 4,275,149 and U.S. Pat. No. 4,318,980, which disclosures are incorporated herein in their entirety by reference. Still further, a signal sequence can comprise a binding site for another molecule, such as, but not limited to, a molecular beacon that results in a signal. Those with knowledge in the art will know many other ways to design a signal sequence for use in target probes of the invention, all of which are part of the present invention.

G. Other Optional Sequences in Target Probes of the Invention

A monopartite or a bipartite target probe of the present invention can optionally comprise other “optional sequences.” Optional sequences, if present, can be 5′-of the target-complementary sequence at the 3′-end and 3′-of the promoter sequence in the 5′-portion of a bipartite target probe. Optional sequences, if present in a monopartite target probe, can be 5′-of the target-complementary sequence in a signal target probe or 3′-of the promoter sequence in a promoter target probe. By way of example, but not of limitation, other optional sequences can comprise one or more transcription termination sequences, one or more capture sequence sites, one or more detection sequence sites, one or more address tag sites, one or more priming sites, one or more sequences for another specific purpose, or one or more intervening sequences that have no function other than to link one portion of a target probe to another portion. A capture sequence site can be a site that is complementary to another oligonucleotide, such as, but not limited to an oligo with a biotin group, that facilitates capture of a target sequence to a surface, such as a surface to which streptavidin is bound. A detection sequence site can be a sequence that is complementary to an oligo used for detection, such as, but not limited to, a molecular beacon. An address tag can be a sequence that is complementary to an oligonucleotide or a polynucleotide that is attached to a surface, such as, but not limited to, a dipstick or a spot on an array or microarray. A priming site can be for a sequence that is complementary to an oligonucleotide primer, such as, but not limited to a primer for use in reverse transcription of an RNA transcript product of an assay or method of the invention. These optional sequences can be of any length that permits stable and specific hybridization for the intended purpose and that does not hinder the performance of an assay or method of the invention.

H. Transcription Substrates of the Invention: Circular Transcription Substrates and Linear Transcription Substrates

A “transcription substrate” of the present invention means a polynucleotide that comprises a target-complementary sequence that is operably joined to a functional promoter for an RNA polymerase that can make a transcription product using the target-complementary sequence as a template under transcription conditions. In most embodiments of the present invention, a transcription substrate is a polynucleotide complex that results from covalent joining in the presence of a target sequence of at least two target-complementary sequences comprising at least two monopartite target probes or at least one bipartite target probe, wherein the 3′-end of the target-complementary sequence that anneals to the 5′-end of the target sequence is joined to a sense promoter sequence that is complexed with (or annealed to) an anti-sense promoter oligo to obtain a double-stranded promoter, wherein an RNA polymerase can bind said double-stranded promoter and initiate transcription therefrom under transcription conditions to obtain a transcription product. Optionally, a transcription substrate of the invention can also have additional nucleic acid sequences, such as but not limited to detectable “signal sequences,” that are in the same DNA strand and 5′-of said joined target-complementary sequences. However, a transcription substrate of the invention is not required to have said additional nucleic acid sequences. In some embodiments, a transcription substrate comprises a target sequence that is operably joined to a single-stranded promoter or pseudopromoter for an RNA polymerase that can bind said single-stranded promoter or pseudopromoter and initiate transcription therefrom.

A transcription substrate typically has a transcription initiation site at the 5′-end of the promoter sequence. A transcription substrate of the invention can also have one or more other sequences that are 5′-of the target-complementary sequence. By way of example, but not of limitation, a transcription substrate can have one or more transcription termination sequences, one or more sites for DNA cleavage to permit controlled linearization of a circular transcription substrate, and/or other sequences or genetic elements for a particular purpose, including, but not limited to, sequences that are transcribed by the RNA polymerase so as to provide additional regions of complementarity in the RNA transcription products: (i) for annealing of primers for reverse transcription in order to make cDNA for additional rounds of amplification; or (ii) for annealing of additional target probes for generation of additional transcription substrates by means of additional joining reactions using the RNA transcription product as a ligation template (e.g., by using a different joining enzyme or joining method on the RNA ligation template than the joining enzyme or joining method that was used in the initial joining reaction of target probes on the target sequence).

I. Hybridization or Annealing Processes of the Invention

“Hybridization” or “annealing” refers to the “binding” or “pairing” of complementary nucleic acid bases in one single-stranded nucleic acid, peptide nucleic acid (PNA), or linked nucleic acid-PNA molecule with another single-stranded nucleic acid, PNA, or linked nucleic acid-PNA molecule under “binding” or “annealing” or “hybridization” conditions.” The ability of two polymers of nucleic acid and/or PNA containing complementary sequences to find each other and anneal through base pairing interaction is a well-recognized phenomenon. The initial observations of the “hybridization” process by Marmur and Lane (Proc. Nat. Acad. Sci. USA, 46: 453, 1960) and Doty, et al. (Proc. Nat. Acad. Sci. USA, 46: 461, 1960) have been followed by the refinement of this process into an essential tool of modern biology. Hybridization occurs according to base pairing rules (e.g., adenine pairs with thymine or uracil and guanine pairs with cytosine). Those with skill in the art will be able to develop and make conditions which comprise binding conditions or hybridization conditions for a particular target nucleic acid analytes or target sequence tag joined to a non-nucleic acid analyte and target probes of an assay or method of the invention. In developing and making binding conditions for particular target nucleic acid analytes or target sequence tags joined to non-nucleic acid analytes with target probes an assay of the invention, as well as in developing and making hybridization conditions for other oligonucleotides or polynucleotides which can be used, such as, but not limited to capture probes, or detection probes such as molecular beacons, certain additives can be added in the hybridization solution. By way of example, but not of limitation, dextran sulfate or polyethylene glycol can be added to accelerate the rate of hybridization (e.g., Chapter 9, Sambrook, et al., Molecular Cloning: A Laboratory Manual, 2^{nd} Edition, Cold Spring Harbor Laboratory Press, 1989), or betaine can be added to the hybridization solution to eliminate the dependence of Tₘ. on basepair composition (Rees, W. A., et al., Biochemistry, 32, 137-144, 1993). However, other hybridization conditions that do not use such additives can also be used in an assay or method of the invention.

The terms “degree of homology” or “degree of complementarity” refer to the extent or frequency at which the nucleic acid bases on one strand (e.g., of the affinity molecule) are “complementary with” or “able to pair” with the nucleic acid bases on the other strand (e.g., the analyte). Complementarity may be “partial,” meaning only some of the nucleic acid bases are matched according to base pairing rules, or complementarity may be “complete” or “total.” The length (i.e., the number of nucleic acid bases comprising the nucleic acid and/or PNA affinity molecule and the nucleic acid analyte), and the degree of “homology” or “complementarity” between the affinity molecule and the analyte have significant effects on the efficiency and strength of binding or hybridization when the nucleic acid bases on the affinity molecule are maximally “bound” or “hybridized” to the nucleic acid bases on the analyte. The terms “melting temperature” or “Tₘ” are used as an indication of the degree of complementarity. The Tₘ is the temperature at which a population of double-stranded nucleic acid molecules becomes half dissociated into single strands under defined conditions. Based on the assumption that a nucleic acid molecule that is used in hybridization will be approximately completely homologous or complementary to a target polynucleotide, equations have been developed for estimating the Tₘ for a given single-stranded sequence that is hybridized or “annealed” to a complementary sequence. For example, a common equation used in the art for oligodeoxynucleotides is: Tₘ=81.5° C.+0.41(% G+C) when the nucleic acid is in an aqueous solution containing 1 M NaCl (see e.g., Anderson and Young, Quantitative Filter Hybridization, in Nucleic Acid Hybridization, 1985). Other more sophisticated equations available for nucleic acids take nearest neighbor and other structural effects into account for calculation of the Tₘ. Binding is generally stronger for PNA affinity molecules than for nucleic acid affinity molecules. For example the Tₘ of a 10-mer homothymidine PNA binding to its complementary 10-mer homoadenosine DNA is 73° C., whereas the Tₘ for the corresponding 10-mer homothymidine DNA to the same complementary 10-mer homoadenosine DNA is only 23° C. Equations for calculating the Tₘ for a nucleic acid are not appropriate for PNA. Preferably, a Tₘ that is calculated using an equation in the art, is checked empirically and the hybridization or binding conditions are adjusted by empirically raising or lowering the stringency of hybridization as appropriate for a particular assay.

As used herein the term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required when it is desired that nucleic acids that are not completely complementary to one another be hybridized or annealed together.

As used herein, “hybridization,” “hybridizes” or “capable of hybridizing” is understood to mean the forming of a double or triple stranded molecule or a molecule with partial double or triple stranded nature. The term “hybridization,” “hybridize(s)” or “capable of hybridizing” encompasses the terms “stringent condition(s)” or “high stringency” and the terms “low stringency” or “low stringency condition(s).”

As used herein “stringent conditions” or “high stringency conditions” comprise conditions that allow hybridization between or within one or more nucleic acid strands containing complementary sequences, but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethyl-ammonium chloride, betaine or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suit a particular application. For example, in other embodiments, hybridization may be achieved under conditions of 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

With regard to complementarity, it is important for some assays of the invention to determine whether the hybridization represents complete or partial complementarity. For example, where it is desired to detect simply the presence or absence of pathogen DNA (such as from a virus, bacterium, fungi, mycoplasma, protozoan), it is only important that the hybridization method ensures hybridization when the relevant sequence is present. In those embodiments of the invention, conditions can be selected where both partially complementary probes and completely complementary probes will hybridize. However, in general, even if the probes are only partially complementary, they must be completely complementary at the terminal nucleotides comprising the ligation junction.

The invention can also be used for assays to detect mutations, or genetic polymorphisms, or single nucleotide polymorphisms (SNPs). These embodiments of the invention require that the hybridization and other aspects of the method distinguish between partial and complete complementarity. For example, human hemoglobin is composed, in part, of four polypeptide chains. Two of these chains are identical chains of 141 amino acids (alpha chains) and two of these chains are identical chains of 146 amino acids (beta chains). The gene encoding the beta chain is known to exhibit polymorphism. The normal allele encodes a beta chain having glutamic acid at the sixth position. The mutant allele encodes a beta chain having valine at the sixth position. This difference in amino acids has a profound (most profound when the individual is homozygous for the mutant allele) physiological impact known clinically as sickle cell anemia. It is well known that the genetic basis of the amino acid change involves a single base difference between the normal allele DNA sequence and the mutant allele DNA sequence. Thus, some embodiments of the invention are used for assays that can detect and distinguish even as small a difference as a single basepair in a target nucleic acid analyte.

J. Ligases and Ligation Processes of the Invention

In general, “ligation” refers to the joining of a 5′-phosphorylated end of one nucleic acid molecule with the 3′-hydroxyl end of another nucleic acid molecule by an enzyme called a “ligase,” although in some methods of the invention, the ligation can be effected by another mechanism. With respect to ligation, a region, portion, or sequence that is “adjacent to” or “contiguous to” or “contiguous with” another sequence directly abuts that region, portion, or sequence.

The invention is not limited to a specific ligase. However, preferably the ligase is not active in ligating blunt ends and is highly selective for ligation of a deoxyribonucleotide having a 5′-phosphate and a deoxyribonucleotide having 3′-hydroxyl group when these respective 5′- and 3′-nucleotides are adjacent to each other when annealed to a target sequence of a target nucleic acid. Ampligase® Thermostable DNA Ligase Tth DNA ligase, and Tfl DNA Ligase (EPICENTRE Technologies, Madison, Wis., USA), or Tsc DNA Ligase (Prokaria Ltd., Reykjavik, Iceland) are NAD-dependent thermostable ligases that are not active on blunt ends and that ligate the 5′-phosphate and 3′-hydroxyl termini of DNA ends that are adjacent to one another when annealed to a complementary DNA molecule; these enzymes are preferred ligases in embodiments of the invention wherein a target sequence comprises DNA. Another DNA ligase that can be used in the methods of the invention for target sequences comprising DNA is Pfu DNA ligase as described by Mathur et al. (U.S. Pat. Nos. 5,700,672 and 6,280,998, which are incorporated herein by reference. Thermostable DNA ligases are preferred in some embodiments because they can be cycled through multiple annealing-ligation-melting cycles, permitting multiple target probe ligations for every target sequence present in a sample, and thus, increasing the sensitivity of the assay or method. However, the invention is not limited to the use of a particular ligase, or to the use of a thermostable ligase and other suitable ligases that function in the assays and methods of the invention can also be used. For example, T4 DNA ligase can be used in some embodiments of the invention for target sequences that comprise DNA.

In addition, Faruqui discloses in U.S. Pat. No. 6,368,801, incorporated herein by reference, that T4 RNA ligase can efficiently ligate DNA ends of nucleic acids that are adjacent to each other when hybridized to an RNA strand. Thus, T4 RNA ligase is a preferred ligase of the invention in embodiments in which DNA ends are ligated on a target sequence that comprises RNA. However, because of the high potential for “background” ligation reactions, T4 RNA ligase is not preferred when high specificity and/or high sensitivity is desired.

Other ligases that ligate DNA ends of nucleic acids that are adjacent to each other when hybridized to an RNA strand are preferred for target nucleic acids comprising RNA. The invention is also not limited to the use of a ligase for covalently joining target probe ends in the various embodiments of the invention. By way of example, other ligation methods such as, but not limited to, topoisomerase-mediated ligation (e.g., U.S. Pat. No. 5,766,891, incorporated herein by reference) can be used, although topoisomerase-mediated ligation is not preferred in most embodiments because of the high potential for background ligation. In some other embodiments, chemical ligation methods can be used, such as, but not limited to, the use of a target probe with a 5′-end sequence that comprises a 5′-iodo-nucleotide and a 3′-end comprising a nucleotide with phosphorothioate, as disclosed by Xu, Y., and Kool, E. T. (Nucleic Acids Res., 27: 875-881, 1999), which is incorporated herein by reference. The invention is not limited with respect to the ligation method used except that the ligation should occur efficiently in the presence of a target sequence to which the target probes anneal contiguously and ligation should occur rarely or not at all in the absence of a target sequence. As used herein, “ligation” refers to any suitable method for joining adjacent 5′- and 3′-ends of target probes that are adjacent or contiguous to each other when annealed to a target sequence. In preferred ligation processes of the present invention, all of the target probes that anneal to a target sequence have a similar melting temperature (Tₘ) with respect to the target sequence, and the lowest temperature at which a ligation process is performed is near the Tₘ of the target probe having the lowest Tₘ when it is annealed to the target sequence.

K. Release of Circular ssDNA Molecules That are Catenated to a Target Sequence Following Ligation Using the Target Sequence as a Ligation Template

Bipartite probes that are ligated when annealed to a target sequence create circular DNA molecules catenated to the target sequence (Nilsson, M. et al., Science, 265:2085-2088, 1994, incorporated herein by reference). Nilsson et al. showed that, if the target sequence was less than about 150-200 nucleotides from the 3′-end of the target nucleic acid, the catenated circular ssDNA molecules obtained by ligation of a linear probe on a target sequence were able to slip off of the target strand during denaturing washes, whereas circular molecules catenated on the target sequence 850 nucleotides from the 3′-end of the target nucleic acid were not removed during denaturing washes.

The present invention comprises some embodiments in which a TSA circle is replicated by rolling circle replication while catenated to target nucleic acid or a target sequence tag using a DNA polymerase, such as but not limited to IsoTherm™ DNA polymerase (EPICENTRE Technologies, Madison, Wis.), Bst DNA polymerase large fragment, or another DNA polymerase that can efficiently replicate catenated templates. The present invention also comprises embodiments in which circular transcription substrates are transcribed by rolling circle transcription while they are catenated to a target nucleic acid or target sequence tag so long as the assay or method functions for its intended purpose. It can be beneficial that the respective molecules remain catenated to the target nucleic acid, and it is beneficial if the number of steps and the time to perform an assay is kept to the minimum to obtain the information for which the assay or method was intended.

However, in other embodiments, it can be desirable for a variety of reasons that circular ssDNA ligation products obtained using a method of the invention does not remain catenated to a target nucleic acid or target sequence tag comprising the target sequence following ligation. By way of example, but not of limitation, catenation of a TSA circle obtained by annealing and ligation of a target sequence amplification probe (TSA probe) on a target sequence or catenation of a circular transcription substrate obtained by annealing and ligation of a bipartite target probe on a target sequence and annealing of an anti-sense promoter oligo may limit the amount of rolling circle replication product (e.g., see Baner, J. et al., Nucleic Acids Research, 26: 5073-5078, 1998) or rolling circle transcription product, respectively, if the catenated circular molecules remain catenated to the target nucleic acid or target sequence tag comprising the target sequence following ligation.

However, the effect of catenation on the target nucleic acid should be determined empirically in view of the results of Kuhn et al. (Nucleic Acids Res., 30: 574-580, 2002), incorporated herein by reference. Kuhn et al. showed that, although rolling circle replication was limited on catenated circular ssDNA molecules using phi29 DNA polymerase (which was used by Baner et al., Nucleic Acids Research, 26: 5073-5078, 1998), the amount of rolling circle replication product obtained using catenated circular ssDNA molecules was not affected when Bst DNA polymerase large fragment, Sequenase® DNA polymerase (USB, Cleveland, Ohio), or Vent (exo-minus) DNA polymerase (New England Biolabs, Beverly, Mass.) was used. Whether or not it is necessary to release catenated circular ssDNA molecules from the target nucleic acid prior to rolling circle replication depends on the DNA polymerase used, indicating that the need to release catenated circular transcription substrates from the target nucleic acid may also depend on the particular RNA polymerase used for rolling circle transcription.

Therefore, if a target nucleic acid comprising a target sequence does not have a free 3′-end that is less than about 150-200 nucleotides from the target sequence, the present invention comprises empirically determining if catenation of a ligation product obtained from ligation of a TSA probe or a bipartite target probe on the target sequence results in a reduction in the amount of product obtained during rolling circle replication or rolling circle transcription, respectively, compared to the amount of product obtained on an oligodeoxyribonucleotide comprising only the target sequence. If the amount of product obtained is found to be decreased by catenation, then an assay or method of the invention will either use additional steps to release the catenated circular ssDNA molecule from the target nucleic acid for the particular assay or method, such as but not limited to one of the methods to release catenated molecules described herein below, or will use a different polymerase for which the amount of replication product or transcription product is not affected by catenation.

It can also be useful to release catenated circular ssDNA molecules from a target nucleic acid for other reasons than to obtain more efficient rolling circle transcription. In some embodiments, the circular ligation product is annealed to an anti-sense promoter oligo that is attached to a solid support in order to obtain a circular transcription substrate and to separate the circular transcription substrate from other nucleic acids and other components of the sample prior to in vitro transcription. Removal of other nucleic acids and other components decreases the possibility of non-specific transcription or replication of other sequences that result in a high “false positive” signal, and also decreases the possibility of inhibitory sequences or components being present that result in a decreased target-dependent transcription signal or even potentially, a “false negative” signal. Therefore, it is also preferred that catenated circular ligation product is released from a target nucleic acid or target sequence tag of the present invention so as not to interfere with other steps of the method or assay. Therefore, preferably the target sequence comprising a target nucleic acid or a target sequence tag that is joined to an analyte-binding substance is less than about 150-200 nucleotides from the 3′-end of the respective target nucleic acid or target sequence tag.

In general, a target sequence tag of the present invention will comprise a sequence that has a 3′-end that is less than about 150-200 nucleotides from the target sequence. Preferably, the 3′-end of the target sequence tag is less than 100 nucleotides from the target sequence and most preferably, the 3′-end of the target sequence tag is less than 50 nucleotides from the target sequence.

With respect to a target sequence comprising a target nucleic acid in a sample, if the target sequence is more than about 150-200 nucleotides from the 3′-end of the target nucleic acid, it is obvious to a person with knowledge in the art that there are a number of methods for breaking or cutting or shortening the target nucleic acid in order to obtain a fragmented target nucleic acid comprising the target sequence and any suitable method can be used to obtain a target nucleic acid for an assay or method of the present invention. Preferably, the target nucleic acid is fragmented to a size that has a 3′-end that is less than about 150-200 nucleotides from the target sequence prior to use of the target nucleic acid in an assay or method of the invention.

By way of example, but not of limitation, a DNA in a sample comprising a dsDNA molecule or a ssDNA molecule to which an appropriate complementary DNA oligo is annealed can be digested with a restriction endonuclease, provided that a suitable restriction site is present within less than about 150-200 nucleotides from the 3′-end of the target sequence and no restriction sites for the enzyme are present within the target sequence. Alternatively, if a suitable restriction site is not present on the target nucleic acid, one or more DNA oligonucleotides having a double-stranded segment that contains a FokI restriction enzyme site and a single-stranded segment that binds to the desired cleavage site on a first-strand cDNA can be used. As is well known in the art, this type of oligonucleotide can be used with the restriction enzyme FokI to cut a single-stranded DNA at almost any desired sequence (Szybalski, W., Gene 40:169-173, 1985; Podhajska A. J. and Szybalski W., Gene 40:175, 1985, incorporated herein by reference).

Still further, either RNA or DNA nucleic acids of known sequence can be cleaved at specific sites using a 5′-nuclease or Cleavase™ enzyme and specific oligonucleotides, as described by Kwiatkowski, et al., (Molecular Diagnosis 4:353-364, 1999) and in U.S. Pat. No. 6,001,567 and related patents assigned to Third Wave Technologies (Madison, Wis., USA), which are incorporated herein by reference.

If the target nucleic acid is first-strand cDNA obtained by reverse transcription of RNA using a primer, the RNA can be cleaved with RNase H at a site to which a DNA oligo is annealed in order to define the 3′-end of the reverse transcription product that is obtained. Alternatively, the length of the reverse transcription product can be kept within a desired size range by limiting the time of the reverse transcription reaction, which reverse transcription reaction can be optimized for the particular primer, template sequence and reaction conditions used to obtain a target nucleic acid comprising a target sequence, if present in the sample.

Still another method that can be used is to incorporate dUMP randomly into the first-strand cDNA during reverse transcription or primer extension to prepare a target nucleic acid comprising a target sequence. In these embodiments, dUTP (deoxyribouridine triphosphate) is used in place of a portion of the dTTP (thymidine triphosphate) in the reaction. Also, dUTP can be incorporated in place of a portion of the dTTP in rolling circle replication of TSA circles that are used to increase the number of target sequences available for annealing and ligation of target probes for target-dependent transcription. As discussed elsewhere herein, TSA circles are obtained by annealing and ligation of target sequence amplification probes (TSA probes) on a target sequence in a sample. When dUTP is used in a reverse transcription, primer extension or rolling circle replication reaction in addition to dTTP, dUMP will be incorporated randomly in place of TMP at a frequency based on the ratio of dUTP to dTTP. Then, the respective first-strand cDNA, primer extension product or rolling circle replication product can be cleaved at sites of dUMP incorporation by treatment (e.g., see U.S. Pat. No. 6,048,696, incorporated herein by reference) with uracil-N-glycosylase (UNG) and endonuclease IV (endo IV), which are available from EPICENTRE Technologies (Madison, Wis., USA). UNG hydrolyzes the N-glycosidic bond between the deoxyribose sugar and uracil in single- and double-stranded DNA that contains uracil in place of thymidine. UNG has no activity on dUTP or in cleaving uracil from UMP residues in RNA. Endo IV cleaves the phosphodiester linkage at the abasic site. It may be useful to use a thermolabile UNG (e.g., HK™-UNG from EPICENTRE Technologies, Madison, Wis., USA) for some applications. (Also, incorporation of dUMP at one or more specific sites within a synthetic oligonucleotide introduces a specific cleavage site which can be used at any time to cleave a resulting nucleic acid which contains the site by treatment with UNG and endo IV.)

Further, the 3′-end of a first-strand cDNA that is to become the template sequence for a transcription reaction can be defined by first amplifying the target nucleic acid sequence using any suitable amplification method, such as but not limited to PCR or RT-PCR, that delimits the end sequence.

If a 3′-end of a target sequence need not be at an exact location, and can be random or imprecise, which is the case in some embodiments of the invention, there are a number of other methods that can be used for making smaller fragments of a DNA molecule, whether for a target nucleic acid, a target sequence, or otherwise. By way of example, but not of limitation, a target nucleic acid can be fragmented by physical means, such as by movement in and out of a syringe needle or other orifice or by sonication. If desired, the ends of physically fragmented double-stranded DNA can be made blunt prior to denaturation and use in an assay or method of the present invention using a T4 DNA polymerase or a kit, such as the End-It™ DNA End Repair Kit (EPICENTRE Technologies, Madison, Wis., USA).

Although it is preferred that a target nucleic acid comprising a target sequence is short enough so that its 3′-end will easily be released from the catenated circular molecules that result from ligation of a bipartite target probe annealed to the target sequence, the present invention also includes embodiments of methods, assays, compositions and kits for detecting target sequences comprising larger target nucleic acids, wherein the catenated ligation product is not substantially released from the target nucleic acid. In those embodiments, the invention comprises additional steps for release of the catenated circular ligation product after annealing and ligation on the target sequence.

Baner, J. et al. (Nucleic Acids Research, 26: 5073-5078, 1998, incorporated herein by reference) showed that ligation of a linear DNA having two target-complementary end sequences that anneal adjacently on a target sequence resulted in a catenated molecule that was not efficiently replicated by rolling circle replication using phi29 DNA polymerase unless there was a free 3′-end of the target nucleic acid near the ligation site. Baner et al. showed that, in order to obtain efficient rolling circle replication by phi29 DNA polymerase of circular ssDNA molecules that had been ligated on a target sequence, the topological link of the circular DNA with the target molecules needed to be released. U.S. Pat. No. 6,558,928, incorporated herein by reference, provided methods for release of catenated circular DNA molecules in order to improve the efficiency of rolling circle replication reactions. The present invention comprises the use of the methods described in U.S. Pat. No. 6,558,928, which methods are incorporated herein by reference, in order to release catenated circular ligation products for rolling circle transcription as described herein.

In some embodiments, the circular ssDNA ligation product is released from catenation with the target sequence by digestion with an exonuclease after ligation of the bipartite probe on the target sequence. Preferred exonucleases are those that digest single-stranded DNA and that do not have endonuclease activity. One enzyme that can be used is exonuclease I (exo I) (EPICENTRE Technologies, Madison, Wis.), which has 3′-to-5′ single-stranded exonuclease activity in the presence of Mg²⁺ cations. Another enzyme that can be used is exonuclease VII (exo VII) (EPICENTRE Technologies, Madison, Wis.), which has both 3′-to-5′ and 5′-to-3′ single-stranded exonuclease activity. Exo VII is active in the absence of Mg²⁺ cations, which makes it a preferred embodiment for many applications. Rec J nuclease (EPICENTRE Technologies, Madison, Wis.), which has 5′-to-3′ single-stranded exonuclease activity in the presence of Mg²⁺ cations, can also be used in some embodiments. Still further, it is preferable to also use a double-stranded exonuclease, such as but not limited to exonuclease III (exo III) in addition to a single-stranded exonuclease, such as but not limited to exo I, in order to release catenated circular DNA molecules from a target nucleic acid comprising a target sequence. In some embodiments, the target sequence that has been digested with an exonuclease can prime rolling circle replication after exonuclease removal of the non-base-paired 3′ end.

L. DNA Polymerases and Processes of the Invention for Filling “Gaps” Between Target Probes

DNA polymerases are used in some embodiments of the present invention in order to fill by DNA polymerase extension one or more “gaps” between non-contiguous target-complementary sequences of target probes that are annealed to a target sequence. The invention is not limited to a particular DNA polymerase to accomplish this purpose, and the invention includes use of any DNA polymerase that is active in filling a gap under suitable reaction conditions. A suitable DNA polymerase fills the gap by DNA polymerase extension from the 3′-hydroxyl end of one target-complementary target probe to the 5′-end of the next target-complementary target probe, without strand-displacement of the target-complementary 5′-end portion of a target probe. The “strand displacement” activity of a DNA polymerase is an operational definition and depends on reaction conditions, such as, but not limited to, reaction temperature, buffer, salt concentration, pH, Mg²⁺ concentration, use of cosolvents such as DMSO, or DNA polymerase enhancers such as betaine, as well as on the intrinsic properties of a DNA polymerase. Thus, even though a particular DNA polymerase may have strand-displacement activity under certain reaction conditions, it may not have significant strand-displacement activity under other reaction conditions. Thus, it is preferred that a DNA polymerase is evaluated for strand-displacement activity under the desired reaction conditions of an assay or method of the invention. Strand displacement and DNA polymerase processivity can be assayed using methods described in Kong et al. (J. Biol. Chem., 268: 1965-1975, 1993) and references cited therein, all of which are incorporated herein by reference. Preferred DNA polymerases lack 5′-to '3′ and 3′-to-5′ exonuclease activity under the reaction conditions used. It is also important that the DNA polymerase used to fill a gap lacks a 5′ structure-dependent nuclease, such as Cleavase™ or Invader™ nucleases used by Third Wave Technologies (Madison, Wis.) because these enzymes could cleave off an unpaired nucleotide, especially at the 5′-end of a sequence that is partially annealed to a target sequence 3′-of another target probe, and then the DNA polymerase could fill in the gap formed. Therefore, a DNA polymerase with 5′ structure-dependent nuclease activity could result in inaccurate results in the assay. Most preferred DNA polymerases are thermostable so that activity is more consistent during the course of a method or assay of the invention and in order to be more easily stored without loss of polymerase activity. A preferred DNA polymerase of the invention for filling gaps between target probes annealed to a target sequence is T4 DNA polymerase. Another DNA polymerase that can be used is T7 DNA polymerase (EPICENTRE Technologies, Madison, Wis., USA).

M. RNA Polymerases, Transcription Promoters and Transcription Processes of the Invention

1. RNA Polymerases and Transcription Promoters of the Invention

The present invention is not limited with respect to an RNA polymerase (RNAP). The invention comprises the use of any RNA polymerase that can be used to make a transcription product using a transcription substrate obtained using the methods of the invention described herein. In order to make a transcription substrate of the invention, a cognate transcription promoter must be known and obtained, wherein the RNA polymerase recognizes and binds to said promoter with specificity and initiates transcription therefrom. A “cognate promoter” for a particular RNA polymerase is a promoter that is recognized by that particular RNA polymerase with specifity. Similarly, a “cognate RNA polymerase” for a particular promoter is one that recognizes the particular promoter with substantially greater specifity than another RNA polymerase that recognizes one or more other promoter sequences, thus permitting transcription of the template that is joined with the promoter with specificity even in the presence of other sequences that are not recognized as a promoter by the particular RNA polymerase. A “transcription promoter” or a “promoter sequence” or a “promoter” is a specific nucleic acid sequence that is recognized by a DNA-dependent RNA polymerase (or simply an “RNA polymerase” or “RNAP”) of the invention as a signal to bind to the nucleic acid and begin the transcription of RNA at a specific site. Most naturally occurring RNA polymerases known in the art, including but not limited to T7-type RNA polymerases, E. coli RNAP, Thermus thermophilus RNAP, mitochondrial RNA polymerases and eukaryotic RNA polymerases, recognize a double-stranded sequence as the promoter sequence. With respect to RNA polymerases that recognize a double-stranded promoter it will be understood that, when one refers to “a promoter sequence,” the promoter comprises both that sequence and its complementary sequence. More specifically, the promoter sequence that is joined to the template strand for transcription is referred to as the “sense promoter sequence” and its complementary sequence is referred to as the “anti-sense promoter sequence.” A functional double-stranded promoter therefore comprises a complex between a sense promoter sequence and an anti-sense promoter sequence, and a transcription substrate for an RNA polymerase that uses a double-stranded promoter is obtained only after annealing of an anti-sense promoter sequence to a sense promoter sequence that is joined to the 3′-end of a template for transcription. Phage N4 vRNAP and N4 mini-vRNAP deletion mutants are exceptional in that they bind and initiate transcription from single-stranded promoters. Therefore, a “promoter sequence” for an N4 vRNAP or mini-vRNAP comprises only a single-stranded sense promoter sequence that is joined to the 3′-end of a template for transcription, which comprises a functional “transcription substrate.” Still further, the invention also comprises embodiments that use single-stranded “pseudopromoters” or “synthetic promoters.” As discussed in greater detail elsewhere herein, pseudopromoters are single-stranded sense promoter sequences that are artificially obtained by an in vitro process of “molecular evolution” and selection of a sequence for promoter activity for a particular RNA polymerase. Thus, a pseudopromoter is a sequence that is able to serve the same function as a single-stranded sense promoter like an N4 promoter for its cognate RNAP.

A wide variety of RNA polymerases and their cognate promoters are known in the art. For example, Inspection of the sequences of phage, archaebacterial, eubacterial, eukaryotic and viral DNA-dependent RNA polymerases has revealed the existence of two enzyme families. The eubacterial, eukaryotic, archaebacterial, chloroplast and the vaccinia virus RNA polymerases are complex multisubunit enzymes (5-14 subunits) composed of two large subunits, one to several subunits of intermediate molecular weight (30-50-kDa) and none to several subunits of small molecular weight (<30-kDa) (Archambault and Friesen, Microbiol. Rev. 57:703-724, 1993; Record et al., Cell and Molecular Biology 1:792-821, 1995. Eubacterial RNA polymerases are the simplest with an α₂ββ′ core structure. Sequence comparison of the genes coding for the different subunits of these enzymes has revealed: 1-sequence homology in eight segments (A to H) between β′ and the largest subunit of other RNA polymerases, 2-sequence homology in nine segments (A to I) between β and the next largest subunit of other RNA polymerases, 3-sequence homology in 3 segments (1.1, 1.2 and 2) between a and a subunit in RNA polymerases I, II and III (Puhler, et al., Proc. Natl. Acad. Sci. USA 86:4569-4573, 1989; Sweetser, et al., Proc. Natl. Acad. Sci. USA 84:1192-1196, 1987). Not surprisingly, the crystal structures of yeast RNAP II and E. coli RNAP core revealed remarkable similarities (Zhang, et al., Cell 98:811-824, 1999; Cramer, et al., Sciencexpress, www.sciencexpress.org. 19 Apr. 2001).

Members of the phage T7-like (T7, T3, SP6) family of RNA polymerases consist of a single (˜100 kDa) polypeptide which catalyzes all functions required for accurate transcription (Cheetham, et al., Curr. Op. In Struc. Biol. 10:117-123, 2000). The heterodimeric bacteriophage N4 RNAP II, nuclear-coded mitochondrial, and Arabidopsis chloroplast RNA polymerases show sequence similarity to the phage RNA polymerases (Cermakian, et al., Nuc. Acids Res. 24:648-654, 1996; Hedtke, et al., Science 277:809-811, 1997; Zehring, et al., J. Biol. Chem. 258:8074-8080, 1983). Three sequence motifs -A and C, which contain the two aspartic acids required for catalysis, and motif B- are conserved in polymerases that use DNA as a template (Delarue, et al., Protein Engineering 3:461-467, 1990). The crystal structure of T7 RNAP resembles a “cupped right hand” with “palm,” “fingers” and “thumb” subdomains (Sousa, et al., Nature 364:593-599, 1993). The two catalytic aspartates are present in the “palm” of the structure. This structure is shared by the polymerase domains of E. coli DNA polymerase I and HIV reverse transcriptase (Sousa, Trends in Biochem. Sci. 21:186-190, 1996). Genetic, biochemical and structural information indicates that T7 RNA polymerase contains additional structures dedicated to nascent RNA binding, promoter recognition, dsDNA unwinding and RNA:DNA hybrid unwinding (Cheetham, et al., Curr. Op. In Struc. Biol. 10:117-123, 2000; Sousa, Trends in Biochem. Sci. 21:186-190, 1996). This unwinding activity of T7 RNAP and T7-like RNAPs is described in Japanese Patent Nos. JP4304900 and JP4262799 as “helicase-like activity.”

Both Class I and Class II RNA polymerases recognize specific sequences, called promoters, on B form double-stranded DNA. Eubacterial promoters (except those recognized by σ⁵⁴) are characterized by two regions of sequence homology: the −10 and the −35 hexamers (Gross, et al., Cold Spring Harbor Symp. Quant. Biol. 63:141-156, 1998). Specificity of promoter recognition is conferred to the core enzyme by the σ subunit, which makes specific interactions with the −10 and −35 sequences through two distinct DNA binding domains (Gross, et al., Cold Spring Harbor Symp. Quant. Biol. 63:141-156, 1998). This modular promoter structure is also present at the promoters for eukaryotic RNA polymerases I, II and III. Transcription factors TFIIIA and TFIIIC direct recognition of RNAP III to two separate sequences (boxes A and C, separated by defined spacing) at the 5S gene promoter, while transcription factors TFIIIB and TFIIIC direct recognition of this enzyme to blocks A and B, separated by variable distance (31-74 bp) at the tRNA promoters (Paule, et al., Nuc. Acids Res. 28:1283-1298, 2000). Sequences important for RNAP I transcription initiation at the human rRNA promoters are also restricted to two regions: the “core” region located at −40 to +1 and the “upstream” region present at −160 to −107 (Paule, et al., Nuc. Acids Res. 28:1283-1298, 2000). Assembly of the initiation complex at RNAP II promoters requires several general transcription factors (TFIIA, TFIIB, TFIID, TFIIE, TFIIF and TFIIH). Recognition involves three core elements: the TATA box located at position −30 and recognized by TBP, the initiator element located near −1, and the downstream promoter element near +30 (Roeder, Trends Biochem. Sci. 21:327-335, 1996).

Promoters for the T7-like and mitochondrial RNAPs are simpler. The T7-type RNAP promoters span a continuous highly conserved 23 bp region extending from position −17 to +6 relative to the start site of transcription (+1) (Rong, et al., Proc. Natl. Acad. Sci. USA 95:515-519, 1998). The yeast mitochondrial RNAP promoters are even smaller, extending from −8 to +1 (Shadel, et al., J. Biol. Chem. 268:16083-16086, 1993). One exception are the promoters for N4 RNAP II, which are restricted to two blocks of conserved sequence: a/tTTTA at +1 and AAGACCTG present 18-26 bp upstream of +1 (Abravaya, et al., J. Mol. Biol. 211:359-372, 1990).

The activity of the multisubunit class of RNA polymerases is enhanced by activators at weak promoters. Transcription activators generally bind at specific sites on double-stranded DNA upstream of the −35 region (with the exception of the T4 sliding clamp activator), or at large distances in the cases of enhancers (Sanders, et al., EMBO Journal 16:3124-3132, 1997). Activators modulate transcription by increasing the binding (formation of closed complex) or isomerization (formation of open complex) steps of transcription through interactions with the α or σ subunits of RNAP (Hochschild, et al., Cell 92:597-600, 1998). An exception is N4SSB, the activator of E. coli RNAPσ⁷⁰ at the bacteriophage N4 late promoters, which activates transcription through direct interactions with the, β′ subunit of RNAP in the absence of DNA binding (Miller, et al., Science 275:1655-1657, 1997).

Proteins that bind to ssDNAs with high affinity but without sequence specificity have been purified and characterized from several prokaryotes, eukaryotes, and their viruses (Chase, et al., Ann. Rev. Biochem. 55:130-136, 1986). These proteins (SSBs), which are required for replication, recombination and repair, bind stoichiometrically and, in many cases, cooperatively to ssDNA to cover the transient single-stranded regions of DNA that normally arise in vivo as a result of replication, repair and recombination. Binding to DNA results in the removal of hairpin structures found on ssDNA, providing an extended conformation for proteins involved in DNA metabolism. Several lines of evidence suggest that single-stranded DNA binding proteins play a more dynamic role in cellular processes. Genetic and biochemical evidence indicates that these proteins are involved in a multitude of protein-protein interactions including transcription activation (Rothman-Denes, et al., Genes Devepmnt. 12:2782-2790, 1999).

Bacteriophage N4 virion RNA polymerase (N4 VRNAP) is present in N4 virions and is injected into the E. coli cell at the beginning of infection, where it is responsible for transcription of the N4 early genes (Falco, et al., Proc. Natl. Acad. Sci. (USA) 74:520-523, 1977; Falco, et al., Virology 95:454-465, 1979; Malone, et al., Virology 162:328-336, 1988). The N4 vRNAP gene maps to the late region of the N4 genome (Zivin, et al., J. Mol. Biol. 152:335-356, 1981). N4 vRNAP purified from virions is composed of a single polypeptide with an apparent molecular mass of approximately 320,000 kDa (Falco, et al., Biol. Chem. 255:4339-4347, 1980). In contrast to other DNA-dependent RNAPases, N4 vRNAP recognizes promoters on single-stranded templates (Falco, et al., Proc. Natl. Acad. Sci. USA 75:3220-3224, 1978). These promoters are characterized by conserved sequences and a 5 bp stem, 3 base loop hairpin structure (FIG. 1) (Haynes, et al., Cell 41:597-605, 1985; Glucksmann, et al., Cell 70:491-500, 1992). N4 vRNAP lacks unwinding or helicase-like activity on dsDNA and also lacks unwinding activity on RNA:DNA hybrids. In vivo, E. coli gyrase and single-stranded binding protein are required for transcription by N4 vRNAP (Falco, et al., J. Biol. Chem. 255:4339-4347, 1980; Markiewicz, et al., Genes and Dev. 6:2010-2019, 1992). RNA synthesis requires RNA polymerase, a DNA template, an activated precursor (the ribonucleoside triphosphates ATP, GTP, UTP and CTP (XTP)), and divalent metal ions such as Mg⁺² or Mn⁺². The metal ion Mg⁺² is strongly preferred. Synthesis of RNA begins at the promoter site on the DNA. This site contains a sequence which the RNA polymerase recognizes and binds. The RNA synthesis proceeds until a termination site is reached. N4 vRNAP termination signals comprise a hairpin loop that forms in the newly synthesized RNA which is followed by a string of uracils (poly U). These N4 vRNAP termination signals possess all of the characteristics of eubacterial sequence-dependent terminators. Single-stranded DNA of varying lengths can be used as a template for RNA synthesis using the N4 vRNAP or mini-vRNAP. EcoSSB is essential for N4 vRNAP transcription in vivo (Falco et al., Proc. Natl. Acad. Sci. (USA) 75:3220-3224, 1978; Glucksmann, et al., Cell 70:491-500, 1992). EcoSSB is a specific activator of N4 vRNAP on single-stranded and supercoiled double-stranded DNA templates. EcoSSB, unlike other SSBs, does not melt the N4 vRNAP promoter hairpin structure (Glucksmann-Kuis, et al., Cell 84:147-154, 1996; Dai et al., Genes Development, 12: 2782-2790, 1998). EcoSSB mediates template recycling during transcription by N4 vRNAP (Davidova, E K and Rothman-Denes, L B, Proc. Natl. Acad. Sci. USA 100:9250-9255, 2003).

Preferred RNA polymerases of the invention are T7 RNAP (e.g., see Studier, F W et al., pp. 60-89 in Methods in Enzymology, Vol. 185, ed. by Goeddel, D V, Academic Press, 1990, incorporated herein by reference) and other “T7-like” or “T7-type” RNA polymerases. The genetic organization of all T7-like bacteriophage that have been examined has been found to be essentially the same as that of T7. Examples of T7-like bacteriophages according to the invention include, but are not limited to Escherichia coli phages T3, phi I, phi II, W31, H, Y, A1, 122, cro, C21, C22, and C23; Pseudomonas putida phage gh-1; Salmonella typhimurium phage SP6; Serratia marcescens phages IV; Citrobacter phage VIII; and Klebsiella phage No. 11 (Hausmann, Current Topics in Microbiology and Immunology 75:77-109, 1976; Korsten et al., J. Gen. Virol. 43:57-73, 1975; Dunn, et al., Nature New Biology 230:94-96, 1971; Towle, et al., J. Biol. Chem. 250:1723-1733, 1975; Butler and Chamberlin, J. Biol. Chem. 257:5772-5778, 1982). Mutant RNAPs (Sousa et al., U.S. Pat. No. 5,849,546; Padilla, R and Sousa, R, Nucleic Acids Res., 15: e138, 2002; Sousa, R and Mukherjee, S, Prog Nucleic Acid Res Mol. Biol., 73: 1-41, 2003), such as, but not limited to, T7 RNAP Y639F mutant enzyme, T3 RNAP Y573F mutant enzyme, SP6 RNAP Y631F mutant enzyme, T7 RNAP having altered amino acids at both positions 639 and 784, T3 RNAP having altered amino acids at both positions 573 and 785, or SP6 RNAP having altered amino acids at both positions 631 and 779 can also be used in some embodiments of methods or assays of the invention. In particular, such mutant enzymes can corporate dNTPs and 2′-F-dNTPs, in addition to ddNTPs and certain other substrates, which are advantageous for synthesis of RNA molecules with specific properties and uses. By way of example, but not of limitation, modified RNA molecules that contain 2′-F-dCMP and 2′-F-dUTP: (i) are resistant to RNase A-type ribonucleases (Sousa et al., U.S. Pat. No. 5,849,546, incorporated herein by reference); (ii) can be delivered into cells without complexing with a transfection agent and in the presence of serum (Capodici et al., J. Immunology, 169: 5196-5201, 2002); and (iii) may be less likely than unmodified RNA to not induce an interferon response in vivo in animals or humans (see Kakiuchi et al., J. Biol. Chem., 257: 1924-1928, 1982). However, in most embodiments of methods of the invention in which a DNA polymerase is present in a reaction in addition to an RNA polymerase of the invention, a mutant RNAP enzyme is not preferred. In those embodiments, the dNTPs in a reaction mixture as substrates for a DNA polymerase can be incorporated into a transcription product by the mutant RNAP enzyme, although less efficiently than an NTP. Thus, in those embodiments, unless there is a particular reason for doing otherwise, the “wild-type” enzyme is preferred.

Promoter sequences may be used that that are recognized specifically by a DNA-dependent RNA polymerase, such as, but not limited to, those described by Chamberlin and Ryan, In: The Enzymes. San Diego, Calif., Academic Press, 15:87-108, 1982, and by Jorgensen et al., J. Biol. Chem. 266:645-655, 1991. Several RNA polymerase promoter sequences are especially useful, including, but not limited to, promoters derived from SP6 (e.g., Zhou and Doetsch, Proc. Nat. Acad. Sci. USA 90:6601-6605, 1993), T7 (e.g., Martin, and Coleman, Biochemistry 26:2690-2696, 1987) and T3 (e.g., McGraw et al., Nucl. Acid. Res. 13:6753-6766, 1985). The length of the promoter sequence will vary depending upon the promoter chosen. For example, the T7 RNA polymerase promoter can be only about 25 bases in length and act as a functional promoter, while other promoter sequences require 50 or more bases to provide a functional promoter.

Another RNA polymerase promoter sequence that can be used is derived from Thermus thermophilus (see, e.g., Wendt et al., Eur. J. Biochem. 191:467-472, 1990; Faraldo et al., J. Bact. 174:7458-7462, 1992; Hartmann et al., Biochem. 69:1097-1104, 1987; Hartmann et al., Nucl. Acids Res. 19:5957-5964, 1991) with the corresponding Thermus thermophilus RNAP (EPICENTRE Technologies, Madison, Wis.).

Most embodiments of methods of the present invention use a double-stranded promoter. However, in some embodiments, the promoters of the invention comprise single-stranded pseudopromoters or synthetic promoters that are recognized by an RNAP so as to function in a method or assay of the invention. A “pseudopromoter” or “synthetic promoter” of the present invention can be any single-stranded sequence that is identified and/or selected to be functional as a promoter for in vitro transcription by an RNAP that recognizes said promoter with specificity and which functions as a promoter for said RNAP in a method or assay of the invention. A promoter comprising a pseudopromoter or synthetic promoter of the invention can be made as described by Ohmichi et al. (Proc. Natl. Acad. Sci. USA. 99: 54-59, 2002), which reference is incorporated herein by reference. If a pseudopromoter or synthetic promoter is used as a promoter in a method or assay of the invention, then the corresponding RNAP for which the pseudopromoter or synthetic promoter was identified and/or selected is used in the method or assay. By way of example, but not of limitation, a target probe with a promoter comprising a ssDNA pseudopromoter can be obtained and used in a method or assay of the invention that uses E. coli RNAP or a T7-type phage RNAP, such as, but not limited to, T7 RNAP, T3 RNAP, or SP6 RNAP, as described by Ohmichi et al. (Proc. Natl. Acad. Sci. USA. 99: 54-59, 2002) and incorporated herein by reference. Suitable pseudopromoters for E. coli RNAP that can be used in embodiments of the present invention are those found by Ohmichi et al. (Proc. Natl. Acad. Sci. USA. 99: 54-59, 2002).

A single-stranded promoter can also comprise a single-stranded N4 vRNAP promoter (Haynes, et al., Cell 41:597-605, 1985; Glucksmann, et al., Cell 70:491-500, 1992), such as a P1 promoter (3′-CAACGAAGCGTTGAATACC T-5′),

a P2 promoter (3′-TTCTTCGAGGCGAAGAAAACCT-5′) or a P3 promoter (3′-CGACGAGGCGTCGAAAACCA-5′) in some embodiments, in which case a transcriptionally active 1,106-amino acid domain of the N4 vRNAP (“mini-vRNAP”), which corresponds to amino acids 998-2103 of N4 vRNAP (Kazmierczak, K. M., et al., EMBO J., 21: 5815-5823, 2002; U.S. Patent Application No. 20030096349, incorporated herein by reference) can be used. Alternatively, an N4 mini-vRNAP Y678F mutant enzyme (U.S. Patent Application No. 20030096349) can be used. If a single-stranded promoter is used, the cognate RNA polymerase for the promoter is used for transcription, but an anti-sense promoter oligo is not needed to obtain a circular or linear transcription substrate in those embodiments. Use of compositions comprising a single-stranded promoter is not suitable for embodiments of the invention in which an anti-sense promoter oligo that is attached to a solid support is used to obtain a transcription substrate comprising a double-stranded promoter.

The promoter sequence that is joined to the 3′-end of the target-complementary sequence at the 5′-end of a monopartite promoter target probe or a bipartite target probe of the present invention, as described in greater detail elsewhere herein, comprises a “sense promoter sequence” or a “sense promoter.” If the RNA polymerase used in a method of the invention requires a double-stranded promoter to obtain a functional promoter, the “sense promoter sequence” refers to the promoter sequence of the double-stranded promoter that is operably joined to the template strand for transcription (i.e., the strand that is copied to make a transcription product). As used herein, the sense promoter sequence that is joined to a target-complementary sequence in a target probe can also include one, two or a small number of additional nucleotides that serve as sites for initiation of transcription, which nucleotides are designated respectively as “the +1 nucleotide,” “the +2 nucleotide,” etc. By way of example, but without limiting the invention, with respect to a functional double-stranded promoter sequence for T7 RNAP, a corresponding sense T7 promoter sequence and +1 base that can be used in a target probe of the present invention is:

(5′CTATAGTGAGTCGTATTA 3′).

Following annealing of the target probes to a target sequence and ligation of the target probes with a ligase under ligation conditions, it is still necessary to anneal an “anti-sense promoter oligo” under hybridization conditions to the sense promoter sequence in order to obtain a transcription substrate of the invention that has a functional double-stranded promoter that can be used for in vitro transcription under transcription conditions. By way of example, but without limiting the invention, if the sense T7 promoter sequence and +1 base above is used in a target probe of the invention, a corresponding anti-sense promoter oligo can comprise the following anti-sense T7 promoter sequence and +1 base to obtain a functional double-stranded promoter sequence for T7 RNAP:

(5′ TAATACGACTCACTATAG 3′).

As discussed herein, another composition of the invention can be an anti-sense promoter oligo that is annealed to a complementary sense promoter in order to obtain a circular or linear transcription substrate having a functional double-stranded promoter. In general, an anti-sense promoter oligo comprises deoxyribonucleotides. Modified nucleotides or modified linkages should be used in an anti-sense promoter oligo only after carefully determining that they do not substantially affect the ability of the anti-sense promoter oligo to complex with a sense promoter sequence or to bind the RNA polymerase or to affect the ability of the RNA polymerase to initiate transcription using the template strand. However, modified nucleotides can be used for a particular purpose. Similarly, modified linkages, such as, but not limited to alpha-thiophosphate sugar linkages that are resistant to certain nucleases can be used for a particular purpose. An anti-sense promoter oligo can be of any length so long as it has sufficient length to comprise an anti-sense promoter sequence that, when annealed to a sense promoter, makes a functional double-stranded promoter that can be used by an RNA polymerase under transcription conditions to make a transcription product. The oligo comprising the anti-sense promoter can comprise additional nucleotides that are 3′-of or 5′-of the anti-sense promoter sequence so long as the additional nucleotides do not bind the intended target sequence or another component used in a method of the invention in a manner that is independent of complexing of the anti-sense promoter sequence with the sense promoter sequence in a ligation product of target probes annealed to the target sequence, or otherwise negatively affect the results of the method. If modified nucleotides are used in anti-sense promoter oligo for a purpose such as, but not limited to for attaching a biotin or other moiety, it is preferred that the modified nucleotide is in a nucleotide that does not comprise the anti-sense promoter sequence if possible. If an anti-sense promoter oligo is present in a reaction when steps such as primer extension with a DNA polymerase or ligation with a ligase are performed and it is not intended to primer extend the anti-sense promoter oligo annealed to the ligation product, the anti-sense promoter oligo is designed so that it cannot participate in these reactions. This is accomplished, for example, by synthesizing an anti-sense promoter oligo that has a dideoxynucleotide or another termination nucleotide on its 3‘-end so that it can’t be primer-extended. An anti-sense promoter oligo also typically does not have a phosphate group on its 5′-end so that it cannot participate in a ligation reaction.

Another composition of an anti-sense promoter oligo of the invention can be an oligonucleotide comprising an anti-sense promoter that is immobilized or attached to a solid support. Alternatively, in other embodiments, a composition of the invention can be an anti-sense promoter oligo having a moiety, such as but not limited to a biotin moiety that permits binding of the anti-sense promoter oligo to a solid support after annealing to the ligation product obtained by ligation of target probes annealed to a target sequence. Annealing of the resulting ligation product to the anti-sense promoter oligo thus generates a transcription substrate of the present invention. One reason to attach the anti-sense promoter oligo to a solid support after annealing to the sense promoter sequence of a ligation product is that solution hybridization is generally more efficient than hybridization on a surface. An anti-sense promoter oligo that is to be attached to a solid support can comprise a biotin moiety at or near its 5′-end, in which case, the anti-sense promoter oligo can be attached to a solid support that is covalently or non-covalently joined to an avidin or streptavidin moiety using any of the variety of joining methods known in the art. Whether the anti-sense promoter oligo is attached to a solid support prior to annealing to the ligation product or is attached to the solid support after annealing to the ligation product, preferably the anti-sense promoter oligo comprising the anti-sense promoter is immobilized on the solid support at or near its 5′-end and the anti-sense promoter sequence is at a sufficient distance from the surface of the solid support so that the sense promoter in a circular ligation product resulting from ligation of a bipartite target probe annealed to a target sequence or in a linear ligation product resulting from ligation of monopartite target probes annealed to a target sequence can complex with or anneal to the anti-sense sequence so as to make a functional immobilized circular or linear transcription substrate, respectively, when the support is incubated with an RNA polymerase that uses the double-stranded promoter to make a transcription product in a reaction medium under suitable transcription conditions. A biotin may be attached to the anti-sense promoter oligo, for example, but without limitation, by using a ribonucleoside triphosphate that is derivatized with biotin. Exemplary methods for making derivatized nucleoside triphosphates are disclosed in detail in Rashtchian et al., “Nonradioactive Labeling and Detection of Biomolecules,” C. Kessler, Ed., Springer-Verlag, New York, pp. 70-84, 1992, herein incorporated by reference. However, a number of other methods for attaching an oligo to a solid support, including but not limited to chemical synthesis, are known in the art and any suitable method for attaching an anti-sense promoter oligo so that its 3′-end is not attached and so that its anti-sense promoter sequence can complex with a sense promoter sequence so as to form a transcription substrate of the invention can be used.

Preferably, the solid support has a chemical composition and structure so that it does not non-specifically bind nucleic acid from a sample or that comprises a composition of the invention, such as, but not limited to a sense promoter primer. Preferably, the solid support has a chemical composition and structure so that it does not non-specifically bind enzymes, co-factors or other substances in reactions comprising methods of the invention. Without limiting the invention, solid supports can comprise dipsticks, membranes, such as nitrocellulose or nylon membranes, beads, chips or slides used for making arrays or microarrays, and the like. Some solid supports and methods for immobilizing or attaching an anti-sense promoter oligo on a surface or solid support, which can be used for the present invention, are disclosed by Marble et al. in U.S. Pat. No. 5,700,667 and in references therein, all of which methods are incorporated herein by reference. Other solid supports which can be used for the present invention are also known in the art and can be used. Numerous other methods for attaching a molecule comprising an oligonucleotide to a surface or other substance are known in the art, and any known method for attaching or immobilizing a molecule comprising an anti-sense promoter oligo can be used to make a composition comprising an immobilized anti-sense promoter oligo is included in the present invention.

If a single-stranded promoter, such as the P2 promoter for an N4 mini-vRNAP (PCT Publication No. WO 02/095002 A2), or pseudopromoters, such as the pseudopromoter identified by Ohmichi et al. (Proc. Natl. Acad. Sci. USA. 99: 54-59, 2002) for E. coli RNAP is used, these promoter sequences are joined to the template strand of a transcription substrate of the invention and are therefore, “sense promoter sequences” of the invention. However, a corresponding anti-sense promoter oligo is not used in these embodiments.

It is preferred in many embodiments that a kit is used according to the instructions of the manufacturer to obtain appropriate reaction media and conditions for carrying out in vitro transcription for the methods of the present invention. With respect to transcription reactions of the invention with a wild-type or mutant T7 RNAP enzymes, the reaction conditions for in vitro transcription are those provided with the AmpliScribe™ T7-Flash™ Transcription Kit, or the AmpliScribe™ T7 High Yield Transcription Kit, or the DuraScribe™ T7 Transcription Kit or, for incorporation of 2′-substituted deoxyribonucleotides other than 2′-fluorine-substituted deoxyribonucleotides, with the T7 R&DNA™ Polymerase, in each case according to the instructions of the manufacturer EPICENTRE Technologies, Madison, Wis.). If a T3 or SP6 RNAP is used for transcription using a method of the invention, the reaction conditions for in vitro transcription are those provided with the AmpliScribe™ T3 High Yield Transcription Kit or with the AmpliScribe™ T3-Flash™ High Yield Transcription Kit, or with the AmpliScribe™ SP6 High Yield Transcription Kit, in each case according to the instructions of the manufacturer EPICENTRE Technologies, Madison, Wis.). Kits or individual enzymes, including reaction buffers and instructions for use are also available. For example, products are commercially available for E. coli RNA polymerase and Thermus RNAP (EPICENTRE Technologies, Madison, Wis.).

Alternatively, users can prepare their own reagents based on published conditions known in the art for a particular RNA polymerase. By way of example, the conditions below can be used for in vitro transcription with T7 RNAP, an exemplary T7-like RNAP. An in vitro transcription reaction is prepared by setting up a reaction mixture containing the following final concentrations of components, added in the order given: 0.1 micromolar of a T7 RNAP sense promoter-containing DNA oligo; 1×transcription buffer comprising 40 mM Tris-HCl (pH 7.5), 6 mM MgCl₂, 2 mM spermidine, and 10 mM NaCl; 1 mM DTT; 0.5 mM of each NTP (ATP, CTP, GTP and UTP); deionized RNase-free water so the final volume will be 50 microliters after addition of an RNAP. In some embodiments of the invention, such as using the T7 RNAP Y639F mutant enzyme, 2′-F-dUTP and 2′-F-dCTP are used at a final concentration of 0.5 mM each in place of UTP and CTP in order to obtain synthesis of modified RNA which is resistant to ribonuclease A-type enzymes. An RNase inhibitor, such as placental RNase inhibitor or an antibody inhibitor, which are commercially available, can be added to the reaction. Inorganic pyrophosphatase can be added to the reaction to prevent pyrophosphorolysis of the transcription product. Other modified nucleoside triphosphates can be used in place of or in addition to the canonical NTPs for specific applications. The reaction mixture is then incubated at 37° C. to permit synthesis of RNA from the template. The reaction can be followed by gel electrophoresis on a PAGE gel.

The invention is not limited to these reaction conditions or concentrations of reactants. Transcription reaction conditions can be altered to accommodate reactions conditions for other enzymes and reactants used in a method. Preferred conditions of a transcription process herein include a pH of between 6 and 9, with a pH of between 7.5 and 8.5 more preferred. Mg⁺² or Mn⁺², preferably Mg⁺² may be admixed. Preferred temperatures for the reaction are 25° C. to 50° C. with the range of 30° C. to 45° C. being more preferred and the range of 32° C. to 42° C. being most preferred. Those with skill in the art will know that other suitable reaction conditions under which an RNA polymerase of the invention can be used can be found by simple experimentation, and any of these reaction conditions are also included within the scope of the invention.

If mini-vRNAP or mini-vRNAP Y678F enzymes are used for in vitro transcription of a transcription substrate having a single-stranded N4 promoter, the following in vitro transcription reaction can be prepared by setting up a reaction mixture containing the following final concentrations of components, added in the order given: 0.1 micromolar of a N4 vRNAP promoter-containing DNA oligo; 1.0 micromolar EcoSSB Protein; 1×transcription buffer comprising 40 mM Tris-HCl (pH 7.5), 6 mM MgCl₂., 2 mM spermidine, and 10 mM NaCl; 1 mM DTT; 0.5 mM of each NTP (ATP, CTP, GTP and UTP); deionized RNase-free water so the final volume will be 50 microliters after addition of an RNAP; and 0.1 micromolar of mini-vRNAP or mini-vRNAP Y678F enzyme. In some embodiments of the invention, 2′-F-dUTP and 2′-F-dCTP are used at a final concentration of 0.5 mM each in place of UTP and CTP in order to obtain synthesis of modified RNA which is resistant to ribonuclease A-type enzymes. Other modified nucleoside triphosphates can also be used in place of or in addition to the canonical NTPs for specific applications. The reaction mixture is then incubated at 37° C. to permit synthesis of RNA from the template. The reaction can be followed by gel electrophoresis on a PAGE gel. Other components and reaction conditions, including those discussed above for T7-type RNA polymerases, can also be used without undue experimentation those with knowledge in the art to obtain suitably optimized conditions.

Reaction conditions for in vitro transcription using other RNA polymerases are known in the art and can be obtained from the public literature.

The term “transcription product” as used herein can comprise RNA or, in view of the ability of certain polymerases of the invention, including, without limitation, a T7 RNAP Y639F mutant enzyme or a T7 RNAP mutant enzyme having altered amino acids at both positions 639 and 784 (Sousa et al., U.S. Pat. No. 5,849,546; Padilla, R and Sousa, R, Nucleic Acids Res., 15: e138, 2002; Sousa, R and Mukherjee, S, Prog Nucleic Acid Res Mol Biol., 73: 1-41, 2003), to use base-substituted ribonucleotides, such as 5-allylamino-UTP, or non-canonical nucleotide substrates such as dNTPs or 2′-substituted 2′-deoxyribonucleotides such as, but not limited to 2′-fluoro-, 2′-amino-, 2′-methoxy-, or 2′-azido-substituted 2′-deoxyribonucleotides, a transcription product can comprise, in addition to RNA, DNA or modified DNA, or modified RNA, or a mixture thereof. The synthesized transcription product may comprise a detectable label such as a fluorescent tag, biotin, digoxigenin, 2′-fluoro nucleoside triphosphate, or a radiolabel such as a ³⁵S- or ³²P-label. The synthesized transcription product may be adapted for use as a probe for blotting experiments or in-situ hybridization. Nucleoside triphosphates (NTPs) or derivatized NTPs may be incorporated into the transcription product, and may optionally have a detectable label.

The target-complementary sequence at the 5′-end of a bipartite target probe or the 5′-end of a promoter target probe is a template for transcription by the cognate RNA polymerase that recognizes the promoter. However, the target-complementary sequence at the 5′-end of a bipartite target probe or a promoter target probe is kept short, with only about 4 to about 100 nucleotides and preferably with about 8 to about 30 nucleotides and the reaction conditions of the method are adjusted to minimize the amount of transcription product obtained in the absence of a target sequence. Still further, no transcription product is synthesized using the target-complementary sequence that anneals to the 3′-end of the target sequence or the signal sequence of a target probe as a template since these sequences are not joined to the promoter in the absence of a target sequence. Therefore, a method of the present invention detects, directly or indirectly, synthesis of transcription product that is complementary to the target-complementary sequence at the 3′-end of a bipartite target probe or, when monopartite target probes are used, that is joined to the 5′-end of a promoter target probe.

Yet another aspect of the current invention comprises delivering a transcription product into a cell after transcription. The delivery may be by microinjection, transfection, electroporation or another method in the art. In one embodiment, the transcription product comprises RNAi.

2. Separation, Quantitation, and Identification Methods for Transcription Products

In some embodiments, which are preferred embodiments, the transcription products made as a result of an assay or method of the invention are detected without separating the transcription products from other reaction components. However, following in vitro transcription, it may be desirable in some embodiments to separate the transcription products of several different lengths from each other and from the transcription substrate and the excess target probes, TSA probes (if used), and other reaction components. A variety of separation and detection methods can be used.

a. Gel Electrophoresis

In one embodiment, amplification products are separated by agarose, agarose-acrylamide or polyacrylamide gel electrophoresis using standard methods.

b. Chromatographic Techniques

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography. In yet another alternative, molecules, such as but not limited to transcription substrates, transcription products, analyte-binding substances or analytes that are labeled, such as but not limited to biotin-labeled or antigen-labeled, can be captured with beads bearing avidin or antibody, respectively.

c. Microfluidic Techniques

Microfluidic techniques include separation on a platform such as microcapillaries, designed by ACLARA BioSciences Inc., or the LabChip™ “liquid integrated circuits” made by Caliper Technologies Inc. These microfluidic platforms require only nanoliter volumes of sample, in contrast to the microliter volumes required by other separation technologies. Miniaturizing some of the processes involved in genetic analysis has been achieved using microfluidic devices. For example, published PCT Application No. WO 94/05414, to Northrup and White, incorporated herein by reference, reports an integrated micro-PCR apparatus for collection and amplification of nucleic acids from a specimen. U.S. Pat. Nos. 5,304,487 to Wilding et al., and 5,296,375 to Kricka et al., discuss devices for collection and analysis of cell containing samples and are incorporated herein by reference. U.S. Pat. No. 5,856,174, incorporated herein by reference, describes an apparatus which combines the various processing and analytical operations involved in nucleic acid analysis.

d. Capillary Electrophoresis

In some embodiments, it may be desirable to provide an additional or alternative means for analyzing the amplified genes. In these embodiments, micro capillary arrays are contemplated to be used for the analysis.

Microcapillary array electrophoresis generally involves the use of a thin capillary or channel which may or may not be filled with a particular separation medium. Electrophoresis of a sample through the capillary provides a size based separation profile for the sample. Microcapillary array electrophoresis generally provides a rapid method for size-based sequencing, PCR product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods, these methods provide sensitivity in the range of attomoles, which is comparable to the sensitivity of radioactive sequencing methods. Typically, these methods comprise photolithographic etching of micron scale channels on a silica, silicon or other crystalline substrate or chip, and can be readily adapted for use in the present invention. In some embodiments, the capillary arrays may be fabricated from the same polymeric materials described for the fabrication of the body of the device, using the injection molding techniques described herein.

Rectangular capillaries are known as an alternative to the cylindrical capillary glass tubes. Some advantages of these systems are their efficient heat dissipation due to the large height-to-width ratio and, hence, their high surface-to-volume ratio and their high detection sensitivity for optical on-column detection modes. These flat separation channels have the ability to perform two-dimensional separations, with one force being applied across the separation channel, and with the sample zones detected by the use of a multi-channel array detector.

In many capillary electrophoresis methods, the capillaries, e.g., fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g., hydroxyethyl cellulose, polyacrylamide, agarose and the like. Generally, the specific gel matrix, running buffers and running conditions are selected to maximize the separation characteristics of the particular application, e.g., the size of the nucleic acid fragments, the required resolution, and the presence of native or undenatured nucleic acid molecules. For example, running buffers may include denaturants, chaotropic agents such as urea or the like, to denature nucleic acids in the sample.

e. Mass Spectroscopy

Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). For low molecular weight molecules, mass spectrometry has been part of the routine physical-organic repertoire for analysis and characterization of organic molecules by the determination of the mass of the parent molecular ion. In addition, by arranging collisions of this parent molecular ion with other particles (e.g., argon atoms), the molecular ion is fragmented forming secondary ions by the so-called collision induced dissociation (CID). The fragmentation pattern/pathway very often allows the derivation of detailed structural information. Other applications of mass spectrometric methods known in the art can be found summarized in Methods in Enzymology, Vol. 193: “Mass Spectrometry” (J. A. McCloskey, editor), 1990, Academic Press, New York.

Due to the apparent analytical advantages of mass spectrometry in providing high detection sensitivity, accuracy of mass measurements, detailed structural information by CID in conjunction with an MS/MS configuration and speed, as well as on-line data transfer to a computer, there has been considerable interest in the use of mass spectrometry for the structural analysis of nucleic acids. The biggest hurdle to applying mass spectrometry to nucleic acids is the difficulty of volatilizing these very polar biopolymers. Therefore, “sequencing” had been limited to low molecular weight synthetic oligonucleotides by determining the mass of the parent molecular ion and through this, confirming the already known sequence, or alternatively, confirming the known sequence through the generation of secondary ions (fragment ions) via CID in an MS/MS configuration utilizing, in particular, for the ionization and volatilization, the method of fast atomic bombardment (FAB mass spectrometry) or plasma desorption (PD mass spectrometry).

Two ionization/desorption techniques are electrospray/ionspray (ES) and matrix-assisted laser desorption/ionization (MALDI). As a mass analyzer, a quadrupole is most frequently used. The determination of molecular weights in femtomole amounts of sample is very accurate due to the presence of multiple ion peaks, which all could be used for the mass calculation.

MALDI mass spectrometry, in contrast, can be particularly attractive when a time-of-flight (TOF) configuration is used as a mass analyzer. Since, in most cases, no multiple molecular ion peaks are produced with this technique, the mass spectra, in principle, look simpler compared to ES mass spectrometry. DNA molecules up to a molecular weight of 410,000 Daltons could be desorbed and volatilized. More recently, the use of infra red lasers (IR) in this technique (as opposed to UV-lasers) has been shown to provide mass spectra of larger nucleic acids such as synthetic DNA, restriction enzyme fragments of plasmid DNA, and RNA transcripts up to a size of 2180 nucleotides.

In Japanese Patent No. 59-131909, an instrument is described which detects nucleic acid fragments separated either by electrophoresis, liquid chromatography or high speed gel filtration. Mass spectrometric detection is achieved by incorporating into the nucleic acids atoms which normally do not occur in DNA such as S, Br, I or Ag, Au, Pt, Os, Hg.

f. Energy Transfer

Labeling hybridization oligonucleotide probes with fluorescent labels is a well known technique in the art and is a sensitive, nonradioactive method for facilitating detection of probe hybridization. More recently developed detection methods employ the process of fluorescence energy transfer (FET) rather than direct detection of fluorescence intensity for detection of probe hybridization. FET occurs between a donor fluorophore and an acceptor dye (which may or may not be a fluorophore) when the absorption spectrum of one (the acceptor) overlaps the emission spectrum of the other (the donor) and the two dyes are in close proximity. Dyes with these properties are referred to as donor/acceptor dye pairs or energy transfer dye pairs. The excited-state energy of the donor fluorophore is transferred by a resonance dipole-induced dipole interaction to the neighboring acceptor. This results in quenching of donor fluorescence. In some cases, if the acceptor is also a fluorophore, the intensity of its fluorescence may be enhanced. The efficiency of energy transfer is highly dependent on the distance between the donor and acceptor, and equations predicting these relationships have been developed. The distance between donor and acceptor dyes at which energy transfer efficiency is 50% is referred to as the Forster distance (R_{O}). Other mechanisms of fluorescence quenching are also known including, for example, charge transfer and collisional quenching.

Energy transfer and other mechanisms which rely on the interaction of two dyes in close proximity to produce quenching are an attractive means for detecting or identifying nucleotide sequences, since such assays may be conducted in homogeneous formats. Homogeneous assay formats are simpler than conventional probe hybridization assays which rely on detection of the fluorescence of a single fluorophore label, as heterogeneous assays generally require additional steps to separate hybridized label from free label.

Homogeneous methods employing energy transfer or other mechanisms of fluorescence quenching for detection of nucleic acid amplification have also been described. Higuchi et al. (Biotechnology, 10: 413-417, 1992) disclose methods for detecting DNA amplification in real-time by monitoring increased fluorescence of ethidium bromide as it binds to double-stranded DNA. The sensitivity of this method is limited because binding of the ethidium bromide is not target specific and background amplification products are also detected. WO 96/21144 discloses continuous fluorometric assays in which enzyme-mediated cleavage of nucleic acids results in increased fluorescence. Fluorescence energy transfer is suggested for use in the methods, but only in the context of a method employing a single fluorescent label which is quenched by hybridization to the target.

Signal primers or detector probes which hybridize to the target sequence downstream of the hybridization site of the amplification primers have been described for use in detection of nucleic acid amplification (U.S. Pat. No. 5,547,861). The signal primer is extended by the polymerase in a manner similar to extension of the amplification primers. Extension of the amplification primer displaces the extension product of the signal primer in a target amplification-dependent manner, producing a double-stranded secondary amplification product which may be detected as an indication of target amplification. The secondary amplification products generated from signal primers may be detected by means of a variety of labels and reporter groups, restriction sites in the signal primer which are cleaved to produce fragments of a characteristic size, capture groups, and structural features such as triple helices and recognition sites for double-stranded DNA binding proteins.

Many donor—acceptor dye pairs known in the art and may be used in the present invention. These include, for example: fluorescein isothiocyanate (FITC)—tetramethylrhodamine isothiocyanate (TRITC); FITC—Texas Red (Molecular Probes); FITC—N-hydroxysuccinimidyl 1-pyrenebutyrate (PYB); FITC—eosin isothiocyanate (EITC); N-hydroxysuccinimidyl 1-pyrenesulfonate (PYS)—FITC; FITC—Rhodamine X; FITC—tetramethylrhodamine (TAMRA); and others. The selection of a particular donor—acceptor fluorophore pair is not critical. For energy transfer quenching mechanisms, it is only necessary that the emission wavelengths of the donor fluorophore overlap the excitation wavelengths of the acceptor, i.e., there must be sufficient spectral overlap between the two dyes to allow efficient energy transfer, charge transfer or fluorescence quenching. P-(dimethyl aminophenylazo) benzoic acid (DABCYL) is a non-fluorescent acceptor dye which effectively quenches fluorescence from an adjacent fluorophore, e.g., fluorescein or 5-(2′-aminoethyl) aminonaphthalene (EDANS). Any dye pair which produces fluorescence quenching in the detector nucleic acids of the invention are suitable for use in the methods of the invention, regardless of the mechanism by which quenching occurs. Terminal and internal labeling methods are both known in the art and may be routinely used to link the donor and acceptor dyes at their respective sites in the detector nucleic acid.

3. Comparison of Transcription Processes of the Present Invention With Other Methods in the Art

Paul Lizardi discusses the optional use of a transcription promoter in an open circle probe (“OCP”) for use in rolling circle amplification (“RCA”), as disclosed in U.S. Pat. Nos. 6,344,329; 6,210,884; 6,183,960; 5,854,033; 6,329,150; 6,143,495; 6,316,229; 6,287,824. However, in contrast to the methods of the present invention, Lizardi disclosed that a promoter portion can be included in an open circle probe so that RNA transcripts can be generated from tandem sequence DNA (“TS-DNA”), which is a product of rolling circle amplification. In contrast, in the methods of the present invention, the RNA transcripts are primary amplification products and are synthesized by in vitro transcription of transcription substrates obtained by target-dependent joining of target probes. Thus, the RNA transcripts of the present invention are complementary to the target probes used in an assay or method. Preferred promoters in the methods of Lizardi are T7 or SP6 RNA polymerase promoters, which are double-stranded promoters, and the cognate polymerase for the promoter is used for transcriptional amplification. Thus, in embodiments of Lizardi's invention that contain a promoter sequence, Lizardi's open circle probes actually contain a protopromoter sequence, to which a complementary sequence must be annealed or a second DNA strand needs to be synthesized in order to obtain a functional promoter. Lizardi further states that a promoter on an open circle probe, if present, is preferably immediately adjacent to the left target probe (i.e., the promoter is 5′-of the target-complementary sequence on the 3′-end of the open circle probe) and is oriented to promote transcription toward the 3′-end of the open circle probe so the orientation results in transcripts that are complementary to TS-DNA. Thus, the position and orientation of a promoter sequence in the methods disclosed by Lizardi are completely different and would not be workable for the methods of the present invention. As discussed elsewhere herein, a sense promoter sequence of the present invention must be located 3′-of the target-complementary sequence at the 5′-end of a bipartite target probe or must be located 3′-of the target-complementary sequence at the 5′-end of a monopartite promoter target probe in order to function in the assays and methods of the invention. In short, promoters, if present at all, are included in open circle probes for the methods of Lizardi in order to obtain secondary amplification of DNA replication products, rather than for the purpose of primary amplification as is the case in the methods and assays of the present invention.

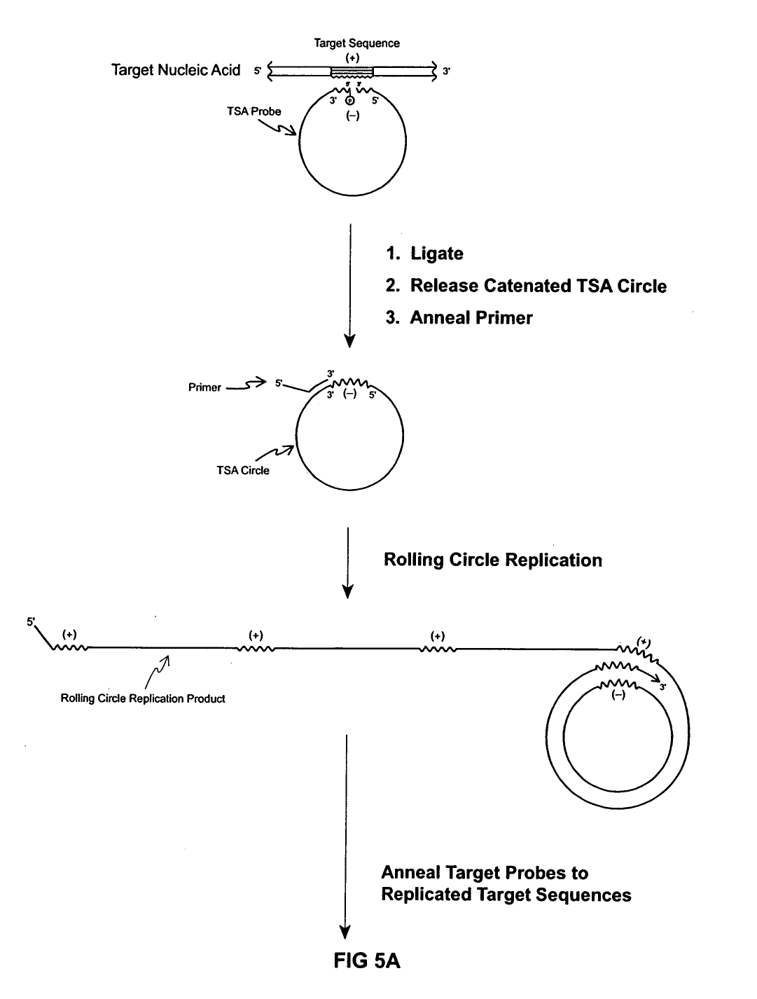

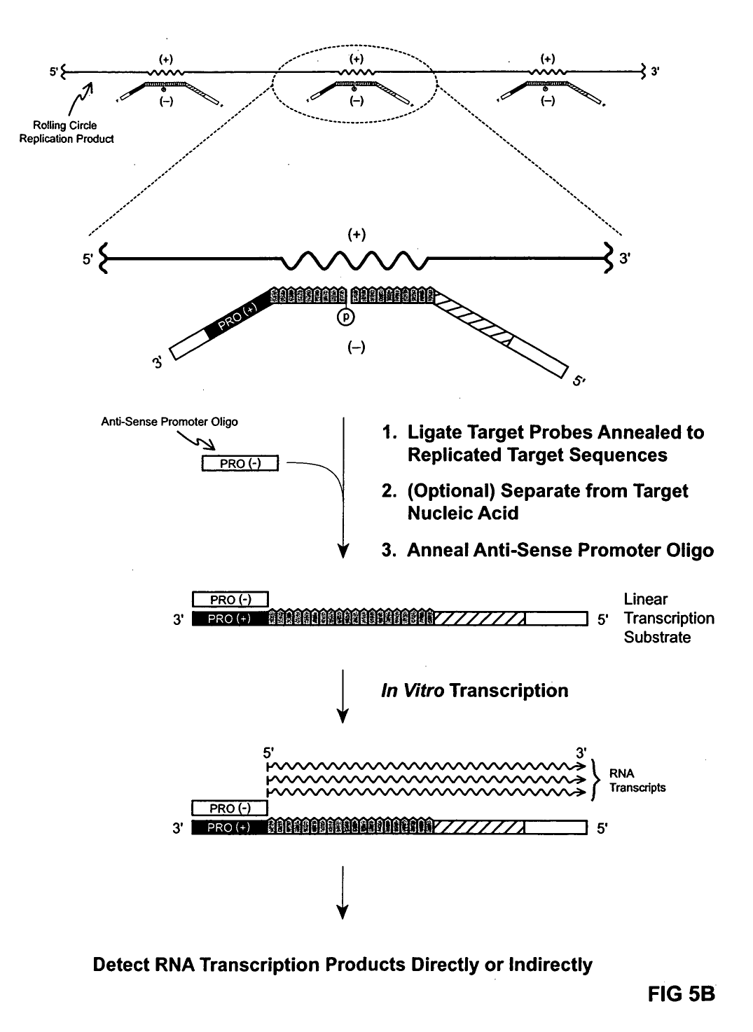

The present invention also differs in many respects from the methods disclosed in Japanese Patent Nos. JP4304900 and JP4262799 of Aono Toshiya et al. For example, Toshiya et al. did not disclose target-dependent transcription using monopartite target probes that generate linear transcription substrates in the presence of a target sequence. Japanese Patent Nos. JP4304900 and JP4262799 also did not disclose a reaction comprising coupled rolling circle replication and target-dependent transcription, an example of which is shown in FIG. 5 herein, wherein a first target sequence amplification probe (TSA probe) is used to amplify the number of target sequences that can serve as annealing and ligation sites for target probes that are used to obtain a transcription substrate for by target-dependent transcription, thus increasing the sensitivity of the methods and assays of the present invention. Toshiya et al. also did not disclose other methods for amplifying the amount of transcription product obtained, such as the method shown in FIG. 9 herein.

Also, Toshiya et al. did not disclose the use of an anti-sense promoter oligo that is either attached to a solid support or that has a moiety, such as a biotin moiety, that permits binding to a solid support that is joined to another moiety, such as a streptavidin moiety, during the processes of a method or assay of the present invention.

The present invention also comprises target-dependent transcription methods that use target probes and target-dependent transcription to detect non-nucleic analytes by detecting a target sequence comprising a target sequence tag that is joined to an analyte-binding substance that binds the analyte. Significantly, Japanese Patent Nos. JP4304900 and JP4262799 did not disclose methods for detecting analytes other than nucleic acids.

Further, the present invention also discloses methods that use a target probe that has a signal sequence such as, but not limited to a sequence for a substrate for Q-beta replicase, that permits a significant additional increase in sensitivity and speed of an assay or method for detecting a target sequence, such as by incubating a transcription product comprising a Q-beta replicase substrate with Q-beta replicase under replication conditions. Toshiya et al. did not disclose use of a signal sequence to increase speed or sensitivity of an assay or method. In addition, use of a signal sequence of the present invention permits easier detection of the transcription product, whether by an indirect means, such as by detecting the amount of a Q-beta substrate replicated by Q-beta replicase, or by a direct means, such as using a molecular beacon to detect a specific sequence comprising the transcription product. In contrast, Toshiya et al. only described detection of transcription product using a laborious and time-consuming procedure of separating high molecular weight RNA from low molecular weight RNA (presumably background transcription) by gel electrophoresis, then transferring the size-separated RNA to a nylon membrane and exposing the membrane to X-ray film for one day to detect the amount of radioactively-labeled high molecular weight product.

Still further, based on the figures in Japanese Patent Nos. JP4304900 and JP4262799, Toshiya et al. did not disclose that the target-complementary sequences at the ends of the straight chain nucleotide probe of their invention should be adjacent to or joined to the 5′-end of a sense promoter sequence and did not specify the distance of the promoter sequence from the target-complementary sequences. The illustrations of Toshiya et al. show the promoter sequence at some distance from the target-complementary sequences. If the sense promoter sequence is far from the target-complementary sequence, as shown in the figures of Toshiya et al., the amount of background transcription product obtained from the unligated probe will be increased. In contrast, the present invention discloses that the sense promoter sequence of a bipartite target probe is joined to the 3′-end of the target-complementary sequence that anneals to the 5′-end of the target sequence, so that background transcription is minimized.

The methods of the present invention which pertain to the use of bipartite target probes to generate circular transcription substrates also comprise additional embodiments that differ from the methods of Toshiya et al. in certain important ways. Thus, although the methods disclosed by Toshiya et al. specified that only an RNA polymerase with helicase-like activity should be used, some embodiments of the present invention use an RNA polymerase that lacks helicase-like activity. For example, some embodiments of the present invention use an N4 bacteriophage-derived mini-vRNAP ((PCT Publication No. WO 02/095002 A2) that lacks helicase-like activity. Mini-vRNAP enzymes use single-stranded DNA templates and are unable to unwind or transcribe double-stranded DNA. Mini-vRNAP enzymes require EcoSSB Protein to displace the RNA product from the RNA:DNA hybrid obtained from in vitro transcription of linear templates (Davidova, E K and Rothman-Denes, L B, Proc. Natl. Acad. Sci. USA, 100: 9250-9255, 2003). The lack of helicase-like activity and lack of activity in displacing transcription products results in low background transcription of the linear target probes in embodiments in which an N4 min-vRNAP is used in an assay or method of the current invention. On the other hand, single-stranded circular transcription substrates, such as those obtained by annealing and ligation of a bipartite target probe of the invention on a target sequence, are efficiently transcribed by a rolling circle transcription mechanism using an N4 mini-vRNAP enzyme. Also, since mini-vRNAP enzymes recognize single-stranded sense promoters, annealing of an anti-sense promoter oligo is not used in methods that use a mini-vRNAP enzyme.

Unlike the methods described in Japanese Patent Nos. JP4304900 and JP4262799, the present invention also comprises other embodiments that use target probes that comprise a single-stranded pseudopromoter or synthetic promoter that is obtained for an RNA polymerase such, but not limited to E. coli RNAP or T7 RNAP. An anti-sense promoter oligo is not needed in these embodiments, which use a single-stranded pseudopromoter or synthetic promoter obtained as described by Ohmichi et al. (Proc. Natl. Acad. Sci. USA, 99: 54-59, 2002). In still other embodiments that differ from the methods of Toshiya et al., which are described herein in the section entitled “Other Embodiments of Bipartite Target Probes and Circular Transcription Substrates of the Invention: Simple Bipartite Target Probes and Simple Circular Transcription Substrates,” bipartite target probes that lack a transcription promoter sequence are used to generate circular ssDNA transcription substrates for rolling circle transcription using an RNA polymerase such as but not limited to an E. coli or T7-type RNA polymerase.

Still further, although Japanese Patent Nos. JP4304900 and JP4262799 disclosed use of phi29 DNA polymerase to replicate a circular ligation product, it was since shown that ligation of a similar probe when annealed to a target sequence created a circular DNA molecule catenated to the target nucleic acid comprising the target sequence and that rolling circle replication by phi 29 DNA polymerase was limited if the 3′-end of the target nucleic acid was more than about 150-200 bases from the target sequence (Nilsson, M. et al., Science, 265:2085-2088, 1994; Baner, J. et al., Nucleic Acids Research, 26: 5073-5078, 1998). Since this was not known by Toshiya et al. and since they used a target nucleic acid comprising bacterial genomic DNA, catenation of the ligated circular DNA to the target nucleic acid probably limited the amount of replication product obtained from their method. It is not known if catenation also affected the results they obtained for transcription of catenated circular DNA molecules on the target nucleic acid. As discussed previously, the present invention comprises steps to avoid problems due to catenation of the transcription substrate on the target nucleic acid.

These various problems may explain why the methods disclosed in Japanese Patent Nos. JP4304900 and JP4262799 did not appear to have been pursued. The present invention discloses a variety of processes and methods that overcome these problems.

N. Amplification and Detection Processes of the Invention: Amplifying a Target Sequence and Amplifying a Signal Sequence

The terms “amplifying a target” or “amplifying a target nucleic acid” or “amplifying a target nucleic acid sequence” or “amplifying a target sequence” herein mean increasing the number of copies of that portion of the sequence of a target nucleic acid for which a complementary sequence is present in a target probe of the invention, including, but not limited to, a target-complementary sequence that is present in a target probe that also comprises a sequence for a transcription promoter for an RNA polymerase. An “amplified target” or an “amplified target sequence” comprises only that portion of the sequence of a target nucleic acid for which a complementary sequence is present in a target probe of the invention. The use of the terms “amplifying a target” or “amplifying a target nucleic acid” or “amplifying a target nucleic acid sequence” or “amplifying a target sequence” herein is not intended to imply that all of the sequence of a target nucleic acid is amplified. The use of these terms is also not intended to imply that the amplification of that portion of the sequence of a target nucleic acid for which a complementary sequence is present in a target probe of the invention is actually directly observed or detected in a method or assay of the invention. The invention comprises embodiments in which the amplified target sequence is directly detected, such as, but not limited to, embodiments in which the target sequence is detected by measuring a fluorescent signal following annealing of a transcript-complementary detection probe such as, but not limited to a molecular beacon. The invention also comprises embodiments in which the amplified target sequence is detected only indirectly by generation of another signal, such as, but not limited to, embodiments in which a signal is generated as a result of transcription of another DNA sequence that is covalently attached to a target-complementary sequence and that is transcribed along with a target-complementary sequence. By way of example, but not of limitation, in one embodiment, which is a preferred embodiment, the amplification of a target sequence is detected by detecting a substrate for Q-beta replicase. The substrate is replicated by Q-beta replicase using replication conditions well known in the art following synthesis of said RNA substrate by transcription of a signal sequence portion of a target probe that encodes said Q-beta substrate. The term “amplification signal” as used herein is intended to describe the output or result of any method, whether direct or indirect, for detecting if amplification of a target sequence has occurred. By way of example, but not of limitation, an amplification signal can comprise a fluorescent signal that results from annealing of a molecular beacon to an RNA transcript that is complementary to a target probe, or an amplification signal can comprise a Q-beta substrate that is replicated by Q-beta replicase following transcription of a DNA portion of a target probe that encodes said Q-beta substrate. As discussed previously with respect to a signal sequence, the invention comprises any signal sequence and any detection method that detects target-dependent transcription of a target sequence or a signal sequence.

O. Reverse Transcriptases and Reverse Transcription Processes of the Invention

In some embodiments in which a target nucleic acid in a sample comprises RNA, reverse transcription is used to obtain a target sequence comprising DNA. Also, some embodiments of methods and assays of the present invention use reverse transcription processes in conjunction with other processes in order to obtain additional amplification of a target sequence and/or a signal sequence. These embodiments use a reverse transcriptase. A “reverse transcriptase” or “RNA-dependent DNA polymerase” is an enzyme that synthesizes a complementary DNA copy (“cDNA”) from an RNA template. All known reverse transcriptases also have the ability to make a complementary DNA copy from a DNA template; thus, they are both RNA- and DNA-dependent DNA polymerases. A primer is required to initiate synthesis with both RNA and DNA templates. Examples of reverse transcriptases that can be used in methods of the present invention include, but are not limited to, AMV reverse transcriptase, MMLV reverse transcriptase, Tth DNA polymerase, rBst DNA polymerase large fragment, also called IsoTherm™ DNA Polymerase (EPICENTRE Technologies, Madison, Wis., USA), and BcaBEST™ DNA polymerase (Takara Shuzo Co, Kyoto, Japan). In some cases, a mutant form of a reverse transcriptase, such as, but not limited to, an AMV or MMLV reverse transcriptase that lacks RNase H activity can be used. In other embodiments, a wild-type enzyme is preferred. In some embodiments of the invention, a separate RNase H enzyme, such as but not limited to, E. coli RNase H or Hybridase™ Thermostable RNase H (EPICENTRE Technologies, Madison, Wis. 53713, USA) can also be used in reverse transcription reactions. MMLV reverse transcriptase (wild-type, RNase H-positive) is preferred for some embodiments of the invention in which it can be used without a separate RNase H enzyme. In some other embodiments, IsoTherm™ DNA polymerase or AMV reverse transcriptase can be used. The processes of the invention include conducting experiments to determine the effects on amplification of RNase H activity of a reverse transcriptase and/or separate RNase H enzyme(s) used, including, but not limited to, AMV reverse transcriptase, IsoTherm DNA polymerase, and both RNase H-plus and RNase H-minus MMLV reverse transcriptase, and E. coli RNase H or thermostable RNase H enzymes that are stable for more than 10 minutes at 70° C. (U.S. Pat. Nos. 5,268,289; 5,459,055; and 5,500,370, incorporated herein by reference), such as, but not limited to, Hybridase™ thermostable RNase H, Tth RNase H, and Tfl RNase H (EPICENTRE Technologies, Madison, Wis., USA), or by different combinations of a reverse transcriptase and a separate RNase H. Kacian et al. (U.S. Pat. No. 5,399,491), incorporated herein by reference, discloses information related to the effects of adding different amounts of a separate RNase H enzyme to transcription-mediated amplification assays that used T7 RNAP and dsDNA templates and either MMLV or AMV reverse transcriptase, which information is useful in suggesting how to vary and evaluate reaction conditions related to use of reverse transcriptases and RNase H enzymes in methods and assays of the present invention.

P. Strand-Displacing DNA Polymerases for Rolling Circle Replication Processes of the Invention

Some DNA polymerases are able to displace the strand complementary to the template strand as a new DNA strand is synthesized by the polymerase. This process is called “strand displacement” and the DNA polymerases that have this activity are referred to herein as “strand-displacing DNA polymerases.” If the DNA template is a single-stranded circle, primed DNA synthesis procedes around and around the circle, with continual displacement of the strand ahead of the replicating strand, a process called “rolling circle replication.” Rolling circle replication results in synthesis of tandem copies of the circular template. The suitability of a DNA polymerase for use in an embodiment of the invention that comprises rolling circle replication can be readily determined. By way of example, but not of limitation, the ability of a polymerase to carry out rolling circle replication can be determined by using the polymerase in a rolling circle replication assay as described by Fire and Xu (Proc. Natl. Acad. Sci. USA, 92: 4641-4645, 1995), incorporated herein by reference. It is preferred that a DNA polymerase be a strand displacing DNA polymerase and lack a 5′-to-3′ exonuclease activity for strand displacement polymerization reactions using both linear or circular templates since a 5′-to-3′ exonuclease activity, if present, might result in the destruction of the synthesized strand. It is also preferred that DNA polymerases for use in the disclosed strand displacement synthesis methods are highly processive. The ability of a DNA polymerase to strand-displace can vary with reaction conditions, in addition to the particular enzyme used. Strand displacement and DNA polymerase processivity can also be assayed using methods described in Kong et al. (J. Biol. Chem., 268: 1965-1975, 1993), incorporated herein by reference.

Preferred strand displacing DNA polymerases of the invention are RepliPHI™ phi29 DNA polymerase (EPICENTRE Technologies, Madison, Wis., USA), phi29 DNA polymerase, rBst DNA polymerase large fragment (also called IsoTherm™ DNA polymerase (EPICENTRE Technologies, Madison, Wis., USA), BcaBEST™ DNA polymerase (Takara Shuzo Co., Kyoto, Japan), and SequiTherm™ DNA polymerase (EPICENTRE Technologies, Madison, Wis., USA). Other strand-displacing DNA polymerases which can be used include, but are not limited to phage M2 DNA polymerase (Matsumoto et al., Gene, 84: 247, 1989), phage phi PRD1 DNA polymerase (Jung et al., Proc. Natl. Acad. Sci. USA, 84: 8287, 1987), VENT® DNA polymerase (Kong et al., J. Biol. Chem. 268: 1965-1975, 1993), Klenow fragment of DNA polymerase I (Jacobsen et al., Eur. J. Biochem. 45: 623-627, 1974), T5 DNA polymerase (Chatterjee et al., Gene 97:13-19, 1991), PRD1 DNA polymerase (Zhu and Ito, Biochim. Biophys. Acta, 1219: 267-276, 1994), or T7 DNA polymerase in the presence of a T7 helicase/primase complex (Tabor and Richardson, Abstact No. 11, presented at the meeting “New Horizons in Genomics,” Mar. 30-Apr. 1, 2003 in Santa Fe, N. Mex., sponsored by the DOE Joint Genome Institute), all of which references, are incorporated herein by reference. Strand displacing DNA polymerases are also useful in some embodiments of the invention for strand displacement replication of linear first-strand cDNA, and in other embodiments, for rolling circle replication of circular first-strand cDNA.

In general, it is desirable that the amount of strand-displacing DNA polymerase in the reaction be as high as possible without inhibiting the reaction. By way of example, but without limitation, RepliPHI™ phi29 DNA Polymerase can be used at about 0.05 microgram to about one microgram of protein in a 20-microliter reaction and IsoTherm™ DNA Polymerase can be used at about 50 units to about 300 units in a 50-microliter reaction. Since definitions for units vary for different DNA polymerases and even for similar DNA polymerases from different vendors or sources, and also because the activity for each enzyme varies at different temperatures and under different reaction conditions, it is desirable to optimize the amount of strand-displacing DNA polymerase and reaction conditions for each target sequence and particular assay or method of the invention. Although not required for all DNA polymerases, strand displacement can be facilitated for some DNA polymerases through the use of a strand displacement factor, such as a helicase. It is considered that any DNA polymerase that can perform rolling circle replication in the presence of a strand displacement factor is suitable for use in embodiments of the invention that comprise rolling circle replication, even if the DNA polymerase does not perform rolling circle replication in the absence of such a factor. Strand displacement factors useful in rolling circle replication include, but are not limited to, BMRF1 polymerase accessory subunit (Tsurumi et al., J. Virology, 0.67: 7648-7653, 1993), adenovirus DNA-binding protein (Zijderveld and van der Vliet, J. Virology, 68: 1158-1164, 1994), herpes simplex viral protein ICP8 (Boehmer and Lehman, J. Virology, 67: 711-715, 1993); Skaliter and Lehman, Proc. Natl. Acad. Sci. USA, 91: 10,665-10,669, 1994), single-stranded DNA binding proteins (SSB; Rigler and Romano, J. Biol. Chem., 270: 8910-8919, 1995), and calf thymus helicase (Siegel et al., J. Biol. Chem., 267: 13,629-13,635, 1992), all of which are incorporated herein by reference.

Q. Other Embodiments of Monopartite Target Probes and Linear Transcription Substrates of the Invention

1. Immobilizing a Linear Transcription Substrate on a Solid Support

Still another embodiment of the invention comprises a composition of promoter target probe that is immobilized or attached to a solid support. Alternatively, in other embodiments, the promoter target probe can have a moiety, such as but not limited to a biotin moiety on or near to its 3′-end that permits attachment of the promoter target probe to a solid support after annealing to the target sequence and ligation to an adjacently annealed target probe, such as a signal target probe or a simple target probe. Once complexed with an anti-sense promoter oligo, the ligation product obtained by ligating the promoter target probe to the adjacently annealed target probe on the target sequence generates a transcription substrate of the present invention, whether the promoter target probe is attached to a solid support or has a biotin or other moiety that permits attachment to a solid support. One reason to attach the promoter target probe to a solid support after ligating to an adjacently annealed target probe on a target sequence is that solution hybridization is generally more efficient than hybridization on a surface. If the ligation product is attached to a solid support, the complexing of an anti-sense promoter oligo with the ligation product can be before or after the ligation product is attached to the solid support. A promoter target probe that is to be attached to a solid support can comprise a biotin moiety at or near its 3′-end, in which case, the promoter target probe can be attached to a solid support that is covalently or non-covalently joined to an avidin or streptavidin moiety using any of the variety of joining methods known in the art. Whether the promoter target probe is attached to a solid support prior to annealing to the target sequence and ligating to one or more other target probes or is attached to the solid support after annealing to the target sequence and being ligated to one or more other target probes and/or complexing with an anti-sense promoter oligo, preferably the promoter target probe or the resulting ligation product is immobilized on the solid support at or near its 3′-end and is at a sufficient distance from the surface of the solid support so that the promoter in double-stranded form can bind a cognate RNA polymerase and initiate transcription therefrom under suitable transcription conditions. A biotin may be attached to the promoter target probe, for example, but without limitation, by using a ribonucleoside triphosphate that is derivatized with biotin. Exemplary methods for making derivatized nucleoside triphosphates are disclosed in detail in Rashtchian et al., “Nonradioactive Labeling and Detection of Biomolecules,” C. Kessler, Ed., Springer-Verlag, New York, pp. 70-84, 1992, herein incorporated by reference. Preferably, the solid support has a chemical composition and structure so that it does not non-specifically bind nucleic acid from a sample or that comprises a composition of the invention, such as, but not limited to a sense promoter primer. Preferably, the solid support has a chemical composition and structure so that it does not non-specifically bind enzymes, co-factors or other substances in reactions comprising methods of the invention. Without limiting the invention, solid supports can comprise dipsticks, membranes, such as nitrocellulose or nylon membranes, beads, chips or slides used for making arrays or microarrays, and the like. Some solid supports which can be used for the present invention are disclosed by Marble et al. in U.S. Pat. No. 5,700,667. Other solid supports which can be used for the present invention are also known in the art and can be used. Numerous methods for attaching a molecule comprising an oligonucleotide to a solid support are known in the art, and any known method for attaching or immobilizing a molecule comprising a promoter target probe or a ligation product derived therefrom can be used to make a composition comprising an immobilized promoter target probe or a resulting immobilized ligation product or linear transcription substrate and is included in the present invention.

2. Obtaining a Circular Transcription Subtrate by Circularizing a Ligation Product Obtained Using Monopartite Target Probes

In some embodiments of the present invention, a circular transcription substrate is obtained using monopartite target probes rather than a bipartite target probe. In these embodiments, the monopartite target probes anneal to the target sequence and are ligated in the presence of a target sequence to form a linear ligation product as described previously. However, in these embodiments, the linear ligation product is denatured from the target sequence and subsequently circularized by ligation of its 3′-end to its 5′-end. The 5′-end of the linear ligation product has a 5′-phosphate group or is phosphorylated using a kinase, such as but not limited to T4 polynucleotide kinase, in the presence of ATP. This 5′-phosphorylated linear ligation product is then complexed with a ligation splint oligo that has ends that are complementary to the 3′-end and the 5′-end of the linear ligation product and the ends are ligated under ligation conditions with a ligase that has little or no activity in ligating blunt ends and that is substantially more active in ligating ends that are adjacent when annealed to a contiguous complementary sequence than if the ends are not annealed to the complementary sequence, such as but not limited to Ampligase® DNA Ligase (EPICENTRE Technologies, Madison, Wis.). The use of a ligation splint and a ligase, such as Ampligase® DNA Ligase, that is not active in ligating blunt ends or non-homologous ligation minimizes “background,” such as background rolling circle transcription that could result from a circular molecule obtained by intramolecular ligation of a promoter target probe if a non-homologous ligase were used. Preferably, the same ligase is used both for ligation of the target probes annealed to the target sequence and for subsequent ligation of the 3′-end to the 5′-end of the ligation product using a ligation splint. After annealing an anti-sense promoter oligo to the circularized ligation product, a circular transcription substrate of the invention is obtained.

One reason to circularize a linear ligation product obtained from monopartite target probes is because rolling circle transcription is often more efficient and generates more transcription product than transcription of linear transcription substrates. Since initiation of transcription (rather than elongation) is usually a rate-limiting step for transcription, the efficiency of transcription of circular versus linear transcription substrates is particularly increased for small transcription substrates. Still further, transcription is also greatly enhanced for circular transcription substrates in embodiments that use an N4 mini-vRNAP because the transcription product is not efficiently displaced from linear transcription substrates (Davidova, E K and Rothman-Denes, L B, Proc. Natl. Acad. Sci. USA 100:9250-9255, 2003), whereas the transcription product of rolling circle transcription of small circular transcription substrates by an N4 min-vRNAP is displaced and transcription is therefore much more efficient and productive.

R. Other Embodiments of Bipartite Target Probes and Circular Transcription Substrates of the Invention: Simple Bipartite Target Probes and Simple Circular Transcription Substrates

Daubendiek et al. (J. Am. Chem. Soc., 117: 7818-7819, 1995) and Eric T. Kool (U.S. Pat. Nos. 5,714,320; 6,077,668; 6,096,880; and 6,368,802 B1), all of which are incorporated herein in their entirety by reference, disclose that very small (18 to ˜110 nucleotides) circular, usually pyrimidine-rich, single-stranded DNA (ssDNA) molecules that lack a known transcription promoter sequence can be transcribed by E. coli bacterial and T7-type phage RNA polymerases, with transcription occurring at different efficiencies by each polymerase depending on the sequence of the circular ssDNA. In some cases, initiation of RNA synthesis with these circular ssDNAs occurs primarily with pppG, as is usually the case for promoter-initiated transcription using these RNA polymerases. In some cases, a linear precursor of a circular ssDNA is transcribed little or not at all under conditions in which the corresponding circular ssDNA is transcribed efficiently. Thus, in some embodiments of the present invention, a “simple bipartite target probe” is used that lacks a known promoter sequence. In these embodiments, a “simple bipartite target probe” comprises a linear ssDNA precursor to a circular ssDNA molecule, wherein the 3′-end portion and the 5′-end portion of said linear ssDNA precursor comprise sequences that are complementary, respectively, to the most 5′-portion and the most 3′-portion of a target nucleic acid sequence. A simple bipartite target probe of this embodiment of an assay or method of the invention comprises a linear ssDNA precursor of a circular ssDNA molecule, wherein said simple bipartite target probe is transcribed little or not at all, but said circular ssDNA molecule is an efficient template for rolling circle transcription by an RNA polymerase used in said assay or method. Thus, annealing of said simple bipartite target probe to a target sequence and target sequence-dependent ligation of said simple bipartite target probe during a process of an assay or method of the invention yields a circular ssDNA molecule comprising a “simple circular transcription substrate” of the invention. In order to make a simple bipartite target probe of this embodiment, a circular ssDNA molecule, such as, but not limited to, those reported by Daubendiek et al. and by Kool, is identified as an efficient substrate for rolling circle transcription. Then a target-complementary sequence is inserted into different sites of said circular ssDNA molecule until a circular ssDNA molecule comprising said target-complementary sequence is identified to be an efficient substrate for rolling circle transcription. Circular ssDNA molecules can be made as described (Prakash, G and Kool, E. T., J. Am. Chem. Soc., 114: 3523-3527, 1992; Wang, S. and Kool, E. T., Nucleic Acids Res., 22: 2326-2333, 1994; Kool, E. T. in U.S. Pat. Nos. 5,714,320; 6,077,668; 6,096,880; and 6,368,802 B1, all of which are incorporated herein in their entirety by reference). If an efficient substrate is not identified comprising said target-complementary sequence, then another target-complementary sequence is evaluated until an efficient substrate for rolling circle transcription is found.

Thus, one embodiment of the invention is a method for detecting a target nucleic acid sequence, the method comprising: (a) providing a simple bipartite target probe comprising linear single-stranded DNA (ssDNA) that lacks a sequence for a known promoter for an RNA polymerase, the simple bipartite target probe comprising two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, wherein said simple bipartite target probe is transcribed little or not at all by an RNA polymerase under conditions in which a circular ssDNA obtained by intramolecular ligation of the simple bipartite target probe is transcribed efficiently by said RNA polymerase; (b) contacting the simple bipartite target probe with the target nucleic acid sequence and incubating under hybridization conditions, wherein the ends of the target-complementary sequences anneal adjacently on the target nucleic acid sequence to form a complex; (c) contacting the complex with a ligase under ligation conditions so as to obtain a circular ssDNA ligation product comprising a circular transcription substrate for the RNA polymerase; (d) contacting the circular transcription substrate with the RNA polymerase under transcription conditions to obtain a transcription product; and (f) detecting the transcription product.

Preferably, the ligase has little or no activity in ligating blunt ends and is substantially more active in ligating ends that are adjacent when annealed to two contiguous regions of a target sequence compared to ends that are not annealed to the target sequence. One suitable ligase that can be used is Ampligase® Thermostable DNA Ligase (EPICENTRE Technologies, Madison, Wis.). In preferred embodiments, the RNA polymerase comprises an RNA polymerase chosen from among a T7 RNAP, a T3 RNAP, an SP6 RNAP or another T7-like RNA polymerase, including mutant forms thereof, or E. coli RNA polymerase or Thermus thermophilus RNA polymerase. Another suitable RNA polymerase is an N4 mini-vRNAP.

In some embodiments, the target sequence comprises a target nucleic acid in a sample, whereas in other embodiments the target sequence comprises a target sequence tag that is joined to an analyte-binding substance that binds an analyte in the sample. In some embodiments, the method is used to detect a single-nucleotide polymorphism (SNP) or mutation, in which case the 5′-nucleotide of the first target-complementary sequence or the 3′-end of the second target-complementary sequence of said simple bipartite target probe is complementary to the intended target nucleotide of the target sequence, and ligation only occurs when the ends of both target-complementary sequences are adjacently annealed on the target sequence, including the target nucleotide, under the stringent ligation conditions of the assay or method. The target sequence is preferably less than about 150 to about 200 nucleotides from the 3′-end of the target nucleic acid or target sequence tag. In embodiments in which the target sequence is greater than about 150 to about 200 nucleotides from the 3′-end of the target nucleic acid or target sequence tag comprising the target sequence, one or more additional steps is used in order to release the catenated circular ligation product from the target sequence prior to transcription, as described elsewhere herein. In still other embodiments in which a bipartite target probe is used, the circular transcription substrate that is transcribed remains catenated to a target nucleic acid.

In some embodiments of the invention, a simple bipartite target probe can also serve as a target sequence amplification probe (or TSA probe) that is used as described elsewhere herein to obtain additional target sequences for target-dependent ligation of the simple bipartite target probe to make additional simple circular transcription substrates. If the simple bipartite target probe also is used as a TSA probe in an assay or method of the invention, a primer that anneals to the sequence between the target-complementary portions is provided to prime rolling circle transcription of a TSA circle that results from ligation of a TSA probe annealed to a target sequence.

Simple bipartite probes can also be used in other embodiments for secondary amplification of an RNA transcription product or of a reverse transcription product derived therefrom. Thus, in those embodiments, a simple bipartite secondary amplification probe comprising end sequences that are complementary to other sequences than target sequences are used and the simple bipartite secondary amplification probe is annealed and ligated on either an RNA template resulting from transcription of a target-dependent transcription substrate or on a cDNA reverse transcription product derived from said RNA transcription product. By way of example, but not of limitation, the simple bipartite secondary amplification probe can be annealed and ligated on an RNA transcript or its cDNA product corresponding to a signal sequence portion of a transcription substrate. This embodiment is better understood following a complete reading of the description of the invention herein

S. Methods and Assays of the Invention for Detecting a Target Sequence

1. Methods and Assays of the Invention That Use a Target Probe Comprising a Double-Stranded Promoter for Detecting a Target Sequence

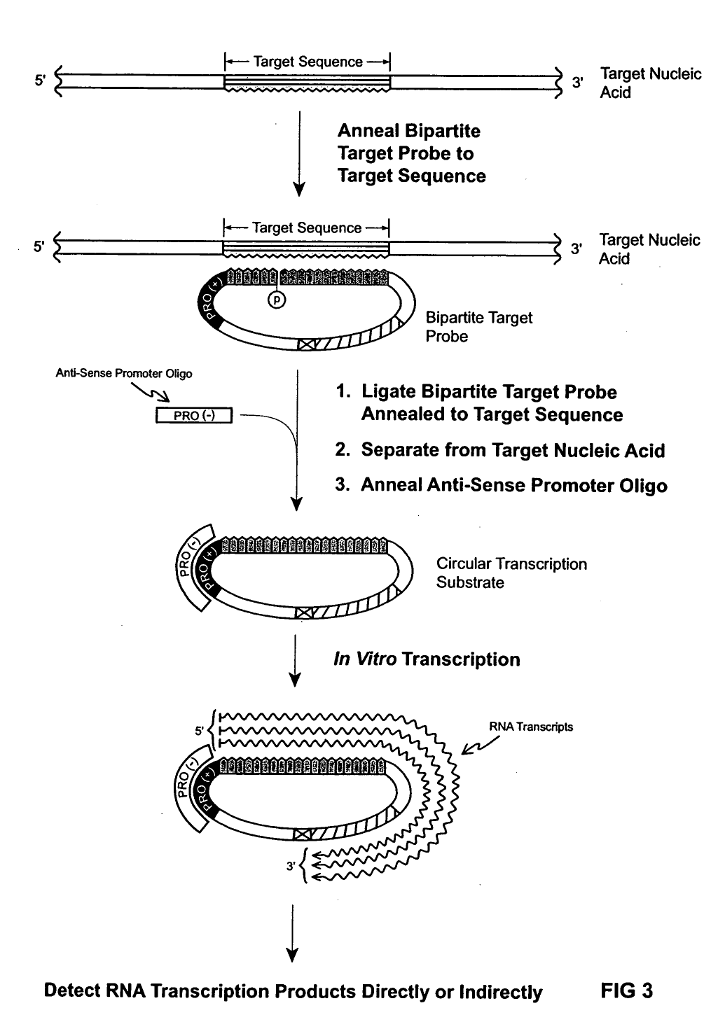

The present invention comprises methods, compositions and kits for detecting one or multiple specific target sequences in a sample by target-dependent transcription. FIG. 3 shows one basic embodiment of a method of the present invention. This embodiment uses a bipartite target probe. A bipartite target probe is a linear single-stranded DNA molecule that has sequences on both ends of the probe that are complementary to different portions of a target sequence. In the embodiment shown in FIG. 3, the target-complementary sequences of the bipartite target probe are contiguous or adjacent or abut to each other when annealed to the target sequence. The sequence at the 5′-end of the bipartite target probe preferably has a 5′-phosphate group or is phosphorylated by a polynucleotide kinase during the course of a method of the invention. The 5′-portion of the bipartite target probe also has sequence for a sense promoter sequence for a functional promoter for a DNA-dependent RNA polymerase, which upon complexing with an anti-sense promoter oligo, can bind to this double-stranded promoter and initiate transcription of RNA therefrom in a 5′-to-3′ direction using single-stranded DNA that is 5′-of and covalently linked to the promoter as a template. The promoter is oriented within the single-stranded DNA of the bipartite target probe 3′-of the target-complementary sequence at the 5′-end of the 5′-portion. The sequence at the 3′-end of the bipartite target probe preferably has a 3′-hydroxyl group.

Referring to FIG. 3, in the presence of a a target sequence comprising a single-stranded DNA target or one strand of a double-stranded DNA target, a bipartite target probe anneals to the target sequence under hybridization conditions, wherein the 5′-phosphorylated end of the bipartite target probe is adjacent to its 3′-hydroxyl end. Then, the ends of the bipartite target probe are ligated under ligation conditions by contacting the target-complementary ends annealed to a target sequence with a ligase that has little or no activity in ligating free ends that are not annealed to a complementary sequence but is active in joining a 5′-phosphorylated end to a 3′-hydroxylated end when the ends are adjacent when annealed to a complementary DNA sequence. Ligation of the ends of the bipartite target probe generates a “circular transcription substrate,” meaning a circular single-stranded DNA molecule that is a template for transcription by an RNA polymerase that recognizes a promoter sequence in said circular transcription substrate.

Thus, again referring to FIG. 3, one embodiment of the present invention comprises a method for detecting a target sequence, said method comprising:

a. providing a bipartite target probe, wherein said bipartite target probe comprises a linear single-stranded DNA comprising two separate target-complementary end portions that are complementary to a contiguous target sequence;

b. annealing said bipartite target probe to said target sequence under hybridization conditions;

c. ligating said bipartite target probe annealed to said target sequence under ligation conditions with a ligase, wherein said ligase has little or no activity in ligating blunt ends and is substantially more active in ligating said ends of said bipartite target probe if said ends are adjacent when annealed to two contiguous regions of a target sequence than if said ends are not annealed to said target sequence, so as to obtain a circular ssDNA ligation product;

d. contacting the ligation product with an anti-sense promoter oligo and incubating under hybridization conditions, wherein the anti-sense promoter oligo anneals to the sense promoter sequence of the ligation product to form a transcription substrate;

e. obtaining said circular transcription substrate, wherein said circular transcription substrate comprises a sequence that is complementary to said target sequence;

f. contacting said circular transcription substrate with an RNA polymerase under transcription conditions so as to synthesize transcription product that is complementary to said circular transcription substrate; and

g. detecting the synthesis of transcription product resulting from transcription of said circular transcription substrate, wherein said synthesis of said transcription product indicates the presence of the target sequence.

In some embodiments, the anti-sense promoter oligo is attached to a solid support. In other embodiments, the anti-sense promoter oligo comprises a moiety, such as, but not limited to a biotin moiety that permits binding of the anti-sense promoter oligo to a solid support after annealing to the ligation product to obtain a transcription substrate.

Also, since transcription of said circular transcription substrate increases the number of copies of the target sequence, the invention also comprises a method for amplifying a target sequence, said method comprising:

a. providing a bipartite target probe, wherein said bipartite target probe comprises a linear single-stranded DNA (ssDNA) comprising two separate target-complementary end portions that are complementary to a contiguous target sequence;

b. annealing said bipartite target probe to said target sequence under hybridization conditions;

c. ligating said bipartite target probe annealed to said target sequence under ligation conditions with a ligase, wherein said ligase has little or no activity in ligating blunt ends and is substantially more active in ligating said ends of said bipartite target probe if said ends are adjacent when annealed to two contiguous regions of a target sequence than if said ends are not annealed to said target sequence, so as to obtain a circular ssDNA ligation product;

d. contacting the ligation product with an anti-sense promoter oligo and incubating under hybridization conditions, wherein the anti-sense promoter oligo anneals to the sense promoter sequence of the ligation product to form a transcription substrate;

e. obtaining said circular transcription substrate, wherein said circular transcription substrate comprises a sequence that is complementary to said target sequence;

f. contacting said circular transcription substrate with an RNA polymerase under transcription conditions so as to synthesize transcription product that is complementary to said circular transcription substrate; and

g. obtaining transcription product comprising multiple copies of said target sequence.

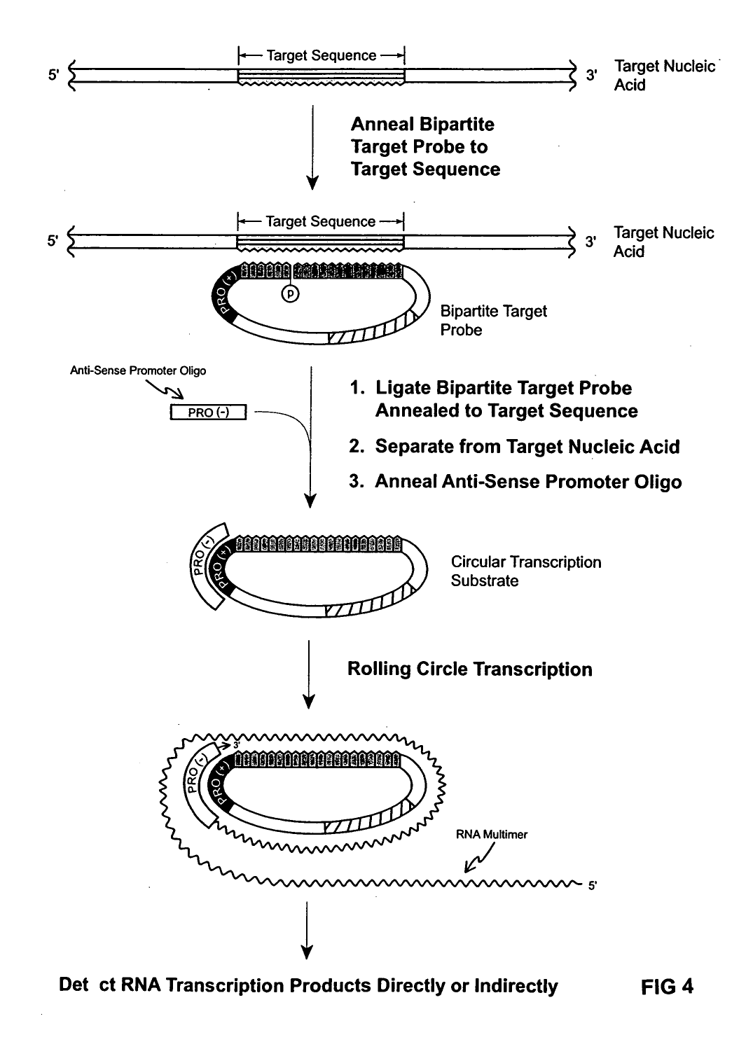

FIG. 4 shows an embodiment of a method or assay of the invention that is similar to the embodiment shown in FIG. 3 except that the bipartite target probe used in the method shown in FIG. 4 does not have a transcription termination sequence and transcription of the circular transcription substrate resulting therefrom generates a transcription product comprising a transcription product multimer by rolling circle transcription.

Thus, referring to FIG. 4, one embodiment of the present invention comprises a method for detecting a target sequence, said method comprising:

a. providing a bipartite target probe, wherein said bipartite target probe comprises a linear single-stranded DNA comprising two separate target-complementary end portions that are complementary to a contiguous target sequence;

b. annealing said bipartite target probe to said target sequence under hybridization conditions;

c. ligating said bipartite target probe annealed to said target sequence under ligation conditions with a ligase, wherein said ligase has little or no activity in ligating blunt ends and is substantially more active in ligating said ends of said bipartite target probe if said ends are adjacent when annealed to two contiguous regions of a target sequence than if said ends are not annealed to said target sequence, so as to obtain a circular ssDNA ligation product;

d. contacting the ligation product with an anti-sense promoter oligo and incubating under hybridization conditions, wherein the anti-sense promoter oligo anneals to the sense promoter sequence of the ligation product to form a transcription substrate;

e. obtaining said circular transcription substrate, wherein said circular transcription substrate comprises a sequence that is complementary to said target sequence;

f. contacting said circular transcription substrate with an RNA polymerase under rolling circle transcription conditions so as to synthesize transcription product multimers, wherein a transcription product multimer comprises multiple tandem copies of an oligomer that is complementary to one copy of said circular transcription substrate; and

g. detecting the synthesis of said transcription product resulting from rolling circle transcription of said circular transcription substrate, wherein said synthesis of said transcription product indicates the presence of said target sequence.

In preferred embodiments, the target sequence is less than about 150 to about 200 nucleotides from the 3′-end of the target nucleic acid or target sequence tag. If the target sequence is greater than about 150 to about 200 nucleotides from the 3′-end of the target nucleic acid or target sequence tag comprising the target sequence, then one or more additional steps (as described elsewhere herein) is required in order to release the catenated circular ligation product from the target sequence prior to transcription. In other embodiments, the circular transcription substrate that is transcribed remains catenated to a target nucleic acid.

In some embodiments, the anti-sense promoter oligo is attached to a solid support. In other embodiments, the anti-sense promoter oligo comprises a moiety, such as, but not limited to a biotin moiety that permits binding of the anti-sense promoter oligo to a solid support after annealing to the ligation product to obtain a transcription substrate.

Other embodiments of the present invention, as shown in FIG. 5, comprise compositions, methods and kits for detecting one or multiple specific target sequences in a sample by coupled target-dependent rolling circle replication (RCR) and rolling circle transcription (RCT). These embodiments use bipartite target probes to generate circular transcription substrates as shown in either FIG. 3 or FIG. 4, which result in circular transcription substrate either with a transcription terminator or lacking a transcription terminator, respectively. If the circular transcription substrate lacks a transcription terminator sequence, transcription comprises rolling circle transcription as described elsewhere herein. In addition to using a bipartite target probe, these embodiments also use a “bipartite target sequence amplification probe,” which is also referred to as a “bipartite TSA probe” or simply as a “TSA probe” herein. The purpose of a TSA probe in an assay or method is to obtain a target-dependent amplification of the number of copies of the target sequence and thereby, to provide additional sites for annealing and ligation of the bipartite target probe.

A TSA probe is a linear single-stranded DNA molecule that comprises two target-complementary sequences that are connected by an intervening sequence that is not complementary to the target sequence. The target-complementary portions on the ends are complementary to different portions of a target sequence in a target nucleic acid or a target sequence tag of an analyte-binding substance. Each of the 5′ and 3′ target-complementary sequences in a TSA probe for a particular assay or method is identical to the corresponding target-complementary sequence at the 5′-end or the 3′-end of a bipartite target probe used in the assay or method. That is, the 5′-end of a TSA probe anneals to the same nucleotides in the target sequence as the 5′-end of the corresponding bipartite target probe that is used to obtain a circular transcription substrate and similarly, the 3′-end of the TSA probe anneals to the same nucleotides of the target sequence as the 3′-end of the bipartite target probe. Thus, as shown in FIG. 5, the target-complementary sequences of the TSA probe are adjacent to each other when annealed to the target sequence in exactly the same manner as described previously for bipartite target probes. Similarly, the sequence at the 5′-end of the TSA probe preferably has a 5′-phosphate group or is phosphorylated by a polynucleotide kinase during the course of a method of the invention and the sequence at the 3′-end of a TSA probe preferably has a 3′-hydroxyl group. After annealing to a target sequence, if present in a sample, the adjacent target-complementary sequences of a TSA probe are ligated in a method of the invention with a ligase that has little or no activity in ligating blunt ends and that is substantially more active in ligating said ends that are adjacent when annealed to two contiguous regions of a target sequence than if said ends are not so annealed. Ligation of a TSA probe results in formation of a “TSA circle,” which, upon annealing to a primer, is a substrate for rolling circle replication.

The target-complementary sequences of a TSA probe are connected by an intervening sequence. The sequence and nucleotide composition of the intervening sequence can vary, but it should comprise a sequence of sufficient length and sequence specificity to provide a primer-binding site for specific priming by a primer for rolling circle replication. The intervening sequence should also be of sufficient length to permit the target-complementary sequences of the TSA probe to anneal to the target sequence with specificity. In addition, the length of the intervening sequence should be optimized to obtain the optimal target-dependent ligation efficiency with the ligase and the maximum rolling circle replication rate and maximum end-point level of RCR product with the strand-displacing DNA polymerase under the assay conditions used. Although a bipartite target probe could also be used as a TSA probe, it is preferable that the TSA probe is not a bipartite target probe. Preferably, a TSA probe does not have a transcription promoter sequence, a transcription termination sequence, or a signal sequence, and preferably the primer-binding site in a TSA probe for a strand-displacing DNA polymerase primer used for rolling circle replication is not present in the corresponding bipartite target probe. The lack of a promoter sequence in the TSA probe or the resulting TSA circle permits maximum rolling replication because there is no promoter to bind an RNA polymerase or initiate transcription. Similarly, the lack of a primer-binding site for priming by a strand-displacing DNA polymerase on the bipartite target probe or the resulting circular transcription substrate permits maximum transcription because there is not site for priming a competitive rolling circle transcription reaction.

Referring to FIG. 5, in the presence of a target sequence, a TSA probe anneals to the target sequence under hybridization conditions, wherein the 5′-phosphorylated end of the TSA probe is adjacent to its 3′-hydroxyl end. Then, the ends of the TSA probe are ligated under ligation conditions by contacting the target-complementary ends annealed to a target sequence with a ligase that has little or no activity in ligating free ends that are not annealed to a complementary sequence but is active in joining a 5′-phosphorylated end to a 3′-hydroxylated end when the ends are adjacent when annealed to a complementary DNA sequence. Ligation of the ends of the TSA probe generates a TSA circle. Upon annealing of a primer to the TSA circle and contacting the resulting complex with a strand-displacing DNA polymerase under strand-displacing polymerization conditions, rolling circle replication occurs, thereby generating multiple tandem copies of the target sequence to which the target-complementary sequences of a bipartite target probe can anneal under hybridization conditions. The adjacent 5′-phosphorylated end and the 3′-hydroxyl end of the bipartite target probes annealed to the tandem target sequences of the rolling circle replication products are ligated by the ligase under ligation conditions, thereby generating a ligation product which, upon annealing of an anti-sense promoter oligo results in a circular transcription substrate. Transcription products are obtained by contacting the circular transcription substrates with an RNA polymerase that can bind the double-stranded promoter and initiate transcription therefrom, and the transcription products are obtained or detected by a suitable means.

Thus, referring to FIG. 5, one embodiment of the present invention comprises a method for obtaining a transcription product complementary to a target nucleic acid sequence (target sequence or target), said method comprising:

a. providing a target sequence amplification probe (TSA probe), wherein said TSA probe comprises a linear single-stranded DNA comprising two end portions that are not joined, which end portions are connected by an intervening sequence, wherein the 5′-end target-complementary sequence is complementary to the 5′-end of the target sequence, and wherein the 3′-end target-complementary sequence is complementary to the 3′-end of the target sequence, and wherein joining of the ends of said TSA probe forms a TSA circle;

b. contacting the TSA probe to the target sequence and incubating under hybridization conditions, wherein the target-complementary sequences anneal adjacently to the target sequence;

c. contacting said TSA probe annealed to said target sequence with a ligase under ligation conditions so as to obtain a TSA circle;

d. providing a primer that is complementary to the intervening sequence of the TSA probe;

e. contacting the TSA circle with the primer that is complementary to the intervening sequence of the TSA probe under hybridization conditions so as to obtain a TSA circle-primer complex;

f. contacting said TSA circle-primer complex with a strand-displacing DNA polymerase under strand-displacing polymerization conditions so as to obtain a rolling circle replication product comprising multiple copies of the target sequence;

g. providing target probes comprising linear single-stranded DNA, the target probes comprising at least two target-complementary sequences that are not joined to each other, wherein the 5′-end of a first target-complementary sequence is complementary to the 5′-end of the target sequence, and wherein the 3′-end of a second target-complementary sequence is complementary to the 3′-end of the target sequence, and wherein the 3′-end of the first target-complementary sequence is joined to the 5′-end of a sense promoter sequence for an RNA polymerase;

h. contacting the target probes with the target sequence and incubating under hybridization conditions, wherein the target-complementary sequences anneal adjacently to the target sequence to form a target probe-target complex;

i. contacting the target probe-target complex with a ligase under ligation conditions to obtain a ligation product comprising the target-complementary sequences of the target probes annealed to the target nucleic acid sequence; j. contacting the ligation product with an anti-sense promoter oligo and incubating under hybridization conditions, wherein the anti-sense promoter oligo anneals to the sense promoter sequence of the ligation product to form a transcription substrate;

k. contacting the transcription substrate with an RNA polymerase that can bind the promoter and incubating under transcription conditions to obtain a transcription product; and

l. detecting the transcription product, wherein said transcription product indicates the presence of said target sequence.

Preferably, only one ligase is used for ligating both the TSA probe and the target probes. Preferably, the ligase has little or no activity in ligating blunt ends and is substantially more active in ligating ends that are adjacent when annealed to two contiguous regions of a target sequence compared to ends that are not annealed to the target sequence. One suitable ligase that can be used is Ampligase® Thermostable DNA Ligase (EPICENTRE Technologies, Madison, Wis.). A preferred strand-displacing DNA polymerase that can be used is IsoTherm™ DNA Polymerase (EPICENTRE Technologies, Madison, Wis.). Another suitable strand-displacing DNA polymerase that can be used is RepliPHI™ phi29 DNA polymerase (EPICENTRE Technologies, Madison, Wis.). Some preferred RNA polymerases are T7 RNAP, T3 RNAP, SP6 RNAP or another T7-like RNA polymerase or a mutant form of one of these T7-like RNA polymerases. Preferably, AmpliScribe T7-Flash™ Transcription Kit is used for in vitro transcription of the transcription substrate (EPICENTRE Technologies, Madison, Wis.).

In some embodiments, the target probes comprise monopartite target probes comprising a promoter target probe and a signal target probe and/or one or more simple target probes. In other embodiments, the target probes comprise a bipartite target probe and optionally, one or more simple target probes.

Thus, again referring to FIG. 5, one embodiment of the present invention comprises a method for obtaining a transcription product complementary to a target nucleic acid sequence (target or target sequence), said method comprising:

a. providing a target sequence amplification probe (TSA probe), wherein said TSA probe comprises a linear single-stranded DNA comprising two end portions that are not joined, which end portions are connected by an intervening sequence, wherein the 5′-end target-complementary sequence is complementary to the 5′-end of the target sequence, and wherein the 3′-end target-complementary sequence is complementary to the 3′-end of the target sequence, and wherein joining of the ends of said TSA probe forms a TSA circle;

b. providing a primer that is complementary to the intervening sequence of said TSA probe;

c. annealing said TSA probe to said target sequence under hybridization conditions;

d. ligating said TSA probe annealed to said target sequence with a ligase under ligation conditions so as to obtain a TSA circle;

e. annealing the primer that is complementary to the intervening sequence of the TSA probe to the TSA circle under hybridization conditions;

f. contacting said TSA circle to which said primer is annealed with a strand-displacing DNA polymerase under strand-displacing polymerization conditions so as to obtain a rolling circle replication product comprising multiple copies of the target sequence;

g. providing a bipartite target probe, wherein said bipartite target probe comprises a linear ssDNA comprising two end portions that are not joined, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, and wherein the 3′-end of the first target-complementary sequence is joined to the 5′-end of a sense promoter sequence for an RNA polymerase;

h. annealing said bipartite target probe to said multiple copies of the target sequence of said rolling circle replication product under hybridization conditions;

i. ligating said bipartite target probe annealed to said multiple copies of the target sequence of said rolling circle replication product with the ligase under ligation conditions so as to obtain a circular ssDNA ligation product;

j. annealing an anti-sense promoter oligo to said circular ssDNA ligation product to obtain a circular transcription substrate;

k. obtaining said circular transcription substrate, wherein said circular transcription substrate comprises a sequence that is complementary to said target sequence;

l. contacting said circular transcription substrate with an RNA polymerase under transcription conditions so as to obtain a transcription product that is complementary to said circular transcription substrate; and

m. obtaining said transcription product that is complementary to said circular transcription substrate, wherein said transcription product indicates the presence of said target sequence.

Preferably, only one ligase is used for ligating both the TSA probe and the bipartite target probe. Preferably, the ligase has little or no activity in ligating blunt ends and is substantially more active in ligating ends that are adjacent when annealed to two contiguous regions of a target sequence compared to ends that are not annealed to the target sequence. One suitable ligase that can be used is Ampligase® Thermostable DNA Ligase (EPICENTRE Technologies, Madison, Wis.). A preferred strand-displacing DNA polymerase that can be used is IsoTherm™ DNA Polymerase (EPICENTRE Technologies, Madison, Wis.). Another suitable strand-displacing DNA polymerase that can be used is RepliPHI™ phi29 DNA polymerase (EPICENTRE Technologies, Madison, Wis.). Some preferred RNA polymerases are T7 RNAP, T3 RNAP, SP6 RNAP or another T7-like RNA polymerase or a mutant form of one of these T7-like RNA polymerases. Preferably, AmpliScribe T7-Flash™ Transcription Kit is used for in vitro transcription of the transcription substrate (EPICENTRE Technologies, Madison, Wis.).

In some embodiments, the anti-sense promoter oligo is attached to a solid support. In other embodiments, the anti-sense promoter oligo comprises a moiety, such as, but not limited to a biotin moiety that permits binding of the anti-sense promoter oligo to a solid support after annealing to the ligation product to obtain a transcription substrate. In some embodiments, the target sequence comprises a target nucleic acid in a sample, whereas in other embodiments the target sequence comprises a target sequence tag that is joined to an analyte-binding substance that binds an analyte in the sample. In some embodiments, the TSA circle that is replicated remains catenated to a target nucleic acid or target sequence tag. However, preferably, the target sequence is less than about 150 to about 200 nucleotides from the 3′-end of the target nucleic acid or target sequence tag. In embodiments of methods in which the target sequence is greater than about 150 to about 200 nucleotides from the 3′-end of the target nucleic acid or target sequence tag, then one or more additional steps is used in order to release the catenated TSA circles from the target sequence prior to rolling circle replication, as described elsewhere herein. Similarly, one or more additional steps can be used in order to release the catenated circular ssDNA ligation products that result from ligation of bipartite target probes that are annealed to target sequences in the rolling circle replication product more than about 150 nucleotides to about 200 nucleotides from the 3′-end of to the rolling circle replication product. In one preferred embodiment, rolling circle replication is carried out using a ratio of dUTP to dTTP that results in incorporation of a dUMP residue about every 100-400 nucleotides and a composition comprising uracil-N-glycosylase and endonuclease IV is used to release catenated DNA molecules that are ligated on the linear rolling circle replication product following annealing of bipartite target probes to the replicated target sequences.

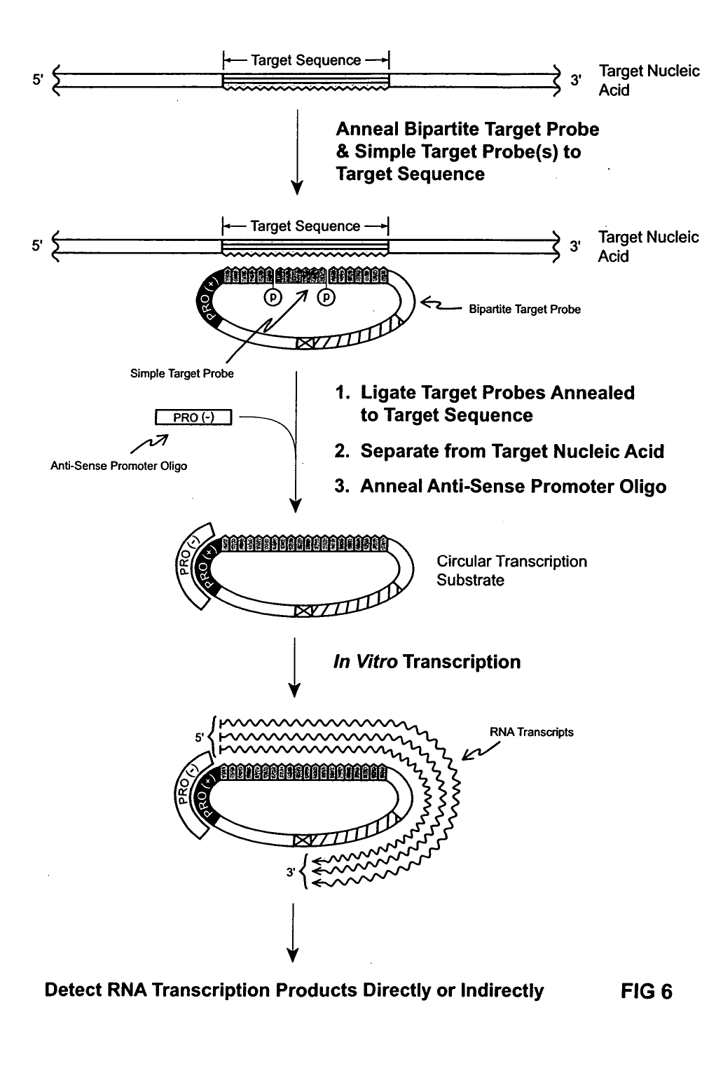

FIG. 6 shows one aspect of another embodiment of a method of the present invention. This embodiment also uses a bipartite target probe that is similar to a bipartite target probe used in the method shown in FIG. 3, except that the target-complementary sequences of the bipartite target probe used in the embodiment shown in FIG. 6 are not contiguous or adjacent to each other when annealed to the target sequence. Rather, the target-complementary sequences of a bipartite target probe of this embodiment are separated from each other when they are annealed to the target sequence. The gap between the two target-complementary sequences can comprise from about four nucleotides to about 1000 nucleotides or more. Although the invention is not limited to a particular distance between the target-complementary sequences when annealed to a target sequence, preferably the gap in this embodiment of the invention comprises from about six nucleotides to about 100 nucleotides, and most preferably, the gap comprises from about six nucleotides to about 25 nucleotides. As in the previous embodiments, the 5′-end of the bipartite target probe in the embodiment in FIG. 6 preferably has a 5′-phosphate group or is phosphorylated by a polynucleotide kinase during the course of a method of the invention, and the 3′-end preferably has a 3′-hydroxyl group. Also as in previously discussed embodiments, the 5′-portion of the bipartite target probe in the embodiment of FIG. 6 has sequence for a sense transcription promoter which, upon annealing to an anti-sense promoter oligo, is a functional promoter for a DNA-dependent RNA polymerase that can bind this double-stranded promoter and initiate transcription therefrom in a 5′-to-3′ direction, wherein the sense promoter sequence is joined to the 3′-end of the target-complementary sequence that anneals to the 5′-end of the target sequence. That is, the sense promoter is oriented within the single-stranded DNA of a bipartite target probe 3′-of the target-complementary sequence at the 5′-end of the 5′-portion.

In the aspect of the embodiment of the method shown in FIG. 6, the gap between the target-complementary sequences of a bipartite target probe annealed to a target sequence is filled by also annealing one or more simple target probes comprising target-complementary sequences that anneal to the target sequence between portions of the target to which the target-complementary sequences of the bipartite target probe anneal. The simple target probes used anneal to the target sequence so as to fill the gap completely so as to abut with or to be contiguous with each other and with the target-complementary sequences of the bipartite target probe. All 5′-ends of simple target probes and of the bipartite target probe have a 5′-phosphate group and all 3′-ends have hydroxyl groups. Thus, ligation of the bipartite target probe and simple target probes that are annealed to a target sequence with a ligase, which ligase has little or no activity in ligating free ends that are not annealed to a complementary sequence but is active in joining a 5′-phosphorylated end to an adjacent 3′-hydroxylated end when the ends are annealed to a complementary DNA sequence, generates a circular ligation product, which upon annealing to an anti-sense promoter oligo, generates a circular transcription substrate. Transcription of the circular transcription substrate results in synthesis of transcription product that is complementary to the circular transcription substrate and that can be used to detect the presence of the target sequence.

Thus, again referring to FIG. 6, one embodiment of the present invention comprises a method for detecting a target sequence, said method comprising:

a. providing a bipartite target probe, wherein said bipartite target probe comprises a linear single-stranded DNA comprising two end portions that are not joined and that are complementary to different non-contiguous 5′- and 3′-end portions of the target sequence;

b. providing one or more simple target probes, wherein said simple target probes are complementary to the target sequence so as to anneal to said target sequence in the gap between the target-complementary sequences of said bipartite target probe so as to completely fill said gap and so that each of the ends of said simple target probes are contiguous with an end of a simple target probe or with an end of said bipartite target probe;

c. annealing said bipartite target probe and said simple target probes to said target sequence under hybridization conditions;

d. ligating said bipartite target probe and said simple target probes annealed to said target sequence under ligation conditions with a ligase so as to obtain a circular ssDNA ligation product;

e. contacting the ligation product with an anti-sense promoter oligo and incubating under hybridization conditions, wherein the anti-sense promoter oligo anneals to the sense promoter sequence of the ligation product to form a circular transcription substrate;

f. obtaining said circular transcription substrate, wherein said substrate comprises a sequence that is complementary to said target sequence;

g. contacting said circular transcription substrate with an RNA polymerase under transcription conditions so as to synthesize transcription product that is complementary to said circular transcription substrate; and

h. detecting the synthesis of transcription product resulting from transcription of said circular transcription substrate, wherein said synthesis of said transcription product indicates the presence of said target sequence.

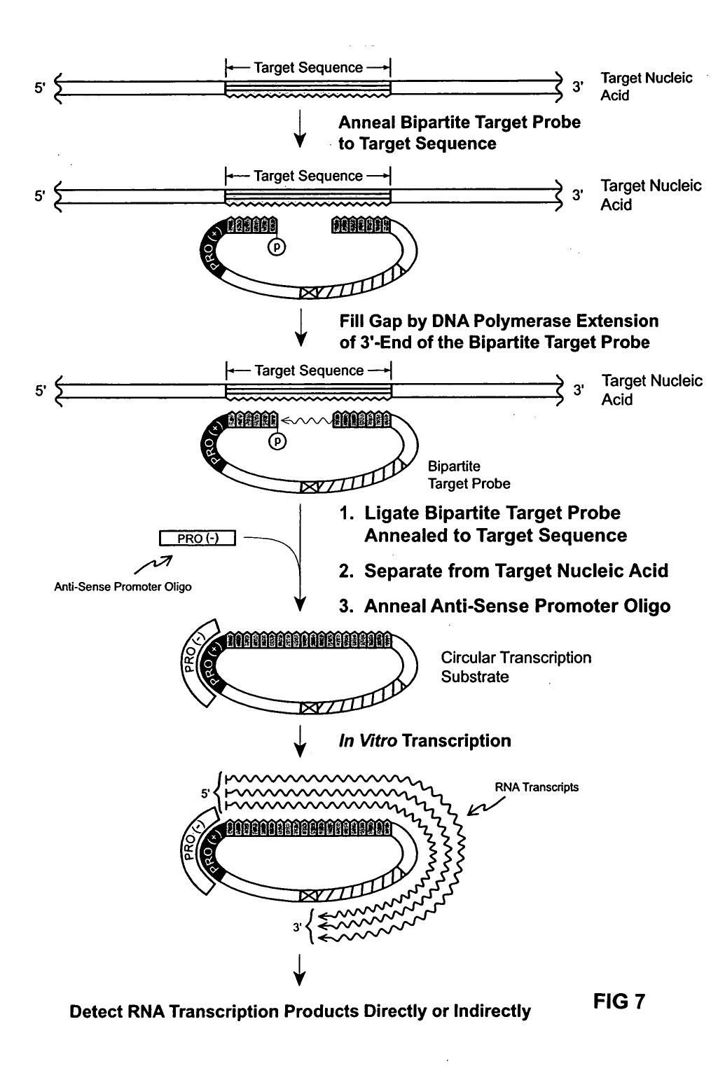

FIG. 7 shows another aspect of an embodiment of a method of the present invention that uses a bipartite target probe that comprises target-complementary sequences that are separated from each other when they are annealed to a target sequence. However, in this embodiment, the gap between the target-complementary sequences of a bipartite target probe annealed to a target sequence is filled by primer extension using a DNA polymerase and subsequently joined by ligation with a ligase if and only if both the 3′-end of the target probe that is annealed to the target sequence 3′-of the gap and the target probe that is annealed to the target sequence 5′-of the gap are complementary to and correctly base paired with the target sequence. If the 3′-end of the target probe that is 3′-of the gap is not annealed to the target sequence, then the DNA polymerase will be unable to fill the gap by primer extension. Also, if the 5′-end of the target probe that is 5′-of the gap is not annealed to the target sequence, then the 3′-end of the primer extension product will not be adjacent to a 5′-end on the target sequence and it will not be possible to join the 3′-end of the primer-extended target probe with the 5′-phosphorylated end of the target probe annealed 5′-of the gap.

The gap between the two target-complementary sequences can comprise from one nucleotide to about 1000 nucleotides or more. Although the invention is not limited to a particular distance between the target-complementary sequences when annealed to a target sequence, preferably the gap comprises from one nucleotide to about 100 nucleotides, and most preferably, the gap in most embodiments comprises from one nucleotide to about 25 nucleotides. The 5′-end of a bipartite target probe in the embodiment in FIG. 7 preferably has a 5′-phosphate group or is phosphorylated by a polynucleotide kinase during the course of a method of the invention, and the 3′-end preferably has a 3′-hydroxyl group. Also, the 5′-portion of the bipartite target probe in the embodiment of FIG. 7 has sense promoter sequence which, upon complexing with an anti-sense promoter oligo, forms a functional promoter for a DNA-dependent RNA polymerase that can bind to this double-stranded promoter and initiate transcription therefrom in a 5′-to-3′ direction using as a template a single-stranded DNA that is 5′-of and covalently linked to the promoter. The sense promoter sequence is oriented within the single-stranded DNA of a bipartite target probe 3′-of the target-complementary sequence at the 5′-end of the 5′-portion.

In the embodiment of a method shown in FIG. 7, the gap between the target-complementary sequences of a bipartite target probe annealed to a target sequence is filled by contacting the target sequence to which a bipartite target probe is annealed with a DNA polymerase under polymerization conditions. Then, the 5′-phosphorylated end of a bipartite target probe annealed to a target sequence is joined to the 3′-end of the DNA polymerase-extended 3′-end of said bipartite target probe with a ligase, which ligase has little or no activity in ligating free ends that are not annealed to a complementary sequence but is active in joining a 5′-phosphorylated end to an adjacent 3′-hydroxylated end when the ends are annealed to a complementary DNA sequence, generates a circular transcription substrate. Transcription of the circular transcription substrate results in synthesis of transcription product that is complementary to the circular transcription substrate and that can be used to detect the presence of the target sequence.

Thus, referring to FIG. 7, one embodiment of the present invention comprises a method for detecting a target sequence, said method comprising:

a. providing a bipartite target probe, wherein said bipartite target probe comprises a linear single-stranded DNA comprising two end portions that are complementary to different non-contiguous 5′- and 3′-end portions of a target sequence;

b. annealing said bipartite target probe to said target sequence under hybridization conditions;

c. contacting said complex comprising said bipartite target probe annealed to said target sequence with a DNA polymerase under non-strand-displacing DNA polymerization conditions so as to obtain a DNA polymerase extension product that is complementary to the target sequence between the target-complementary sequences of said annealed bipartite target probe so as to completely fill said gap and so that the 3′-end of said synthesized DNA is contiguous with the 5′-end of said bipartite target probe;

d. ligating the 5′-end of said bipartite target probe annealed to said target sequence with the 3′-end of said DNA polymerase extension product under ligation conditions with a ligase so as to obtain a circular ssDNA ligation product;

e. contacting the ligation product with an anti-sense promoter oligo and incubating under hybridization conditions, wherein the anti-sense promoter oligo anneals to the sense promoter sequence of the ligation product to form a circular transcription substrate;

f. obtaining said circular transcription substrate, wherein said substrate comprises a sequence that is complementary to said target sequence;

g. contacting said circular transcription substrate with an RNA polymerase under transcription conditions so as to synthesize transcription product that is complementary to said circular transcription substrate; and

h. detecting the synthesis of transcription product resulting from transcription of said circular transcription substrate, wherein said synthesis of said transcription product indicates the presence of said target sequence.

In addition to the embodiments disclosed above for filling a gap between target-complementary sequences of a bipartite target probe that are not contiguous when annealed to a target sequence, the invention also comprises methods that use a combination of both one or more simple target probes and DNA polymerase extension in order to fill the gap so as to obtain adjacent target-complementary sequences prior to the ligation step.

A suitable non-strand-displacing DNA polymerase for filling a gap according to this embodiment of the invention is T4 DNA polymerase.

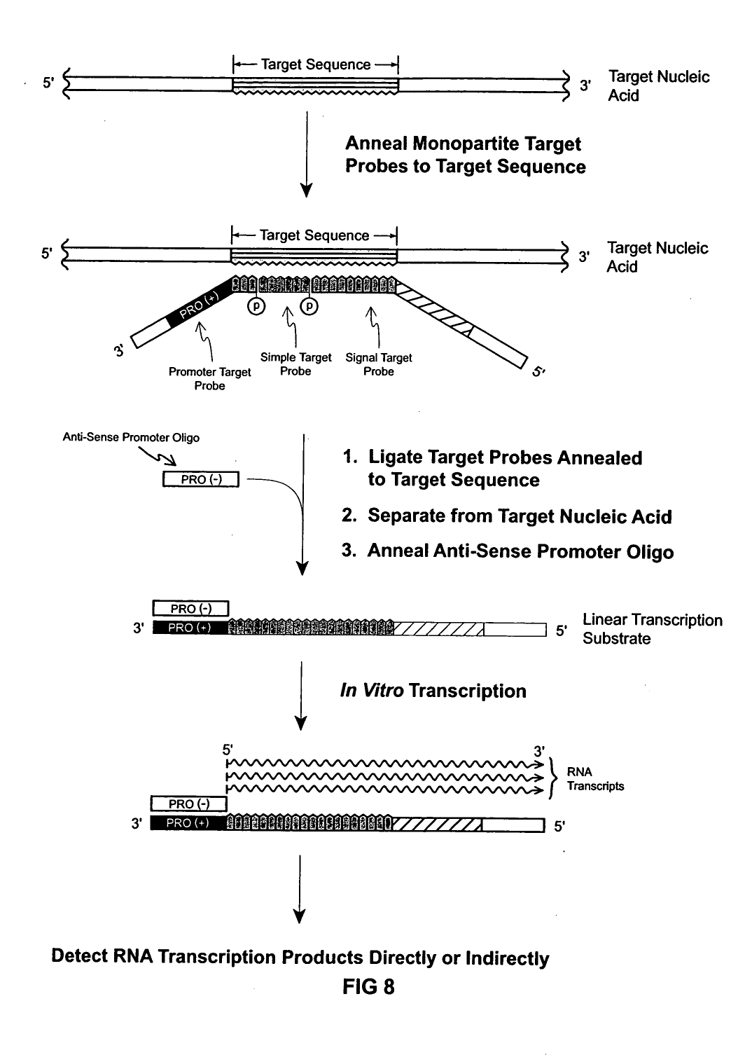

Other embodiments of methods of the invention generate a linear transcription substrate for amplifying, detecting and quantifying one or multiple target nucleic acid sequences in a sample, including target sequences that differ by as little as one nucleotide. FIG. 8 shows a basic embodiment of a method that generates a linear transcription substrate. This embodiment uses only monopartite target probes. A monopartite target probe is a single-stranded DNA molecule that comprises only one target-complementary sequence, although a monopartite target probe can comprise other sequences that are not complementary to a target sequence. By way of example, but not of limitation, a method of the invention that generates a linear transcription substrate always uses a monopartite target probe called a “promoter target probe.” A “promoter target probe” has a 5′-portion that is complementary to the most 5′-portion of a target sequence. The 3′-end of this 5′-target-complementary portion is joined to the 5′-end of a sense promoter sequence, which upon complexing with an anti-sense promoter sequence, serves as a functional transcription promoter for a DNA-dependent RNA polymerase that can bind to this promoter and initiate transcription of RNA therefrom in a 5′-to-3′ direction under transcription conditions using single-stranded DNA that is 5′-of (with respect to the same strand) and covalently linked to the promoter as a template. The sequence at the 5′-end of the promoter target probe preferably has a 5′-phosphate group or is phosphorylated by a polynucleotide kinase during the course of a method of the invention. The embodiment of the method shown in FIG. 8 also uses another monopartite target probe called a “signal target probe.” A “signal target probe” has a 3′-portion and a 5′-portion. At least the 3′-end portion of a signal target probe comprises a sequence that is complementary to the most 3′-portion of a target sequence. As shown in the embodiment in FIG. 8, the 3′-end of the signal target probe has a 3′-hydroxyl group. The 5′-portion of a signal target probe comprises a “signal sequence.” A signal sequence is a sequence that is detectable in some way following its transcription during a method of the invention. The invention does not require the use of a signal target probe having a signal sequence. By way of example, but not of limitation, a simple target probe could be used in an assay of the invention in place of a signal target probe. If a signal target probe is used in a method of the invention, the signal sequence can comprise any sequence that is detectable following transcription. By way of example, but not of limitation, a signal sequence can comprise a sequence that is detectable using a molecular beacon as described by Tyagi et al. (U.S. Pat. Nos. 5,925,517 and 6,103,476 of Tyagi et al. and U.S. Pat. No. 6,461,817 of Alland et al., all of which are incorporated herein by reference). A preferred signal sequence of the invention is a sequence that results in an additional amplification of the signal following its transcription, thus making the detection of a target sequence more sensitive. The signal target probe used in the method shown in FIG. 8 can be, for example, a signal sequence that encodes a substrate for Q-beta replicase (EPICENTRE Technologies, Madison, Wis.), which permits additional amplification of the signal by incubating the transcription product with Q-beta replicase under replication conditions. However, as discussed elsewhere herein, many other signal sequences can be used in a signal target probe, all of which are incorporated as part of the present invention.

Thus, again referring to FIG. 8, one embodiment of the present invention comprises a method for detecting a target sequence, said method comprising:

a. providing a promoter target probe comprising a linear single-stranded DNA having a 5′-end comprising a target-complementary sequence that is complementary to the most 5′-portion of the target sequence, the 3′-end of which target-complementary sequence is joined to 5′-end of a sense promoter sequence for an RNA polymerase;

b. providing a signal target probe comprising a linear ssDNA having a 3′-end comprising a target-complementary sequence that is complementary to the most 3′-portion of the target sequence and a signal sequence that is 5′-of the target-complementary sequence;

c. optionally, if the target-complementary sequences of said promoter target probe and said signal target probe are not contiguous when annealed to the target sequence, providing one or more simple target probes, wherein said simple target probes anneal to the target sequence in a gap between the target-complementary sequences of the promoter target probe and the signal target probe so as to completely fill said gap and so that each of the ends of said simple target probes are contiguous with an end of a simple target probe or with the 5′-end of the promoter target probe or the 3′-end of the signal target probe;

d. annealing said promoter target probe, said simple target probes, if present, and said signal target probe to said target sequence under hybridization conditions;

e. ligating said promoter target probe, said simple target probes, if present, and said signal target probe that are annealed to the target sequence with a ligase under ligation conditions so as to obtain a linear ssDNA ligation product;

f. contacting the ligation product with an anti-sense promoter oligo and incubating under hybridization conditions, wherein the anti-sense promoter oligo anneals to the sense promoter sequence of the ligation product to form a linear transcription substrate;

g. obtaining said linear transcription substrate, wherein said linear transcription substrate comprises a sequence that is complementary to the target sequence;

h. contacting the linear transcription substrate with an RNA polymerase under transcription conditions so as to synthesize transcription product that is complementary to said target-complementary sequence and said signal sequence of said linear transcription substrate; and

i. detecting the synthesis of transcription product resulting from transcription of said linear transcription substrate, wherein said synthesis of said transcription product indicates the presence of the target sequence.

In addition to the embodiment shown in FIG. 8 in which a simple target probe is used to fill a gap on a target sequence between the target-complementary sequences of a promoter target probe and a signal target probe, the invention also comprises embodiments in which a DNA polymerase is used to fill the gap between a promoter target probe and a signal target probe, wherein said target probes are not adjacent when annealed to a target sequence. The invention further comprises use of a combination of a promoter target probe, one or more simple target probes, a signal target probe and DNA polymerase extension of the 3′-hydroxyl end of said signal target probe or of one or more simple target probes so that said target probes, including said DNA polymerase-extended target probes, completely fill the gap on a target sequence between the target-complementary portions of said promoter probe and said signal target probe. Thus any combination of simple target probes and DNA polymerase extension can be used to fill the gap between the target-complementary portions of the promoter probe and the signal target probe so as to obtain adjacent target-complementary sequences prior to the ligation step.

The invention also comprises methods for obtaining secondary or additional amplification by using the transcription products synthesized by transcription of a circular transcription substrate or a linear transcription substrate as a template for ligation of the same or different bipartite or monopartite target probes, thus generating additional circular transcription substrates or linear transcription substrates, respectively. By way of example, but not of limitation, one embodiment of a method of the invention for obtaining secondary amplification uses bipartite target probes and two ligases—one ligase that can ligate target-complementary sequences of a bipartite target probe annealed to a DNA target sequence to form a first circular ligation product that, upon annealing of an anti-sense promoter oligo, results in a first circular transcription substrate, and one that can ligate the same target-complementary sequences of said bipartite target probe annealed to an RNA transcript resulting from transcription of said first circular transcription substrate. By way of example, but not of limitation, one ligase that can be used in a method of the present invention for ligation of contiguous DNA molecules annealed to an RNA ligation template is T4 RNA ligase (EPICENTRE Technologies, Madison, Wis., USA), as disclosed by Faruqi in U.S. Pat. No. 6,368,801 B1, which is incorporated herein by reference. The invention also comprises embodiments that use similar secondary amplification methods with two ligases using monopartite target probes and that generate linear transcription substrates.

In addition to comprising embodiments of methods wherein bipartite target probes are used that anneal to both the target sequence and to the same sequence in the RNA transcripts resulting from transcription of a first circular transcription substrate, the invention also comprises other embodiments of methods and assays wherein a second bipartite target probe is used that anneals to a sequence in the RNA transcript that is complementary to a signal sequence or an optional sequence of the first circular transcription substrate rather than annealing to the target sequence or the identical sequence in the RNA transcript.

In still other embodiments, the invention also comprises use of a reverse transcriptase process to obtain additional amplification of a target sequence and/or a signal sequence in an assay or method of the invention. An example of an embodiment of the invention that uses a reverse transcriptase process is shown in FIG. 9. This example illustrates a number of aspects of the invention that result in improvements over the methods and assays of the prior art.

The first part of the assay or method in FIG. 9 is similar to the embodiment shown in FIG. 4. Thus, a first circular transcription substrate is generated by ligation of a first bipartite target probe annealed to a target sequence in a sample, followed by annealing of an anti-sense promoter oligo. Then, in vitro transcription of the first circular transcription substrate amplifies the target sequence and the signal sequence, if present. In the example, shown in FIG. 9, rolling circle transcription is used to synthesize RNA comprising multimeric copies of an RNA oligomer that is complementary to the first circular transcription substrate. In contrast to run-off transcription of linear transcription substrates, as is used for methods in the prior art such as, but not limited to, NASBA or TMA, rolling circle transcription synthesizes RNA that has sequences that are complementary to the sense promoter sequence in a circular transcription substrate. As discussed below, the presence of these promoter-complementary sequences in the RNA transcription product from rolling circle transcription permits generation of additional single-stranded transcription promoters that can initiate additional in vitro transcription reactions and thereby further amplify the target sequence and/or signal sequence.

Thus, one or more oligonucleotide primers anneal to the multimeric RNA transcription products and first-strand cDNA is synthesized by extension of said primers by a reverse transcriptase under reverse transcription reaction conditions. In the example shown in FIG. 9, only one reverse transcription primer is used that anneals to the same sequence in different repeated sites on the multimeric RNA. However, the invention also comprises embodiments that use multiple reverse transcription primers, each of which is complementary to a different sequence of an RNA oligomer that is, in turn, complementary to a circular transcription substrate. The sequence to which a reverse transcription primer anneals in an RNA multimer can also vary. Preferably, a reverse transcription primer anneals to a sequence in the RNA multimer in a region that is complementary to an optional sequence portion of a circular transcription substrate and that is 3′-of a signal sequence-complementary sequence, if present. The reverse transcription primer shown in FIG. 9 anneals to the RNA multimer at a site that is 3′ of of a signal sequence-complementary sequence of each oligomer of the multimer. The reverse transcription primer shown in FIG. 9 has a 5′-portion comprising a “tail” that is a sequence that is not complementary to the RNA transcript. The use of a tail is optional and is not required for methods and assays of the invention. As discussed below, a tail may be useful for embodiments that use a novel strand-displacement reverse transcription process of the present invention.

Again referring to FIG. 9, following reverse transcription of the RNA multimer, the first-strand cDNA is available in the reaction mixture for at least two subsequent functions. First, the first-strand cDNA has a sense sequence for a transcription promoter and, upon annealing of an anti-sense promoter oligo, is used as a linear transcription substrate for synthesis of RNA using the RNA polymerase that initiates transcription from said promoter under transcription conditions. Synthesis of RNA corresponding to the target sequence and/or the signal sequence in these linear transcription substrates can be detected according to the detection method used in the particular embodiment of an assay or method of the invention. Second, the first strand cDNA can be used as a ligation template for ligation of a second bipartite target probe under ligation conditions. In the embodiment shown in FIG. 9, the second bipartite target probe is identical to the first bipartite target probe except that with respect to the target-complementary sequences at the 3′- and 5′-ends of said second bipartite target probe. The 5′-end portion of the second bipartite target probe comprises a sequence that is complementary to the target-complementary sequence at the 3′-end portion of the first bipartite target probe, and this sequence is in turn covalently attached and 5′-of a promoter sequence in the 5′-portion of the second bipartite target probe. The 3′-end portion of the second bipartite target probe comprises a sequence that is complementary to the target-complementary sequence at the 5′-end portion of the first bipartite target probe, and this sequence is in turn covalently attached and 3′-of a signal sequence in the 3′-portion of the second bipartite target probe, if a signal sequence is present. Thus, the sequences at the 3′- and 5′-ends of said second bipartite target probe are identical to the target sequence and are complementary to the target-complementary sequences in both the first circular transcription substrate and in the first-strand cDNA obtained by reverse transcription of RNA transcripts from said first circular transcription substrate, both of which thus serve as ligation templates for ligation of the second bipartite target probe by a ligase under ligation conditions. Ligation of a second bipartite target probe and annealing of an anti-sense promoter oligo generates a second circular transcription substrate.

The second circular transcription substrate is then a substrate for rolling circle transcription, generating a complementary RNA multimer transcript. The RNA multimer transcript resulting from rolling circle transcription is then a substrate for reverse transcription by a reverse transcriptase under reverse transcription conditions. Since, in the embodiment shown in FIG. 9, the second circular transcription substrate is identical to the first circular transcription substrate in all portions except for the target-complementary portion, the same reverse transcription primer can be used to generate first-strand cDNA that is complementary to the RNA multimer from the second circular transcription substrate. The resulting first-strand cDNA, after annealing of an anti-sense promoter oligo, is a second linear transcription substrate. In vitro transcription of said second linear transcription substrate by an RNA polymerase that initiates transcription using said double-stranded transcription promoter under transcription conditions generates RNA transcripts that can be detected in the assay or method. The sequence corresponding to a target sequence in said first-strand cDNA also serves as a template for ligation of a first bipartite target probe by a ligase under ligation conditions. Ligation of another first bipartite target probe and annealing of an anti-sense promoter oligo to the resulting circular ligation product forms another first circular transcription substrate. Thus, the various annealing, ligation, rolling circle transcription, reverse transcription, and linear run-off transcription processes of this embodiment of an assay or method of the invention can continue, with continual generation of RNA that can be detected according to the particular assay or method until one or more of the reaction components are exhausted. The repeating cycles of processes of this embodiment of an assay or method results in high sensitivity and shorter reaction times, while retaining a high degree of specificity.

Thus, again referring to FIG. 9, one embodiment of the present invention comprises a method for detecting a target sequence, said method comprising:

a. providing a first bipartite target probe comprising linear single-stranded DNA having two target-complementary sequences that are not joined to each other and that are contiguous when annealed to the target sequence, wherein the 5′-end of the first target-complementary sequence is complementary to the 5′-end of the target nucleic acid sequence, and wherein the 3′-end of a second target-complementary sequence is complementary to the 3′-end of the target nucleic acid sequence, and wherein the 3′-end of the first target-complementary sequence is joined to the 5′-end of a sense promoter sequence for an RNA polymerase;

b. providing a second bipartite target probe comprising linear single-stranded DNA having two end sequences that are not joined to each other and that, when joined, are identical to the target sequence, wherein the 5′-end of the first end sequence is complementary to the target-complementary sequence of the 3′-end of the first bipartite target probe and the 3′-end of the second end sequence is complementary to the target-complementary sequence of the 5′-end of the first bipartite target probe; and wherein the 3′-end of the first end sequence is joined to the 5′-end of a sense promoter sequence for an RNA polymerase;

c. annealing said first bipartite target probe to said target sequence under hybridization conditions;

d. ligating said first bipartite target probe annealed to said target sequence with a ligase under ligation conditions so as to obtain a first circular ssDNA ligation product;

e. contacting the first circular ligation product with an anti-sense promoter oligo and incubating under hybridization conditions, wherein the anti-sense promoter oligo anneals to the sense promoter sequence of the first circular ligation product to form a first circular transcription substrate;

f. contacting said first circular transcription substrate with an RNA polymerase under transcription conditions so as to synthesize RNA that is complementary to said first circular transcription substrate;

g. annealing to said RNA that is complementary to said first circular transcription substrate a primer, wherein said primer is complementary to said RNA;

h. contacting said RNA to which said primer is annealed with a reverse transcriptase under reverse transcription conditions so as to obtain a first first-strand cDNA;

i. annealing to said first first-strand cDNA said second bipartite target probe under hybridization conditions;

j. contacting said first first-strand cDNA to which said second bipartite target probe is annealed with a a ligase under ligation conditions so as to obtain a second circular ssDNA ligation product;

k. contacting said second circular ligation product with an anti-sense promoter oligo and incubating under hybridization conditions, wherein the anti-sense promoter oligo anneals to the sense promoter sequence of the second circular ligation product to form a second circular transcription substrate;

l. obtaining said second circular transcription substrate;

m. contacting said second circular transcription substrate with an RNA polymerase under transcription conditions so as to synthesize RNA that is complementary to said second circular transcription substrate;

n. annealing to said RNA that is complementary to said second circular transcription substrate a primer, wherein said primer is complementary to said RNA;

o. contacting said RNA to which said primer is annealed with a reverse transcriptase under reverse transcription conditions so as to obtain a second first-strand cDNA;

p. obtaining said second first-strand cDNA;

q. annealing to said second first-strand cDNA said first bipartite target probe under annealing conditions;

r. contacting said second first-strand cDNA to which said first bipartite target probe is annealed with a a ligase under ligation conditions so as to obtain a third circular ssDNA ligation product that is identical to said first circular ligation product;

s. contacting the third circular ligation product with an anti-sense promoter oligo and incubating under hybridization conditions, wherein the anti-sense promoter oligo anneals to the sense promoter sequence of the third circular ligation product to form a third circular transcription substrate;

t. obtaining said third circular transcription substrate that is identical to said first circular transcription substrate;

u. contacting said first and second first-strand cDNA products with an anti-sense promoter oligo and incubating under hybridization conditions, wherein the anti-sense promoter oligo anneals to the sense promoter sequence of the first and second cDNA products to form first and second linear transcription substrates;

v. contacting the first and second linear transcription substrates with an RNA polymerase under transcription conditions so as to synthesize RNA that is complementary to said first and second linear transcription substrates;

w. repeating steps a through w; and

x. detecting the synthesis of RNA resulting from transcription of said first, second and third circular transcription substrates and from said first and second linear transcription substrates, wherein said synthesis of said RNA indicates the presence of said target sequence. In some embodiments, the circular transcription substrates that are transcribed remain catenated during transcription. In other embodiments, one or more additional steps are used in order to release the catenated circular ligation products from the target sequence when the target probe anneals to a sequence in a linear DNA molecule that is greater than about 150 to about 200 nucleotides from the 3′-end of the linear DNA molecule, as discussed elsewhere herein. In one preferred embodiment, DNA polymerization or reverse transcription is carried out using a ratio of dUTP to dTTP that results in incorporation of one dUMP residue about every 200-400 nucleotides and a composition comprising uracil-N-glycosylase and endonuclease IV is used to release catenated DNA molecules following ligation of bipartite target annealed to the long linear DNA molecules.

Preferably, only one ligase is used for all ligation reactions. Preferably, the ligase has little or no activity in ligating blunt ends and is substantially more active in ligating ends that are adjacent when annealed to a contiguous complementary sequence compared to ends that are not adjacently annealed to acomplementary sequence. One suitable ligase that can be used is Ampligase® Thermostable DNA Ligase (EPICENTRE Technologies, Madison, Wis.). One suitable reverse transcriptase that can be used is MMLV Reverse Transcriptase. Another suitable reverse transcriptase that can be used is IsoTherm™ DNA Polymerase (EPICENTRE Technologies, Madison, Wis.). Some preferred RNA polymerases are T7 RNAP, T3 RNAP, SP6 RNAP or another T7-like RNA polymerase or a mutant form of one of these T7-like RNA polymerases. Preferably, AmpliScribe T7-Flash™ Transcription Kit is used for in vitro transcription of the transcription substrate (EPICENTRE Technologies, Madison, Wis.).

In some embodiments, the anti-sense promoter oligo is attached to a solid support. In other embodiments, the anti-sense promoter oligo comprises a moiety, such as, but not limited to a biotin moiety that permits binding of the anti-sense promoter oligo to a solid support after annealing to the ligation product to obtain a transcription substrate. In some embodiments, the target sequence comprises a target nucleic acid in a sample, whereas in other embodiments the target sequence comprises a target sequence tag that is joined to an analyte-binding substance that binds an analyte in the sample.

The methods and assays of the embodiment of the invention shown in FIG. 9 can be performed in a stepwise manner or, more preferably, in a single reaction mixture in a continuous manner. Thus, one embodiment of the present invention comprises a method for detecting a target sequence, said method comprising:

1. providing a reaction mixture comprising:

a. a first bipartite target probe, wherein said first bipartite target probe comprises a 5′-portion and a 3′-portion, wherein said 5′-portion comprises: (i) a 5′-end portion that comprises a 5′-phosphate group and a sequence that is complementary to a target sequence, and (ii) a sense promoter sequence, wherein said sense promoter sequence is covalently attached to and 3′-of said target-complementary sequence in said 5′-portion; and wherein said 3′-portion comprises: (i) a 3′-end portion that comprises a sequence that is complementary to a target sequence, wherein said target-complementary sequence of said 3′-end portion, when annealed to said target sequence, is adjacent to said target-complementary sequence of said 5′-end portion of said first bipartite target probe, and (ii) optionally, a signal sequence, wherein said signal sequence is 5′-of said target-complementary sequence of said 3′-portion of said first bipartite target probe;

b. a second bipartite target probe, wherein said second bipartite target probe comprises a 5′-portion and a 3′-portion, wherein said 5′-portion comprises: (i) a 5′-end portion that comprises a 5′-phosphate group and sequence that is complementary to said target-complementary sequence of said 3′-end portion of said first bipartite target probe, and (ii) a sense promoter sequence, wherein said sense promoter sequence in said 5′-portion of said second bipartite target probe is 3′-of said target-complementary sequence in said 5′-portion; and wherein said 3′-portion comprises: (i) a 3′-end portion that comprises sequence that is complementary to said target-complementary sequence of said 5′-end portion of said first bipartite target probe, and (ii) optionally, a signal sequence, wherein said signal sequence in said 3′-portion of said second bipartite target probe is 5′-of said target-complementary sequence in said 3′-portion;

c. a ligase, wherein said ligase has little or no activity in ligating blunt ends and is substantially more active in ligating the ends of a bipartite target probe if said ends are adjacent when annealed to two contiguous regions of a complementary sequence than if said ends are not annealed to said complementary sequence, so as to obtain a circular ssDNA molecule that comprises a circular transcription substrate;

d. an anti-sense promoter oligo that anneals to the sense promoter sequence;

e. a reverse transcriptase and one or more primers, wherein at least the 3′-portion of one said primer comprises a sequence that is complementary to a sequence of said first bipartite target probe and of said second bipartite target probe and wherein said complementary portion of said primer is not complementary to said target sequence or the complement of said target sequence;

f. an RNA polymerase, wherein said RNA polymerase recognizes said single-stranded transcription promoters of said first and second bipartite target probes and synthesizes RNA therefrom using as a template single-stranded DNA to which said promoters are functionally attached;

g. optionally, a single strand binding protein;

h. optionally, a detection oligo, wherein said detection oligo anneals to an RNA transcript sequence that is complementary to a signal sequence of said first and/or second bipartite target probe;

i. optionally, compositions that result in release of catenated circular molecules that are ligated when annealed more than about 200 nucleotides from the 3′-end of a linear DNA molecule;

j. reaction conditions wherein said ligase, said reverse transcriptase, and said RNA polymerase are optimally active in combination and wherein said target-complementary sequences of said first bipartite target probe anneal to said target sequence, if present, with specificity, and;

2. contacting said reaction mixture from step 1 above with a sample comprising a target sequence, if present, wherein said reaction mixture containing said sample is maintained at a temperature wherein said ligase, said reverse transcriptase, and said RNA polymerase are optimally active in combination and wherein said target-complementary sequences of said first bipartite target probe anneal to said target sequence, if present, with specificity, and wherein said temperature of said reaction mixture is maintained for a time sufficient to permit synthesis of RNA transcription products complementary to circular transcription substrates and linear transcription substrates obtained if said target sequence is present in said sample; and

3. detecting the synthesis of RNA resulting from transcription of said circular transcription substrates and said linear transcription substrates, wherein said synthesis of said RNA indicates the presence of said target sequence.

In preferred embodiments of this method or assay for detecting a target sequence, said ligase comprises a ligase chosen from among Ampligase® thermostable DNA Ligase, Tth DNA Ligase, Tfl DNA Ligase, Tsc DNA Ligase, or Pfu DNA Ligase. In one preferred embodiment, the compositions that result in release of catenated DNA molecules that are ligated on a linear template comprise nucleotides comprising a ratio of dUTP to dTTP that results in incorporation of a dUMP residue about every 200-400 nucleotides and a composition comprising uracil-N-glycosylase and endonuclease IV.

In preferred embodiments of the above methods or assays for detecting a target sequence, said reverse transcriptase is a reverse transcriptase that has RNase H activity, wherein said reverse transcriptase is chosen from among MMLV reverse transcriptase, AMV reverse transcriptase, another retroviral reverse transcriptase, or a reverse transcriptase encoded by a thermostable phage. In other embodiments of the above methods or assays, said reverse transcriptase comprises a DNA polymerase chosen from among IsoTherm™ DNA polymerase, Bst DNA polymerase large fragment, Bca.BEST™ DNA polymerase, and Tth DNA polymerase.

In preferred embodiments of the above methods or assays for detecting a target sequence, said RNA polymerase is T7 RNAP, T3 RNAP or SP6 RNAP.

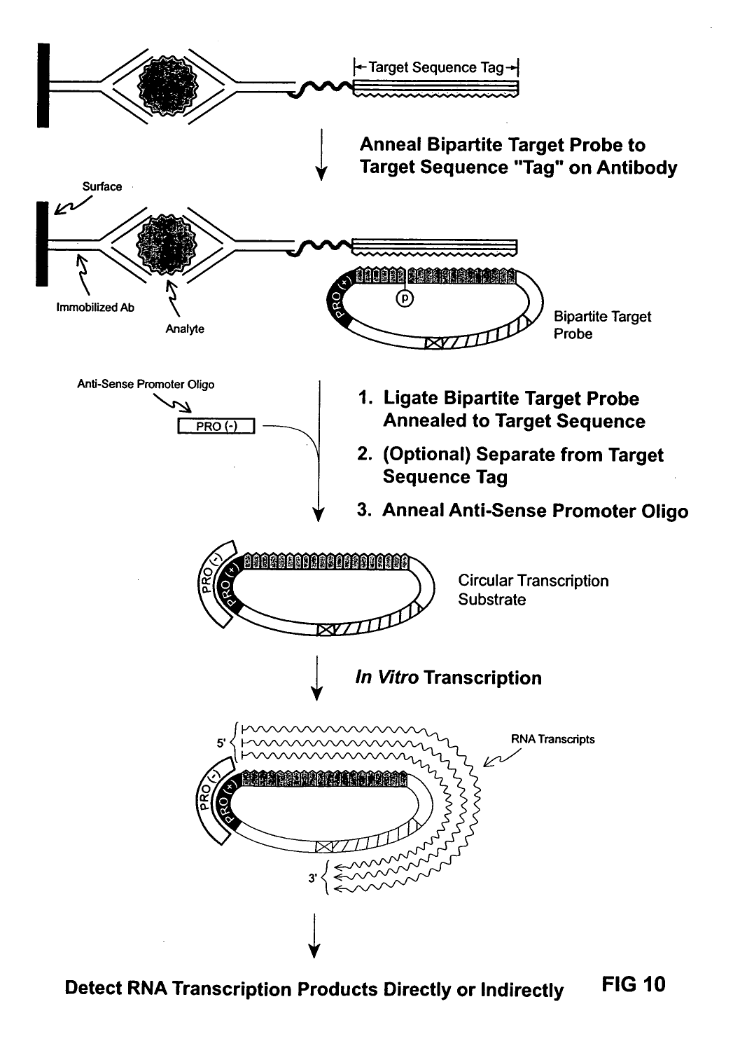

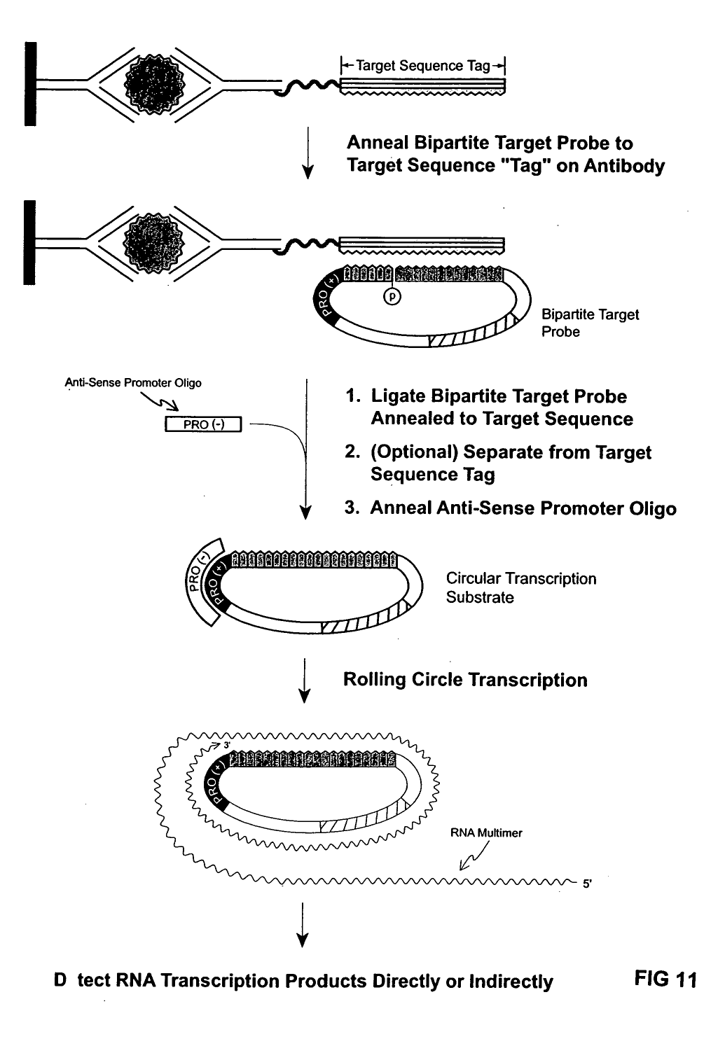

In general, the methods using target probes that comprise a sense promoter sequence for a double-stranded promoter described in this section above can also be used to detect a target sequence tag that is joined to an analtye-binding substance, examples of which are illustrated in FIGS. 10 and 11, and which are described in greater detail in the next section pertaining to detection of non-nucleic acid analytes.

2. Methods and Assays of the Invention That Use a Target Probe Comprising a Single-Stranded Promoter for Detecting a Target Sequence

In addition to the embodiments described above, the present invention also comprises embodiments that use an RNA polymerase that recognizes a cognate single-stranded transcription promoter or a single-stranded pseudopromoter. In these embodiments, the promoter sequence in a monopartite promoter target probe or in a bipartite target probe comprises a sequence for the single-stranded promoter or pseudopromoter recognized by the cognate RNA polymerase. A preferred single-stranded promoter comprises an N4 promoter and the cognate RNA polymerase that is used is an N4 mini-vRNAP or a Y678F mutant of an N4 mini-vRNAP. Preferred single-stranded pseudopromoters include a pseudopromoter or synthetic single-stranded promoter for a T7-type RNA polymerase, chosen from among T7 RNAP, T3 RNAP or SP6 RNAP, or for E. coli RNAP or for Thermus thermophilus RNAP, and the cognate RNA polymerase for the promoter is used. Some pseudopromoters that can be used in a promoter target probe or a bipartite target probe if E. coli RNAP is used are provided in Ohmichi et al. (Proc. Natl. Acad. Sci. USA, 99: 54-59, 2002).

The ability to use a single-stranded promoter or pseudopromoter simplifies an assay or method of the invention since a transcription substrate can be obtained without the need to complex an anti-sense promoter oligo with a sense promoter sequence in a product of ligation of one or more target probes annealed to a target sequence. In general, embodiments that can be performed using a target probe comprising a sense promoter sequence for a double-stranded promoter can also be performed more easily using a target probe comprising a single-stranded promoter or pseudopromoter. The exception to this statement is that, since an anti-sense promoter oligo is not used in embodiments with single-stranded promoters, an anti-sense promoter oligo that is attached to a solid support cannot be used to bind a single-stranded oligo. The assays or methods of the invention that use single-stranded promoters have not previously been known in the art.

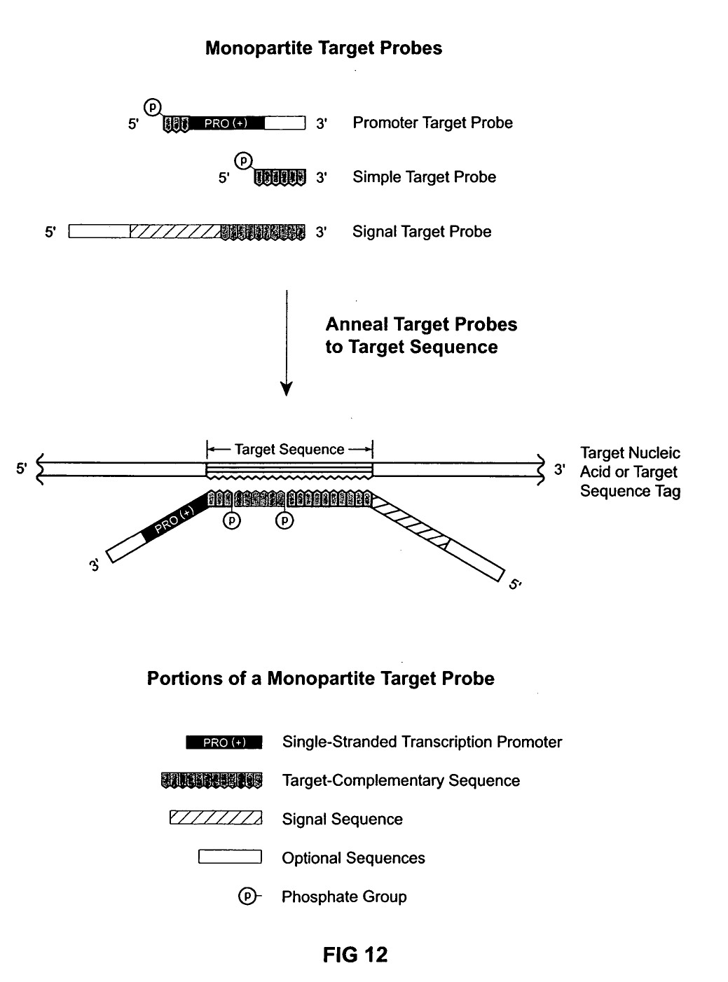

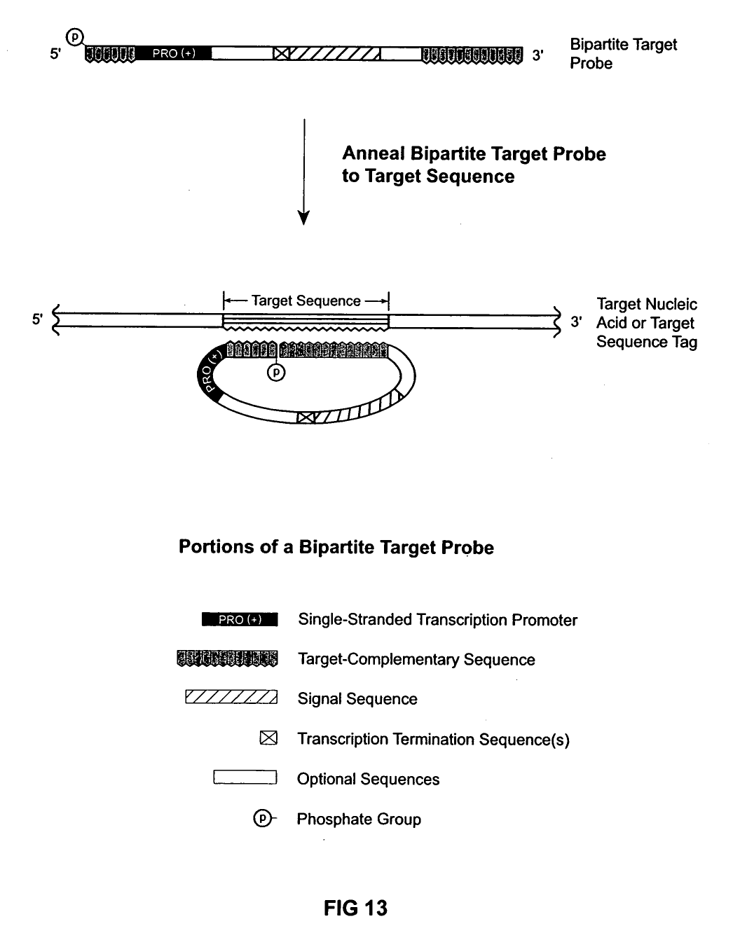

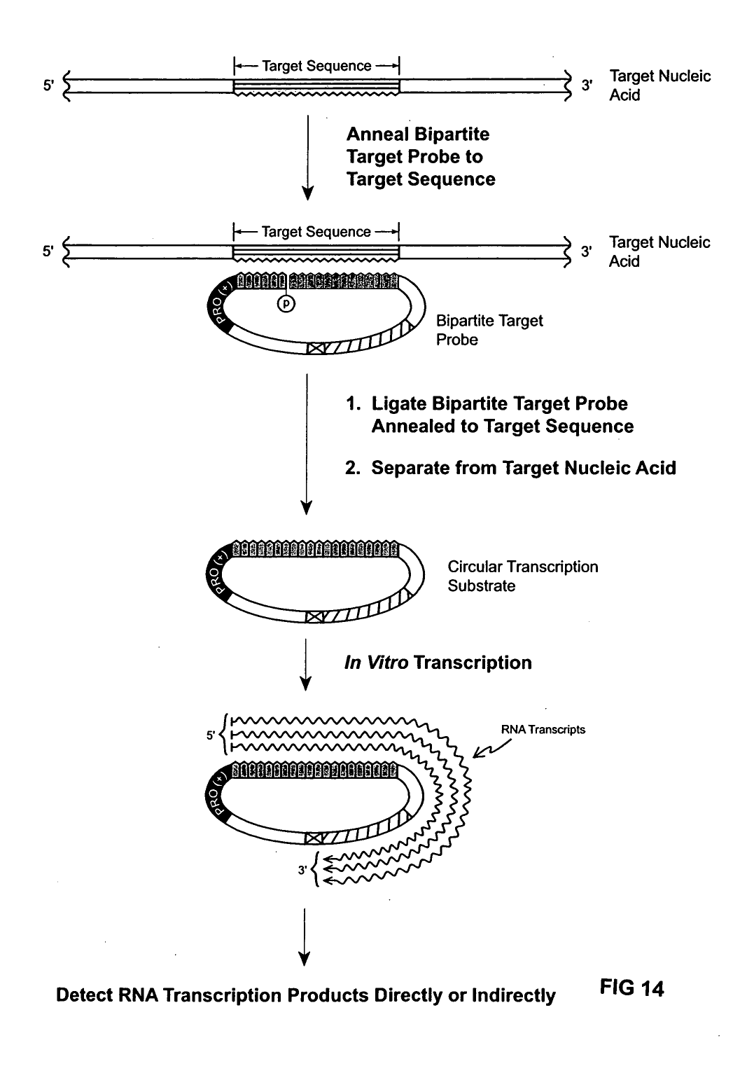

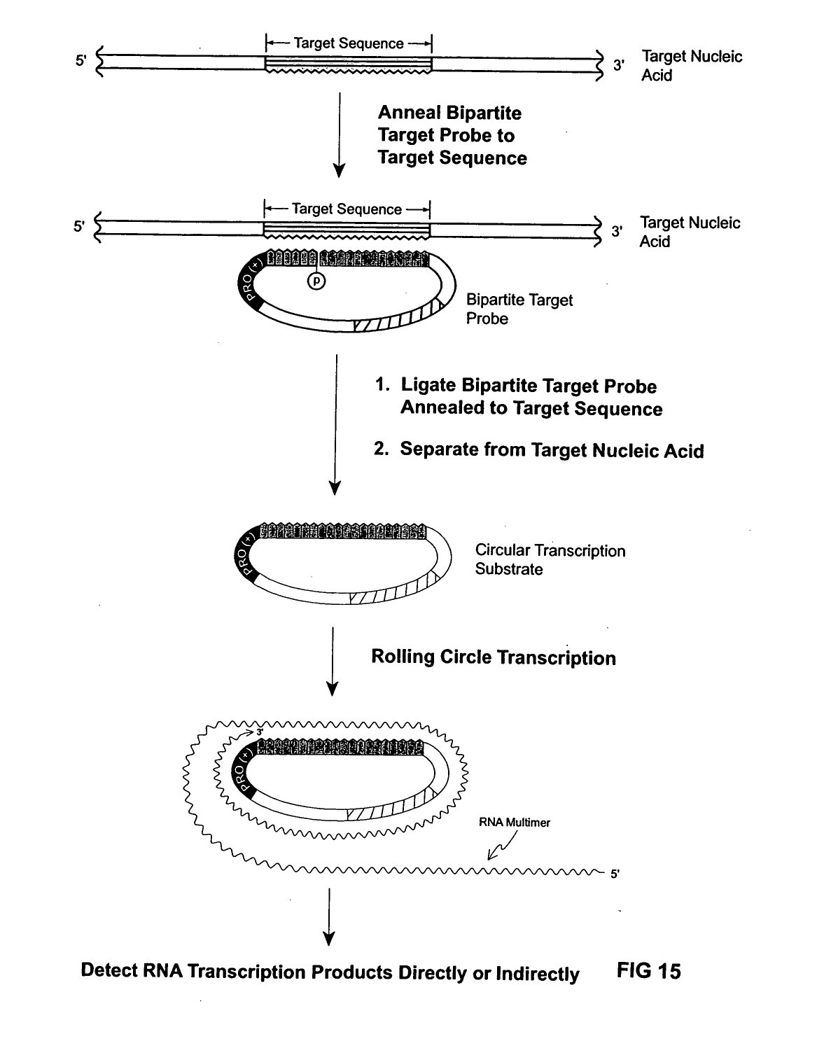

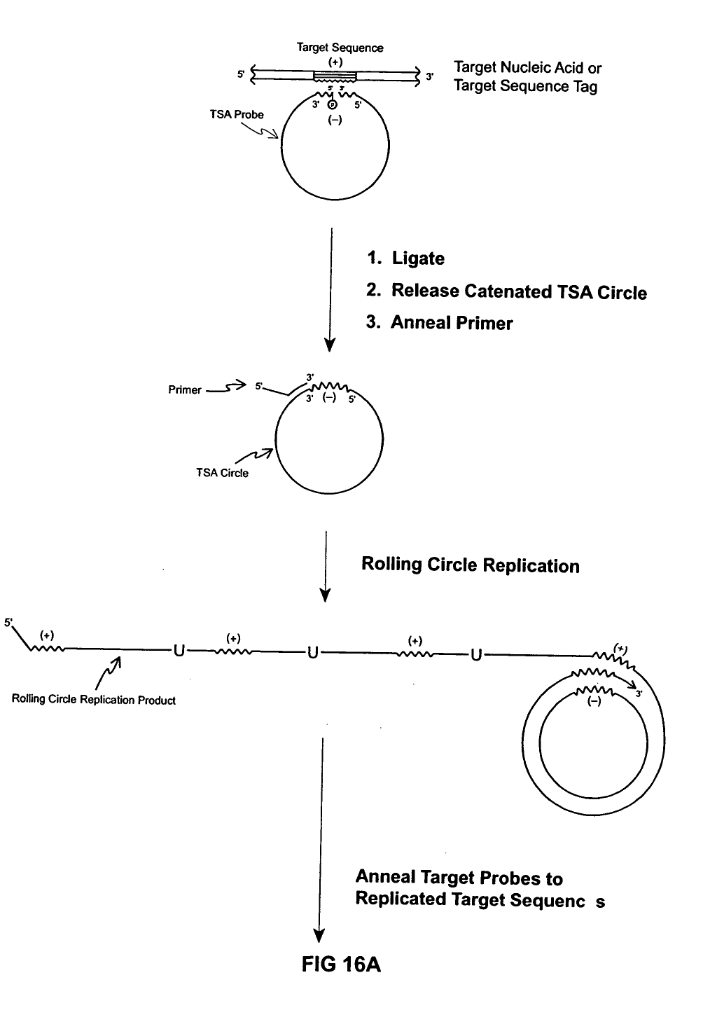

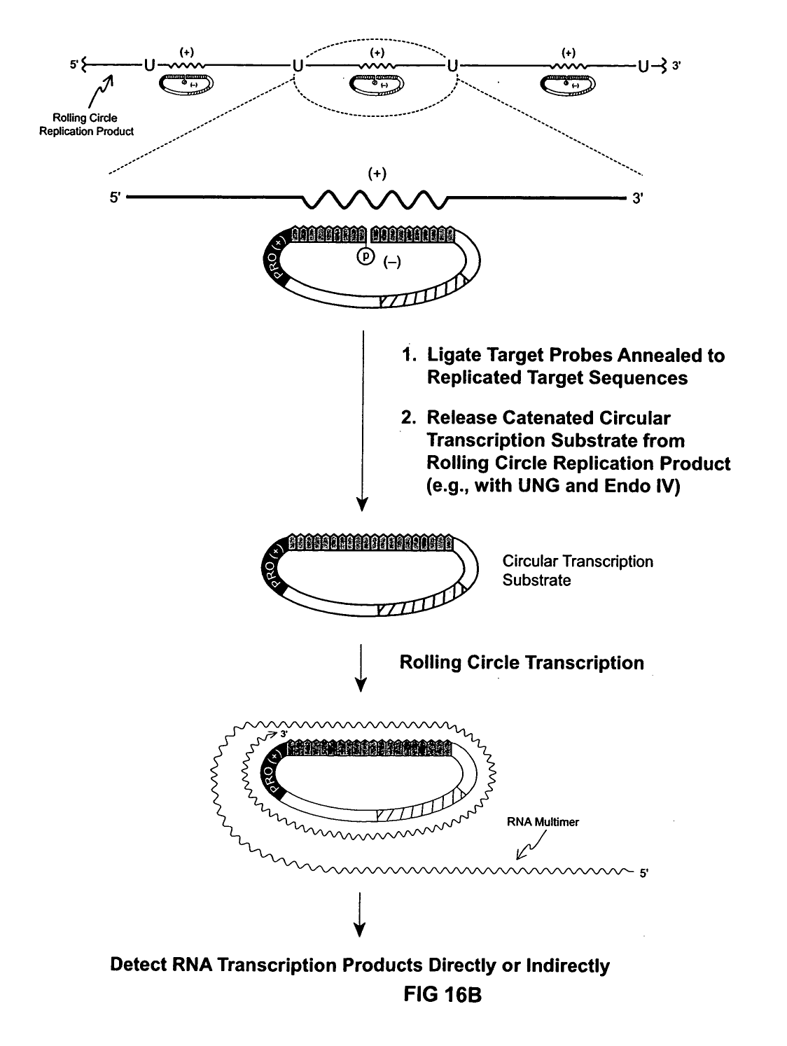

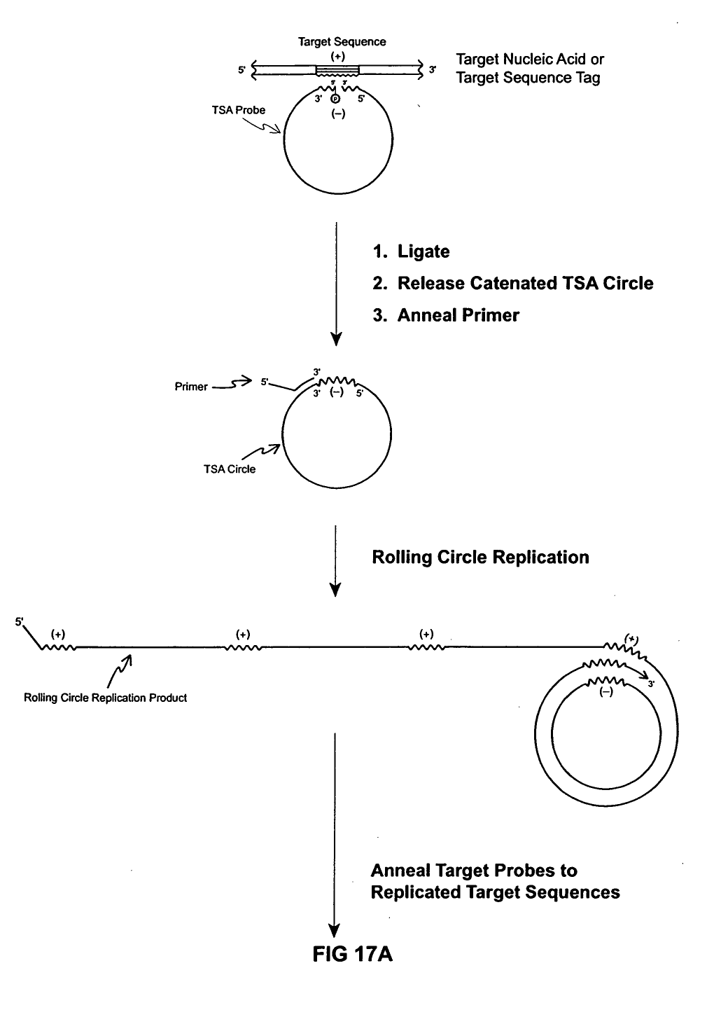

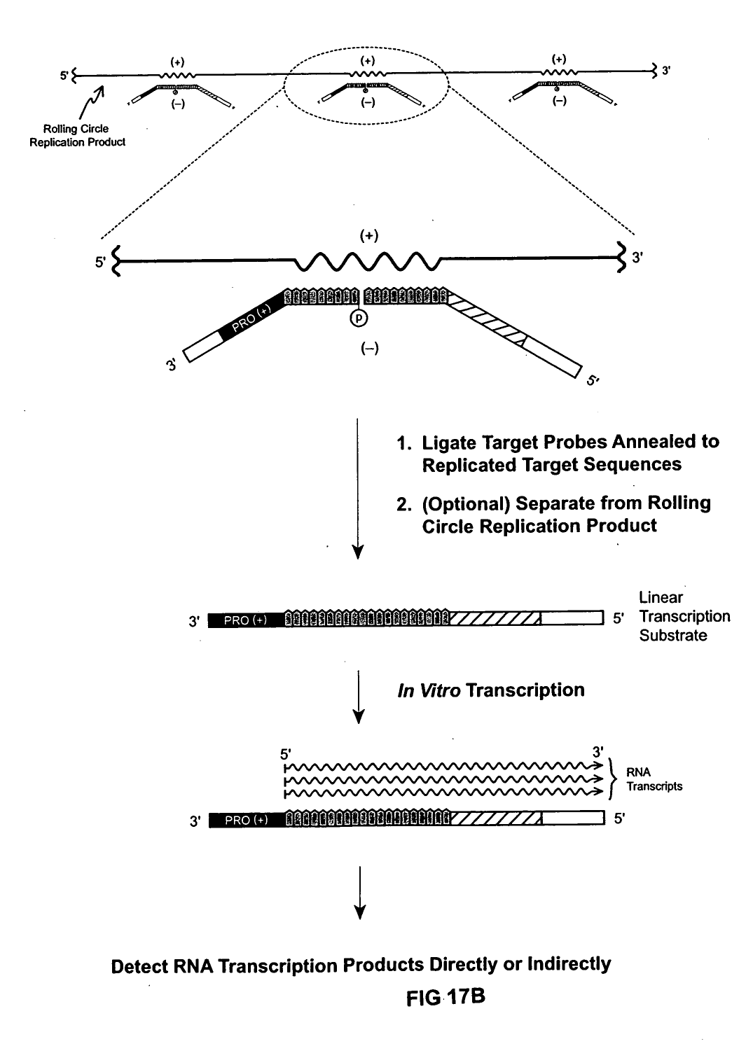

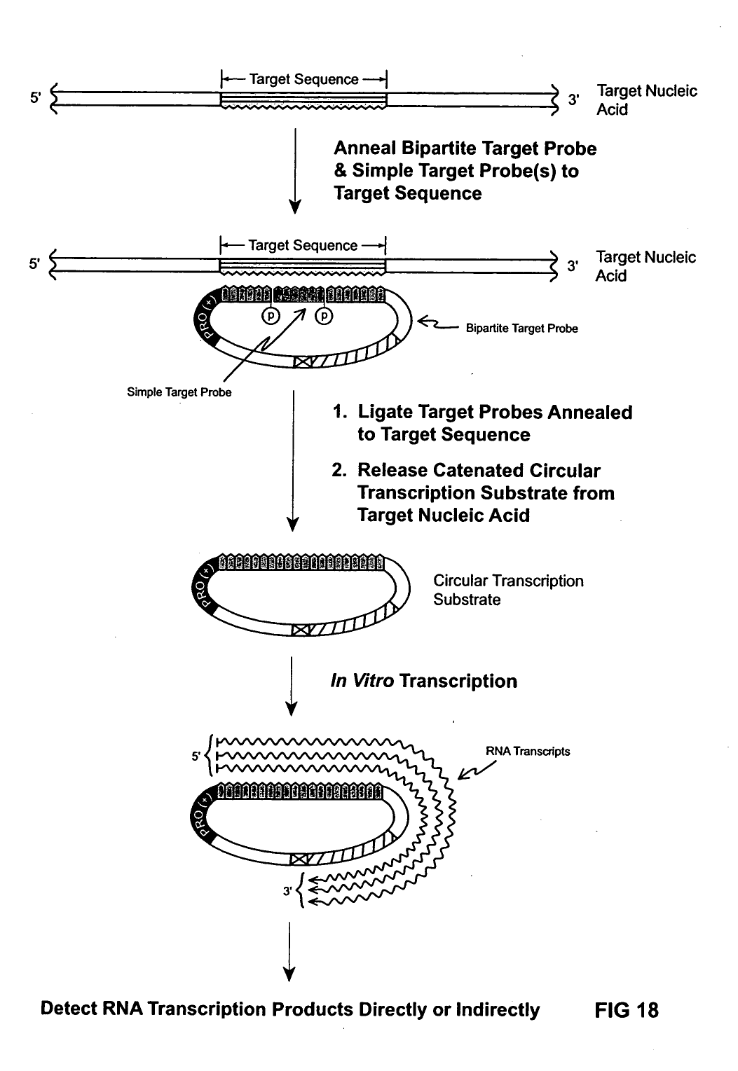

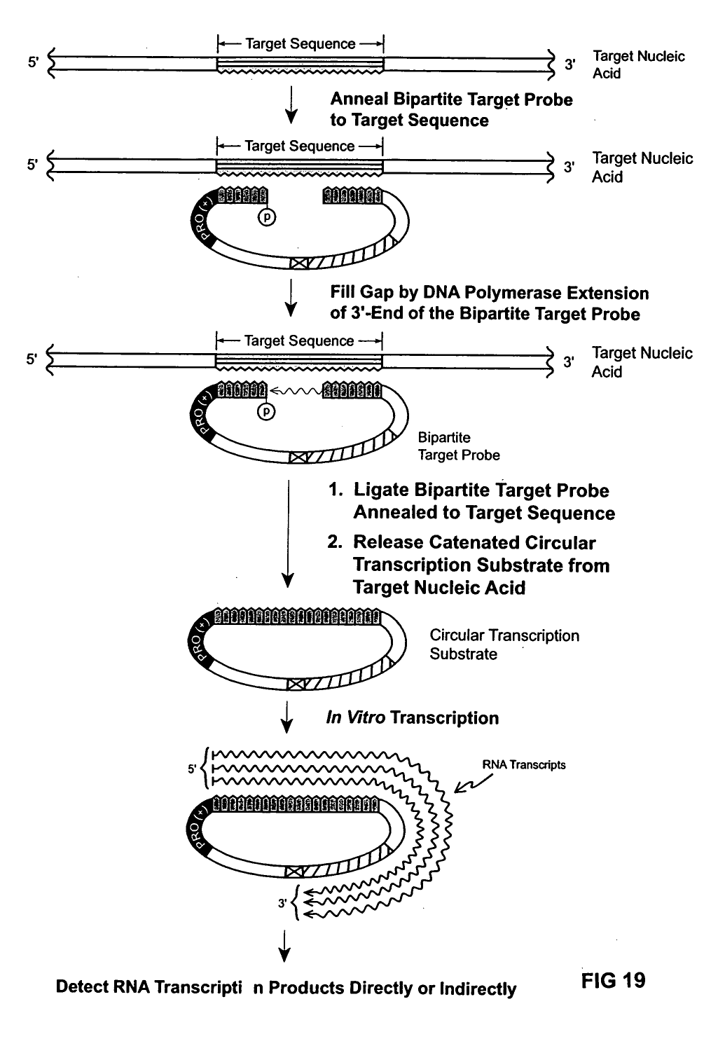

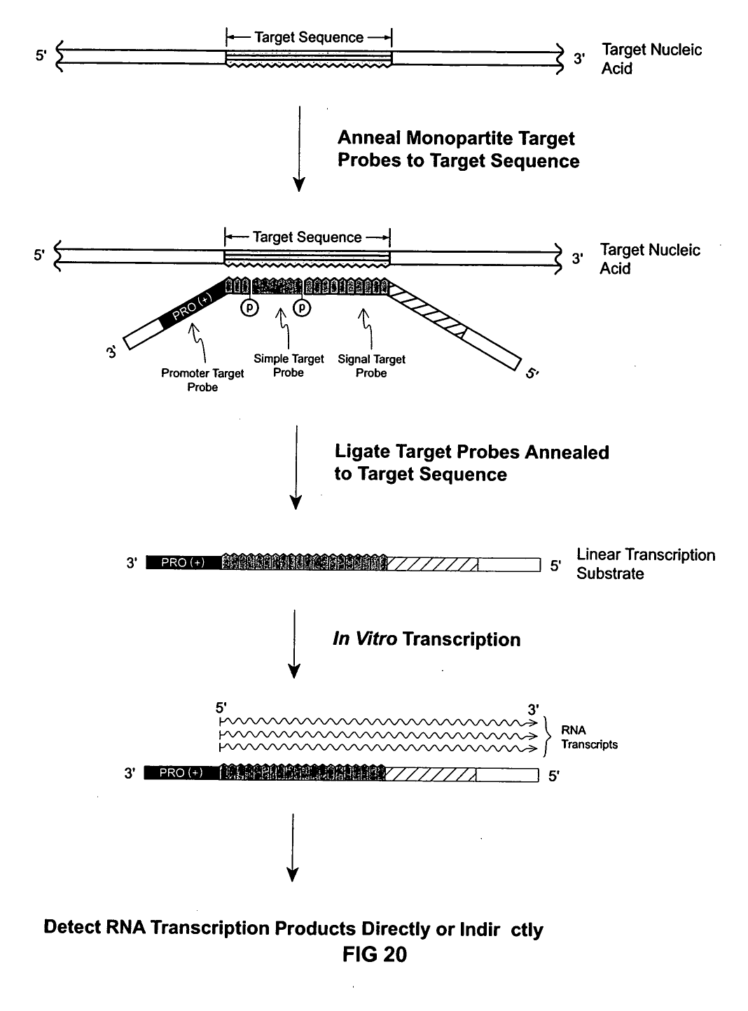

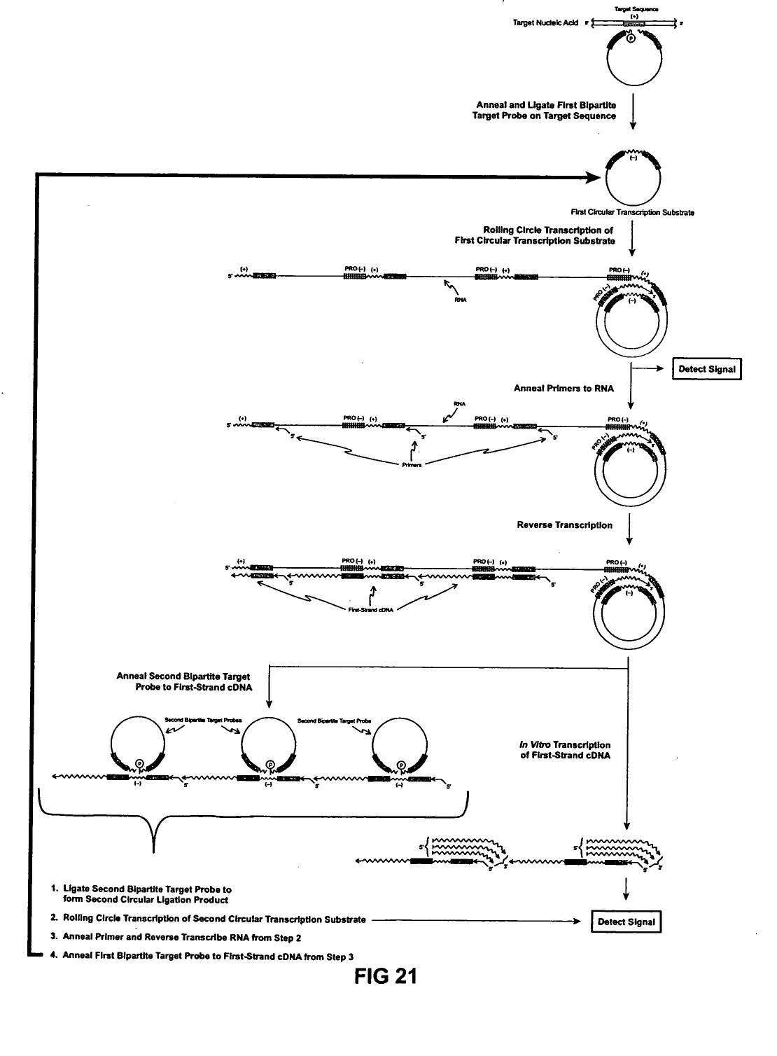

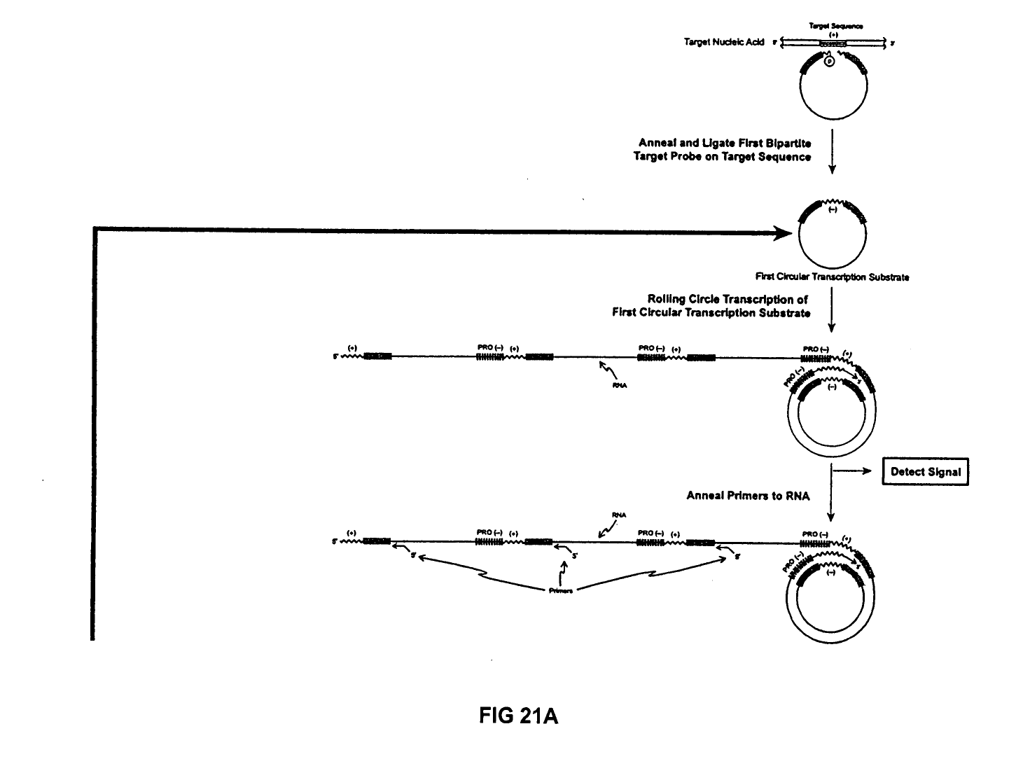

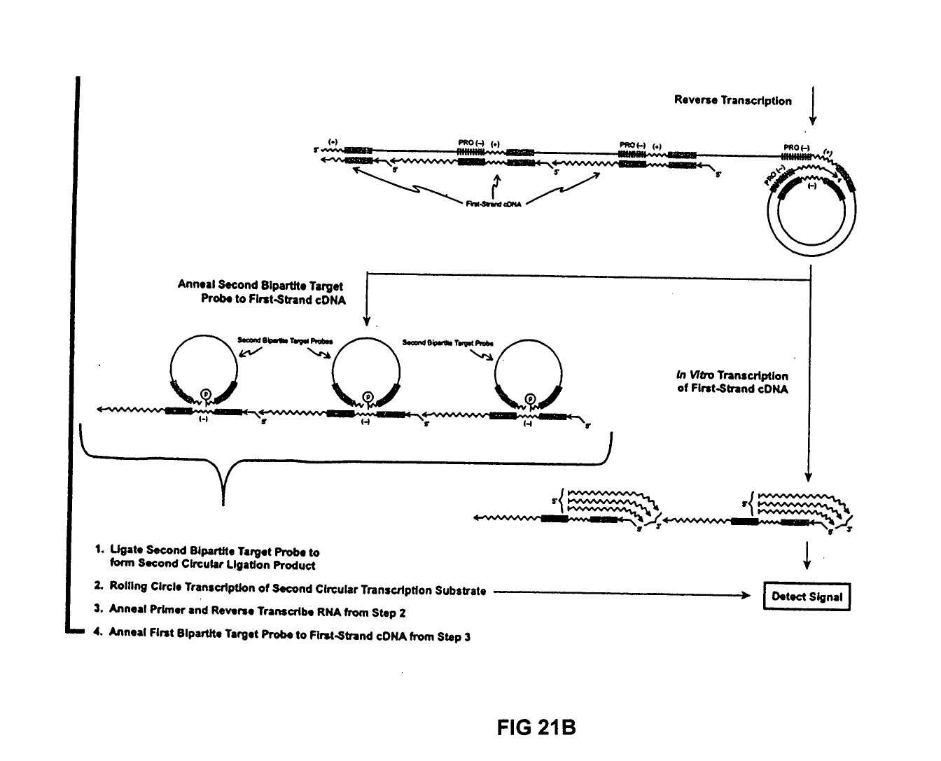

Monopartite and bipartite target probes of the invention that comprise a single-stranded promoter are illustrated in FIGS. 12 and 13. Examples of a number of embodiments of assays and methods of the invention that use target probes comprising single-stranded promoters are presented in FIGS. 14 through 23 to more fully describe the invention. However, the embodiments in these figures are only examples for illustrative purposes and do not limit the scope of the invention, which will be understood to be much broader by a complete reading of the description of the invention herein.

T. Methods and Assays for Detecting and Quantifying Non-Nucleic Acid Analytes Using Target Sequence Tags Comprising or Attached to Analyte-Binding Substances

The present invention includes methods, compositions and kits that use an analyte-binding substance for detecting an analyte in a sample. An “analyte-binding substance” is a substance that binds an analyte that one desires to detect in an assay or method of the invention. An analyte-binding substance is also referred to as an “affinity molecule,” an “affinity substance,” a “specific binding substance,” or a “binding molecule” for an analyte. Usually, an analyte molecule and an analyte-binding substance or affinity molecule for the analyte molecule are related as a specific “binding pair”, i.e., their interaction is only through non-covalent bonds such as hydrogen-bonding, hydrophobic interactions (including stacking of aromatic molecules), van der Waals forces, and salt bridges. Without being bound by theory, it is believed in the art that these kinds of non-covalent bonds result in binding, in part due to complementary shapes or structures of the molecules involved in the binding pair.

The term “binding” according to the invention refers to the interaction between an analyte-binding substance or affinity molecule and an analyte as a result of non-covalent bonds, such as, but not limited to, hydrogen bonds, hydrophobic interactions, van der Waals bonds, and ionic bonds.

In most embodiments of the invention, target probes are used to detect an analyte comprising a target sequence in a target nucleic acid. Following annealing and joining of target probes in the presence of a target sequence in a sample, the resulting transcription substrate is amplified by transcription using an RNA polymerase, and the presence of an RNA complementary to the transcription substrate indicates that the target sequence was present in the sample.

However, a nucleic acid can also be used in a method of the present invention as an analyte-binding substance to detect an analyte that does not comprise a nucleic acid. By way of example, but not of limitation, a method termed “SELEX,” as described by Gold and Tuerk in U.S. Pat. No. 5,270,163, which is incorporated herein by reference, can be used to select a nucleic acid for use as an analyte-binding substance in a method of the invention for detecting an analyte comprising almost any molecule in a sample. SELEX permits selection of a nucleic acid molecule that has high affinity for a specific analyte from a large population of nucleic acid molecules, at least a portion of which have a randomized sequence. For example, a population of all possible randomized 25-mer oligonucleotides (i.e., having each of four possible nucleic acid bases at every position) will contain 4²⁵ (or 10¹⁵) different nucleic acid molecules, each of which has a different three-dimensional structure and different analyte binding properties. SELEX can be used, according to the methods described in U.S. Pat. Nos. 5,270,163; 5,567,588; 5,580,737; 5,587,468; 5,683,867; 5,696,249; 5723,594; 5,773,598; 5,817,785; 5,861,254; 5,958,691; 5,998,142; 6,001,577; 6,013,443; and 6,030,776, all of which are incorporated herein by reference, in order to select an analyte-binding nucleic acid with high affinity for a specific analyte that is not a nucleic acid or polynucleotide for use in a method or assay of the invention. Once selected using SELEX, analyte-binding substances or affinity molecules comprising nucleic acid molecules can be made for use in the methods of the present invention by using any of numerous in vivo or in vitro techniques known in the art, including, by way of example, but not of limitation, automated nucleic acid synthesis techniques, PCR, or in vitro transcription. A nucleic acid molecule that is an analyte-binding substance that has been selected using SELEX can be detected using bipartite or monopartite target probes in a similar way to how such target probes are used to detect a target sequence in a target nucleic acid analyte, as described elsewhere herein. Since an analyte-binding substance that is selected using SELEX comprises a nucleic acid, a continuous sequence within the analyte-binding substance can be used as a “target sequence” and target probes can be designed, wherein the target-complementary sequences in said target probes are complementary to said continuous sequence in said analyte-binding substance. Another important aspect of these embodiments of the invention is that said target sequence in said analyte-binding substance that was selected using SELEX should be capable of annealing to said target probes when said analyte-binding substance is also bound to an analyte; i.e., the binding to the analyte does not block annealing of target probes to the target sequence.

Thus, another embodiment of the present invention is a method for detecting an analyte in a sample, wherein said analyte comprises a biomolecule that is not a nucleic acid, said method comprising:

a. providing an analyte-binding substance comprising a nucleic acid, wherein said nucleic acid binds with selectivity and high affinity to said analyte;

b. providing target probes comprising either (i) a promoter target probe and one or more additional target probes chosen from among a signal target probe and simple target probe; or (ii) a bipartite target probe and, if said target-complementary sequences of said bipartite target probe are not contiguous when annealed to said target sequence in said analyte-binding substance, optionally, one or more simple target probes; wherein said target probes of (i) or (ii) comprise sequences that are complementary to adjacent regions of a target sequence in said analyte-binding substance;

c. contacting said analyte-binding substance to an analyte in a sample;

d. separating said analyte-binding substance molecules that are bound to said analyte from said analyte-binding substance molecules that are not bound to said analyte;

e. contacting said analyte-binding substance molecules that are bound to said analyte with said target probes provided in step b(i) or step b(ii) above under hybridization conditions that permit said target probes that are complementary to said target sequences in said analyte-binding substance to anneal thereto;

f. ligating said adjacent target probes that are annealed to said target sequence of said analyte-binding substance with a ligase under ligation conditions so as to obtain a ligation product;

g. contacting the ligation product with an anti-sense promoter oligo and incubating under hybridization conditions, wherein the anti-sense promoter oligo anneals to the sense promoter sequence of the ligation product to form a transcription substrate;

h. contacting said transcription substrate with an RNA polymerase under transcription conditions so as to synthesize RNA that is complementary to said transcription substrate;

i. optionally, repeating steps a through i; and

j. detecting the synthesis of RNA resulting from transcription of said transcription substrate, wherein said synthesis of said RNA indicates the presence of said analyte in said sample.

Thus, the use of an analyte-binding substance comprising a nucleic acid selected using SELEX permits the methods of the present invention to be used to detect other analyte molecules that are not nucleic acids.

The nucleic acid molecules that contain a randomized sequence that are used to generate a library of molecules for selection of an analyte-binding substance using SELEX can also be made using methods similar to those described by Ohmichi et al. (Proc. Natl. Acad. Sci. USA, 99: 54-59, 2002), incorporated herein by reference. Thus, random sequence circular DNA molecules comprising about 103 nucleotides, of which about 40 nucleotides comprise randomized sequence are repeatedly selected for binding to an analyte by: binding the circular DNAs to an analyte attached to a surface; washing away the unbound circular DNA molecules; recovering the circular DNAs bound to the analyte; obtaining RNA complementary to the recovered circular DNA molecules by rolling circle transcription; amplifying the RNA by RT-PCR using one 5′-biotinylated primer; immobilizing the RT-PCR product on a surface with streptavidin; obtaining the strand of the RT-PCR product that does not contain biotin; and then ligating the single-stranded RT-PCR strand (using a ligation splint) to obtain the first round of selected circular DNA molecules. The first round of circular DNA molecules is then bound to an analyte as just described, and the whole process is again repeated for a total of about 15 rounds of selection of circular DNA molecules for analyte binding. The selected circular DNA molecules are then analyzed for analyte binding in order to obtain an analyte-binding substance for use in an assay or method of the present invention. As described above related to SELEX, a target sequence in the selected analyte-binding substance can be detected using monopartite or bipartite target probes as described elsewhere herein. Thus, an analyte-binding substance is used to bind an analyte in a sample and then, after removing unbound analyte-binding substance (if the analyte-binding substance is attached to a surface or becomes attached to a surface during a process of the assay or method), the analyte-binding substance is detected using target probes that are complementary to a target sequence in the analyte-binding substance. A method for detecting an analyte-binding substance can comprise a step comprising ligation of target probes of the invention as described in the embodiments of the method immediately above herein for detecting a target sequence in an analyte-binding substance that is bound to an analyte. However, in some other embodiments for detecting an analyte-binding substance that is bound to an analyte, a ligation step is omitted and the analyte-binding substance:analyte complex is detected by annealing to said complex a transcription substrate that contains a sequence that is complementary to a target sequence in said analyte-binding substance. After removing unhybridized transcription substrates, transcription substrates that are annealed to said analyte-binding substance: analyte complex are detected by synthesis of RNA resulting from in vitro transcription of the complex-bound transcription substrate.

A “peptide nucleic acid (PNA)” or a molecule comprising both a nucleic acid and a PNA, as described in U.S. Pat. Nos. 5,539,082; 5,641,625; 5,700,922; 5,705,333; 5,714,331; 5,719,262; 5,736,336; 5,773,571; 5,786,461; 5,817,811; 5,977,296; 5,986,053; 6,015,887; and 6,020,126 (and references therein), all of which are incorporated herein by reference, can also be used in methods of the present invention as an analyte-binding substance for a non-nucleic acid analyte. In general, a PNA molecule is a nucleic acid analog consisting of a backbone comprising, for example, N-(2-aminoethyl)glycine units, to each of which a nucleic acid base is linked through a suitable linker, such as, but not limited to an aza, amido, ureido, or methylene carbonyl linker. The nucleic acid bases in PNA molecules bind complementary single-stranded DNA or RNA according to Watson-Crick base-pairing rules. However, the Tₘ's for PNA/DNA or PNA/RNA duplexes or hybrids are higher than the Tₘ's for DNA/DNA, DNA/RNA, or RNA/RNA duplexes. In these embodiments, a “PNA target sequence” is present in said analyte-binding substance comprising PNA, to which, target-complementary sequences of monopartite or bipartite target probes (or transcription substrates) can anneal, permitting detection as described above for analyte-binding molecules selected using SELEX. Thus, PNA used as an analyte-binding substance in an assay or method of the present invention provides tighter binding (and greater binding stability) for target-complementary sequences in target probes or transcription substrates (e.g., see U.S. Pat. No. 5,985,563). Also, since PNA is not naturally occurring, PNA molecules are highly resistant to protease and nuclease activity. PNA for use as an analyte binding substance can be prepared according to methods known in the art, such as, but not limited to, methods described in the above-mentioned patents, and references therein. Antibodies to PNA/analyte complexes can be used in the invention for capture, recognition, detection, identification, or quantitation of nucleic acids in biological samples, via their ability to bind specifically to the respective complexes without binding the individual molecules (U.S. Pat. No. 5,612,458).

The invention also contemplates that a combinatorial library of randomized peptide nucleic acids prepared by a method such as, but not limited to, the methods described in U.S. Pat. Nos. 5,539,083; 5,831,014; and 5,864,010, can be used to prepare analyte-binding substances for use in assays for analytes of all types, including analytes that are nucleic acids, proteins, or other analytes, without limit. As is the case for the SELEX method with nucleic acids, randomized peptide or peptide nucleic acid libraries are made to contain molecules with a very large number of different binding affinities for an analyte. After selection of an appropriate affinity molecule for an analyte from a library, the selected affinity molecule can be used in the invention as an analyte-binding substance in the second portion of the reporter probe.

An analyte-binding substance can also be an oligonucleotide or polynucleotide with a modified backbone that is not an amino acid, such as, but not limited to modified oligonucleotides described in U.S. Pat. Nos. 5,602,240; 6,610,289; 5,696,253; or 6,013,785.

The invention also contemplates that an analyte-binding substance can be prepared from a combinatorial library of randomized peptides (i.e., comprising at least four naturally-occurring amino acids). One way to prepare the randomized peptide library is to place a randomized DNA sequence, prepared as for SELEX, downstream of a phage T7 RNA polymerase promoter, or a similar promoter, and then use a method such as, but not limited to, coupled transcription-translation, as described in U.S. Pat. Nos. 5,324,637; 5,492,817; or 5,665,563, or stepwise transcription, followed by translation. Alternatively, a randomized DNA sequence, prepared as for SELEX, can be cloned into a site in a DNA vector that, once inserted, encodes a recombinant MDV-1 RNA containing the randomized sequence that is replicatable by Q-beta replicase (e.g., between nucleotides 63 and 64 in MDV-1 (+) RNA; see U.S. Pat. No. 5,620,870). The recombinant MDV-1 DNA containing the randomized DNA sequence is downstream from a T7 RNA polymerase promoter or a similar promoter in the DNA vector. Then, following transcription, the recombinant MDV-1 RNA, containing the randomized sequence can be used to make a randomized peptide library comprising at least four naturally occurring amino acids by coupled replication-translation as described in U.S. Pat. No. 5,556,769. An analyte-binding substance can be selected from the library by binding peptides in the library to an analyte, separating the unbound peptides, and identifying one or more peptides that is bound to analyte by means known in the art. Alternatively, high throughput screening methods can be used to screen all individual peptides in the library to identify those that can be used as analyte-binding substances. Although the identification of an analyte-binding peptide by these methods is difficult and tedious, the methods in the art are improving for doing so, and the expenditure of time and effort required may be warranted for identifying analyte-binding substances for use in assays of the invention that will be used routinely in large numbers.

In embodiments of the present invention in which an analyte-binding substance comprises a peptide, a protein, including, but not limited to an antibody, streptavidin, or another biomolecule, a nucleic acid sequence can be attached to said analyte-binding substance, wherein said nucleic acid serves as a “tag” comprising a target sequence that can be detected using target probes or transcription substrates of the invention. In this way, the methods and assays of the invention can be used for sensitive and specific detection of analytes that are not nucleic acids.

Analyte-binding substances for particular analytes and methods of preparing them are well known in the art. Naturally occurring nucleic acid or polynucleotide sequences that have affinity for other naturally occurring molecules such as, but not limited to, protein molecules, are known in the art, and nucleic acid molecules comprising these sequences can be used, both as analyte-binding substances and as tags comprising target sequences for detection using target probes or transcription substrates of the invention. Examples include, but are not limited to certain nucleic acid sequences such as operators, promoters, origins of replication, sequences recognized by steroid hormone-receptor complexes, restriction endonuclease recognition sequences, ribosomal nucleic acids, and so on, which are known to bind tightly to certain proteins. For example, in two well-known systems, the lac repressor and the bacteriophage lambda repressor each bind to their respective specific nucleic acid sequences called “operators” to block initiation of transcription of their corresponding mRNA molecules. Nucleic acids containing such specific sequences can be used in the invention as analyte-binding substances for the respective proteins or other molecules for which the nucleic acid has affinity. In these cases, the nucleic acid with the specific sequence is used as the analyte-binding substance in assays for the respective specific protein, glycoprotein, lipoprotein, small molecule or other analyte that it binds. One of several techniques that is generally called “footprinting” (e.g., see Galas, D. and Schmitz, A, Nucleic Acids Res., 5: 3161, 1978) can be used to identify sequences of nucleic acids that bind to a protein. Other methods are also known to those with skill in the art and can be used to identify nucleic acid sequences for use as specific analyte-binding substances for use in the invention.

A variety of other analyte-binding substances can also be used. By way of example but not of limitation, an analyte-binding substance can be an antibody, including monoclonal, polyclonal, or artificial antibodies which are made using methods well known in the art, and the analyte can be any substance for which a specific-binding antibody can be prepared, including peptides, proteins, carbohydrates, lipids glycoproteins, lipoproteins, and biochemicals, either alone or conjugated to another molecule in order to increase the “antigenicity,” or ability to provoke an antibody response. For an antigen analyte (which itself may be an antibody), antibodies, including monoclonal antibodies, are available as analyte-binding substances. For certain antibody analytes in samples which include only one antibody, an antibody binding protein such as Staphylococcus aureus Protein A can be employed as an analyte-binding substance. For an analyte, such as a glycoprotein or class of glycoproteins, or a polysaccharide or class of polysaccharides, which is distinguished from other substances in a sample by having a carbohydrate moiety that is bound specifically by a lectin, a suitable analyte-binding substance is the lectin. For an analyte that is a hormone, a receptor for the hormone can be employed as an analyte-binding substance. Conversely, for an analyte that is a receptor for a hormone, the hormone can be employed as the analyte-binding substance. For an analyte that is an enzyme, an inhibitor of the enzyme can be employed as an analyte-binding substance. For an analyte that is an inhibitor of an enzyme, the enzyme can be employed as the analyte-binding substance.

Based on the definition for “binding,” and the wide variety of affinity molecules and analytes that can be used in the invention, it is clear that “binding conditions” vary for different specific binding pairs. Those skilled in the art can easily determine conditions whereby, in a sample, binding occurs between affinity molecule and analyte that may be present. In particular, those skilled in the art can easily determine conditions whereby binding between affinity molecule and analyte that would be considered in the art to be “specific binding” can be made to occur. As understood in the art, such specificity is usually due to the higher affinity of affinity molecule for analyte than for other substances and components (e.g., vessel walls, solid supports) in a sample. In certain cases, the specificity might also involve, or might be due to, a significantly more rapid association of affinity molecule with analyte than with other substances and components in a sample.

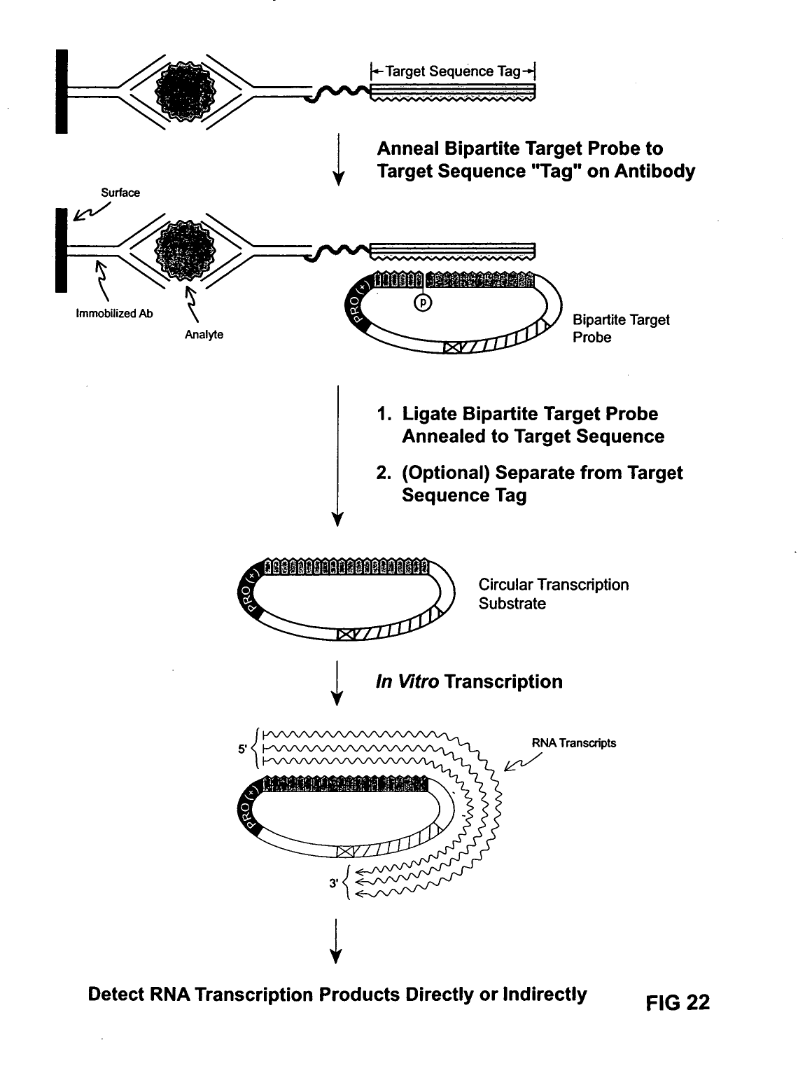

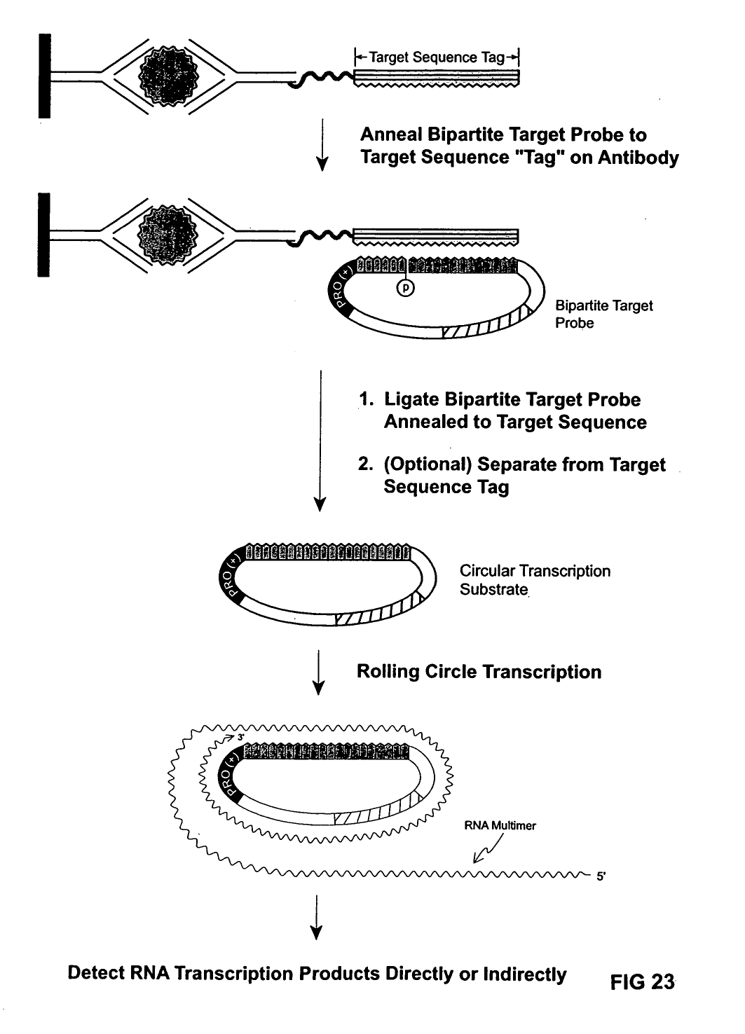

In general, any of the methods and assays described herein to detect and quantify an analyte comprising a target sequence in a target nucleic acid can also be used to detect and quantify a target sequence that comprises a target sequence tag that is attached to an analyte-binding substance for a non-nucleic acid analyte by adjusting the reaction conditions of said assay or method to accommodate the specific analyte and analyte-binding substance. Thus, the methods and assays of the invention permit detection and quantification of any analyte for which there is a suitable analyte-binding substance that either comprises or to which a target sequence tag can be attached. Two methods for using for using target probes that comprise a sense promoter sequence for a double-stranded promoter for detecting an analyte using an analyte-binding substance comprising an antibody having a target sequence tag are illustrated in FIGS. 10 and 11. Similar assays that use target probes comprising a single-stranded promoter or pseudopromoter are illustrated in FIGS. 22 and 23.

U. Use of Transcription Substrates and RNA Polymerases of the Invention as Signaling Systems

The invention also comprises methods, compositions and kits for using ssDNA transcription substrates and RNA polymerases that can transcribe said ssDNA transcription substrates as a signaling system for an analyte of any type, including analytes such as, but not limited to, antigens, antibodies or other substances, in addition to an analyte that is a target nucleic acid.

Thus, the invention comprises a method for detecting an analyte in or from a sample, said method comprising:

1. providing a transcription signaling system, said transcription signaling system comprising a ssDNA comprising: (a) a 5′-portion comprising a sense promoter sequence for a double-stranded promoter for a cognate RNA polymerase; and (b) a signal sequence, wherein said signal sequence, when transcribed by said RNA polymerase, is detectable in some manner;

2. joining said transcription signaling system, either covalently or non-covalently, to an analyte-binding substance, wherein said joining to said substance is not affected by the conditions of the assay and wherein said joining to said substance does not affect the ability of said transcription signaling system to be transcribed using said RNA polymerase under transcription conditions;

3. contacting said analyte-binding substance to which said transcription signaling system is joined with a sample under binding conditions, wherein said analyte, if present in said sample, binds to said analyte-binding substance so as to form a specific binding pair;

4. removing said specific binding pair from said sample so as to separate it from other components in said sample;

5. contacting the specific binding pair with an anti-sense promoter oligo under annealing conditions, wherein the anti-sense promoter oligo anneals to the sense promoter sequence to form a transcription substrate;

6. incubating said specific-binding pair under transcription conditions with an RNA polymerase, wherein said RNA polymerase synthesizes RNA that is complementary to said signal sequence in said ssDNA transcription signaling system under said transcription conditions;

7. obtaining the RNA synthesis product that is complementary to said signal sequence in said ssDNA transcription signaling system; and

8. detecting said RNA synthesis product or a substance that results from said RNA synthesis product.

An analyte or an analyte-binding substance of this aspect of the invention can be any combination of biological molecules that can form a specific binding pair. By way of example, but not of limitation, an analyte-binding substance can be an antibody and the analyte an antigen, or an analyte-binding substance can be a nucleic acid and the analyte can be another complementary nucleic acid. A large number of other substances exist for which a specific-binding pair can be found. Also the signal sequence can vary greatly. By way of example, but not of limitation, a signal sequence can comprise a substrate for Q-beta replicase, which is detectable in the presence of said replicase under replication conditions. It can also comprise a sequence that encodes a protein, such as green fluorescent protein, that is detectable following translation of the signal sequence. Without limitation, it can also comprise a sequence that is detectable by a probe, such as, but not limited to a molecular beacon, as described by Tyagi et al. (U.S. Pat. Nos. 5,925,517 and 6,103,476 of Tyagi et al. and 6,461,817 of Alland et al., all of which are incorporated herein by reference). The present invention with regard to signaling systems also comprises uses such as those for methods described by Zhang et al. (Proc. Natl. Acad. Sci. USA, 98: 5497-5502, 2001, incorporated herein by reference) or by Hudson et al. in U.S. Pat. No. 6,100,024, incorporated herein by reference.

V. Modes of Performance of Methods and Assays of the Invention for Detecting a Target Sequence

Depending on the application and its requirements and constraints, the methods of the invention can be performed in a stepwise fashion, with one set of reactions being performed, followed by purification of a reaction product or removal of reagents or inactivation of enzymes or addition of reagents before proceeding to the next set of reactions, or, in other embodiments, which are preferred embodiments, the methods and assays can be performed as a continuous set of multiple reactions in a single reaction mixture. The invention also comprises methods or assays in which multiple target probes or target probe sets are used in a single reaction mixture in order to detect and/or quantify multiple target sequences in one or multiple target nucleic acids. Thus, the compositions, kits, methods and assays of the invention can be used in a multiplex format.

The invention is not limited to these reaction conditions or concentrations of reactants, except that the reaction conditions must be appropriate for each step of a method or assay of the invention. Those with skill in the art will know how to find and determine suitable reaction conditions under which enzymes, including ligases, RNA polymerases, DNA polymerases, replicases (such as Q-beta replicase) and related enzymes are active for the methods of the invention and will know that optimal combined conditions can be used can be found by simple experimentation, and any of these reaction conditions are included within the scope of the invention. Such media and conditions are known to persons of skill in the art, and are described in various publications such as, but not limited to U.S. Pat. No. 5,679,512 and PCT Pub. No. WO99/42618. For example, a buffer can be Tris buffer, although other buffers can also be used as long as the buffer components are non-inhibitory to enzyme components of the methods of the invention. The pH is preferably from about 5 to about 11, more preferably from about 6 to about 10, even more preferably from about 7 to about 9, and most preferably from about 7.5 to about 8.5. The reaction medium can also include bivalent metal ions such as Mg⁺²or Mn⁺, at a final concentration of free ions that is within the range of from about 0.01 to about 10 mM, and most preferably from about 1 to 6 mM. The reaction medium can also include other salts, such as KCl, that contribute to the total ionic strength of the medium. For example, the range of a salt such as KCl is preferably from about 0 to about 100 mM, more preferably from about 0 to about 75 mM, and most preferably from about 0 to about 50 mM. Cofactors can be supplied for enzymes as appropriate, such as, but not limited to NAD at a final concentration of about 0.5 mM for an NAD-dependent ligase or ATP at a final concentration of about 0.1 to 1.0 mM for an ATP-dependent ligase or a polynucleotide kinase, respectively. The reaction medium can further include additives that could affect performance of the reactions, but that are not integral to the activity of the enzyme components of the methods. Such additives include proteins such as BSA, and non-ionic detergents such as NP40 or Triton. Reagents, such as DTT, that are capable of maintaining activities enzyme with sulfhydryl groups can also be included. Such reagents are known in the art. Where appropriate, an RNase inhibitor, such as, but not limited to a placental ribonuclease inhibitor (e.g., RNasin®, Promega Corporation, Madison, Wis., USA) or an antibody RNase inhibitor, that does not inhibit the activity of an RNase employed in the method can also be included. Any aspect of the methods of the present invention can occur at the same or varying temperatures. Preferably, the reactions are performed isothermally, which avoids the cumbersome thermocycling process. The reactions are carried out at a temperature that permits hybridization of the oligonucleotides of the present invention to the target sequence and/or first-strand cDNA of a method of the invention and that does not substantially inhibit the activity of the enzymes employed. The temperature can be in the range of preferably about 25° C. to about 85° C., more preferably about 30° C. to about 75° C., and most preferably about 37° C. to about 70° C. In the processes that include RNA transcription, the temperature for the transcription steps is lower than the temperature(s) for the preceding steps. In these processes, the temperature of the transcription steps can be in the range of preferably about 25° C. to about 85° C., more preferably about 30° C. to about 75° C., and most preferably about 37° C. to about 55° C.

As disclosed in U.S. Pat. Nos. 6,048,696 and 6,030,814, as well as in German Patent No. DE4411588C1, all of which are incorporated herein by reference and made part of the present invention, it is preferred in many embodiments to use a final concentration of about 0.25 M, about 0.5 M, about 1.0 M, about 1.5 M, about 2.0 M, about 2.5 M or between about 0.25 M and 2.5 M betaine (trimethylglycine) in DNA polymerase or reverse transcriptase reactions in order to decrease DNA polymerase stops and increase the specificity of reactions which use a DNA polymerase.

Nucleotide and/or nucleotide analogs, such as deoxyribonucleoside triphosphates, that can be employed for synthesis of reverse transcription or primer extension products in the methods of the invention are provided in an amount that is determined to be optimal or useful for a particular intended use.

The oligonucleotide components of reactions of the invention are generally in excess of the number of target nucleic acid sequence to be amplified. They can be provided at about or at least about any of the following: 10, 10², 10⁴, 10⁶, 10⁸, 10¹⁰, 10¹² times the amount of target nucleic acid. Target probes, primers, anti-sense promoter oligos, strand-displacement primers, and the like, can each be provided at about or at least about any of the following concentrations: 50 nM, 100 nM, 500 nM, 1000 nM, 2500 nM, 5000 nM, or 10,000 nM, but higher or lower concentrations can also be used. By way of example, but not of limitation, a concentration of one or more oligonucleotides may be desirable for production of one or more target nucleic acid sequences that are used in another application or process. The invention is not limited to a particular concentration of an oligonucleotide, so long as the concentration is effective in a particular method of the invention.

In some embodiments, the foregoing components are added simultaneously at the initiation of the process. In other embodiments, components are added in any order prior to or after appropriate time points during the process, as required and/or permitted by the reaction. Such time points can readily be identified by a person of skill in the art. The enzymes used for nucleic acid reactions according to the methods of the present invention are generally added to the reaction mixture following a step for denaturation of a double-stranded target nucleic acid in or from a sample, and/or following hybridization of primers and/or oligos of a reaction to a denatured double-stranded or single-stranded target nucleic acid, as determined by their thermal stability and/or other considerations known to the person of skill in the art.

The reactions can be stopped at various time points, and resumed at a later time. The time points can readily be identified by a person of skill in the art. Methods for stopping the reactions are known in the art, including, for example, cooling the reaction mixture to a temperature that inhibits enzyme activity. Methods for resuming the reactions are also known in the art, including, for example, raising the temperature of the reaction mixture to a temperature that permits enzyme activity. In some embodiments, one or more of the components of the reactions is replenished prior to, at, or following the resumption of the reactions. Alternatively, the reaction can be allowed to proceed (i.e., from start to finish) without interruption.

The invention also comprises parts or subsets of the methods and compositions of the invention. Thus, the invention comprises all of the individual steps of the methods of the invention that are enabled thereby, in addition to the overall methods.

W. Kits and Compositions of the Invention for Detecting a Target Sequence in a Target Nucleic Acid Analyte or a Target Sequence Tag Comprising or Attached to an Analyte-Binding Substance

The present invention also comprises kits and compositions for carrying out the methods of the invention. A kit of the invention comprises one or, preferably, multiple components or compositions for carrying out the various processes of a method. Different embodiments of kits and compositions of the present invention can comprise one or more of the following:

1. a bipartite target probe for an assay or method for detecting a particular target sequence, and, optionally if the target-complementary sequences of said bipartite target probe are not contiguous when annealed to a target sequence, a monopartite target probe, all of which target probes preferably have a 5′-phosphate group.

2. a set of monopartite target probes for an assay or method for detecting a particular target sequence, wherein said set of monopartite target probes comprises a promoter target probe, preferably having a 5′-phosphate group, and either a signal target probe or a simple target probe and one or more additional simple target probes, which if present, preferably each have a 5′-phosphate group.

3. a ligase, wherein said ligase has little or no activity in ligating blunt ends and is substantially more active in ligating the ends of target probe if said ends are adjacent when annealed to two contiguous regions of a complementary sequence than if said ends are not annealed to said complementary sequence. In preferred embodiments of the above methods or assays for detecting a target sequence, said ligase comprises a ligase chosen from among Ampligase® thermostable DNA Ligase, Tth DNA Ligase, Tfl DNA Ligase, Tsc DNA Ligase, or Pfu DNA Ligase.

4. an RNA polymerase preparation, wherein said RNA polymerase recognizes a transcription promoter in a transcription substrate generated from a method of the invention and initiates transcription therefrom using as a template single-stranded DNA to which said promoter is functionally attached. In preferred embodiments of the above methods or assays for detecting a target sequence, said RNA polymerase is T7 RNAP, T3 RNAP or SP6 RNAP. In some embodiments, E. coli RNAP or Thermus thermophilus RNAP is used. In some embodiments that incorporate non-canonical 2′-modified nucleotides, a mutant enzyme, such as, but not limited to T7 Y639F mutant RNAP is preferred. In embodiments that use a double-stranded promoter, the kit or composition also comprises an anti-sense promoter oligo, which in some embodiments is attached to a solid support. In some embodiments, the RNA polymerase preparation comprises an N4 mini-vRNAP and EcoSSB Protein.

5. a reverse transcriptase, for embodiments that use a reverse transcription process in order to obtain additional amplification of a target sequence and/or a signal sequence. In preferred embodiments, said reverse transcriptase has RNase H activity and is chosen from among MMLV, AMV or another retroviral reverse transcriptase, or a reverse transcriptase encoded by a thermostable phage. In other embodiments, said reverse transcriptase comprises a DNA polymerase chosen from among IsoTherm™, Bst large fragment, Bca.BEST™, and Tth DNA polymerase. Embodiments that use a reverse transcription process also use one or more primers, which can also comprise a composition or kit of the invention.

6. a DNA polymerase, for embodiments that use DNA polymerase extension to fill a gap between target probes. Any DNA polymerase that does not strand displace a downstream target probe can be used in a composition or kit of the invention.

7. an analyte-binding substance that either comprises or has an attached target sequence tag for embodiments of the invention for detecting and/or quantifying an analyte in a sample.

8. detection compositions. A composition or kit of the invention can comprise a wide variety of compositions for detecting an RNA transcript that is complementary to a target sequence and/or a signal sequence. By way of example, but not of limitation, a composition or kit can comprise a detection oligo, such as a molecular beacon. Alternatively, a composition or kit can comprise an enzyme, such as Q-beta replicase, if the signal sequence encodes a Q-beta replicase substrate. The invention comprises kits comprising any suitable detection composition.

9. controls, including quantification standards. Controls are used in assays and methods of the invention in order to verify that the assay or method produces the required specificity and sensitivity, or, in other words, to determine the frequency and conditions that lead to “false positive” and/or “false negative” results. Thus, controls comprise important compositions and kits for assays and methods of the invention. By way of example, but not of limitation, a positive control might be a sample containing a known quantity of a target sequence. A negative control would lack the target sequence. For an assay or method to detect a target nucleotide that is a single nucleotide polymorphism or SNP, positive controls might comprise sample that contain either the mutant or the predominant allele or other known alleles for that nucleotide position in the target sequence. In general, quantification of a target analyte in a sample using an assay or method of the invention, including an analyte comprising a target sequence, is achieved by using controls containing different known quantities of said analyte as a standard. Provided that the control sample is as close in performance as possible to a “real world” sample using the methods or assays of the invention, the amount of the analyte in the sample can be standardized against the results obtained using quantification controls. A composition or kit can also, for example, comprise a control comprising an antigen for an assay or method that uses an analyte-binding substance comprising an antibody with a bound target sequence tag or a molecule selected using SELEX. Most of the embodiments for detecting a target sequence are linear and the side-by-side results obtained compared to quantification standards will be proportional to the amount of said analyte in a sample. However, special care will need to be taken in trying to quantify the amount of analyte in a sample when an embodiment of an assay or method that comprises secondary or additional amplification processes, such as, the embodiment illustrated in FIG. 9.

In general, a kit of the invention will also comprise a description of the components of said kit and instructions for their use in a particular process or method or methods of the invention. In general, a kit of the present invention will also comprise other components, such as, but not limited to, buffers, ribonucleotides and/or deoxynucleotides, including modified nucleotides in some embodiments, DNA polymerization or reverse transcriptase enhancers, such as, but not limited to betaine (trimethylglycine), and salts of monvalent or divalent cations, such as but not limited to potassium acetate or chloride and/or magnesium chloride, enzyme substrates and/or cofactors, such as, but not limited to, ATP or NAD, and the like which are needed for optimal conditions of one or more reactions or processes of a method or a combination of methods for a particular application. A kit of the invention can comprise a a set of individual reagents for a particular process or a series of sets of individual reagents for multiple processes of a method that are performed in a stepwise or serial manner, or a kit can comprise a multiple reagents combined into a single reaction mixture or a small number of mixtures of multiple reagents, each of which perform multiple reactions and/or processes in a single tube. In general, the various components of a kit for performing a particular process of a method of the invention or a complete method of the invention will be optimized so that they have appropriate amounts of reagents and conditions to work together in the process and/or method.

X. Additional Embodiments of the Invention

1. Methods that Use an Open Circle Probe that Lacks a Target-Complementary Sequence

A “ligation splint” or a “ligation splint oligo” is an oligo that is used to provide an annealing site or a “ligation template” for joining two ends of one nucleic acid (i.e., “intramolecular joining”) or two ends of two nucleic acids (i.e., “intermolecular joining”) using a ligase or another enzyme with ligase activity. The ligation splint holds the ends adjacent to each other and “creates a ligation junction” between the 5′-phosphorylated and a 3′-hydroxylated ends that are to be ligated.

The invention also comprises embodiments of target-dependent transcription in which a circular transcription substrate comprising a target-complementary sequence is generated even if there is no target-complementary sequence at either the 3′-end or the 5′-end or at both ends of an “open circle probe” (the word “target” is removed from the name of the probe here because there are no target-complementary sequences). An open circle probe of the invention comprises an oligonucleotide having a 5′-end portion comprising a sequence for a sense promoter sequence for a cognate RNA polymerase that uses a double-stranded promoter or, in other embodiments, a single-stranded promoter or pseudopromoter for a cognate RNA polymerase and a 3′-end portion comprising a sequence that is not a promoter sequence, which sequene can optionally comprise a signal sequence, as discussed elsewhere herein.

In embodiments that use an open circle probe, simple target probes that can anneal to the target sequence are used. Following annealing and ligation of these simple target probes on the target sequence in a target-dependent manner to obtain a linear ligation product, the resulting linear ligation product comprising a target-complementary sequence is joined directly to an open circle probe using two ligation splints, each of which has a portion complementary to a respective end of the open circle probe and to an appropriate end of the target-complementary sequence in the linear ligation product. One ligation splint oligo is used to join a sense promoter of an open circle probe to the 3′-end of a polynucleotide ligation product that was previously obtained by ligation of two or more simple target probes that were annealed to a target sequence. This first ligation splint oligo has a 3′-sequence that is complementary to the 3′-end of the target sequence and a second adjacent 5′-sequence that is complementary to the 5′-end of a the 5′-phosphorylated sense promoter sequence of the open circle probe. The second ligation splint oligo has a 5′-sequence that is complementary to the 5′-end of the target sequence and a second adjacent 3′-sequence that is complementary to the 3′-end of the open circle probe.

Thus, one embodiment of the invention comprises a method for detecting a target nucleic acid sequence, said method comprising:

a. providing at least two simple target probes comprising at least two target-complementary sequences, wherein the target probes comprise a 5′-phosphate and are adjacent when annealed on the target sequence, and wherein a first simple target probe is complementary to the 5′-end of the target nucleic acid and a second simple target probe is complementary to the 3′-end of the target nucleic acid sequence;

b. annealing the target probes to the target nucleic acid sequence under hybridization conditions;

c. contacting the target probes annealed to the target nucleic acid sequence with a ligase under ligation conditions so as to obtain a linear ligation product;

d. denaturing the ligation product from the target nucleic acid sequence;

e. providing an open circle probe, wherein the 5′-end portion of the open circle probe comprises a 5′-phosphate group and a sense promoter sequence for a double-stranded transcription promoter that is recognized by a cognate RNA polymerase;

f. providing a first ligation splint comprising a 5′-end unphosphorylated portion that is complementary to the 5′-end portion of the open circle probe and a 3′-end portion that is complementary to the 3′-end portion of the ligation product;

g. providing a second ligation splint comprising a 5′-end unphosphorylated portion that is complementary to the 5′-end of the ligation product and a 3′-end that is complementary to the 3′-end of the open circle probe;

h. incubating the ligation product, the open circle probe, the first ligation splint and the second ligation splint under hybridization conditions so as to obtain a complex;

i. contacting the complex with a ligase under ligation conditions so as to obtain a circular ligation product comprising the linear ligation product and the open circle probe;

j. annealing an anti-sense promoter oligo to the sense promoter sequence of the circular ligation product so as to obtain a circular transcription substrate;

k. contacting the circular transcription substrate with a cognate RNA polymerase for the promoter under transcription conditions so as to obtain a transcription product; and

l. detecting the transcription product.

Still another embodiment of the invention comprises a method for detecting a target nucleic acid sequence, said method comprising:

a. providing at least two simple target probes comprising at least two target-complementary sequences, wherein the target probes comprise a 5′-phosphate and are adjacent when annealed on the target sequence, and wherein a first simple target probe is complementary to the 5′-end of the target nucleic acid and a second simple target probe is complementary to the 3′-end of the target nucleic acid sequence;

b. annealing the target probes to the target nucleic acid sequence under hybridization conditions;

c. contacting the target probes annealed to the target nucleic acid sequence with a ligase under ligation conditions so as to obtain a linear ligation product;

d. denaturing the ligation product from the target nucleic acid sequence;

e. providing an open circle probe, wherein the 5′-end portion of the open circle probe comprises a 5′-phosphate group and a sequence for single-stranded transcription promoter or pseudopromoter that is recognized by a cognate RNA polymerase;

f. providing a first ligation splint comprising a 5′-end unphosphorylated portion that is complementary to the 5′-end portion of the open circle probe and a 3′-end portion that is complementary to the 3′-end portion of the ligation product;

g. providing a second ligation splint comprising a 5′-end unphosphorylated portion that is complementary to the 5′-end of the ligation product and a 3′-end that is complementary to the 3′-end of the open circle probe;

h. incubating the ligation product, the open circle probe, the first ligation splint and the second ligation splint under hybridization conditions so as to obtain a complex;

i. contacting the complex with a ligase under ligation conditions so as to obtain a circular transcription substrate comprising the linear ligation product and the open circle probe;

j. contacting the circular transcription substrate with a cognate RNA polymerase for the promoter under transcription conditions so as to obtain a transcription product; and

k. detecting the transcription product.

In still other embodiments of the invention, the linear ligation product obtained in step (c) of the embodiments immediately above are ligated to an oligonucleotide comprising a sense promoter sequence for a double-stranded promoter or sequence for a single-stranded promoter for a cognate RNA polymerase, thereby generating linear transcription substrates of the invention, meaning, for example, that simple target probes can be used without using a promoter target probe, and/or a signal target probe, and then, the resulting ligation product comprising the target-complementary sequence can be joined to a suitable sense promoter and a signal sequence, if used, by means of ligation splints and a ligase under ligation conditions.

Ligases that can be used to ligate suitable ends that are annealed to a ligation splint comprising DNA include, but are not limited to, Ampligase® DNA Ligase (EPICENTRE Technologies, Madison, Wis.), Tth DNA ligase, Tfl DNA ligase, Tsc DNA ligase (Prokaria, Ltd., Reykjavik, Iceland), or T4 DNA ligase. These ligases can be used for both intermolecular and intramolecular ligations when a ligation splint comprising DNA is used to bring the respective ends adjacent to each other. If a ligation splint comprising RNA is used, T4 DNA ligase can be used to join the ends that are annealed to the ligation splint. These embodiments of the invention remove all background transcription that could result from run-off transcription of the small target-complementary sequence at the 5′-end of a bipartite target probe.

Still further, in other embodiments, simple target probes that anneal adjacently on a target sequence are ligated, then denatured from the target sequence, then ligated to an oligo comprising a sense promoter sequence using a ligation splint that is complementary to the most 3′-end of the ligation product comprising the target-complementary target probes and to the 5′-end of a sense promoter sequence, and then, finally circularized by non-homologous intramolecular ligation of a 5′-phosphorylated end with a 3′-hydroxyl end, and, if the promoter sequence comprises a sense promoter sequence for a double-stranded promoter, annealed to an anti-sense promoter oligo to obtain a circular transcription substrate. Circularization of a linear single-stranded DNA without a ligation splint can be carried out using ThermoPhage™ RNA Ligase II (Prokaria, Ltd., Reykjavik, Iceland). A reason to circularize a linear ligation product is to obtain a circular transcription substrate for more efficient transcription by a rolling circle transcription mechanism, rather than by linear transcription. This embodiment is used only if steps are taken to assure that only ligation products derived from the target-complementary sequences that were ligated in the presence of the target sequence are circularized by the ligase that catalyzes non-homologous ligation, or that the other non-target-dependent transcription products will not be detected in the assay or method.

2. Strand-Displacement Reverse Transcription: A Novel Amplification Method for Obtaining Additional Amplication in Target-Dependent Transcription Assays

The present inventors' search for methods to detect a target analyte comprising a target nucleic acid sequence led, unexpectedly, to the to a novel concept for a method for strand displacement reverse transcription, which method provides unique possibilities for amplifying a target sequence and/or a signal sequence under certain conditions that are discussed below. The method is useful in conjunction with other methods that utilize an RNA polymerase that can synthesize RNA using said ssDNA transcription substrates, such as, but not limited to the target-dependent transcription assays and methods of the present invention described herein.

Methods for strand displacement amplification of linear and circular ssDNA templates are well known in the art. By way of example, but not of limitation, strand displacement amplification methods are disclosed in PCT Patent Publication Nos. WO 02/16639; WO 00/56877; and AU 00/29742; of Takara Shuzo Company; U.S. Pat. Nos. 5,523,204; 5,536,649; 5,624,825; 5,631,147; 5,648,211; 5,733,752; 5,744,311; 5,756,702; and 5,916,779 of Becton Dickinson and Company; U.S. Pat. Nos. 6,238,868; 6,309,833; and 6,326,173 of Nanogen/Becton Dickinson Partnership; U.S. Pat. Nos. 5,849,547; 5,874,260; and 6,218,151 of Bio Merieux; U.S. Pat. Nos. 5,786,183; 6,087,133; and 6,214,587 of Gen-Probe, Inc.; U.S. Pat. No. 6,063,604 of Wick et al.; U.S. Pat. No. 6,251,639 of Kum; U.S. Pat. No. 6,410,278; and PCT Publication No. WO 00/28082 of Eiken Kagaku Kabushiki Kaishi, Tokyo, Japan; U.S. Pat. Nos. 5,591,609; 5,614,389; 5,773,733; 5,834,202; and 6,448,017 of Auerbach; and U.S. Pat. No. 6,124,120; and 6,280,949 of Lizardi, all of which are incorporated herein by reference. In general, the methods for strand displacement amplification of linear templates in the art use some kind of process to digest a sequence region at or near the 5′-end of a replicating second-strand cDNA in order to liberate at least a portion of the primer binding site on the DNA template so that another primer can anneal to the template and initiate DNA synthesis, which results in displacement of the last-synthesized DNA strand. The methods disclosed in U.S. Pat. Nos. 5,523,204; 5,536,649; 5,624,825; 5,631,147; 5,648,211; 5,733,752; 5,744,311; 5,756,702; and 5,916,779 of Becton Dickinson and Company use a restriction enzyme to liberate the primer-binding site at the 5′-end. The methods disclosed in U.S. Pat. Nos. 5,786,183; 6,087,133; and 6,214,587 of Gen-Probe, Inc. use multiple primers, preferably with a 5′-flap, in the absence of a restriction enzyme to liberate the primer-binding site at the 5′-end. The methods disclosed in U.S. Pat. No. 6,063,604 of Wick et al. use primers designed to have a restriction endonuclease nick site to liberate the primer-binding site at the 5′-end. The methods disclosed by Sagawa et al. in PCT Patent Publication No. WO 02/16639; and in PCT Patent Publications Nos. WO 00/56877 and AU 00/29742 use a composite primer having a 5′-portion comprising deoxynucleotides and a 3′-portion comprising ribonucleotides, and then use RNase H to liberate the primer-binding site at the 5′-end. The methods disclosed in U.S. Pat. No. 6,251,639 of Kurn use a composite primer having a 5′-portion comprising ribonucleotides and a 3′-portion comprising deoxynucleotides, and then use RNase H to liberate the primer-binding site at the 5′-end of the replicating DNA strand. Rolling circle amplification (“RCA”), as disclosed in U.S. Pat. Nos. 6,344,329; 6,210,884; 6,183,960; 5,854,033; 6,329,150; 6,143,495; 6,316,229; 6,287,824; all of which are incorporated herein by reference, including references therein, involve strand-displacement DNA polymerization using ssDNA templates.

Although strand-displacement amplification of DNA templates is well known in the art, strand-displacement reverse transcription of RNA templates has never been disclosed prior to the disclosure of the present invention.

Thus, another embodiment of the present invention comprises a method for amplifying a target nucleic acid comprising a linear single-stranded RNA (ssRNA) by strand displacement reverse transcription, said method comprising:

1. providing a reaction mixture comprising:

a. a reverse transcriptase with strand-displacement activity;

b. optionally, a single-strand binding protein;

c. multiple oligonucleotide primers, wherein at least the 3′-portion of each said primer comprises a sequence that is complementary to a sequence in said ssRNA;

2. contacting said reaction mixture from step 1 above with a sample comprising a target nucleic acid comprising a ssRNA, wherein said reaction mixture containing said sample is maintained at a temperature wherein said reverse transcriptase, and optionally said single-strand binding protein, are optimally active in combination for strand-displacement reverse transcription and wherein said reverse transcription primers anneal to said target sequence, if present, with specificity, and wherein said temperature of said reaction mixture is maintained for a time sufficient to permit synthesis of first-strand cDNA reverse transcription products complementary to said target nucleic acid, if present in said sample; and

3. obtaining multiple copies of said first-strand cDNA that is complementary to said RNA target nucleic acid.

In contrast to a linear ssRNA template, multiple primers are not required for strand-displacement reverse transcription of circular ssRNA templates because, under strand-displacement conditions, synthesis of first-strand cDNA proceeds around and around the circular ssRNA template, continually displacing first-strand cDNA synthesized during the previous round of reverse transcription, and generating a first-strand cDNA multimer comprising multiple tandem copies of a first-strand cDNA oligomer, each of which is complementary to one copy of said circular ssRNA molecule. Although multiple primers are not required for circular ssRNA templates, the use of multiple primers is preferred in some embodiments in order to increase the rate of first-strand cDNA synthesis. Multiple primers are increasingly preferred as the size of the circular ssRNA template increases.

Thus, another embodiment of the present invention comprises a method for amplifying a target nucleic acid comprising a circular single-stranded RNA (ssRNA) by strand displacement reverse transcription, said method comprising:

1. providing a reaction mixture comprising:

a. a reverse transcriptase with strand-displacement activity;

b. optionally, a single-strand binding protein;

c. at least one, and optionally multiple, oligonucleotide primers, wherein at least the 3′-portion of each said primer comprises a sequence that is complementary to a sequence in said ssRNA;

2. contacting said reaction mixture from step 1 above with a sample comprising a target nucleic acid comprising a circular ssRNA, wherein said reaction mixture containing said sample is maintained at a temperature wherein said reverse transcriptase, and optionally said single-strand binding protein, are optimally active in combination for strand-displacement reverse transcription and wherein said reverse transcription primers anneal to said target sequence, if present, with specificity, and wherein said temperature of said reaction mixture is maintained for a time sufficient to permit synthesis of first-strand cDNA reverse transcription products complementary to said target nucleic acid, if present in said sample; and

3. obtaining first-strand cDNA multimers comprising multiple tandem copies of a first-strand cDNA oligomer, each of which is complementary to one copy of said circular ssRNA target nucleic acid template.

Strand-displacement reverse transcription can be used to generate multiple copies of first-strand cDNA for use in methods and assays such as, but not limited to the embodiment shown in FIG. 9.

The use of a “tail” that is not complementary to the RNA transcript is optional and the use of a tail is not required for other embodiments of the invention. However, the use of a tail is preferred according to the present strand-displacement reverse transcription method to facilitate strand displacement. Thus, some embodiments of the present method comprise synthesis of first-strand cDNA by strand-displacement reverse transcription, wherein one or more reverse transcription primers comprising tail sequences that are not complementary to the RNA template are used. In most embodiments of the invention, the oligonucleotides used as strand-displacement primers comprise deoxyribonucleotides. However, the invention also comprises other embodiments in which oligoribonucleotides are used for strand-displacement primers in the various embodiments of strand-displacement reverse transcription. Still further, the invention also comprises the use of 2′-fluoro-containing modified oligoribonucleotides or “DuraScript™ RNA,” which can be made using the DuraScribe™ T7 Transcription Kit (EPICENTRE Technologies, Madison, Wis., USA) or purchased from oligonucleotide companies such as Integrated DNA Technologies, Coralville, Iowa, in the various embodiments of strand-displacement reverse transcription. Strand-displacement primers comprising DuraScript™ RNA are resistant to RNase A-type ribonucleases. Primers for strand-displacement reverse transcription can comprise a specific sequence that is complementary to only one RNA sequence. Alternatively, the multiple strand-displacement primers of a strand-displacement reverse transcription reaction of the present invention can also comprise random sequence primers, including but not limited to random hexamers, random octamers, random nonamers, random decamers, random dodecamers and the like, with the length based on considerations such as the temperature optimum of the reverse transcriptase and the Tm random sequence primer. When random sequence primers are used, the primers can also prime synthesis of second-strand cDNA using first-strand cDNA as a template, and subsequently, can prime the synthesis of third, fourth and other cDNA strands, thereby resulting in additional amplification. Random sequence primers are commercially available from oligonucleotide companies such as Integrated DNA Technologies, Coralville, Iowa. In some preferred embodiments, the random sequence primers comprise alpha-thio internucleoside linkages, which are resistant to some exonucleases. In some embodiments of this aspect of the invention, a biotin or other binding moiety is covalently attached to a nucleotide in the 5′-portion of a reverse transcription primer used for strand-displacement reverse transcription. The biotin or other binding moiety enables capture of first-strand cDNA obtained by strand-displacement reverse transcription.

Conditions for strand-displacement reverse transcription can be identified by performing assays that measure the ability of a reverse transcriptase to displace a labeled oligo having a 3′-dideoxy nucleotide, wherein said labeled oligo is annealed to an RNA template 3′-of or downstream of an extending first-strand cDNA that is being synthesized from a primer that anneals to said RNA template 5′-of the site to which said labeled oligo anneals. Using this assay, different reverse transcriptases, different reaction temperatures that cover the range for which each particular reverse transcriptase is active, and other reaction conditions are varied systematically in order to identify conditions that result in strand displacement reverse transcription.

Strand-displacing reverse transcriptases that can be used include, but are not limited to RNaseH-Minus MMLV reverse transcriptase (SuperScript™ reverse transcriptases from Invitrogen, Carlsbad, Calif.), IsoTherm™ DNA Polymerase (EPICENTRE Technologies, Madison, Wis.), or BcaBEST™ DNA Polymerase (Takara Shuzo Co., Japan). One reverse transcription reaction condition that can increase displacement of first-strand cDNA, and which is included as part of the present invention, is addition of a single-strand binding protein, such as, but not limited to EcoSSB Protein or an SSB Protein from a thermostable bacterium, such as Tth or Bst SSB Protein, to a reverse transcription reaction. However, use of a single-strand binding protein is optional. Betaine can also be added to a reverse transcription reaction in order to increase strand displacement. As disclosed in U.S. Pat. Nos. 6,048,696 and 6,030,814, and in German Patent No. DE4411588C1, all of which are incorporated herein by reference and made part of the present invention, it is preferred in many embodiments to use a final concentration of about 0.25 M, about 0.5 M, about 1.0 M, about 1.5 M, about 2.0 M, about 2.5 M or between about 0.25 M and 2.5 M betaine (trimethylglycine) in DNA polymerase or reverse transcriptase reactions in order to decrease DNA polymerase stops and increase the specificity of reactions that use a DNA polymerase.

Reaction conditions that result in strand-displacement reverse transcription, provided that said reaction conditions do not decrease the activities of other reactions of an assay or method, will result in further amplification of a target sequence and/or a signal sequence in a target-dependent transcription assay or method of the present invention.

Y. Use of Circular Transcription Substrates to Synthesize Double-Stranded RNA by Rolling Circle Transcription That Can be Used for RNA Interference

If a target sequence in a target nucleic acid is present in a sample, the methods of the invention that use a bipartite target probe, as disclosed herein, can be used to generate a circular transcription substrate of the present invention. This circular transcription substrate can be used as a substrate for rolling circle transcription by an RNA polymerase that binds to a promoter and synthesizes RNA therefrom in order to synthesize double-stranded RNA (dsRNA) that can be used to silence a gene by RNA interference. That is the dsRNA is used as RNAi. By way of example, but not of limitation, dsRNA for use as RNAi can be synthesized using the embodiment of the invention illustrated in FIG. 9. If the target sequence comprises a target nucleic acid that is encoded by a pathogen or by an oncogene, for example, the dsRNA can be a therapeutic composition.

In another embodiment, a new circular transcription substrate is prepared for synthesis of dsRNA for use as RNAi, wherein each oligomer of the RNA multimer transcription product obtained using said circular transcription substrate for rolling circle transcription comprises a self-complementary double-stranded hairpin structure with a non-complementary loop between the self-complementary regions, such that each oligomer corresponds to the desired RNAi and the loop structure. Preferably, said circular transcription substrate is designed so that said RNA oligomers can be cleaved from the RNA multimer obtained from rolling circle transcription, for example, using a ribozyme or an RNase H and DNA oligo complementary to the cleavage site.

Preferred RNA polymerases for rolling circle transcription comprise T7 RNAP, T3 RNAP, or SP6 RNAP or mutant enzymes, such as but not limited to T7 RNAP Y639F, T3 RNAP Y573F or SP6 RNAP Y63° F. mutant enzymes (Sousa et al., U.S. Pat. No. 5,849,546). Alternatively, some embodiments of this aspect of the invention use an N4 mini-vRNAP enzyme, such as but not limited to an N4 mini-vRNAP Y678F mutant enzyme (U.S. Patent Application No. 20030096349), and a circular transcription substrate having a single-stranded N4 promoter that binds the mini-vRNAP enzyme to make RNA multimers comprising double-stranded hairpins for use in RNAi. Most preferred embodiments of of this aspect of the invention use one of the T7-type RNAPs or the N4 mini-vRNAP Y678F mutant enzyme to synthesize RNA containing 2′-fluoro-pyrimidine nucleotides by using 2′-fluoro-dCTP and 2′-fluoro-dUTP, in addition to ATP and GTP in the rolling circle transcription reaction. Modified RNA molecules that contain 2′-F-dCMP and 2′-F-dUMP are resistant to RNase A-type ribonucleases (Sousa et al., U.S. Pat. No. 5,849,546), included herein by reference. Capodici et al, (J. Immunology, 169: 5196-5201, 2002 showed that 2′-fluoro-containing dsRNA molecules made using the DuraScribe™ Transcription Kit (EPICENTRE Technologies, Madison, Wis., USA) did not require transfection reagents for delivery into cells, even in the presence of serum. Kakiuchi et al. (J. Biol. Chem., 257: 1924-1928, 1982) showed that use of [(2′-F-dI)ₙ: (2′-F-dC)ₙ duplexes were 40-100 times less antigenic than [(rI)ₙ: (rC)ₙ] duplexes, and did not induce an interferon response like [(rI)ₙ: (rC)ₙ] duplexes.

### EXAMPLES

The following examples are included to demonstrate preferred embodiments of the invention. It should be appreciated by those of skill in the art that the techniques disclosed in the examples which follow represent techniques discovered by the inventors to function in the practice of the invention. However, those of skill in the art should, in light of the present disclosure, appreciate that many changes can be made in the specific embodiments which are disclosed and still obtain a like or similar result without departing from the spirit and scope of the invention.

### Example 1

Use of Target-Dependent Transcription Using Monopartite Target Probes Comprising a T7 Promoter to Detect the Human β-globin Gene Sequence in Which a Single Nucleotide Mutation Results in Sickle Cell Anemia

Monopartite target probes were designed to anneal to the gene encoding human β-globin. The ligation junction of the adjacent probes when annealed to the denatured globin gene is the site of a single-base difference responsible for the sickle-cell phenotype (an A to T transversion). The globin promoter target probe and the globin signal target probe should only ligate when annealed to the wild-type globin allele, but not when the ligation junction is annealed to a target nucleotide comprising a single-base mismatch that results in the sickle-cell phenotype.

Oligonucleotide target probes and an anti-sense promoter oligo, with the sequences given below, were obtained from Integrated DNA Technologies, Coralville, Iowa. The target-complementary sequences are underlined. The T7 sense promoter sequence is in italics. The remaining portion of the globin signal target probe serves as a signal sequence.

A. Globin Promoter Target Probe (37 nucleotides, 15 target-complementary nucleotides):

5′Phos/TCAGGAGTCAGGTGCCTATAGTGAGTCGTATTACTAG3′

B. Globin Signal Target Probe (50 nucleotides; 25 target-complementary nucleotides):

5′GGCCAACGACTACGCACTAGCCAACCAGGGCAGTAACGGCAGACTTCTCC3′

C. T7 Anti-sense Promoter Oligo (18 nucleotides)

5′TAATACGACTCACTATAG3′

A promoter target probe and signal target probe for detection of a beta-globin gene sequence were designed with a goal to be optimal for both hybridization specificity and thermostable ligase activity under hybridization and ligation reaction conditions. Thus, the target-complementary portions of the target probes were long enough to hybridize preferentially to the target sequence and not elsewhere in the human genome at a hybridization temperature that would still provide sufficient thermostable ligase activity. The probe sequences were compared to the Genbank database to verify homology to the targets and to identify any secondary targets. Thus, the promoter target probe had 15 target-complementary bases, 17 bases of T7 sense promoter sequence and +1 nucleotide, and 4 additional non-homologous bases upstream of the promoter for improved transcription, and the 5′ end was phosphorylated. The signal target probe contained 25 target-complementary bases at the 3′ end and 25 bases of a signal sequence at the 5′ end for detection of the transcription product.

The globin target probes were incubated with subcloned plasmid DNA containing the wild-type globin gene sequence. Target probes were annealed and ligated to the target sequence as follows: From 10-400 nanograms of plasmid DNA comprising the target sequence were denatured and hybridized to 2 to 50 picomoles of each target probe in 20 mM Tris-HCl (pH 8.3 at 25° C.), 25 mM KCl, 10 mM MgCl₂, 0.5 mM NAD, 0.01% Triton X-100 at 94° C. for 2 minutes. Target probes annealed to the target sequence were ligated using 100 Units of Ampligase® Thermostable Ligase (EPICENTRE Technologies, Madison, Wis.) by thermocycling for 20 to 50 cycles of 94° C. for 30 seconds and 40° C. for 4 minutes. Then, in order to generate a double-stranded T7 promoter, an excess of the anti-sense promoter oligo (5-50 picomoles) was annealed to the unpurified ligation products by heating to 94° C. for 2 minutes and slow cooling to room temperature.

Transcription reactions were analyzed as follows: 10-25% of the ligation reaction was used as template in the transcription reaction. Thus, the equivalent of 0.5 to 5 picomoles of starting target probe from the ligation reaction was used as template in an AmpliScribe™ T7-Flash™ in vitro transcription reaction (EPICENTRE Technologies, Madison, Wis.), without purification of the linear transcription substrate. The 20 ul reaction contained 9 mM each NTP, 1×AmpliScribe T7-Flash buffer, 10 mM DTT, and AmpliScribe T7-Flash Enzyme Mix. Reactions were incubated at 37° C. for 2 hours. The transcription reactions were treated with 2 Units DNase I, for 30 minutes at 37° C. Samples were heat denatured in formamide loading buffer and were analyzed by denaturing polyacrylamide gel electrophoresis using 15% gels, 6 M urea in 1×TBE.

Upon ligation of the globin promoter target probe to the globin signal target probe in the presence of the target sequence, an 87-nucleotide ligation product would be obtained. Then, after annealing of the T7 anti-sense promoter oligo to form a linear transcription substrate, in vitro transcription with T7 RNAP should result in synthesis of a 66-nucleotide transcript. In the absence of ligation to the globin signal target probe, annealing of the T7 anti-sense promoter oligo to the globin promoter target probe would yield only a 16-nucleotide transcript and no signal sequence would be obtained.

As expected, a 66-nucleotide RNA transcription product was observed only in reactions in which the globin target probes were incubated under hybridization and ligation conditions in the presence of the wild-type β-globin sequence. No transcription product was observed in the absence of the wild-type β-globin sequence, in the absence of ligase, or in the presence of only one target probe.

### Example 2

Use of Target-Dependent Transcription Using Monopartite Target Probes Comprising a T7 Promoter to Detect Human Papilloma Virus (HPV) Gene Sequences

Human papilloma virus (HPV) is believed to be responsible for human diseases including cervical cancer and warts. Certain strains appear to be related to higher cancer risk than others. Detection of the presence of the virus and the strain of the virus is useful for research and diagnostics. Target-dependent transcription reactions were performed using monopartite target probes comprising a T7 promoter in order to make an initial evaluation of the sensitivity and specificity of this method for detection of an HPV DNA sequence.

Oligonucleotide target probes and an anti-sense promoter oligo, with the sequences given below, were obtained from Integrated DNA Technologies, Coralville, Iowa. The target-complementary sequences are underlined. The T7 sense promoter sequence is in italics. The remaining portion of the HPV signal target probe serves as a signal sequence.

A. HPV Promoter Target Probe (38 nucleotides, 16 target-complementary nucleotides):

5′Phos/CTGTGCCTCCTGGGGGCTATAGTGAGTCGTATTACTAG3′

B. HPV Signal Target Probe (50 nucleotides; 25 target-complementary nucleotides):

5′CCAACGACTACGCACTAGCCAACGTTACAAACCTATAAGTATCTTCTA3′

C. T7 Anti-sense Promoter Oligo (18 nucleotides)

5′TAATACGACTCACTATAG3′

A promoter target probe and signal target probe for detection of the major capsid protein L1 gene of HPV type 16, commonly found in cervical cancer specimens, were designed with a goal to be optimal for both hybridization specificity and thermostable ligase ligation activity under hybridization and ligation reaction conditions. Thus, the target-complementary portions of the target probes were long enough to hybridize preferentially to the target sequence and not elsewhere in the human genome at a hybridization temperature that would still provide sufficient thermostable ligase activity. The probe sequences were compared to the Genbank database to verify homology to the targets and to identify any secondary targets. Thus, the promoter target probe had 16 target-complementary bases, 18 bases of T7 sense promoter sequence and +1 nucleotide, and 4 additional non-homologous bases upstream of the promoter for improved transcription, and the 5′ end was phosphorylated. The signal target probe contained 25 target-complementary bases at the 3′ end and 25 bases of a signal sequence at the 5′ end for detection of the transcription product.

The HPV target probes were incubated with denatured HPV16 L1 gene PCR product containing 456 bases of major capsid protein L1 sequence. Target probes were annealed and ligated to the target sequence as follows: From 10-400 nanograms of PCR product comprising the target sequence were denatured and hybridized to 2 to 50 picomoles of each target probe in 20 mM Tris-HCl (pH 8.3 at 25° C.), 25 mM KCl, 10 mM MgCl₂, 0.5 mM NAD, 0.01% Triton X-100 at 94° C. for 2 minutes. Target probes annealed to the target sequence were ligated using 100 Units of Ampligase® Thermostable Ligase (EPICENTRE Technologies, Madison, Wis.) by thermocycling for 20 to 50 cycles of 94° C. for 30 seconds and 40° C. for 4 minutes. Then, in order to generate a double-stranded T7 promoter, an excess of the anti-sense promoter oligo (5-50 picomoles) was annealed to the unpurified ligation products by heating to 94° C. for 2 minutes and slow cooling to room temperature.

Transcription reactions were analyzed as follows: 10-25% of the ligation reaction was used as template in the transcription reaction. Thus, the equivalent of 0.5 to 5 picomoles of starting target probe from the ligation reaction was used as template in an AmpliScribe™ T7-Flash™ in vitro transcription reaction (EPICENTRE Technologies, Madison, Wis.), without purification of the linear transcription substrate. The 20 ul reaction contained 9 mM each NTP, 1×AmpliScribe T7-Flash buffer, 10 mM DTT, and AmpliScribe T7-Flash Enzyme Mix. Reactions were incubated at 37° C. for 2 hours. The transcription reactions were treated with 2 Units DNase I, for 30 minutes at 37° C. Samples were heat denatured in formamide loading buffer and were analyzed by denaturing polyacrylamide gel electrophoresis using 15% gels, 6 M urea in 1×TBE.

Upon ligation of the HPV promoter target probe to the HPV signal target probe in the presence of the target sequence, an 88-nucleotide ligation product was obtained. The formation of the 88-nucleotide ligation product required both monopartite target probes, the HPV target DNA, and Ampligase® thermostable DNA ligase. Then, after annealing of the T7 anti-sense promoter oligo to form a linear transcription substrate, in vitro transcription with T7 RNAP resulted in synthesis of a 68-nucleotide transcript. In the absence of ligation to the HPV signal target probe, annealing of the T7 anti-sense promoter oligo to the globin promoter target probe yields only a 18-nucleotide transcript, and no signal sequence would be obtained. The 68-nucleotide transcript was specifically transcribed only in the reactions containing full-length ligation products. No 68-nucleotide transcription products were observed without the ligase or in the absence of either HPV target probe. The 68-nucleotide transcription product could be detected at all levels of target probes tested between 2 and 20 picomoles per reaction.

The detection of HPV 16 DNA in mock-patient DNA samples was also performed with mixtures of HPV16 L1 gene PCR product with human genomic DNA. The HPV target probes ligation product and resulting RNA transcript were not produced with human genomic DNA alone, but were produced with samples containing both human genomic DNA and different dilutions of HPV16 DNA. The transcript from the template-dependent ligation probe was detectable by gel electrophoresis when as little as 33 femtomoles of HPV 16 target sequence was present in 100 ng of human genomic DNA. This implies that the currently described target-dependent transcription method could be used to detect viral DNA in patient samples. The sensitivity of the reaction could be increased still further by using an amplifiable signal sequence in the HPV signal target probe. That this method detected the presence of a target sequence in a mixed DNA population indicates that DNA viruses such as human papilloma virus (HPV) can be detected in complex human DNA samples using monopartite target probes for target-dependent transcription.

### Example 3

Rolling Circle Transcription of Model ssDNA Transcription Substrates

Each oligonucleotides (50 picomoles), comprising a sense P2 promoter sequence (or, in control reactions, an anti-sense sequence to the P2 promoter or no promoter) at its 5′-end, which was phosphorylated, and up to 52 additional nucleotides corresponding to a model target sequence (e.g., for the human beta actin gene) in its 3′-portion, was ligated in a reaction mixture containing 0.2 mM ATP, 1 mM DTT, and 50 micrograms per ml of BSA for 2 hours at 60° C. using 200 units of ThermoPhage™ RNA Ligase II (Prokaria, Rejkjavik, Iceland, #Rlig122) in 1×ThermoPhage™ RNA Ligase II Buffer comprising 50 mM MOPS, pH 7.5, 5 mM MgCl₂, and 10 mM KCl. Then, linear oligos were removed by digestion with Exonuclease I (EPICENTRE Technologies, Madison, Wis.), the Exo I was heat-inactivated, and the circular ssDNA oligos were ethanol precipitated using standard techniques.

One picomole of circular ssDNA oligonucleotide, prepared as just described, was then incubated for four hours at 37° C. in a 60-microliter reaction mixture comprising one microgram of mini-vRNAP (EPICENTRE Technologies, Madison, Wis.), 1 mM each NTP, 1 mM DTT, and 5 micromolar E. coli SSB Protein (EPICENTRE Technologies, Madison, Wis.), in 1×Transcription Buffer comprising 40 mM Tris HCl, pH 7.5, 10 mM NaCl, 6 mM MgCl₂, and 1 mM spermidine. The resulting mini-vRNAP transcription products were then analyzed by electrophoresis in a 1% agarose gel containing 0.22 M formaldehyde. Transcription products, including products having a length many-fold greater than the starting oligonucleotide, were observed on the the gel using the transcription substrate having a sense P2 promoter sequence, indicating efficient rolling circle transcription. No transcription products were observed if the oligo did not contain a P2 promoter, if an anti-sense sequence to the P2 promoter was used instead of the sense P2 promoter, or if an unligated linear oligo with a sense P2 promoter was used.

### Example 4

Use of Target-Dependent Transcription Using Bipartite Target Probes Comprising a P2 Promoter to Detect the Human β-globin Gene Sequence in Which a Single Nucleotide Mutation Results in Sickle Cell Anemia

Bipartite target probes were designed to anneal to the gene encoding human hemoglobin β chain. The ligation junction of the adjacent probes when annealed to the denatured globin gene is the site of a single-base difference responsible for the sickle-cell phenotype (an A to T transversion leading to Glu→Val change in the β-globin). The probe can be circularized by DNA ligase only when annealed to the wild-type globin allele, but not when the ligation junction is annealed to a target nucleotide comprising a single-base mismatch that results in the sickle-cell phenotype.

Oligonucleotide target probes were obtained from Integrated DNA Technologies, Coralville, Iowa and were 5′ phosphorylated during synthesis. All human β-globin bipartite target probes consisted of two target-complementary arms in the 5′ and 3′ terminal regions connected by a spacer of a specific size that contained P2 promoter sequence, as well as (optional) binding sites for amplification primers, restriction sites, signal sequences, etc. The 5′ arm length was from 11 to 18 nucleotides and was designed to anneal immediately upstream of the single-base mismatch that results in the sickle-cell phenotype. The 3′ arm was from 14 to 20 nucleotides long and was complementary to the region immediately downstream of this mutation. In most probes the 3′-terminal base was complementary to the nucleotide that differed in the wild type and mutant alleles of the β-globin gene. This was done to improve allele discrimination, since base mismatches at the 3′ terminus are more inhibitory to ligation than those at 5′ terminus (Luo et al., Nucleic Acids Res., 24:3071-3078, 1996). The length of the spacer region should allow circularization of the oligo while its 5′ and 3′ terminal arms are annealed to the target and cannot be shorter then 1.26-times the combined length of target-complementary arms.

The sequence of one of the human β-globin bipartite target probes is given below. The target-complementary sequences are underlined. The P2 promoter hairpin sequence is in italics.

5′Phos-GTCCTCAGTCCCAAAAGAAGCGGAGCTTCT₍₂₄₎CCGTCTGAAGAGGA3′ (67 bases, 25 are complementary to the target)

Bipartite target probes for detection of a beta-globin gene sequence were designed with a goal to be optimal for (i) target recognition specificity and thermostable ligase activity under hybridization and ligation reaction conditions and (ii) for N4 mini-vRNAP-catalyzed rolling circle transcription. Thus, the probe was designed so that the P2 promoter hairpin was the only stable secondary structure at 37° C., the target-complementary portions of the target probes were long enough to hybridize preferentially to the target sequence at a hybridization temperature that would still provide sufficient thermostable ligase activity. At the same time the overall length of the probe was kept to a minimum (under 100 nucleotides) to ensure efficient rolling circle transcription.

The β-globin bipartite target probes were incubated with subcloned plasmid DNA containing the wild-type β-globin gene sequence, digested with Apa LI restriction endonuclease. Target probes were annealed and ligated to the target sequence as follows: 2.5 micrograms of plasmid DNA comprising the target sequence were denatured and hybridized to 2 to 50 picomoles of each target probe in 20 mM Tris-HCl (pH 8.3 at 25° C.), 25 mM KCl, 10 mM MgCl₂, 0.5 mM NAD, 0.01% Triton X-100 at 94° C. for 1.5 minutes in the total volume of 50 ul. Target probes annealed to the target sequence were ligated using 50 Units of Ampligase® Thermostable Ligase (EPICENTRE Technologies, Madison, Wis.) by thermocycling for 20 to 50 cycles of 94° C. for 30 seconds and 40° C. for 6 minutes. The unligated probe was then removed by digestion with 40 units of E. coli Exonuclease I (EPICENTRE) for 30 minutes at 37° C. Ligation reactions were ethanol-precipitated or used directly as substrates for the N4 mini-vRNAP transcription.

Transcription reactions were analyzed as follows: 10-25% of the ligation reaction was used as template in the transcription reaction. The 20 ul reactions contained 1 mM each NTP, 1 mM DTT, 5 uM EcoSSB Protein (EPICENTRE), 1 U/ul RNasin (Promega, Fitchburg, Wis.), and 8 pmol N4 mini-vRNAP (EPICENTRE) in 1×transcription buffer comprising 40 mM Tris HCl, pH 7.5, 10 mM NaCl, 6 mM MgCl₂, and 1 mM spermidine. Reactions were incubated at 37° C. for 2 to 6 hours. The transcription reactions were treated with 2 Units DNAse I for 30 minutes at 37° C. Samples were heat denatured in formamide loading buffer with 0.1% SDS and were analyzed by denaturing 1% agarose gel electrophoresis in 1×TAE buffer.

Upon hybridization with the target sequence, a bipartite probe oligo circularizes and serves as efficient template for N4 mini-vRNAP-catalyzed rolling circle transcription, yielding high molecular weight RNA products. The unligated linear probe yields only a 11-18-nucleotide transcript (and no signal sequence would be obtained). As expected, high molecular weight RNA transcription products were observed only in reactions in which the wild type β-globin target probes were incubated under hybridization and ligation conditions in the presence of the wild-type β-globin sequence. No high molecular weight transcription product was observed in the absence of the wild-type β-globin sequence, in the absence of ligase, or in the presence of only one target probe.

### Example 5

Strand Displacement Reverse Transcription/Rolling Circle Reverse Transcription

The RNA template for strand displacement reverse transcription was first obtained by in vitro transcription of a PCR product that contained a T7 promoter sequence that was incorporated using a promoter sequence-containing PCR primer. The PCR product was in turn obtained by amplifying a linearized plasmid using the following two primers:

Forward Primer: T7 Promoter Primer:

5′GAATTGTAATACGACTCACTATAGGG 3′

Reverse Primer: RNA1000 Primer:

5′ ACTTACACCGCTTCTCAACCCG 3′

The PCR reaction mixture was prepared with a final volume of 50 ul and contained 1 ng of the linearized plasmid, 12.5 pmoles of each of the above two primers, 25 ul of 2×High Fidelity Long PCR PreMix 4 (EPICENTRE Technologies, Madison, Wis.) and 2.5 Units of MasterAmp™ Extra Long DNA Polymerase Mix (EPICENTRE). The PCR reaction mixture was heated to 95° C. for 1 min and then subjected to 35 reaction cycles of 95° C. for 45 sec, 50° C. for 45 sec, and 70° C. for 3 min. The PCR products were extracted and ethanol-precipitated using standard techniques, and resuspended in 25 ul of 10 mM Tris.HCl (pH 8.0), 1 mM EDTA (TE).

The resulting PCR product was then used to prepare a 1-Kb linear RNA transcript as follows: In vitro transcription with was performed using 5 ul of PCR product DNA as a template and the reagents supplied with the Ampliscribe™ T7 Flash™ Transcription Kit (EPICENTRE). The transcription products were treated with DNAse I, extracted and ethanol-precipitated using standard techniques, and resuspended in TE buffer at a concentration of 1.5 ug/ul (4.5 pmoles/ul).

The 1-Kb linear RNA transcript was then treated with tobacco acid pyrophosphatase (TAP) and T4 RNA ligase in order to obtain a 1-Kb circular RNA transcript as follows: The 5′-end of the RNA transcript (20 pmoles in 20 ul) containing pppG was converted to pG using 20 Units of Tobacco Acid Pyrophosphatase (EPICENTRE) at 37° C. for 45 minutes. Two picomoles of the RNA from this reaction mix (2 ul) was incubated in 10 ul of a ligation mixture containing 33 mM Tris acetate (pH 7.8), 66 mM Potassium acetate, 10 mM magnesium acetate, 1 mM DTT and 5.0 U of T4 RNA ligase (EPICENTRE) at 37° C. for 1 hour.

Then, the 1-Kb circular RNA transcript or the 1-Kb linear RNA transcript was incubated under reverse transcription reaction conditions as follows: The reverse transcription was performed by preparing a reaction mixture containing 2.0 ul of the ligation mix, 50 nM Tris-HCl (pH 9.0), 12.5 mM NaCl, 20 mM (NH₄)₂SO₄, 1×MasterAMP™ PCR Enhancer with betaine (EPICENTRE), 250 uM of each of dATP, dCTP, dGTP and dTTP, 200 Units of Isotherm™ DNA Polymerase (EPICENTRE), and 12.5 pmoles of a strand-displacement primer comprising the same RNA1000 Primer as used above for PCR. The reaction mixture was incubated at 50° C. for 90 minutes. The products of the rolling circle reverse transcription reaction were analyzed on a 1.0% formaldehyde agarose gel and visualized by staining with SYBR® gold (Molecular Probes).

Reverse transcription of the unligated 1-Kb linear RNA (i.e., without T4 RNA ligase treatment) yielded a cDNA band on the gel with a size of about 1 Kb that was less than about five-fold the intensity of the I-Kb RNA template. However, reverse transcription of the T4 RNA ligase-treated circular 1-Kb RNA yielded a very large amount of cDNA product having a size up to about 10 Kb.

Various modifications and variations of the described method and system of the invention will be apparent to those skilled in the art without departing from the scope and spirit of the invention. It is understood, however, that examples and embodiments of the present invention set forth above are illustrative and not intended to confine the invention. The invention embraces all modified forms of the examples and embodiments as come within the scope of the following claims.

We claim:

1) A method for detecting a target nucleic acid sequence, the method comprising:
a) providing one or more target probes comprising a linear single-stranded DNA molecule, the target probes comprising at least two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, and wherein the target probe that comprises the first target-complementary sequence also comprises a sense promoter sequence that is joined to the 3′-end of the first target-complementary sequence;
b) contacting the target probes with the target nucleic acid sequence and incubating under hybridization conditions, such that the target-complementary sequences anneal adjacently to the target nucleic acid sequence to form a target probe-target complex;
c) contacting the target probe-target complex with a ligase under ligation conditions to form a ligation product;
d) contacting the ligation product with an anti-sense promoter oligo and incubating under hybridization conditions, such that the anti-sense promoter oligo anneals to the sense promoter sequence to form a transcription substrate;
e) contacting the transcription substrate with an RNA polymerase under transcription conditions to form a transcription product;
f) optionally, repeating steps (a) through (f); and
g) detecting the transcription product.

2) The method of claim 1, wherein the target nucleic acid sequence comprises a single-stranded DNA molecule obtained by reverse transcription of RNA.

3) The method of claim 1, wherein the target nucleic acid sequence comprises a DNA target nucleic acid in a sample.

4) The method of claim 1, wherein the one or more target probes comprise a bipartite target probe.

5) The method of claim 1, wherein the target probe comprising the second target-complementary sequence also comprises a signal sequence 5′-of the target-complementary sequence.

6) The method of claim 5, wherein the signal sequence comprises a substrate for Q-beta replicase.

7) The method of claim 5, wherein the signal sequence comprises a sequence that encodes a detectable protein.

8) The method of claim 7, wherein the detectable protein is green fluorescent protein.

9) The method of claim 5, wherein the signal sequence comprises a sequence that is detectable by a probe.

10) The method of claim 5, wherein the signal sequence comprises a sequence that is detectable by a molecular beacon.

11) The method of claim 1, wherein the ligase is selected from the group consisting of Ampligase® Thernostable DNA Ligase, Tfl DNA Ligase, Tsc DNA Ligase, Pfu DNA ligase, T4 DNA ligase and Tth DNA ligase.

12) The method of claim 1, wherein the anti-sense promoter oligo is attached to a solid support.

13) The method of claim 1, wherein the RNA polymerase is a T7-type RNA polymerase.

14) The method of claim 1, wherein the RNA polymerase is selected from the group consisting of T7 RNA polymerase, T3 RNA polymerase, SP6 RNA polymerase, Tth RNA polymerase, E. coli RNA polymerase, and SP6 or T7 R&DNA™ Polymerase.

15) The method of claim 1 comprising an additional step following step (c), the additional step comprising releasing the ligation product from the target nucleic acid sequence.

16) A method for detecting a target nucleic acid sequence, the method comprising:
a) providing a promoter target probe, wherein the 5′-end of the promoter target probe comprises a first target-complementary sequence that is complementary to the 5′-end of the target nucleic acid sequence, and wherein a sense promoter sequence is joined to the 3′-end of the first target-complementary sequence;
b) providing a signal target probe comprising a second target complementary sequence, wherein the 3′-end of the second target-complementary sequence is complementary to the 3′-end of the target nucleic acid sequence;
c) optionally, providing at least one additional target probe comprising a target-complementary sequence;
d) contacting the target probes with the target nucleic acid sequence and incubating under hybridization conditions, such that the target-complementary sequences anneal adjacently to the target nucleic acid sequence to form a target probe-target complex;
e) contacting the target probe-target complex with a ligase under ligation conditions to form a ligation product;
f) contacting the ligation product with an anti-sense promoter oligo and incubating under hybridization conditions, such that the anti-sense promoter oligo anneals to the sense promoter sequence to form a transcription substrate;
g) contacting the transcription substrate with an RNA polymerase under transcription conditions to form a transcription product;
h) optionally, repeating steps (a) through (h); and
i) detecting the transcription product.

17) The method of claim 16, wherein the target nucleic acid sequence comprises a single-stranded DNA molecule obtained by reverse transcription of RNA.

18) The method of claim 16, wherein the target nucleic acid sequence comprises a DNA target nucleic acid in a sample.

19) The method of claim 16, wherein the signal target probe comprises a signal sequence 5′-of the target-complementary sequence.

20) The method of claim 19, wherein the signal sequence comprises a substrate for Q-beta replicase.

21) The method of claim 19, wherein the signal sequence comprises a sequence that encodes a detectable protein.

22) The method of claim 21, wherein the detectable protein is green fluorescent protein.

23) The method of claim 19, wherein the signal sequence comprises a sequence that is detectable by a probe.

24) The method of claim 19, wherein the signal sequence comprises a sequence that is detectable by a molecular beacon.

25) The method of claim 16, wherein the ligase is selected from the group consisting of Ampligase® Thermostable DNA Ligase, Tfl DNA Ligase, Tsc DNA Ligase, Pfu DNA ligase, T4 DNA ligase and Tth DNA ligase.

26) The method of claim 16, wherein the anti-sense promoter oligo is attached to a solid support.

27) The method of claim 16, wherein the RNA polymerase is a T7-type RNA polymerase.

28) The method of claim 16, wherein the RNA polymerase is selected from the group consisting of T7 RNA polymerase, T3 RNA polymerase, SP6 RNA polymerase, Tth RNA polymerase, E. coli RNA polymerase, and SP6 or T7 R&DNA™ Polymerase.

29) A method for detecting a target nucleic acid sequence, the method comprising:
a) providing one or more target probes comprising a linear single-stranded DNA molecule, the target probes comprising at least two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, and wherein the target probe that comprises the first target-complementary sequence also comprises a sense promoter sequence that is joined to the 3′-end of the first target-complementary sequence;
b) contacting the target probes with the target nucleic acid sequence and incubating under hybridization conditions, such that the target probes anneal to the target nucleic acid sequence to form a target probe-target complex;
c) contacting the target probe-target complex with a DNA polymerase under DNA polymerization conditions to form one or more DNA polymerase extension products that are adjacent to the 5′-end of a target-probe, such that a complex is formed;
d) contacting the complex with a ligase under ligation conditions to form a transcription substrate;
e) contacting the transcription substrate with an RNA polymerase;
f) optionally, repeating steps (a) through (f); and
g) detecting the transcription product.

30) A method for detecting a target nucleic acid sequence, the method comprising:
a) providing a target sequence amplification probe (TSA probe) comprising a linear single-stranded DNA molecule comprising a 5′-end portion and a 3′-end portion that are not joined, wherein the 5′-end portion is complementary to the 5′-end of the target sequence, and wherein the 3′-end portion is complementary to the 3′-end of the target sequence;
b) providing a primer that is complementary to the TSA probe;
c) providing one or more target probes comprising a second linear single-stranded DNA molecule, the target probes comprising at least two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, and wherein the target probe that comprises the first target-complementary sequence also comprises a sense promoter sequence that is joined to the 3′-end of the first target-complementary sequence;
d) contacting the TSA probe with the target nucleic acid sequence and incubating under hybridization conditions, such that the end portions anneal adjacently to the target nucleic acid sequence to form a complex;
e) contacting the complex with a ligase under ligation conditions, such that a target sequence amplification circle (TSA circle) is formed;
f) contacting the TSA circle with the primer and incubating under hybridization conditions to form a TSA circle-primer complex;
g) contacting the TSA circle-primer complex with a strand-displacing DNA polymerase under strand-displacing polymerization conditions, such that a rolling circle replication product comprising multiple copies of the target nucleic acid sequence is formed;
h) contacting the target probes with the rolling circle replication product and incubating under hybridization conditions, such that the target-complementary sequences anneal adjacently to the rolling circle replication product to form a target probe-rolling circle replication product complex;
i) contacting the target probe-rolling circle replication product complex with the ligase under ligation conditions to form a ligation product;
j) optionally, releasing the ligation product from the rolling circle replication product complex,
k) contacting the ligation product with an anti-sense promoter oligo and incubating under hybridization conditions, such that the anti-sense promoter oligo anneals to the sense promoter sequence to form a transcription substrate;
l) contacting the transcription substrate with an RNA polymerase under transcription conditions to form a transcription product;
m) optionally, repeating steps (a) through (m); and
n) detecting the transcription product.

31) The method of claim 30, wherein the target nucleic acid sequence comprises a single-stranded DNA molecule obtained by reverse transcription of RNA.

32) The method of claim 30, wherein the target nucleic acid sequence comprises a DNA target nucleic acid in a sample.

33) The method of claim 30, wherein the target probe comprising the second target-complementary sequence also comprises a signal sequence 5′-of the target-complementary sequence.

34) The method of claim 33, wherein the signal sequence comprises a substrate for Q-beta replicase.

35) The method of claim 33, wherein the signal sequence comprises a sequence that encodes a detectable protein.

36) The method of claim 35, wherein the detectable protein is green fluorescent protein.

37) The method of claim 33, wherein the signal sequence comprises a sequence that is detectable by a probe.

38) The method of claim 33, wherein the signal sequence comprises a sequence that is detectable by a molecular beacon.

39) The method of claim 30, wherein the ligase is selected from the group consisting of Ampligase® Thermostable DNA Ligase, Tfl DNA Ligase, Tsc DNA Ligase, Pfu DNA ligase, T4 DNA ligase and Tth DNA ligase.

40) The method of claim 30, wherein the strand-displacing DNA polymerase is selected from the group consisting of RepliPHI™ phi29 DNA polymerase, phi29 DNA polymerase, rBst DNA polymerase large fragment, IsoTherm™ DNA polymerase, BcaBEST™ DNA polymerase, SequiTherm™ DNA polymerase, phage M2 DNA polymerase, phage phi PRD1 DNA polymerase, VENT® DNA polymerase, Klenow fragment of DNA polymerase I, T5 DNA polymerase, PRD1 DNA polymerase, and T7 DNA polymerase in the presence of a T7 helicase/primase complex.

41) The method of claim 30, wherein the anti-sense promoter oligo is attached to a solid support.

42) The method of claim 30, wherein the RNA polymerase is a T7-type RNA polymerase.

43) The method of claim 30, wherein the RNA polymerase is selected from the group consisting of T7 RNA polymerase, T3 RNA polymerase, SP6 RNA polymerase, Tth RNA polymerase, E. coli RNA polymerase, and SP6 or T7 R&DNA™ Polymerase.

44) The method of claim 30, comprising an additional step following step (e), the additional step comprising releasing the TSA circle from the target nucleic acid sequence.

45) A method for detecting a target nucleic acid sequence, the method comprising:
a) providing a bipartite target probe comprising a linear single-stranded DNA molecule, the target probes comprising at least two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence;
b) optionally, providing at least one additional target probe comprising a target-complementary sequence;
c) contacting the target probe with the target nucleic acid sequence and incubating under hybridization conditions, such that the target-complementary sequences anneal adjacently to the target nucleic acid sequence to form a target probe-target complex;
d) contacting the target probe-target complex with a ligase under ligation conditions to form a transcription substrate;
e) contacting the transcription substrate with an RNA polymerase under transcription conditions to form a transcription product;
f) optionally, repeating steps (a) through (f); and
g) detecting the transcription product.

46) A method for detecting a target nucleic acid sequence, the method comprising:
a) providing one or more target probes comprising a linear single-stranded DNA molecule, the target probes comprising at least two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, and wherein the target probe that comprises the first target-complementary sequence also comprises a pseudopromoter that is joined to the 3′-of the first target-complementary sequence;
b) contacting the target probes with the target nucleic acid sequence and incubating under hybridization conditions, such that the target-complementary sequences anneal adjacently to the target nucleic acid sequence to form a target probe-target complex;
c) contacting the target probe-target complex with a ligase under ligation conditions to form a transcription substrate;
d) contacting the transcription substrate with an RNA polymerase;
e) optionally, repeating steps (a) through (e); and
f) detecting the transcription product.

47) A method for detecting an analyte in a sample, the method comprising:
a) providing a target nucleic acid sequence comprising a target sequence tag that is joined to an analyte-binding substance;
b) contacting the analyte-binding substance with the analyte to form a specific binding pair;
c) separating the specific binding pair from analyte-binding substance molecules that are not bound to the analyte;
d) providing one or more target probes comprising a linear single-stranded DNA molecule, the target probes comprising at least two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, and wherein the target probe that comprises the first target-complementary sequence also comprises a promoter that is joined to the 3′-end of the first target-complementary sequence;
e) contacting the target probes with the target nucleic acid sequence and incubating under hybridization conditions, such that the target-complementary sequences anneal adjacently to the target nucleic acid sequence to form a target probe-target complex;
f) contacting the target probe-target complex with a ligase under ligation conditions to form a ligation product;
g) contacting the ligation product with an anti-sense promoter oligo and incubating under hybridization conditions, such that the anti-sense promoter oligo anneals to the sense promoter sequence to form a transcription substrate;
h) contacting the transcription substrate with an RNA polymerase under transcription conditions to form a transcription product;
i) optionally, repeating steps (d) through (i); and
j) detecting the transcription product.

48) The method of claim 47, wherein the analyte is selected from the group consisting of a biochemical molecule, a biopolymer, a protein, a glycoprotein, a lipoprotein, an enzyme, a hormone, a biochemical metabolite, a receptor, an antigen, an antibody, a nucleic acid, a DNA molecule, an RNA molecule, a polysaccharide and a lipid.

49) The method of claim 47, wherein the analyte-binding substance is selected from the group consisting of a nucleic acid, a polynucleotide, an oligonucleotide, a segment of a nucleic acid or polynucleotide, a DNA molecule, an RNA molecule, a molecule comprising both DNA and RNA mononucleotides, modified DNA mononucleotides, a molecule obtained by a method termed “SELEX”, a nucleic acid molecule having an affinity for protein molecules, a polynucleotide molecule having an affinity for protein molecules, an operator, a promoter, an origin of replication, a ribosomal nucleic acid sequence, a sequence recognized by steroid hormone-receptor complexes, a peptide nucleic acid (PNA), a nucleic acid and a PNA, a molecule prepared by using a combinatorial library of randomized peptide nucleic acids, an oligonucleotide or polynucleotide with a modified backbone that is not an amino acid, a lectin, a receptor for a hormone, a hormone, and an enzyme inhibitor.

50) The method of claim 47, wherein the target probe comprising the second target-complementary sequence also comprises a signal sequence 5′-of the target-complementary sequence.

51) The method of claim 50, wherein the signal sequence comprises a substrate for Q-beta replicase.

52) The method of claim 50, wherein the signal sequence comprises a sequence that encodes a detectable protein.

53) The method of claim 52, wherein the detectable protein is green fluorescent protein.

54) The method of claim 50, wherein the signal sequence comprises a sequence that is detectable by a probe.

55) The method of claim 50, wherein the signal sequence comprises a sequence that is detectable by a molecular beacon.

56) The method of claim 47, wherein the ligase is selected from the group consisting of Ampligase® Thermostable DNA Ligase, Tfl DNA Ligase, Tsc DNA Ligase, Pfu DNA ligase, T4 DNA ligase and Tth DNA ligase.

57) The method of claim 47, wherein the anti-sense promoter oligo is attached to a solid support.

58) The method of claim 47, wherein the RNA polymerase is a T7-type RNA polymerase.

59) The method of claim 47, wherein the RNA polymerase is selected from the group consisting of T7 RNA polymerase, T3 RNA polymerase, SP6 RNA polymerase, Tth RNA polymerase, E. coli RNA polymerase, and SP6 or T7 R&DNA™ Polymerase.

60) The method of claim 47, comprising an additional step following step (c) the additional step comprising releasing the ligation product from the target nucleic acid sequence.

61) A method for selectively transcribing a target nucleic acid sequence, the method comprising a DNA ligation operation and a transcription operation, wherein the DNA ligation operation comprises ligation of one or more target probes comprising a sense promoter sequence that is joined to the 3′-end of a target complementary sequence to form a ligation product, wherein the ligation is dependent on hybridization of the target probes to the target nucleic acid sequence, and wherein the transcription operation comprises contacting the ligation product with an RNA polymerase.

62) A kit for detecting a target nucleic acid sequence, the kit comprising:
a) one or more target probes comprising a linear single-stranded DNA molecule, the target probes comprising at least two target-complementary sequences that are not joined to each other, wherein the 5′-end of a first target-complementary sequence is complementary to the 5′-end of the target nucleic acid sequence, and wherein the 3′-end of a second target-complementary sequence is complementary to the 3′-end of the target nucleic acid sequence, and wherein the target probe that comprises the first target-complementary sequence also comprises a promoter that is joined to the 3′-of the first target-complementary sequence;
b) a ligase; and
c) an RNA polymerase.

63) The kit of claim 62, further comprising a reverse transcriptase.

64) The kit of claim 62, further comprising a target sequence amplification probe (TSA probe) comprising a linear single-stranded DNA molecule comprising a 5′-end portion and a 3′-end portion that are not joined, wherein the 5′-end portion is complementary to the 5′-end of the target sequence, and wherein the 3′-end portion is complementary to the 3′-end of the target sequence.

65) The kit of claim 62, further comprising a DNA polymerase.

66) The kit of claim 62, further comprising a target sequence tag that is joined to an analyte-binding substance.


SEQUENCE LISTING (14 sequences)

SEQ ID NO: 1 - DNA, 20 bp, Artificial
  synthetic oligonucleotide
caacgaagcgttgaatacct

SEQ ID NO: 2 - DNA, 22 bp, Artificial
  synthetic oligonucleotide
ttcttcgaggcgaagaaaacct

SEQ ID NO: 3 - DNA, 20 bp, Artificial
  synthetic oligonucleotide
cgacgaggcgtcgaaaacca

SEQ ID NO: 4 - DNA, 18 bp, Artificial
  synthetic oligonucleotide
ctatagtgagtcgtatta

SEQ ID NO: 5 - DNA, 18 bp, Artificial
  synthetic oligonucleotide
taatacgactcactatag

SEQ ID NO: 6 - DNA, 37 bp, Artificial
  synthetic oligonucleotide
tcaggagtcaggtgcctatagtgagtcgtattactag

SEQ ID NO: 7 - DNA, 50 bp, Artificial
  synthetic oligonucleotide
ggccaacgactacgcactagccaaccagggcagtaacggcagacttctcc

SEQ ID NO: 8 - DNA, 18 bp, Artificial
  synthetic oligonucleotide
taatacgactcactatag

SEQ ID NO: 9 - DNA, 38 bp, Artificial
  synthetic oligonucleotide
ctgtgcctcctgggggctatagtgagtcgtattactag

SEQ ID NO: 10 - DNA, 48 bp, Artificial
  synthetic oligonucleotide
ccaacgactacgcactagccaacgttacaaacctataagtatcttcta

SEQ ID NO: 11 - DNA, 18 bp, Artificial
  synthetic oligonucleotide
taatacgactcactatag

SEQ ID NO: 12 - DNA, 67 bp, Artificial
  synthetic oligonucleotide
gtcctcagtcccaaaagaagcggagcttcttttttttttttttttttttttttccgtctgaagagga

SEQ ID NO: 13 - DNA, 26 bp, Artificial
  synthetic oligonucleotides
gaattgtaatacgactcactataggg

SEQ ID NO: 14 - DNA, 22 bp, Artificial
  synthetic oligonucleotide
acttacaccgcttctcaacccg